SEQUENCE LISTING (300 sequences)

SEQ ID NO: 1 - protein, 306 aa, Artificial Sequence
  /note=“Description of Artificial Sequence Synthetic polypeptide”
MSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSGSGSAGGSGSGGGSLPETGGHHHHHH

SEQ ID NO: 2 - protein, 100 aa, Artificial Sequence [sequence omitted]

SEQ ID NO: 3 - protein, 184 aa, Artificial Sequence
  /note=“Description of Artificial Sequence Synthetic polypeptide”
MDYKDDDDKGSSGDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQLPETGGHHHHHH

SEQ ID NO: 4 - protein, 398 aa, Artificial Sequence
  /note=“Description of Artificial Sequence Synthetic polypeptide”
MGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSGSGSAGGSFESGPGAEYCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 5 - protein, 212 aa, Artificial Sequence
  /note=“Description of Artificial Sequence Synthetic polypeptide”
MIKIATRKYLGKQNVYDIGVERDHNFALKNGFIASNCFNVDDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQGSTGDYKDDDDK

SEQ ID NO: 6 - protein, 156 aa, Artificial Sequence
  /note=“Description of Artificial Sequence Synthetic polypeptide”
MQAKPQIPKDKSKVAGYIEIPDADIKEPVYPGPATREQLNRGVSFAEENESLDDQNISIAGHTFIDRPNYQFTNLKAAKKGSMVYFKVGNETRKYKMTSIRNVKPTAVEVLDEQKGKDKQLTLITCDDYNEETGVWETRKIFVATEVKLEHHHHHH

SEQ ID NO: 7 - protein, 9 aa, Artificial Sequence
  /note=“Description of Artificial Sequence Synthetic peptide”
  MOD_RES (8)..(8): 3-amino-3-(2-nitro)phenyl-propionic acid
GILGFVFXL

SEQ ID NO: 8 - protein, 9 aa, Artificial Sequence
  /note=“Description of Artificial Sequence Synthetic peptide”
NLVPMVATV

SEQ ID NO: 9 - protein, 9 aa, Artificial Sequence
  /note=“Description of Artificial Sequence Synthetic peptide”
NLVPMVGTV

SEQ ID NO: 10 - protein, 9 aa, Artificial Sequence
  /note=“Description of Artificial Sequence Synthetic peptide”
VTEHDTLLY

SEQ ID NO: 11 - protein, 9 aa, Artificial Sequence
  /note=“Description of Artificial Sequence Synthetic peptide”
  MOD_RES (4)..(4): 3-amino-3-(2-nitro)phenyl-propionic acid
VYGXVRACL

SEQ ID NO: 12 - protein, 9 aa, Artificial Sequence
  /note=“Description of Artificial Sequence Synthetic peptide”
QYDPVAALF

SEQ ID NO: 13 - protein, 11 aa, Artificial Sequence
  /note=“Description of Artificial Sequence Synthetic peptide”
RPHERNGFTVL

SEQ ID NO: 14 - protein, 9 aa, Artificial Sequence
  /note=“Description of Artificial Sequence Synthetic peptide”
  MOD_RES (5)..(5): 3-amino-3-(2-nitro)phenyl-propionic acid
AARGXTLAM

SEQ ID NO: 15 - protein, 9 aa, Artificial Sequence
  /note=“Description of Artificial Sequence Synthetic peptide”
  MOD_RES (8)..(8): 3-amino-3-(2-nitro)phenyl-propionic acid
KILGFVFXV

SEQ ID NO: 16 - protein, 9 aa, Artificial Sequence
  /note=“Description of Artificial Sequence Synthetic peptide”
  MOD_RES (6)..(6): 3-amino-3-(2-nitro)phenyl-propionic acid
STAPGXLEY

SEQ ID NO: 17 - protein, 9 aa, Artificial Sequence
  /note=“Description of Artificial Sequence Synthetic peptide”
  MOD_RES (5)..(5): 3-amino-3-(2-nitro)phenyl-propionic acid
RIYRXGATR

SEQ ID NO: 18 - protein, 9 aa, Artificial Sequence
  /note=“Description of Artificial Sequence Synthetic peptide”
  MOD_RES (5)..(5): 3-amino-3-(2-nitro)phenyl-propionic acid
RVFAXSFIK

SEQ ID NO: 19 - protein, 9 aa, Artificial Sequence
  /note=“Description of Artificial Sequence Synthetic peptide”
  MOD_RES (7)..(7): 3-amino-3-(2-nitro)phenyl-propionic acid
KPIVVLXGY

SEQ ID NO: 20 - protein, 10 aa, Artificial Sequence
  /note=“Description of Artificial Sequence Synthetic peptide”
  MOD_RES (5)..(5): 3-amino-3-(2-nitro)phenyl-propionic acid
FVYGXSKTSL

SEQ ID NO: 21 - protein, 9 aa, Artificial Sequence
  /note=“Description of Artificial Sequence Synthetic peptide”
  MOD_RES (7)..(7): 3-amino-3-(2-nitro)phenyl-propionic acid
FLRGRAXGL

SEQ ID NO: 22 - protein, 9 aa, Artificial Sequence
  /note=“Description of Artificial Sequence Synthetic peptide”
  MOD_RES (4)..(4): 3-amino-3-(2-nitro)phenyl-propionic acid
VRIXHLYIL

SEQ ID NO: 23 - protein, 9 aa, Artificial Sequence
  /note=“Description of Artificial Sequence Synthetic peptide”
  MOD_RES (4)..(4): 3-amino-3-(2-nitro)phenyl-propionic acid
QYDXAVYKL

SEQ ID NO: 24 - protein, 9 aa, Artificial Sequence
  /note=“Description of Artificial Sequence Synthetic peptide”
  MOD_RES (5)..(5): 3-amino-3-(2-nitro)phenyl-propionic acid
ILGPXGSVY

SEQ ID NO: 25 - protein, 9 aa, Artificial Sequence
  /note=“Description of Artificial Sequence Synthetic peptide”
  MOD_RES (7)..(7): 3-amino-3-(2-nitro)phenyl-propionic acid
TEADVQXWL

SEQ ID NO: 26 - protein, 9 aa, Artificial Sequence
  /note=“Description of Artificial Sequence Synthetic peptide”
  MOD_RES (7)..(7): 3-amino-3-(2-nitro)phenyl-propionic acid
ISARGQXLF

SEQ ID NO: 27 - protein, 10 aa, Artificial Sequence
  /note=“Description of Artificial Sequence Synthetic peptide”
  MOD_RES (4)..(4): 3-amino-3-(2-nitro)phenyl-propionic acid
KAAXDLSHFL

SEQ ID NO: 28 - protein, 365 aa, Homo sapiens
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 29 - protein, 365 aa, Homo sapiens
MAVMAPRTLLLLLSGALALTQTWAGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 30 - protein, 365 aa, Homo sapiens
MAVMAPRTLLLLLSGALALTQTWAGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 31 - protein, 365 aa, Homo sapiens [sequence omitted]

SEQ ID NO: 32 - protein, 362 aa, Homo sapiens
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 33 - protein, 366 aa, Homo sapiens
MRVMAPRTLILLLSGALALTETWAGSHSMRYFSTSVSWPGRGEPRFIAVGYVDDTQFVRFDSDAASPRGEPREPWVEQEGPEYWDRETQKYKRQAQADRVNLRKLRGYYNQSEDGSHTLQRMFGCDLGPDGRLLRGYNQFAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWKPSSQPTIPIVGIVAGLAVLAVLAVLGAMVAVVMCRRKSSGGKGGSCSQAASSNSAQGSDESLIACKA

SEQ ID NO: 34 - protein, 366 aa, Homo sapiens
MRVMAPRALLLLLSGGLALTETWACSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKA

SEQ ID NO: 35 - protein, 362 aa, Homo sapiens
MLVMAPRTVLLLLSAALALTETWAGSHSMRYFDTAMSRPGRGEPRFISVGYVDDTQFVRFDSDAASPREEPRAPWIEQEGPEYWDRNTQIFKTNTQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 36 - protein, 362 aa, Homo sapiens [sequence omitted]

SEQ ID NO: 37 - protein, 362 aa, Homo sapiens [sequence omitted]

SEQ ID NO: 38 - protein, 362 aa, Homo sapiens [sequence omitted]

SEQ ID NO: 39 - protein, 358 aa, Homo sapiens
MVDGTLLLLLSEALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 40 - protein, 366 aa, Homo sapiens
MRVMAPRTLILLLSGALALTETWACSHSMRYFYTAVSRPGRGEPRFIAVGYVDDTQFVRFDSDAASPRGEPRAPWVEQEGPEYWDRETQKYKRQAQTDRVSLRNLRGYYNQSEAGSHTLQWMYGCDLGPDGRLLRGYDQSAYDGKDYIALNEDLRSWTAADTAAQITQRKWEAARAAEQQRAYLEGTCVEWLRRYLENGKETLQRAEHPKTHVTHHLVSDHEATLRCWALGFYPAEITLTWQRDGEDQTQDTELVETRPAGDGTFQKWAAVVVPSGEEQRYTCHVQHEGLPEPLTLRWEPSSQPTIPIVGIVAGLAVLAVLAVLGAVVAVVMCRRKSSGGKGGSCSQAASSNSAQGSDESLIACKA

SEQ ID NO: 41 - protein, 366 aa, Homo sapiens [sequence omitted]

SEQ ID NO: 42 - protein, 366 aa, Homo sapiens
MRVMAPRALLLLLSGGLALTETWACSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKA

SEQ ID NO: 43 - protein, 366 aa, Homo sapiens
MRVMAPRTLILLLSGALALTETWACSHSMRYFYTAVSRPGRGEPRFIAVGYVDDTQFVQFDSDAASPRGEPRAPWVEQEGPEYWDRETQKYKRQAQTDRVNLRKLRGYYNQSEAGSHTLQRMYGCDLGPDGRLLRGYNQFAYDGKDYIALNEDLRSWTAADKAAQITQRKWEAAREAEQRRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGPSSQPTIPIVGIVAGLAVLAVLAVLGAVMAVVMCRRKSSGGKGGSCSQAASSNSAQGSDESLIACKA

SEQ ID NO: 44 - protein, 362 aa, Homo sapiens
MRVTAPRTLLLLLWGAVALTETWAGSHSMRYFYTAMSRPGRGEPRFITVGYVDDTLFVRFDSDATSPRKEPRAPWIEQEGPEYWDRETQISKTNTQTYRENLRTALRYYNQSEAGSHIIQRMYGCDVGPDGRLLRGYDQDAYDGKDYIALNEDLSSWTAADTAAQITQRKWEAARVAEQDRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 45 - protein, 365 aa, Homo sapiens
MAVMAPRTLLLLLLGALALTQTWAGSHSMRYFTTSVSRPGRGEPRFIAVGYVDDTQFVRFDSDAASQRMEPRAPWIEQEGPEYWDLQTRNVKAQSQTDRANLGTLRGYYNQSEAGSHTIQMMYGCDVGSDGRFLRGYRQDAYDGKDYIALNEDLRSWTAADMAAQITQRKWEAARVAEQLRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEPSSQPTIPIVGIIAGLVLFGAVFAGAVVAAVRWRRKSSDRKGGSYSQAASSDSAQGSDMSLTACKV

SEQ ID NO: 46 - protein, 362 aa, Homo sapiens
MRVTAPRTLLLLLWGAVALTETWAGSHSMRYFYTAMSRPGRGEPRFITVGYVDDTLFVRFDSDATSPRKEPRAPWIEQEGPEYWDRETQISKTNTQTYRENLRTALRYYNQSEAGSHIIQRMYGCDVGPDGRLLRGYDQDAYDGKDYIALNEDLSSWTAADTAAQITQRKWEAARVAEQLRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 47 - protein, 366 aa, Homo sapiens
MRVMAPRTLILLLSGALALTETWAGSHSMRYFYTAVSRPGRGEPHFIAVGYVDDTQFVRFDSDAASPRGEPRAPWVEQEGPEYWDRETQKYKRQAQTDRVSLRNLRGYYNQSEAGSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEPSSQPTIPIVGIVAGLAVLAVLAVLGAVVAVVMCRRKSSGGKGGSCSQAASSNSAQGSDESLIACKA

SEQ ID NO: 48 - protein, 362 aa, Homo sapiens [sequence omitted]

SEQ ID NO: 49 - protein, 366 aa, Homo sapiens
MRVMAPRTLILLLSGALALTETWACSHSMRYFDTAVSRPGRGEPRFISVGYVDDTQFVRFDSDAASPRGEPRAPWVEQEGPEYWDRETQKYKRQAQADRVNLRKLRGYYNQSEDGSHTLQWMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEPSSQPTIPIVGIVAGLAVLAVLAVLGAVMAVVMCRRKSSGGKGGSCSQAASSNSAQGSDESLIACKA

SEQ ID NO: 50 - protein, 362 aa, Homo sapiens
MRVTAPRTVLLLLSGALALTETWAGSHSMRYFYTAMSRPGRGEPRFIAVGYVDDTQFVRFDSDAASPRMAPRAPWIEQEGPEYWDRETQISKTNTQTYRESLRNLRGYYNQSEAGSHTLQRMYGCDVGPDGRLLRGHDQSAYDGKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTIPIVGIVAGLAVLAVVVIGAVVATVMCRRKSSGGKGGSYSQAASSDSAQGSDVSLTA

SEQ ID NO: 51 - protein, 366 aa, Homo sapiens [sequence omitted]

SEQ ID NO: 52 - protein, 365 aa, Homo sapiens [sequence omitted]

SEQ ID NO: 53 - protein, 362 aa, Homo sapiens
MRVTAPRTLLLLLWGAVALTETWAGSHSMRYFYTAMSRPGRGEPRFITVGYVDDTQFVRFDSDATSPRMAPRAPWIEQEGPEYWDRETQISKTNTQTYRENLRTALRYYNQSEAGSHTWQTMYGCDLGPDGRLLRGHNQLAYDGKDYIALNEDLSSWTAADTAAQITQLKWEAARVAEQLRAYLEGECVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 54 - protein, 366 aa, Homo sapiens [sequence omitted]

SEQ ID NO: 55 - protein, 365 aa, Homo sapiens
MAVMAPRTLVLLLSGALALTQTWAGSHSMRYFYTSVSRPGRGEPRFIAVGYVDDTQFVRFDSDAASQRMEPRAPWIEQEGPEYWDRNTRNVKAHSQTDRANLGTLRGYYNQSEDGSHTIQRMYGCDVGPDGRFLRGYQQDAYDGKDYIALNEDLRSWTAADMAAQITQRKWETAHEAEQWRAYLEGRCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEPSSQPTIPIVGIIAGLVLFGAVIAGAVVAAVMWRRKSSDRKGGSYSQAASSDSAQGSDMSLTACKV

SEQ ID NO: 56 - protein, 362 aa, Homo sapiens [sequence omitted]

SEQ ID NO: 57 - protein, 362 aa, Homo sapiens
MLVMAPRTVLLLLSAALALTETWAGSHSMRYFYTAVSRPGRGEPRFISVGYVDDTQFVRFDSDAASPREEPRAPWIEQEGPEYWDRNTQICKTNTQTDRESLRNLRGYYNQSEAGSHTLQWMYGCDVGPDGRLLRGYNQFAYDGKDYIALNEDLSSWTAADTAAQITQRKWEAAREAEQLRAYLEGTCVEWLRRHLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAASSDSAQGSDVSLTA

SEQ ID NO: 58 - protein, 365 aa, Homo sapiens [sequence omitted]

SEQ ID NO: 59 - protein, 365 aa, Homo sapiens [sequence omitted]

SEQ ID NO: 60 - protein, 365 aa, Homo sapiens
MAVMAPRTLVLLLSGALALTQTWAGSHSMRYFYTSVSRPGRGEPRFIAVGYVDDTQFVRFDSDAASQRMEPRAPWIEQEGPEYWDRNTRNVKAHSQTDRESLRIALRYYNQSEDGSHTIQRMYGCDVGPDGRFLRGYQQDAYDGKDYIALNEDLRSWTAADMAAQITQRKWETAHEAEQWRAYLEGRCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEPSSQPTIPIVGIIAGLVLFGAVIAGAVVAAVMWRRKSSDRKGGSYSQAASSDSAQGSDMSLTACKV

SEQ ID NO: 61 - protein, 362 aa, Homo sapiens
MRVTAPRTLLLLLWGAVALTETWAGSHSMRYFHTSVSRPGRGEPRFISVGYVDGTQFVRFDSDAASPRTEPRAPWIEQEGPEYWDRNTQISKTNTQTYRESLRNLRGYYNQSEAGSHTLQRMYGCDVGPDGRLLRGHDQSAYDGKDYIALNEDLSSWTAADTAAQITQRKWEAARVAEQLRAYLEGTCVEWLRRHLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTIPIVGIVAGLAVLAVVVIGAVVATVMCRRKSSGGKGGSYSQAASSDSAQGSDVSLTA

SEQ ID NO: 62 - protein, 362 aa, Homo sapiens [sequence omitted]

SEQ ID NO: 63 - protein, 362 aa, Homo sapiens [sequence omitted]

SEQ ID NO: 64 - protein, 366 aa, Homo sapiens [sequence omitted]

SEQ ID NO: 65 - protein, 366 aa, Homo sapiens [sequence omitted]

SEQ ID NO: 66 - protein, 366 aa, Homo sapiens [sequence omitted]

SEQ ID NO: 67 - protein, 362 aa, Homo sapiens [sequence omitted]

SEQ ID NO: 68 - protein, 365 aa, Homo sapiens [sequence omitted]

SEQ ID NO: 69 - protein, 365 aa, Homo sapiens [sequence omitted]

SEQ ID NO: 70 - protein, 362 aa, Homo sapiens
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 71 - protein, 372 aa, Homo sapiens
MRVMAPQALLLLLSGALALIETWAGSHSMRYFYTAVSRPGRGEPRFIAVGYVDDTQFVRFDSDAASPRGEPRAPWVEQEGPEYWDRETQKYKRQAQADRVNLRKLRGYYNQSEAGSHTIQRMYGCDLGPDGRLLRGYNQFAYDGKDYIALNEDLRSWTAADTAAQISQRKLEAAREAEQLRAYLEGECVEWLRGYLENGKETLQRAERPKTHVTHHPVSDHEATLRCWALGFYPAEITLTWQRDGEDQTQDTELVETRPAGDGTFQKWAAVVVPSGQEQRYTCHVQHEGLQEPCTLRWKPSSQPTIPNLGIVSGPAVLAVLAVLAVLAVLGAVVAAVIHRRKSSGGKGGSCSQAASSNSAQGSDESLIACKA

SEQ ID NO: 72 - protein, 362 aa, Homo sapiens [sequence omitted]

SEQ ID NO: 73 - protein, 362 aa, Homo sapiens [sequence omitted]

SEQ ID NO: 74 - protein, 366 aa, Homo sapiens [sequence omitted]

SEQ ID NO: 75 - protein, 362 aa, Homo sapiens [sequence omitted]

SEQ ID NO: 76 - protein, 365 aa, Homo sapiens [sequence omitted]

SEQ ID NO: 77 - protein, 365 aa, Homo sapiens [sequence omitted]

SEQ ID NO: 78 - protein, 366 aa, Homo sapiens
MRVMAPRTLILLLSGALALTETWACSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWEPSSQPTIPIVGIVAGLAVLAVLAVLGAVVAVVMCRRKSSGGKGGSCSQAASSNSAQGSDESLIACKA

SEQ ID NO: 79 - protein, 366 aa, Homo sapiens [sequence omitted]

SEQ ID NO: 80 - protein, 365 aa, Homo sapiens [sequence omitted]

SEQ ID NO: 81 - protein, 365 aa, Homo sapiens
MAVMAPRTLLLLLLGALALTQTWAGSHSMRYFFTSVSRPGRGEPRFIAVGYVDDTQFVRFDSDAASQRMEPRAPWIEQEGPEYWDQETRNVKAHSQTDRESLRIALRYYNQSEAGSHTIQMMYGCDVGPDGRLLRGYQQDAYDGKDYIALNEDLRSWTAADMAAQITQRKWEAARVAEQLRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEPSSQPTIPIVGIIAGLVLFGAMFAGAVVAAVRWRRKSSDRKGGSYSQAASSDSAQGSDMSLTACKV

SEQ ID NO: 82 - protein, 362 aa, Homo sapiens [sequence omitted]

SEQ ID NO: 83 - protein, 362 aa, Homo sapiens [sequence omitted]

SEQ ID NO: 84 - protein, 362 aa, Homo sapiens [sequence omitted]

SEQ ID NO: 85 - protein, 365 aa, Homo sapiens [sequence omitted]

SEQ ID NO: 86 - protein, 362 aa, Homo sapiens [sequence omitted]

SEQ ID NO: 87 - protein, 362 aa, Homo sapiens [sequence omitted]

SEQ ID NO: 88 - protein, 362 aa, Homo sapiens [sequence omitted]

SEQ ID NO: 89 - protein, 366 aa, Homo sapiens
MRVMAPRTLILLLSGALALTETWACSHSMRYFYTAVSRPGRGEPRFIAVGYVDDTQFVRFDSDAASPRGEPRAPWVEQEGPEYWDRETQKYKRQAQADRVSLRNLRGYYNQSEAGSHTLQRMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEPSSQPTIPIVGIVAGLAVLAVLAVLGAVMAVVMCRRKSSGGKGGSCSQAASSNSAQGSDESLIACKA

SEQ ID NO: 90 - protein, 362 aa, Homo sapiens [sequence omitted]

SEQ ID NO: 91 - protein, 362 aa, Homo sapiens [sequence omitted]

SEQ ID NO: 92 - protein, 362 aa, Homo sapiens [sequence omitted]

SEQ ID NO: 93 - protein, 365 aa, Homo sapiens [sequence omitted]

SEQ ID NO: 94 - protein, 278 aa, Homo sapiens
GSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSS

SEQ ID NO: 95 - protein, 278 aa, Homo sapiens
GSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSS

SEQ ID NO: 96 - protein, 278 aa, Homo sapiens
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSS

SEQ ID NO: 97 - protein, 278 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEPSS

SEQ ID NO: 98 - protein, 278 aa, Homo sapiens
GSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 99 - protein, 278 aa, Homo sapiens
GSHSMRYFSTSVSWPGRGEPRFIAVGYVDDTQFVRFDSDAASPRGEPREPWVEQEGPEYWDRETQKYKRQAQADRVNLRKLRGYYNQSEDGSHTLQRMFGCDLGPDGRLLRGYNQFAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWKPSS

SEQ ID NO: 100 - protein, 278 aa, Homo sapiens
CSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSS

SEQ ID NO: 101 - protein, 278 aa, Homo sapiens
GSHSMRYFDTAMSRPGRGEPRFISVGYVDDTQFVRFDSDAASPREEPRAPWIEQEGPEYWDRNTQIFKTNTQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 102 - protein, 278 aa, Homo sapiens
GSHSMRYFYTAMSRPGRGEPRFIAVGYVDDTQFVRFDSDAASPRTEPRAPWIEQEGPEYWDRNTQIFKTNTQTYRESLRNLRGYYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEPSS

SEQ ID NO: 103 - protein, 278 aa, Homo sapiens
GSHSMRYFYTAMSRPGRGEPRFIAVGYVDDTQFVRFDSDAASPRMAPRAPWIEQEGPEYWDGETRNMKASAQTYRENLRIALRYYNQSEAGSHIIQVMYGCDVGPDGRLLRGHDQSAYDGKDYIALNEDLSSWTAADTAAQITQRKWEAARVAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 104 - protein, 278 aa, Homo sapiens
GSHSMRYFYTAMSRPGRGEPRFIAVGYVDDTQFVRFDSDAASPRMAPRAPWIEQEGPEYWDGETRNMKASAQTYRENLRIALRYYNQSEAGSHIIQVMYGCDVGPDGRLLRGHNQYAYDGKDYIALNEDLSSWTAADTAAQITQRKWEAARVAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 105 - protein, 278 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPAS

SEQ ID NO: 106 - protein, 278 aa, Homo sapiens
CSHSMRYFYTAVSRPGRGEPRFIAVGYVDDTQFVRFDSDAASPRGEPRAPWVEQEGPEYWDRETQKYKRQAQTDRVSLRNLRGYYNQSEAGSHTLQWMYGCDLGPDGRLLRGYDQSAYDGKDYIALNEDLRSWTAADTAAQITQRKWEAARAAEQQRAYLEGTCVEWLRRYLENGKETLQRAEHPKTHVTHHLVSDHEATLRCWALGFYPAEITLTWQRDGEDQTQDTELVETRPAGDGTFQKWAAVVVPSGEEQRYTCHVQHEGLPEPLTLRWEPSS

SEQ ID NO: 107 - protein, 278 aa, Homo sapiens
CSHSMRYFYTAVSRPGRGEPRFIAVGYVDDTQFVQFDSDAASPRGEPRAPWVEQEGPEYWDRETQKYKRQAQTDRVSLRNLRGYYNQSEAGSHTLQRMYGCDLGPDGRLLRGYNQFAYDGKDYIALNEDLRSWTAADKAAQITQRKWEAAREAEQRRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGPSS

SEQ ID NO: 108 - protein, 278 aa, Homo sapiens
CSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSS

SEQ ID NO: 109 - protein, 278 aa, Homo sapiens
CSHSMRYFYTAVSRPGRGEPRFIAVGYVDDTQFVQFDSDAASPRGEPRAPWVEQEGPEYWDRETQKYKRQAQTDRVNLRKLRGYYNQSEAGSHTLQRMYGCDLGPDGRLLRGYNQFAYDGKDYIALNEDLRSWTAADKAAQITQRKWEAAREAEQRRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGPSS

SEQ ID NO: 110 - protein, 278 aa, Homo sapiens
GSHSMRYFYTAMSRPGRGEPRFITVGYVDDTLFVRFDSDATSPRKEPRAPWIEQEGPEYWDRETQISKTNTQTYRENLRTALRYYNQSEAGSHIIQRMYGCDVGPDGRLLRGYDQDAYDGKDYIALNEDLSSWTAADTAAQITQRKWEAARVAEQDRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEPSS

SEQ ID NO: 111 - protein, 278 aa, Homo sapiens
GSHSMRYFTTSVSRPGRGEPRFIAVGYVDDTQFVRFDSDAASQRMEPRAPWIEQEGPEYWDLQTRNVKAQSQTDRANLGTLRGYYNQSEAGSHTIQMMYGCDVGSDGRFLRGYRQDAYDGKDYIALNEDLRSWTAADMAAQITQRKWEAARVAEQLRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEPSS

SEQ ID NO: 112 - protein, 278 aa, Homo sapiens
GSHSMRYFYTAMSRPGRGEPRFITVGYVDDTLFVRFDSDATSPRKEPRAPWIEQEGPEYWDRETQISKTNTQTYRENLRTALRYYNQSEAGSHIIQRMYGCDVGPDGRLLRGYDQDAYDGKDYIALNEDLSSWTAADTAAQITQRKWEAARVAEQLRAYLEGLCVESLRRYLENGKETLQRADPPKTHVTHHPISDHEVTLRCWALGFYPAEITLTWQRDGEDQTQDTELVETRPAGDRTFQKWAAVVVPSGEEQRYTCHVQHEGLPKPLTLRWEPSS

SEQ ID NO: 113 - protein, 278 aa, Homo sapiens
GSHSMRYFYTAVSRPGRGEPHFIAVGYVDDTQFVRFDSDAASPRGEPRAPWVEQEGPEYWDRETQKYKRQAQTDRVSLRNLRGYYNQSEAGSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEPSS

SEQ ID NO: 114 - protein, 278 aa, Homo sapiens
GSHSMRYFHTAMSRPGRGEPRFITVGYVDDTLFVRFDSDATSPRKEPRAPWIEQEGPEYWDRETQISKTNTQTYRESLRNLRGYYNQSEAGSHTLQRMYGCDVGPDGRLLRGHNQYAYDGKDYIALNEDLRSWTAADTAAQISQRKLEAARVAEQLRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 115 - protein, 278 aa, Homo sapiens
CSHSMRYFDTAVSRPGRGEPRFISVGYVDDTQFVRFDSDAASPRGEPRAPWVEQEGPEYWDRETQKYKRQAQADRVNLRKLRGYYNQSEDGSHTLQWMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEPSS

SEQ ID NO: 116 - protein, 278 aa, Homo sapiens
GSHSMRYFYTAMSRPGRGEPRFIAVGYVDDTQFVRFDSDAASPRMAPRAPWIEQEGPEYWDRETQISKTNTQTYRESLRNLRGYYNQSEAGSHTLQRMYGCDVGPDGRLLRGHDQSAYDGKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 117 - protein, 278 aa, Homo sapiens
GSHSMRYFYTAVSRPGRGEPHFIAVGYVDDTQFVRFDSDAASPRGEPRAPWVEQEGPEYWDRETQKYKRQAQTDRVSLRNLRGYYNQSEARSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEPSS

SEQ ID NO: 118 - protein, 278 aa, Homo sapiens
GSHSMRYFSTSVSRPGSGEPRFIAVGYVDDTQFVRFDSDAASQRMEPRAPWIEQERPEYWDQETRNVKAQSQTDRVDLGTLRGYYNQSEAGSHTIQIMYGCDVGSDGRFLRGYEQHAYDGKDYIALNEDLRSWTAADMAAQITQRKWEAARWAEQLRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSS

SEQ ID NO: 119 - protein, 278 aa, Homo sapiens
GSHSMRYFYTAMSRPGRGEPRFITVGYVDDTQFVRFDSDATSPRMAPRAPWIEQEGPEYWDRETQISKTNTQTYRENLRTALRYYNQSEAGSHTWQTMYGCDLGPDGRLLRGHNQLAYDGKDYIALNEDLSSWTAADTAAQITQLKWEAARVAEQLRAYLEGECVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 120 - protein, 278 aa, Homo sapiens
CSHSMRYFYTAVSRPGRGEPRFIAVGYVDDTQFVRFDSDAASPRGEPRAPWVEQEGPEYWDRETQKYKRQAQADRVSLRNLRGYYNQSEAGSHTLQWMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEPSS

SEQ ID NO: 121 - protein, 278 aa, Homo sapiens
GSHSMRYFYTSVSRPGRGEPRFIAVGYVDDTQFVRFDSDAASQRMEPRAPWIEQEGPEYWDRNTRNVKAHSQTDRANLGTLRGYYNQSEDGSHTIQRMYGCDVGPDGRFLRGYQQDAYDGKDYIALNEDLRSWTAADMAAQITQRKWETAHEAEQWRAYLEGRCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEPSS

SEQ ID NO: 122 - protein, 278 aa, Homo sapiens
GSHSMRYFYTSVSRPGRGEPRFISVGYVDDTQFVRFDSDAASPREEPRAPWIEQEGPEYWDRNTQICKTNTQTYRENLRIALRYYNQSEAGSHTLQRMYGCDVGPDGRLLRGHNQFAYDGKDYIALNEDLSSWTAADTAAQITQRKWEAARVAEQLRTYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 123 - protein, 278 aa, Homo sapiens
GSHSMRYFYTAVSRPGRGEPRFISVGYVDDTQFVRFDSDAASPREEPRAPWIEQEGPEYWDRNTQICKTNTQTDRESLRNLRGYYNQSEAGSHTLQWMYGCDVGPDGRLLRGYNQFAYDGKDYIALNEDLSSWTAADTAAQITQRKWEAAREAEQLRAYLEGTCVEWLRRHLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 124 - protein, 278 aa, Homo sapiens
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRHLENGKETLQRTDPPRTHMTHHAVSDHEATLRCWALSFYPAEITLTWQRDGEDQTQDTELVETRPAGDGTFQKWASVVVPSGQEQRYTCHVQHEGLPKPLTLRWEPSS

SEQ ID NO: 125 - protein, 278 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITQRKWEAARVAEQLRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEPSS

SEQ ID NO: 126 - protein, 278 aa, Homo sapiens
GSHSMRYFYTSVSRPGRGEPRFIAVGYVDDTQFVRFDSDAASQRMEPRAPWIEQEGPEYWDRNTRNVKAHSQTDRESLRIALRYYNQSEDGSHTIQRMYGCDVGPDGRFLRGYQQDAYDGKDYIALNEDLRSWTAADMAAQITQRKWETAHEAEQWRAYLEGRCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEPSS

SEQ ID NO: 127 - protein, 278 aa, Homo sapiens
GSHSMRYFHTSVSRPGRGEPRFISVGYVDGTQFVRFDSDAASPRTEPRAPWIEQEGPEYWDRNTQISKTNTQTYRESLRNLRGYYNQSEAGSHTLQRMYGCDVGPDGRLLRGHDQSAYDGKDYIALNEDLSSWTAADTAAQITQRKWEAARVAEQLRAYLEGTCVEWLRRHLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 128 - protein, 278 aa, Homo sapiens
GSHSMRYFHTSVSRPGRGEPRFISVGYVDDTQFVRFDSDAASPRTEPRAPWIEQEGPEYWDRETQISKTNTQTYREDLRTLLRYYNQSEAGSHTIQRMSGCDVGPDGRLLRGYNQFAYDGKDYIALNEDLSSWTAADTAAQITQRKWEAARVAEQDRAYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 129 - protein, 278 aa, Homo sapiens
GSHSMRYFYTAMSRPGRGEPRFIAVGYVDDTQFVRFDSDAASPRTEPRAPWIEQEGPEYWDRNTQIFKTNTQTYRENLRIALRYYNQSEAGSHTWQTMYGCDVGPDGRLLRGHNQYAYDGKDYIALNEDLSSWTAADTAAQITQRKWEAAREAEQLRAYLEGLCVEWLRRHLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEPSS

SEQ ID NO: 130 - protein, 278 aa, Homo sapiens
CSHSMRYFSTSVSRPGRGEPRFIAVGYVDDTQFVRFDSDAASPRGEPRAPWVEQEGPEYWDRETQKYKRQAQTDRVSLRNLRGYYNQSEAGSHTLQWMFGCDLGPDGRLLRGYDQSAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEPSS

SEQ ID NO: 131 - protein, 278 aa, Homo sapiens
CSHSMRYFYTAVSRPGRGEPHFIAVGYVDDTQFVRFDSDAASPRGEPRAPWVEQEGPEYWDRETQNYKRQAQTDRVNLRKLRGYYNQSEAGSHIIQRMYGCDLGPDGRLLRGHDQLAYDGKDYIALNEDLRSWTAADTAAQITQRKWEAAREAEQLRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEPSS

SEQ ID NO: 132 - protein, 278 aa, Homo sapiens
CSHSMRYFYTAVSRPSRGEPHFIAVGYVDDTQFVRFDSDAASPRGEPRAPWVEQEGPEYWDRETQKYKRQAQTDRVNLRKLRGYYNQSEAGSHTLQRMYGCDLGPDGRLLRGYDQSAYDGKDYIALNEDLRSWTAADTAAQITQRKWEAAREAEQWRAYLEGECVEWLRRYLENGKETLQRAEHPKTHVTHHPVSDHEATLRCWALGFYPTEITLTWQRDGEDQTQDTELVETRPAGDGTFQKWAAVVVPSGEEQRYTCHVQHEGLPEPLTLRWEPSS

SEQ ID NO: 133 - protein, 278 aa, Homo sapiens
GSHSMRYFHTSVSRPGRGEPRFITVGYVDDTLFVRFDSDAASPREEPRAPWIEQEGPEYWDRETQICKAKAQTDREDLRTLLRYYNQSEAGSHTLQNMYGCDVGPDGRLLRGYHQDAYDGKDYIALNEDLSSWTAADTAAQITQRKWEAARVAEQLRAYLEGECVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 134 - protein, 278 aa, Homo sapiens
GSHSMRYFTTSVSRPGRGEPRFIAVGYVDDTQFVRFDSDAASQRMEPRAPWIEQERPEYWDQETRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWEPSS

SEQ ID NO: 135 - protein, 278 aa, Homo sapiens
GSHSMRYFSTSVSRPGSGEPRFIAVGYVDDTQFVRFDSDAASQRMEPRAPWIEQERPEYWDQETRNVKAHSQTDRENLGTLRGYYNQSEAGSHTIQIMYGCDVGSDGRFLRGYEQHAYDGKDYIALNEDLRSWTAADMAAQITQRKWEAARRAEQLRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSS

SEQ ID NO: 136 - protein, 278 aa, Homo sapiens
GSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 137 - protein, 278 aa, Homo sapiens
GSHSMRYFYTAVSRPGRGEPRFIAVGYVDDTQFVRFDSDAASPRGEPRAPWVEQEGPEYWDRETQKYKRQAQADRVNLRKLRGYYNQSEAGSHTIQRMYGCDLGPDGRLLRGYNQFAYDGKDYIALNEDLRSWTAADTAAQISQRKLEAAREAEQLRAYLEGECVEWLRGYLENGKETLQRAERPKTHVTHHPVSDHEATLRCWALGFYPAEITLTWQRDGEDQTQDTELVETRPAGDGTFQKWAAVVVPSGQEQRYTCHVQHEGLQEPCTLRWKPSS

SEQ ID NO: 138 - protein, 278 aa, Homo sapiens
GSHSMRYFYTAMSRPGRGEPRFIAVGYVDDTQFVRFDSDAASPRTEPRAPWIEQEGPEYWDRNTQIFKTNTQTYRESLRNLRGYYNQSEAGSHIIQRMYGCDLGPDGRFLRGHNQYAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEPSS

SEQ ID NO: 139 - protein, 278 aa, Homo sapiens
GSHSMRYFYTSVSRPGRGEPRFISVGYVDDTQFVRFDSDAASPREEPRAPWIEQEGPEYWDRNTQICKTNTQTDRESLRNLRGYYNQSEAGSHTWQTMYGCDVGPDGRLLRGHNQFAYDGKDYIALNEDLSSWTAADTAAQITQRKWEAARVAEQLRTYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 140 - protein, 278 aa, Homo sapiens
GSHSMRYFYTAVSRPGRGEPHFIAVGYVDDTQFVRFDSDAASPRGEPRAPWVEQEGPEYWDRETQKYKRQAQTDRVSLRNLRGYYNQSEAGSHILQRMYGCDVGPDGRLLRGYDQSAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEPSS

SEQ ID NO: 141 - protein, 278 aa, Homo sapiens
GSHSMRYFYTAMSRPGRGEPRFIAVGYVDDTQFVRFDSDAASPRTEPRAPWIEQEGPEYWDGETRNMKASAQTYRENLRIALRYYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEPSS

SEQ ID NO: 142 - protein, 278 aa, Homo sapiens
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWEPSS

SEQ ID NO: 143 - protein, 278 aa, Homo sapiens
GSHSMRYFYTSMSRPGRGEPRFIAVGYVDDTQFVRFDSDAASQRMEPRAPWIEQEGPEYWDRNTRNVKAQSQTDRVDLGTLRGYYNQSEAGSHTIQRMYGCDVGPDGRFLRGYHQYAYDGKDYIALKEDLRSWTAADMAAQTTKHKWEAAHVAEQWRAYLEGTCVEWLRRYLENGKETLQRTDAPKTHMTHHAVSDHEATLRCWALSFYPAEITLTWQRDGEDQTQDTELVETRPAGDGTFQKWVAVVVPSGQEQRYTCHVQHEGLPKPLTLRWEPSS

SEQ ID NO: 144 - protein, 278 aa, Homo sapiens
CSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWEPSS

SEQ ID NO: 145 - protein, 278 aa, Homo sapiens
CSHSMRYFDTAVSRPGRGEPRFISVGYVDDTQFVRFDSDAASPRGEPRAPWVEQEGPEYWDRETQKYKRQAQADRVSLRNLRGYYNQSEDGSHTFQRMYGCDLGPDGRLLRGYDQFAYDGKDYIALNEDLRSWTAADTAAQITQRKLEAARAAEQDRAYLEGTCVEWLRRYLENGKKTLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSS

SEQ ID NO: 146 - protein, 278 aa, Homo sapiens
GSHSMRYFYTSVSRPGRGEPRFIAVGYVDDTQFVRFDSDAASQRMEPRAPWIEQEGPEYWDRNTRNVKAQSQTDRVDLGTLRGYYNQSEAGSHTIQMMYGCDVGSDGRFLRGYRQDAYDGKDYIALKEDLRSWTAADMAAQTTKHKWEAAHVAEQWRAYLEGTCVEWLRRYLENGKETLQRTDAPKTHMTHHAVSDHEATLRCWALSFYPAEITLTWQRDGEDQTQDTELVETRPAGDGTFQKWVAVVVPSGQEQRYTCHVQHEGLPKPLTLRWEPSS

SEQ ID NO: 147 - protein, 278 aa, Homo sapiens
GSHSMRYFFTSVSRPGRGEPRFIAVGYVDDTQFVRFDSDAASQRMEPRAPWIEQEGPEYWDQETRNVKAHSQTDRESLRIALRYYNQSEAGSHTIQMMYGCDVGPDGRLLRGYQQDAYDGKDYIALNEDLRSWTAADMAAQITQRKWEAARVAEQLRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEPSS

SEQ ID NO: 148 - protein, 278 aa, Homo sapiens
GSHSMRYFHTAMSRPGRGEPRFITVGYVDDTLFVRFDSDATSPRKEPRAPWIEQEGPEYWDRETQISKTNTQTYRENLRIALRYYNQSEAGSHTWQRMYGCDLGPDGRLLRGYNQLAYDGKDYIALNEDLSSWTAADTAAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 149 - protein, 278 aa, Homo sapiens
GSHSMRYFYTAMSRPGRGEPRFIAVGYVDDTQFVRFDSDAASPRTEPRAPWIEQEGPEYWDRNTQIFKTNTQTYRENLRIALRYYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEPSS

SEQ ID NO: 150 - protein, 278 aa, Homo sapiens
GSHSMRYFHTAMSRPGRGEPRFITVGYVDDTLFVRFDSDATSPRKEPRAPWIEQEGPEYWDRETQISKTNTQTYRESLRNLRGYYNQSEAGSHTWQRMYGCDLGPDGRLLRGYNQLAYDGKDYIALNEDLSSWTAADTAAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 151 - protein, 278 aa, Homo sapiens
GSHSMRYFYTSVSRPGRGEPRFIAVGYVDDTQFVRFDSDAASRRMEPRAPWIEQEGPEYWDGETRKVKAHSQTHRVDLGTLRGYYNQSEAGSHTLQRMYGCDVGSDWRFLRGYHQYAYDGKDYIALKEDLRSWTAADMAAQTTKHKWEAAHVAEQWRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSS

SEQ ID NO: 152 - protein, 278 aa, Homo sapiens
GSHSMRYFYTAMSRPGRGEPRFIAVGYVDDTQFVRFDSDAASPREEPRAPWIEQEGPEYWDRNTQIYKAQAQTDRESLRNLRGYYNQSEAGSHTWQTMYGCDLGPDGRLLRGHNQLAYDGKDYIALNEDLSSWTAADTAAQITQRKWEAAREAEQLRAYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 153 - protein, 278 aa, Homo sapiens
GSHSMRYFHTAMSRPGRGEPRFITVGYVDDTLFVRFDSDATSPRKEPRAPWIEQEGPEYWDRETQISKTNTQTYRESLRNLRGYYNQSEAGSHTWQRMYGCDLGPDGRLLRGYNQLAYDGKDYIALNEDLSSWTAADTAAQITQRKWEAARVAEQDRAYLEGLCVESLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 154 - protein, 278 aa, Homo sapiens
GSHSMRYFYTAMSRPGRGEPRFIAVGYVDDTQFVRFDSDAASPRTEPRAPWIEQEGPEYWDRETQISKTNTQTYRENLRIALRYYNQSEAGSHTWQTMYGCDVGPDGRLLRGHNQYAYDGKDYIALNEDLSSWTAADTAAQITQRKWEAAREAEQLRAYLEGLCVEWLRRHLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEPSS

SEQ ID NO: 155 - protein, 278 aa, Homo sapiens
CSHSMRYFYTAVSRPGRGEPRFIAVGYVDDTQFVRFDSDAASPRGEPRAPWVEQEGPEYWDRETQKYKRQAQADRVSLRNLRGYYNQSEAGSHTLQRMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEPSS

SEQ ID NO: 156 - protein, 278 aa, Homo sapiens
GSHSMRYFYTAMSRPGRGEPRFIAVGYVDDTQFVRFDSDAASPRTEPRAPWIEQEGPEYWDRNTQIFKTNTQTYRESLRNLRGYYNQSEAGSHIIQRMYGCDLGPDGRLLRGHDQFAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEPSS

SEQ ID NO: 157 - protein, 278 aa, Homo sapiens
GSHSMRYFHTSVSRPGRGEPRFITVGYVDDTLFVRFDSDATSPRKEPRAPWIEQEGPEYWDRETQISKTNTQTYRESLRNLRGYYNQSEAGSHTLQSMYGCDVGPDGRLLRGHNQYAYDGKDYIALNEDLRSWTAADTAAQITQRKWEAARVAEQLRAYLEGECVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 158 - protein, 278 aa, Homo sapiens
GSHSMRYFYTAMSRPGRGEPRFISVGYVDDTQFVRFDSDAASPREEPRAPWIEQEGPEYWDRETQISKTNTQTYRESLRNLRGYYNQSEAGSHTLQRMYGCDVGPDGRLLRGHDQSAYDGKDYIALNEDLSSWTAADTAAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEPSS

SEQ ID NO: 159 - protein, 278 aa, Homo sapiens
GSHSMRYFFTSVSRPGRGEPRFIAVGYVDDTQFVRFDSDAASQRMEPRAPWIEQEGPEYWDQETRNVKAHSQTDRVDLGTLRGYYNQSEAGSHTIQMMYGCDVGPDGRLLRGYQQDAYDGKDYIALNEDLRSWTAADMAAQITQRKWEAARVAEQLRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEPSS

SEQ ID NO: 160 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 161 - protein, 40 aa, Artificial Sequence
  /note=“Description of Artificial Sequence Synthetic polypeptide”
GSGSAGGGLNDIFEAQKIEWHEGSTGHHHHHHDYKDDDDK

SEQ ID NO: 162 - protein, 26 aa, Artificial Sequence
  /note=“Description of Artificial Sequence Synthetic peptide”
GSGSAGGSGSGGGSLPETGGHHHHHH

SEQ ID NO: 163 - protein, 5 aa, Artificial Sequence
  /note=“Description of Artificial Sequence Synthetic peptide”
  MOD_RES (3)..(3): Any amino acid
LPXTG

SEQ ID NO: 164 - protein, 5 aa, Artificial Sequence
  /note=“Description of Artificial Sequence Synthetic peptide”
IPKTG

SEQ ID NO: 165 - protein, 5 aa, Artificial Sequence
  /note=“Description of Artificial Sequence Synthetic peptide”
  MOD_RES (3)..(3): Any amino acid
MPXTG

SEQ ID NO: 166 - protein, 5 aa, Artificial Sequence
  /note=“Description of Artificial Sequence Synthetic peptide”
LAETG

SEQ ID NO: 167 - protein, 5 aa, Artificial Sequence
  /note=“Description of Artificial Sequence Synthetic peptide”
  MOD_RES (3)..(3): Any amino acid
LPXAG

SEQ ID NO: 168 - protein, 5 aa, Artificial Sequence
  /note=“Description of Artificial Sequence Synthetic peptide”
LPESG

SEQ ID NO: 169 - protein, 5 aa, Artificial Sequence
  /note=“Description of Artificial Sequence Synthetic peptide”
LPELG

SEQ ID NO: 170 - protein, 5 aa, Artificial Sequence
  /note=“Description of Artificial Sequence Synthetic peptide”
LPEVG

SEQ ID NO: 171 - protein, 5 aa, Artificial Sequence
  /note=“Description of Artificial Sequence Synthetic peptide”
  MOD_RES (1)..(1): Any amino acid
XPKTG

SEQ ID NO: 172 - protein, 5 aa, Artificial Sequence
  /note=“Description of Artificial Sequence Synthetic peptide”
APKTG

SEQ ID NO: 173 - protein, 5 aa, Artificial Sequence
  /note=“Description of Artificial Sequence Synthetic peptide”
DPKTG

SEQ ID NO: 174 - protein, 5 aa, Artificial Sequence
  /note=“Description of Artificial Sequence Synthetic peptide”
SPKTG

SEQ ID NO: 175 - protein, 5 aa, Artificial Sequence
  /note=“Description of Artificial Sequence Synthetic peptide”
  MOD_RES (4)..(4): Any amino acid
LPEXG

SEQ ID NO: 176 - protein, 5 aa, Artificial Sequence
  /note=“Description of Artificial Sequence Synthetic peptide”
LPEAG

SEQ ID NO: 177 - protein, 5 aa, Artificial Sequence
  /note=“Description of Artificial Sequence Synthetic peptide”
LPECG

SEQ ID NO: 178 - protein, 5 aa, Artificial Sequence
  /note=“Description of Artificial Sequence Synthetic peptide”
  SITE (1)..(5): /note=“Variant residues given in the sequence have no preference with respect to those in the annotations for variant positions”
  VARIANT (4)..(4): /replace=“Cys” or “Ser”
LPEAG

SEQ ID NO: 179 - protein, 5 aa, Artificial Sequence
  /note=“Description of Artificial Sequence Synthetic peptide”
WTWTW

SEQ ID NO: 180 - protein, 193 aa, Artificial Sequence
  /note=“Description of Artificial Sequence Synthetic polypeptide”
GSGSAGGSFESGPGAEYCFNVDDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQGSTGDYKDDDDK

SEQ ID NO: 181 - protein, 30 aa, Artificial Sequence
  /note=“Description of Artificial Sequence Synthetic polypeptide”
  SITE (1)..(30): /note=“This sequence may encompass 1-6 ′Gly Gly Gly Gly Ser′ repeating units”
GGGGSGGGGSGGGGSGGGGSGGGGSGGGGS

SEQ ID NO: 182 - protein, 13 aa, Artificial Sequence
  /note=“Description of Artificial Sequence Synthetic peptide”
SSSSGSSSSGSAA

SEQ ID NO: 183 - protein, 5 aa, Artificial Sequence
  /note=“Description of Artificial Sequence Synthetic peptide”
GGGGG

SEQ ID NO: 184 - protein, 51 aa, Artificial Sequence
  /note=“Description of Artificial Sequence Synthetic polypeptide”
  SITE (2)..(51): /note=“This sequence may encompass 1-10 ′Gly Gly Gly Gly Ser′ repeating units”
SGGGGSGGGGSGGGGSGGGGSGGGGSGGGGSGGGGSGGGGSGGGGSGGGGS

SEQ ID NO: 185 - protein, 20 aa, Artificial Sequence
  /note=“Description of Artificial Sequence Synthetic peptide”
  SITE (1)..(20): /note=“This sequence may encompass 1-5 ′Gly Gly Ser Gly′ repeating units”
GGSGGGSGGGSGGGSGGGSG

SEQ ID NO: 186 - protein, 4 aa, Artificial Sequence
  /note=“Description of Artificial Sequence Synthetic peptide”
GSAT

SEQ ID NO: 187 - protein, 30 aa, Artificial Sequence
  /note=“Description of Artificial Sequence Synthetic polypeptide”
  SITE (1)..(30): /note=“This sequence may encompass 1-5 ′Gly Gly Ser Gly Gly Ser′ repeating units”
GGSGGSGGSGGSGGSGGSGGSGGSGGSGGS

SEQ ID NO: 188 - protein, 5 aa, Unknown
  /note=“Description of Unknown Enterokinase cleavage site”
DDDDK

SEQ ID NO: 189 - protein, 16 aa, Artificial Sequence
  /note=“Description of Artificial Sequence Synthetic peptide”
KPVSKMRMATPLLMQA

SEQ ID NO: 190 - protein, 15 aa, Artificial Sequence
  /note=“Description of Artificial Sequence Synthetic peptide”
QIYKANSKFIGITEL

SEQ ID NO: 191 - protein, 208 aa, Artificial Sequence
  /note=“Description of Artificial Sequence Synthetic polypeptide”
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTEGSEQKLISEEDLPETGG

SEQ ID NO: 192 - protein, 252 aa, Artificial Sequence
  /note=“Description of Artificial Sequence Synthetic polypeptide”
KPVSKMRMATPLLMQAGGGGSIEGRGSGGGSGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKLGGLNDIFEAQKIEWHEHHHHHH

SEQ ID NO: 193 - protein, 13 aa, Influenza A virus
PKYVKQNTLKLAT

SEQ ID NO: 194 - protein, 254 aa, Homo sapiens
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGVRKSNAAERRGPL

SEQ ID NO: 195 - protein, 266 aa, Homo sapiens
MVCLKLPGGSCMTALTVTLMVLSSPLALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 196 - protein, 266 aa, Homo sapiens [sequence omitted]

SEQ ID NO: 197 - protein, 266 aa, Homo sapiens
MVCLRLPGGSCMAVLTVTLMVLSSPLALAGDTRPRFLEYSTSECHFFNGTERVRYLDRYFHNQEENVRFDSDVGEFRAVTELGRPDAEYWNSQKDLLEQKRGRVDNYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSKMLSGVGGFVLGLLFLGAGLFIYFRNQKGHSGLQPRGFLS

SEQ ID NO: 198 - protein, 266 aa, Homo sapiens [sequence omitted]

SEQ ID NO: 199 - protein, 266 aa, Homo sapiens [sequence omitted]

SEQ ID NO: 200 - protein, 266 aa, Homo sapiens
MVCLKLPGGSCMAALTVTLMVLSSPLALAGDTQPRFLWQGKYKCHFFNGTERVQFLERLFYNQEEFVRFDSDVGEYRAVTELGRPVAESWNSQKDILEDRRGQVDTVCRHNYGVGESFTVQRRVHPEVTVYPAKTQPLQHHNLLVCSVSGFYPGSIEVRWFRNGQEEKAGVVSTGLIQNGDWTFQTLVMLETVPRSGEVYTCQVEHPSVMSPLTVEWRARSESAQSKMLSGVGGFVLGLLFLGAGLFIYFRNQKGHSGLQPTGFLS

SEQ ID NO: 201 - protein, 266 aa, Homo sapiens [sequence omitted]

SEQ ID NO: 202 - protein, 266 aa, Homo sapiens [sequence omitted]

SEQ ID NO: 203 - protein, 266 aa, Homo sapiens [sequence omitted]

SEQ ID NO: 204 - protein, 266 aa, Homo sapiens [sequence omitted]

SEQ ID NO: 205 - protein, 266 aa, Homo sapiens [sequence omitted]

SEQ ID NO: 206 - protein, 266 aa, Homo sapiens [sequence omitted]

SEQ ID NO: 207 - protein, 266 aa, Homo sapiens [sequence omitted]

SEQ ID NO: 208 - protein, 266 aa, Homo sapiens
MVCLKLPGGSCMTALTVTLMVLSSPLALSGDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 209 - protein, 266 aa, Homo sapiens [sequence omitted]

SEQ ID NO: 210 - protein, 255 aa, Homo sapiens [sequence omitted]

SEQ ID NO: 211 - protein, 261 aa, Homo sapiens [sequence omitted]

SEQ ID NO: 212 - protein, 255 aa, Homo sapiens
MILNKALLLGALALTTVMSPCGGEDIVADHVASCGVNLYQFYGPSGQYTHEFDGDEQFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTETVVCALGLSVGLMGIVVGTVFIIQGLRSVGASRHQGPL

SEQ ID NO: 213 - protein, 261 aa, Homo sapiens
MSWKKALRIPGDLRVATVTLMLAMLSSLLAEGRDSPEDFVFQFKGMCYFTNGTERVRLVTRYIYNREEYARFDSDVGVYRAVTPQGRPDAEYWNSQKEVLEGTRAELDTVCRHNYEVAFRGILQRRVEPTVTISPSRTEALNHHNLLVCSVTDFYPGQIKVRWFRNDQEETAGVVSTPLIRNGDWTFQILVMLEMTPQRGDVYTCHVEHPSLQSPITVEWRAQSESAQSKMLSGVGGFVLGLIFLGLGLIIRQRSQKGLLH

SEQ ID NO: 214 - protein, 255 aa, Homo sapiens
MILNKALMLGALALTTVMSPCGGEDIVADHVASYGVNLYQSYGPSGQYSHEFDGDEEFYVDLERKETVWQLPLFRRFRRFDPQFALTNIAVLKHNLNIVIKRSNSTAATNEVPEVTVFSKSPVTLGQPNTLICLVDNIFPPVVNITWLSNGHSVTEGVSETSFLSKSDHSFFKISYLTFLPSADEIYDCKVEHWGLDEPLLKHWEPEIPTPMSELTETVVCALGLSVGLVGIVVGTVLIIRGLRSVGASRHQGPL

SEQ ID NO: 215 - protein, 261 aa, Homo sapiens [sequence omitted]

SEQ ID NO: 216 - protein, 254 aa, Homo sapiens
MILNKALMLGALALTTVMSPCGGEDIVADHVASYGVNLYQSYGPSGQYTHEFDGDEQFYVDLGRKETVWCLPVLRQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTETVVCALGLSVGLVGIVVGTVFIIRGLRSVGASRHQGPL

SEQ ID NO: 217 - protein, 261 aa, Homo sapiens
MSWKKALRIPGGLRAATVTLMLSMLSTPVAEGRDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKMLSGIGGFVLGLIFLGLGLIIHHRSQKGLLH

SEQ ID NO: 218 - protein, 261 aa, Homo sapiens
MSWKKALRIPGGLRAATVTLMLAMLSTPVAEGRDSPEDFVYQFKAMCYFTNGTERVRYVTRYIYNREEYARFDSDVEVYRAVTPLGPPDAEYWNSQKEVLERTRAELDTVCRHNYQLELRTTLQRRVEPTVTISPSRTEALNHHNLLVCSVTDFYPAQIKVRWFRNDQEETTGVVSTPLIRNGDWTFQILVMLEMTPQHGDVYTCHVEHPSLQNPITVEWRAQSESAQSKMLSGIGGFVLGLIFLGLGLIIHHRSQKGLLH

SEQ ID NO: 219 - protein, 261 aa, Homo sapiens [sequence omitted]

SEQ ID NO: 220 - protein, 261 aa, Homo sapiens [sequence omitted]

SEQ ID NO: 221 - protein, 269 aa, Homo sapiens [sequence omitted]

SEQ ID NO: 222 - protein, 261 aa, Homo sapiens [sequence omitted]

SEQ ID NO: 223 - protein, 261 aa, Homo sapiens [sequence omitted]

SEQ ID NO: 224 - protein, 191 aa, Homo sapiens
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTE

SEQ ID NO: 225 - protein, 198 aa, Homo sapiens
GDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSK

SEQ ID NO: 226 - protein, 198 aa, Homo sapiens
GDTRPRFLWQLKFECHFFNGTERVRLLERCIYNQEESVRFDSDVGEYRAVTELGRPDAEYWNSQKDLLEQRRAAVDTYCRHNYGAVESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSK

SEQ ID NO: 227 - protein, 198 aa, Homo sapiens
GDTRPRFLEYSTSECHFFNGTERVRYLDRYFHNQEENVRFDSDVGEFRAVTELGRPDAEYWNSQKDLLEQKRGRVDNYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 228 - protein, 198 aa, Homo sapiens
GDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSK

SEQ ID NO: 229 - protein, 198 aa, Homo sapiens
GDTRPRFLEQVKHECHFFNGTERVRFLDRYFYHQEEYVRFDSDVGEYRAVTELGRPDAEYWNSQKDLLEQRRAAVDTYCRHNYGVVESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSK

SEQ ID NO: 230 - protein, 198 aa, Homo sapiens
GDTQPRFLWQGKYKCHFFNGTERVQFLERLFYNQEEFVRFDSDVGEYRAVTELGRPVAESWNSQKDILEDRRGQVDTVCRHNYGVGESFTVQRRVHPEVTVYPAKTQPLQHHNLLVCSVSGFYPGSIEVRWFRNGQEEKAGVVSTGLIQNGDWTFQTLVMLETVPRSGEVYTCQVEHPSVMSPLTVEWRARSESAQSK

SEQ ID NO: 231 - protein, 198 aa, Homo sapiens
GDTRPRFLEYSTGECYFFNGTERVRFLDRYFYNQEEYVRFDSDVGEYRAVTELGRPSAEYWNSQKDFLEDRRALVDTYCRHNYGVGESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWSARSESAQSK

SEQ ID NO: 232 - protein, 198 aa, Homo sapiens
GDTRPRFLEEVKFECHFFNGTERVRLLERRVHNQEEYARYDSDVGEYRAVTELGRPDAEYWNSQKDLLERRRAAVDTYCRHNYGVGESFTVQRRVQPKVTVYPSKTQPLQHHNLLVCSVNGFYPGSIEVRWFRNGQEEKTGVVSTGLIQNGDWTFQTLVMLETVPQSGEVYTCQVEHPSVMSPLTVEWRARSESAQSK

SEQ ID NO: 233 - protein, 198 aa, Homo sapiens
GDTRPRFLEYSTSECHFFNGTERVRFLDRYFYNQEEYVRFDSDVGEFRAVTELGRPDEEYWNSQKDFLEDRRAAVDTYCRHNYGVGESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 234 - protein, 198 aa, Homo sapiens
GDTRPRFLEYSTSECHFFNGTERVRFLDRYFYNQEEYVRFDSDVGEFRAVTELGRPDEEYWNSQKDFLEDRRAAVDTYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 235 - protein, 198 aa, Homo sapiens
GDTRPRFLEYSTSECHFFNGTERVRFLDRYFHNQEENVRFDSDVGEFRAVTELGRPDAEYWNSQKDILEDERAAVDTYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 236 - protein, 198 aa, Homo sapiens
GDTRPRFLEYSTSECHFFNGTERVRFLDRYFHNQEENVRFDSDVGEFRAVTELGRPDAEYWNSQKDILEDERAAVDTYCRHNYGVGESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 237 - protein, 198 aa, Homo sapiens
GDTRPRFLEYSTSECHFFNGTERVRFLDRYFHNQEEFVRFDSDVGEYRAVTELGRPAAEHWNSQKDLLERRRAEVDTYCRHNYGVVESFTVQRRVHPKVTVYPSKTQPLQHYNLLVCSVSGFYPGSIEVRWFRNGQEEKTGVVSTGLIHNGDWTFQTLVMLETVPRSGEVYTCQVEHPSVTSPLTVEWRARSESAQSK

SEQ ID NO: 238 - protein, 198 aa, Homo sapiens
GDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSK

SEQ ID NO: 239 - protein, 198 aa, Homo sapiens
GDTRPRFLWQPKRECHFFNGTERVRFLDRHFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSK

SEQ ID NO: 240 - protein, 195 aa, Homo sapiens
EDIVADHVASCGVNLYQFYGPSGQYTHEFDGDEEFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTET

SEQ ID NO: 241 - protein, 199 aa, Homo sapiens
GRDSPEDFVYQFKGLCYFTNGTERVRGVTRHIYNREEYVRFDSDVGVYRAVTPQGRPVAEYWNSQKEVLEGARASVDRVCRHNYEVAYRGILQRRVEPTVTISPSRTEALNHHNLLICSVTDFYPSQIKVRWFRNDQEETAGVVSTPLIRNGDWTFQILVMLEMTPQRGDVYTCHVEHPSLQSPITVEWRAQSESAQSK

SEQ ID NO: 242 - protein, 195 aa, Homo sapiens
EDIVADHVASCGVNLYQFYGPSGQYTHEFDGDEQFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTET

SEQ ID NO: 243 - protein, 199 aa, Homo sapiens
GRDSPEDFVFQFKGMCYFTNGTERVRLVTRYIYNREEYARFDSDVGVYRAVTPQGRPDAEYWNSQKEVLEGTRAELDTVCRHNYEVAFRGILQRRVEPTVTISPSRTEALNHHNLLVCSVTDFYPGQIKVRWFRNDQEETAGVVSTPLIRNGDWTFQILVMLEMTPQRGDVYTCHVEHPSLQSPITVEWRAQSESAQSK

SEQ ID NO: 244 - protein, 195 aa, Homo sapiens
EDIVADHVASYGVNLYQSYGPSGQYSHEFDGDEEFYVDLERKETVWQLPLFRRFRRFDPQFALTNIAVLKHNLNIVIKRSNSTAATNEVPEVTVFSKSPVTLGQPNTLICLVDNIFPPVVNITWLSNGHSVTEGVSETSFLSKSDHSFFKISYLTFLPSADEIYDCKVEHWGLDEPLLKHWEPEIPTPMSELTET

SEQ ID NO: 245 - protein, 199 aa, Homo sapiens
GRDSPEDFVYQFKGMCYFTNGTERVRLVTRYIYNREEYARFDSDVGVYRAVTPLGPPAAEYWNSQKEVLERTRAELDTVCRHNYQLELRTTLQRRVEPTVTISPSRTEALNHHNLLVCSVTDFYPAQIKVRWFRNDQEETTGVVSTPLIRNGDWTFQILVMLEMTPQRGDVYTCHVEHPSLQNPIIVEWRAQSESAQSK

SEQ ID NO: 246 - protein, 194 aa, Homo sapiens
EDIVADHVASYGVNLYQSYGPSGQYTHEFDGDEQFYVDLGRKETVWCLPVLRQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTET

SEQ ID NO: 247 - protein, 199 aa, Homo sapiens
GRDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSK

SEQ ID NO: 248 - protein, 199 aa, Homo sapiens
GRDSPEDFVYQFKAMCYFTNGTERVRYVTRYIYNREEYARFDSDVEVYRAVTPLGPPDAEYWNSQKEVLERTRAELDTVCRHNYQLELRTTLQRRVEPTVTISPSRTEALNHHNLLVCSVTDFYPAQIKVRWFRNDQEETTGVVSTPLIRNGDWTFQILVMLEMTPQHGDVYTCHVEHPSLQNPITVEWRAQSESAQSK

SEQ ID NO: 249 - protein, 199 aa, Homo sapiens
GRDSPEDFVYQFKGMCYFTNGTERVRLVTRYIYNREEYARFDSDVGVYRAVTPLGPPDAEYWNSQKEVLERTRAELDTVCRHNYQLELRTTLQRRVEPTVTISPSRTEALNHHNLLVCSVTDFYPAQIKVRWFRNDQEETTGVVSTPLIRNGDWTFQILVMLEMTPQRGDVYTCHVEHPSLQNPIIVEWRAQSESAQSK

SEQ ID NO: 250 - protein, 199 aa, Homo sapiens
GRDSPEDFVFQFKGMCYFTNGTERVRGVTRYIYNREEYARFDSDVGVYRAVTPLGRLDAEYWNSQKDILEEDRASVDTVCRHNYQLELRTTLQRRVEPTVTISPSRTEALNHHNLLVCSVTDFYPAQIKVRWFRNDQEETTGVVSTPLIRNGDWTFQILVMLEMTPQRGDVYTCHVEHPSLQNPIIVEWRAQSESAQSK

SEQ ID NO: 251 - protein, 199 aa, Homo sapiens
GRDSPEDFVYQFKGLCYFTNGTERVRGVTRHIYNREEYVRFDSDVGVYRAVTPQGRPDAEYWNSQKEVLEGARASVDRVCRHNYEVAYRGILQRRVEPTVTISPSRTEALNHHNLLICSVTDFYPSQIKVRWFRNDQEETAGVVSTPLIRNGDWTFQILVMLEMTPQRGDVYTCHVEHPSLQSPITVEWRAQSESAQSK

SEQ ID NO: 252 - protein, 199 aa, Homo sapiens
GRDSPEDFVYQFKGMCYFTNGTERVRLVTRHIYNREEYARFDSDVGVYRAVTPQGRPDAEYWNSQKEVLEGTRAELDTVCRHNYEVAFRGILQRRVEPTVTISPSRTEALNHHNLLVCSVTDFYPGQIKVRWFRNDQEETAGVVSTPLIRNGDWTFQILVMLEMTPQRGDVYTCHVEHPSLQSPITVEWRAQSESAQSK

SEQ ID NO: 253 - protein, 199 aa, Homo sapiens
GRDSPEDFVYQFKGMCYFTNGTERVRLVTRHIYNREEYARFDSDVGVYRAVTPQGRPVAEYWNSQKEVLERTRAELDTVCRHNYEVGYRGILQRRVEPTVTISPSRTEALNHHNLLVCSVTDFYPGQIKVQWFRNDQEETAGVVSTPLIRNGDWTFQILVMLEMTPQRGDVYTCHVEHPSLQSPITVEWRAQSESAQSK

SEQ ID NO: 254 - protein, 34 aa, Artificial Sequence
  /note=“Description of Artificial Sequence Synthetic polypeptide”
GLNDIFEAQKIEWHEGSGEQKLISEEDLHHHHHH

SEQ ID NO: 255 - protein, 28 aa, Artificial Sequence
  /note=“Description of Artificial Sequence Synthetic peptide”
GLNDIFEAQKIEWHEGSGEQKLISEEDL

SEQ ID NO: 256 - protein, 26 aa, Artificial Sequence
  /note=“Description of Artificial Sequence Synthetic peptide”
GSGSAGGSGSGGGSLPETGGHHHHHH

SEQ ID NO: 257 - protein, 20 aa, Artificial Sequence
  /note=“Description of Artificial Sequence Synthetic peptide”
GSGSAGGSGSGGGSLPETGG

SEQ ID NO: 258 - protein, 14 aa, Artificial Sequence
  /note=“Description of Artificial Sequence Synthetic peptide”
GKPIPNPLLGLDST

SEQ ID NO: 259 - protein, 39 aa, Artificial Sequence
  /note=“Description of Artificial Sequence Synthetic polypeptide”
LTDTLQAETDQLEDEKSALQTEIANLLKEKEKLEFILAA

SEQ ID NO: 260 - protein, 40 aa, Artificial Sequence
  /note=“Description of Artificial Sequence Synthetic polypeptide”
RIARLEEKVKTLKAQNSELASTANMLREQVAQLKQKVMNH

SEQ ID NO: 261 - protein, 35 aa, Artificial Sequence
  /note=“Description of Artificial Sequence Synthetic polypeptide”
TTAPSAQLEKELQALQKENAQLEWELQALEKELAQ

SEQ ID NO: 262 - protein, 35 aa, Artificial Sequence
  /note=“Description of Artificial Sequence Synthetic polypeptide”
TTAPSAQLKKKLQALKKKNAQLKWKLQALKKKLAQ

SEQ ID NO: 263 - protein, 232 aa, Artificial Sequence
  /note=“Description of Artificial Sequence Synthetic polypeptide”
EPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 264 - protein, 232 aa, Artificial Sequence
  /note=“Description of Artificial Sequence Synthetic polypeptide”
EPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 265 - protein, 16 aa, Artificial Sequence
  /note=“Description of Artificial Sequence Synthetic peptide”
RGVPHIVMVDAYKRYK

SEQ ID NO: 266 - protein, 113 aa, Artificial Sequence
  /note=“Description of Artificial Sequence Synthetic polypeptide”
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 267 - protein, 8 aa, Artificial Sequence
  /note=“Description of Artificial Sequence Synthetic peptide”
DYKDDDDK

SEQ ID NO: 268 - protein, 8 aa, Artificial Sequence
  /note=“Description of Artificial Sequence Synthetic peptide”
WSHPQFEK

SEQ ID NO: 269 - protein, 12 aa, Artificial Sequence
  /note=“Description of Artificial Sequence Synthetic peptide”
EDQVDPRLIDGK

SEQ ID NO: 270 - protein, 6 aa, Homo sapiens
VEPKSC

SEQ ID NO: 271 - protein, 9 aa, Artificial Sequence
  /note=“Description of Artificial Sequence Synthetic peptide”
  MOD_RES (5)..(5): 3-amino-3-(2-nitro)phenyl-propionic acid
SVRDXLARL

SEQ ID NO: 272 - protein, 9 aa, Artificial Sequence
  /note=“Description of Artificial Sequence Synthetic peptide”
  MOD_RES (4)..(4): 3-amino-3-(2-nitro)phenyl-propionic acid
LTAXFLIFL

SEQ ID NO: 273 - protein, 9 aa, Artificial Sequence
  /note=“Description of Artificial Sequence Synthetic peptide”
  MOD_RES (6)..(6): 3-amino-3-(2-nitro)phenyl-propionic acid
LLDSDXERL

SEQ ID NO: 274 - protein, 9 aa, Artificial Sequence
  /note=“Description of Artificial Sequence Synthetic peptide”
  MOD_RES (5)..(5): 3-amino-3-(2-nitro)phenyl-propionic acid
KMDIXVPLL

SEQ ID NO: 275 - protein, 9 aa, Artificial Sequence
  /note=“Description of Artificial Sequence Synthetic peptide”
  MOD_RES (4)..(4): 3-amino-3-(2-nitro)phenyl-propionic acid
FYVXGAANR

SEQ ID NO: 276 - protein, 9 aa, Artificial Sequence
  /note=“Description of Artificial Sequence Synthetic peptide”
  MOD_RES (7)..(7): 3-amino-3-(2-nitro)phenyl-propionic acid
ILGPPGXVY

SEQ ID NO: 277 - protein, 9 aa, Artificial Sequence
  /note=“Description of Artificial Sequence Synthetic peptide”
  MOD_RES (7)..(7): 3-amino-3-(2-nitro)phenyl-propionic acid
EEFGAAXSF

SEQ ID NO: 278 - protein, 9 aa, Artificial Sequence
  /note=“Description of Artificial Sequence Synthetic peptide”
  MOD_RES (7)..(7): 3-amino-3-(2-nitro)phenyl-propionic acid
KMKEIAXAY

SEQ ID NO: 279 - protein, 9 aa, Artificial Sequence
  /note=“Description of Artificial Sequence Synthetic peptide”
  MOD_RES (5)..(5): 3-amino-3-(2-nitro)phenyl-propionic acid
KPWDXIPMV

SEQ ID NO: 280 - protein, 13 aa, Artificial Sequence
  /note=“Description of Artificial Sequence Synthetic peptide”
ATPLLMQALPMGA

SEQ ID NO: 281 - protein, 13 aa, Artificial Sequence
  /note=“Description of Artificial Sequence Synthetic peptide”
PKYVKQNTLKLAT

SEQ ID NO: 282 - protein, 10 aa, Artificial Sequence
  /note=“Description of Artificial Sequence Synthetic peptide”
ELAGIGILTV

SEQ ID NO: 283 - protein, 20 aa, Artificial Sequence
  /note=“Description of Artificial Sequence Synthetic peptide”
GGGGSGGGGSGGGGSGGGGS

SEQ ID NO: 284 - protein, 4 aa, Artificial Sequence
  /note=“Description of Artificial Sequence Synthetic peptide”
GGSG

SEQ ID NO: 285 - protein, 6 aa, Artificial Sequence
  /note=“Description of Artificial Sequence Synthetic 6xHis tag”
HHHHHH

SEQ ID NO: 286 - protein, 16 aa, Artificial Sequence
  /note=“Description of Artificial Sequence Synthetic peptide”
SGGGGSGGGGSGGGGS

SEQ ID NO: 287 - protein, 21 aa, Artificial Sequence
  /note=“Description of Artificial Sequence Synthetic peptide”
SGGGGSGGGGSGGGGSGGGGS

SEQ ID NO: 288 - protein, 13 aa, Artificial Sequence
  /note=“Description of Artificial Sequence Synthetic peptide”
DPFRLLQNSQVYS

SEQ ID NO: 289 - protein, 10 aa, Artificial Sequence
  /note=“Description of Artificial Sequence Synthetic peptide”
ELAGIGIMTV

SEQ ID NO: 290 - protein, 10 aa, Artificial Sequence
  /note=“Description of Artificial Sequence Synthetic peptide”
ELAGIGLLTV

SEQ ID NO: 291 - protein, 10 aa, Artificial Sequence
  /note=“Description of Artificial Sequence Synthetic peptide”
ELWGIGILTV

SEQ ID NO: 292 - protein, 10 aa, Artificial Sequence
  /note=“Description of Artificial Sequence Synthetic peptide”
EMAGIGILTV

SEQ ID NO: 293 - protein, 15 aa, Artificial Sequence
  /note=“Description of Artificial Sequence Synthetic peptide”
EQQWNFAGIEAAASA

SEQ ID NO: 294 - protein, 10 aa, Artificial Sequence
  /note=“Description of Artificial Sequence Synthetic peptide”
EVAGIGILTV

SEQ ID NO: 295 - protein, 15 aa, Artificial Sequence
  /note=“Description of Artificial Sequence Synthetic peptide”
GEEYLILSARDVLAV

SEQ ID NO: 296 - protein, 17 aa, Artificial Sequence
  /note=“Description of Artificial Sequence Synthetic peptide”
KRGLFGAIAGFIEGGWQ

SEQ ID NO: 297 - protein, 13 aa, Artificial Sequence
  /note=“Description of Artificial Sequence Synthetic peptide”
QEIYMQHTYPISA

SEQ ID NO: 298 - protein, 15 aa, Artificial Sequence
  /note=“Description of Artificial Sequence Synthetic peptide”
QGQWRGAAGTAAQAA

SEQ ID NO: 299 - protein, 13 aa, Artificial Sequence
  /note=“Description of Artificial Sequence Synthetic peptide”
RFYKTLRAEQASQ

SEQ ID NO: 300 - protein, 15 aa, Artificial Sequence
  /note=“Description of Artificial Sequence Synthetic peptide”
VRIFSTNQGGFMLPI

(19) United States
(12) Patent Application Publication
(10) Pub. No.: US 2023/0287394 A1
(43) Pub. Date: Sep. 14, 2023

(54) BARCODABLE EXCHANGEABLE PEPTIDE-MHC MULTIMER LIBRARIES

(71) Applicant: Repertoire Immune Medicines, Inc., Cambridge, MA (US)

(72) Inventors: Ohad Yosefson, Concord, MA (US); Thomas Malia, Medford, MA (US); Andrew Hamel, Cambridge, MA (US); Joanna Swain, Concord, MA (US); Benjamin Roscoe, Somerville, MA (US); Mohammad Rashidian, Cambridge, MA (US)

(21) Appl. No.: 17/995,074

(22) PCT Filed: Mar. 31, 2021
(86) PCT No.: PCT/US2021/025167

§ 371 Date: Sep. 29, 2022

Related U.S. Application Data
(60) Provisional application No. 63/003,177, filed on Mar. 31, 2020.

(51) Int. Cl. C12N 15/10; C40B 20/04; C07K 1/13; C07K 14/74

(52) U.S. Cl. CPC C12N 15/1065; C12N 15/1037; C40B 20/04; C07K 1/13; C07K 14/70539

(57) ABSTRACT

MHC multimers are provided in which peptide-loaded MHC monomers are covalently linked to a multimerization domain through conjugation moieties on the monomers and the multimerization domain. The multimers can further comprise oligonucleotide barcodes. Peptide exchange can be performed with a plurality of pMHC multimers to create pMHC multimer libraries. Methods of making and using the pMHC multimers and libraries are also provided. Peptide-loaded MHC Class I and MHC Class II multimers, and libraries thereof, are provided.

### RELATED APPLICATIONS

This application claims priority to U.S. Provisional Application No. 63/003,177, filed Mar. 31, 2020, the entire contents of which is hereby incorporated by reference.

### BACKGROUND

Identification of peptides recognized by individual T cells is important for the understanding and treatment of immune-related diseases, as well as vaccine development for prevention of diseases. Techniques for the detection of antigen-responsive T cells exploit the interaction between a given TCR and its peptide-MHC (pMHC) recognition motif. The ability to prepare soluble MHC molecules allowed for the preparation of soluble peptide-MHC complexes, which then can be made into multimeric complexes. T cell detection using multimerized pMHC molecules has become the preferred method for detecting antigen-specific T cells in a wide variety of research and clinical situations.

MHC multimers have been used for detection of antigen-responsive T cells since Altman et al. (Science 274:94-96, 1996) showed that tetramerization of peptide-loaded MHC class I (pMHCI) molecules provided sufficient stability to T cell receptor (TCR)-pMHC interactions, allowing detection of fluorescently-labeled MHC multimer-binding T cells using flow cytometry. However, since MHC Class I molecules are largely unstable when they are not part of a complex with peptide, pMHCI-based technologies were initially restricted by the tedious production of molecules in which each peptide required an individual folding and purification procedure (Bakker et al., Curr. Opin. Immunol. 17:428-433, 2005).

More recently, a variety of MHCI molecules with covalently linked peptides have been reported (e.g., reviewed by Goldberg et al., J. Cell. Mol. Med. 15:1822-1832, 2011). Several types of pMHCI microarrays systems also have been developed, but most work has focused on optimizing the supporting surface and modifying the conditions applied during binding and/or washing. The use of these systems is also limited due to poor detection limits and low reproducibility compared to existing cytometry-based analyses. For example, a general limitation to such array-based strategies is the propensity of a given T cell to pursue all potential pMHCI interactions displayed on a given array. As a consequence, the frequency of antigen-responsive T cells in the cell preparations typically needs to be >0.1% to allow a robust readout.

MHCI multimers, and libraries thereof, have been prepared using biotinylated peptide-MHCI monomers that then associate with the biotin-binding site on streptavidin to form tetramers (see e.g., Leisner et al., PLoS One 3(2):e1678, 2008). For the creation of MHC Class I libraries, approaches have been described in which oligonucleotide barcode labels have been conjugated to the streptavidin. However, existing strategies involve complex and/or costly approaches that limit the facile production of large libraries. For example, in one approach, individual streptavidin precursors must be barcoded individually by overlap extension PCR prior to tetramerization of biotinylated peptide-HLA monomers (Zhang et al., Nature Biotech. 2018; doi:10.1038.nbt.4282). In another approach, streptavidin-conjugated dextran, which is a costly reagent, is used to create a dextramer to which both the biotinylated peptide-HLA monomers and the biotinylated barcode oligonucleotide are complexed (Bentzen et al., Nature Biotech. 34:10: 1037-1045, 2016) via the streptavidin conjugated to the dextran backbone.

Similar to the approach with pMHCI tetramers, soluble MHC class II molecules also have been used to prepare pMHCII tetramers, which have been used in the study of the antigenic specificity of CD4+ T helper cells (as reviewed in, for example, Nepom et al. (2002) Arthrit. Rheumat. 46:5-12; Vollers and Stern (2008) Immunol. 123:305-313; Cecconi et al. (2008) Cytometry 73A:1010-1018). Typically to prepare pMHCII multimers, soluble biotinylated MHCII α/β dimers are recombinantly expressed and then tetramerized by binding to streptavidin or avidin through their biotin-binding sites. Fluorescent labeling of the streptavidin or avidin then allows for isolation of T cells that bind the pMHCII multimers by flow cytometry. With regard to antigenic peptide loading of the MHCII molecules, in one approach, a peptide is attached to the MHCII α/β dimers covalently. Some groups have generated pMHCII loaded with a covalent but cleavable “stuffer” peptide that can be exchanged with a peptide of interest under acidic conditions (Day et al., J Clin Invest. 2003; 112(6):831-842).

In an alternative approach, “empty” MHCII α/β dimers are prepared and then loaded with soluble MHCII-binding peptides (see e.g., Novak et al. (1999) J. Clin. Invest. 104:63-67; Nepom et al. (2002) Arthrit. Rheumat. 46:5-12; Macaubus et al. (2006) J. Immunol. 176:5069-5077). While this approach allows for greater diversity of peptide loading onto the MHCII α/β dimers, the ability to recombinantly express stable “empty” MHCII α/β dimers is limited, thus again hampering the preparation of large scale pMHCII multimer libraries. For example, production of “empty” MHCII α/β dimers by refolding from E. coli inclusion bodies or by insect cell or mammalian cell expression has been reported, but with yields that are too low to support high throughput methods (reviewed in Vollers and Stern (2008) Immunology 123: 305-313).

Accordingly, there remains a need for efficient and cost effective methods of generating peptide-MHC libraries, including barcoded libraries, which may be utilized in a variety of methods, for example, screening of T cell specificity for analyses of T cell recognition, for example, at genome-wide levels rather than analyses restricted to a selection of model antigens.

### SUMMARY

The present disclosure provides methods for producing barcoded, peptide loaded MHC (pMHC) multimers (e.g., tetramers), including libraries thereof. The methods provide high protein yields of pMHC multimers within a short time period using efficient reaction conditions that allow for ease of peptide exchange and barcode labeling of the multimers to thereby allow for efficient preparation of large pMHC multimer libraries. Accordingly, the compositions and methods described herein are suitable for routine laboratory research, as well as large scale industrial and clinical applications, in all circumstances where pMHC multimers are useful. In one embodiment, the pMHC multimer is a pMHC Class I (pMHCI) multimer, which is useful for analysis of CD8+ T cell antigen recognition. In another embodiment, the pMHC multimer is a pMHC Class II (pMHCII) multimer, which is useful for analysis of CD4+ T cell antigen recognition. The MHC multimers of the invention comprise a covalent linkage between the MHC monomers and the multimerization domain, thereby allowing a non-covalent binding site(s) on the multimerization domain to be easily used for barcode labeling.

In one aspect, the disclosure provides a method of producing a peptide-loaded MHC (pMHC) multimer comprising two or more peptide-loaded MHC (pMHC) monomers, wherein each of the pMHC monomers is covalently linked to a multimerization domain. In particular, the pMHC monomers are linked to the multimerization domain through a chemical linkage that is not a biotin/streptavidin or biotin/avidin interaction, which linkage is achieved in an efficient bulk chemical reaction. This chemical linkage is achieved through the use of conjugation moieties on the pMHC monomers and the multimerization domain, which moieties then react to form the chemical linkage. Peptide exchange and oligonucleotide barcode labeling can then easily be performed on the pMHC multimers, allowing for efficient large-scale pMHC library production.

Accordingly, in one aspect, the disclosure provides a method of producing a Major Histocompatibility Complex (MHC) multimer, the method comprising:
(a) providing two or more MHC monomers, wherein each monomer comprises a conjugation moiety;
(b) providing a multimerization domain, wherein each subunit of the multimerization domain comprises a conjugation moiety;
(c) combining the MHC monomers and the multimerization domain under conditions sufficient for covalent conjugation between the MHC monomers and the multimerization domain to produce an MHC multimer.

In one embodiment, the MHC monomers are MHC Class I monomers. In another embodiment, the MHC monomers are MHC Class II monomers. In certain embodiments, the MHC monomers are loaded with a placeholder peptide prior to combining with the multimerization domain.

In one embodiment, the multimerization domain comprising a non-covalent binding site, wherein the method further comprises that the MHC multimer is labeled with an oligonucleotide barcode that binds the non-covalent bindings site of the multimerization domain.

In one embodiment, the multimer is a tetramer. In one embodiment, the multimerization domain is streptavidin. In one embodiment, the multimerization domain is streptavidin and the oligonucleotide barcode binds the biotin-binding site of streptavidin.

With respect to the covalent linkage between the MHC monomers and the multimerization domain, in one embodiment, the conjugation moiety of each MHC monomer comprises X, and the conjugation moiety of each subunit of the multimerization domain comprises Y, wherein
(i) X is a terminal alkyne and Y is an azide;
(ii) X is an azide and Y is a terminal alkyne;
(iii) X is a strained alkyne and Y is an azide;
(iv) X is an azide and Y is a strained alkyne;
(v) X is a diene and Y is a dienophile;
(vi) X is a dienophile and Y is a diene;
(vii) X is a thiol and Y is an alkene; or
(viii) X is an alkene and Y is a thiol.

In one embodiment, the azide is a copper-chelating azide. In one embodiment, the copper-chelating azide is a picolyl azide.

In one embodiment, the conjugation moiety of each MHC monomer and the conjugation moiety of each subunit of the multimerization domain comprise a sortag motif.

In one embodiment, the conjugation moiety of each MHC monomer and the conjugation moiety of each subunit of the multimerization domain comprise an intein sequence.

In one embodiment, the method further comprises exchanging the placeholder peptide with a rescue peptide epitope that binds the MHC monomers.

In another aspect, the disclosure pertains to a barcode-labeled MHC multimer comprising:
(a) two or more MHC monomers;
(b) a multimerization domain comprising two or more subunits and having at least one non-covalent binding site; and
(c) an oligonucleotide barcode;
   wherein each MHC monomer is bound to a subunit of the multimerization domain through a covalent linkage; and wherein the oligonucleotide barcode is bound to the multimerization domain by non-covalent binding to the non-covalent binding site on the multimerization domain.

In one embodiment, the MHC multimer further comprises an MHC-binding peptide loaded onto each MHC monomer of the multimer. In one embodiment, the MHC monomers are MHC Class I monomers. In one embodiment, the MHC monomers are MHC Class II monomers.

In one embodiment, the MHC multimer is a tetramer. In one embodiment, the multimerization domain is streptavidin. In one embodiment, the oligonucleotide barcode is non-covalently bound to the biotin binding site on streptavidin.

In one embodiment of the MHC multimer, each MHC monomer comprises a conjugation moiety X, and each subunit of the multimerization domain comprises a conjugation moiety Y, wherein
(i) X is a terminal alkyne and Y is an azide;
(ii) X is an azide and Y is a terminal alkyne;
(iii) X is a strained alkyne and Y is an azide;
(iv) X is an azide and Y is a strained alkyne;
(v) X is a diene and Y is a dienophile;
(vi) X is a dienophile and Y is a diene;
(vii) X is a thiol and Y is an alkene; or
(viii) X is an alkene and Y is a thiol.

In one embodiment, the azide is a copper-chelating azide. In one embodiment, the copper-chelating azide is a picolyl azide.

In one embodiment of the MHC multimer, each MHC monomer and each subunit of the multimerization domain comprises a conjugation moiety, wherein each conjugation moiety comprises a sortag motif.

In one embodiment of the MHC multimer, each MHC monomer and each subunit of the multimerization domain comprises a conjugation moiety, wherein each conjugation moiety comprises an intein sequence.

In yet another aspect, the disclosure pertains to methods of preparing MHC Class I multimers. In one embodiment, a method of producing a pMHCI multimer is provided, the method comprising: (a) providing two or more placeholder peptide loaded MHCI (p*MHCI) monomers each comprising (i) an MHCI heavy chain polypeptide, or a functional fragment thereof, (ii) a β2-microglobulin polypeptide or functional fragment thereof, (iii) a conjugation moiety, and (iv) a placeholder peptide bound in the peptide binding groove of each MHCI monomer; (b) providing a multimerization domain, wherein each subunit of the multimerization domain comprises a conjugation moiety; (c) combining the p*MHCI monomers and the multimerization domain under conditions sufficient for covalent conjugation between the two or more p*MHCI monomers and the multimerization domain to produce a p*MHCI multimer; and (d) replacing the placeholder peptide bound in the peptide binding groove of each of the p*MHCI monomers in the p*MHCI multimer with a rescue peptide epitope to produce a pMHCI multimer. The method can further comprise labeling the pMHCI multimers with oligonucleotide barcodes by reacting the multimer with barcoded oligonucleotides comprising a binding moiety that binds to the pMHCI multimer, e.g., to the multimerization domain of the pMHCI multimer.

In another aspect, a method of producing a barcoded, peptide pMHCI multimer is provided, the method comprising:
(a) providing two or more placeholder peptide loaded MHCI (p*MHCI) monomers each comprising (i) an MHCI heavy chain polypeptide, or a functional fragment thereof (ii) a β2-microglobulin polypeptide or functional fragment thereof, (iii) a conjugation moiety, and (iv) a placeholder peptide bound in the peptide binding groove of each MHCI monomer;
(b) providing a multimerization domain, wherein each subunit of the multimerization domain comprises a conjugation moiety and the multimerization domain comprises at least one non-covalent binding site;
(c) combining the p*MHCI monomers and the multimerization domain under conditions sufficient for covalent conjugation between the two or more p*MHCI monomers and the multimerization domain to produce a p*MHCI multimer;
(d) replacing the placeholder peptide bound in the peptide binding groove of each of the p*MHCI monomers in the p*MHCI multimer with a rescue peptide epitope to produce a pMHCI multimer; and
(e) binding an oligonucleotide barcode to the non-covalent binding site on the multimerization domain.

In a further aspect, a method of producing a pMHCI multimer is provided, the method comprising:
(a) providing two or more placeholder peptide loaded MHCI (p*MHCI) monomers each comprising (i) an MHCI heavy chain polypeptide, or a functional fragment thereof, (ii) a β2-microglobulin polypeptide or functional fragment thereof, (iii) a peptide linker comprising a conjugation moiety at the C-terminus of (i) or (ii); and (iv) a placeholder peptide bound in the peptide binding groove of each MHCI monomer;
(b) providing a multimerization domain comprising a peptide linker comprising a conjugation moiety at the C-terminus of each subunit of the multimerization domain;
(c) combining the p*MHCI monomers and the multimerization domain under conditions sufficient for covalent conjugation between two or more p*MHCI monomers to the multimerization domain to produce a p*MHCI multimer; and
(d) replacing the placeholder peptide bound in the peptide binding groove of each of the p*MHCI monomers in the p*MHCI multimer with a rescue peptide epitope to produce a pMHCI multimer.

Any suitable p*MHC monomer can be used in the methods described herein. In one embodiment, the p*MHC monomer is of vertebrate origin. In another embodiment, the p*MHCI monomer comprises a human MHCI heavy chain polypeptide or functional fragment thereof, and a human β2-microglobulin polypeptide or functional fragment thereof. In another embodiment, each p*MHCI monomer thereof is HLA-A, HLA-B or HLA-C. In another embodiment, each p*MHCI monomer is HLA-A. In another embodiment, each p*MHCI monomer is soluble.

In another embodiment, the MHCI heavy chain polypeptide, or functional fragment of thereof, of each p*MHCI monomer comprises an MHCI α1 domain. In another embodiment, the MHCI heavy chain polypeptide, or functional fragment thereof, of each p*MHCI monomer comprises an MHCI α1/α2 heterodimer. In another embodiment, the MHCI heavy chain polypeptide, or functional fragment thereof, of each p*MHCI monomer comprises an MHCI α1, α2 and an α3 domain. In another embodiment, the MHCI heavy chain polypeptide, or functional fragment thereof, of each p*MHCI monomer comprises an α domain that is at least 80, 85, 90, 95, or 99% identical to any of the amino acid sequence shown SEQ ID NOs: 28-159.

In another embodiment, each p*MHCI monomer comprises a β2-microglobulin domain. In one embodiment, the β2-microglobulin polypeptide of each p*MHCI monomer is a wild-type human β2-microglobulin. In another embodiment, the β2-microglobulin polypeptide comprises an amino acid sequence that is at least 80, 85, 90, 95, or 99% identical to the amino acid sequence of human β2-microglobulin (such as the amino acid sequence shown in SEQ ID NOs: 2 or 160).

In another embodiment, each p*MHC monomer is a fusion protein. For example, in one embodiment, each p*MHC monomer is a fusion protein comprising an MHCI heavy chain or functional fragment thereof and β2-microglobulin or functional fragment thereof. In another embodiment, the p*MHC fusion protein comprises a peptide linker between the MHCI heavy chain or functional fragment thereof and the β2-microglobulin polypeptide or functional fragment thereof.

Any suitable placeholder peptide can be used in the methods described herein. In one embodiment, the placeholder peptide is a peptide or peptide-like compound which promotes folding of the MHCI polypeptide. In one embodiment, the placeholder peptide is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 amino acids. In another embodiment, the placeholder peptide is 2 to 25 amino acids. In another embodiment, the placeholder peptide is 8 to 11 amino acids. In another embodiment, the placeholder peptide is 9 amino acids. In another embodiment, the placeholder peptide is 10 amino acids. In another embodiment the placeholder peptide comprises GILGFVFJL (SEQ ID NO:7). In another embodiment the placeholder peptide consists of GILGFVFJL (SEQ ID NO:7)). In other embodiments, the placeholder peptide has a sequence shown in any one of SEQ ID NOs: 8-27 or 271-279.

In another embodiment, the placeholder peptide has a lower affinity for the MHCI peptide binding groove than the exchanged peptide epitope, and wherein step (d) comprises contacting the p*MHCI monomer with an excess of peptide epitope in a competition assay. In another embodiment, the placeholder peptide has a KD that is about 4-fold, 5-fold, 6-fold, 7-fold, 8-fold, 9-fold, 10-fold, 11-fold, 12-fold, 13-fold, 14-fold, or 15-fold lower than the exchanged peptide epitope. In another embodiment, the placeholder peptide has a KD that is about 10-fold lower than the exchanged peptide epitope. In another embodiment, the placeholder peptide has a higher affinity for the MHCI binding groove than the exchange peptide epitope.

In another embodiment, the placeholder peptide is labile at a temperature between about 30-37° C., and step (d) comprises exposing the p*MHCI monomer to a temperature of between about 30-37° C. (e.g., 30° C., 31° C., 32° C., 33° C., 34° C., 35° C., 36° C., or 37° C.) in the presence of peptide epitope. In another embodiment, the placeholder peptide is labile at an acidic pH of between about pH 2.0-5.5 (e.g., pH 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2. 5.3, 5.4, or 5.5). In another embodiment, the p*MHCI monomer is exposed to a pH of between about pH 2.0-5.5 (e.g., pH 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2. 5.3, 5.4, or 5.5) in the presence of peptide epitope. In another embodiment, the placeholder peptide is labile at an acidic pH of between about pH 2.0-5.5 (e.g., pH 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2. 5.3, 5.4, or 5.5), and step (d) comprises exposing the p*MHCI monomer to a pH of between about pH 2.0-5.5 (e.g., pH 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2. 5.3, 5.4, or 5.5) in the presence of peptide epitope.

In another embodiment, the placeholder peptide is labile at a basic pH of between about pH 9-11 (e.g., 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, or 11). In another embodiment, the placeholder peptide is labile at a basic pH of between about pH 9-11 (e.g., 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, or 11) and the p*MHCI monomer is exposed to a pH of between about pH 9-11 (e.g., 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, or 11) in the presence of peptide epitope. In another embodiment, the placeholder peptide is labile at a basic pH of between about pH 9-11 (e.g., 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, or 11) and step (d) comprises exposing the p*MHCI monomer to a pH of between about pH 9-11 (e.g., 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, or 11) in the presence of peptide epitope.

In some embodiments, the placeholder peptide comprises GILGFVFJL (SEQ ID NO:7). In some embodiments, the placeholder peptide consists of GILGFVFJL (SEQ ID NO:7). In other embodiments, the placeholder peptide has a sequence shown in any one of SEQ ID NOs: 8-27 or 271-279.

In one embodiment, the placeholder peptide comprises a cleavable moiety. In one embodiment, the method comprises contacting the p*MHCI monomer with peptide epitope under conditions sufficient to cleave the placeholder peptide. Any suitable cleavable moiety can be used. In one embodiment, the cleavable moiety is a photocleavable amino acid, and the method (e.g., step (d)) comprises exposing the p*MHCI monomer to UV-light under conditions sufficient to induce cleavage of the photocleavable moiety in the placeholder peptide and binding of the peptide epitope to the MHCI monomer. In one embodiment, the photocleavable amino acid comprises a (2-nitro)phenyl side chain. In another embodiment, the photocleavable amino acid comprises 3-amino-3-(2-nitrophenyl)proprionic acid. In another embodiment, the photocleavable amino acid is (2-nitro)phenylglycine.

In other embodiments, the photocleavable placeholder peptide, and the corresponding MHC molecule to which it binds, is selected from A*02:01, KILGFVFJV (SEQ ID NO: 15) or GILGFVFJL (SEQ ID NO: 7), A*01:01, STAPGJLEY (SEQ ID NO: 16); A*02:03, SVRDJLARL (SEQ ID NO: 271); A*02:06, LTAJFLIFL (SEQ ID NO: 272); A*02:07, LLDSDJERL (SEQ ID NO: 273); A*02:11, KMDIJVPLL (SEQ ID NO: 274); A*03:01, RIYRJGATR (SEQ ID NO:17); A*11:01, RVFAJSFIK (SEQ ID NO: 18); A*24:02, VYGJVRACL (SEQ ID NO: 11); A*33:03, FYVJGAANR (SEQ ID NO: 275); B*07:02, AARGJTLAM (SEQ ID NO: 14); B*15:02, ILGPPGJVY (SEQ ID NO: 276); B*35:01, KPIVVLJGY (SEQ ID NO: 19); B*44:05, EEFGAAJSF (SEQ ID NO: 277); B*46:01, KMKEIAJAY (SEQ ID NO: 278); B*55:02, KPWDJIPMV (SEQ ID NO: 279); C*03:04, FVYGJSKTSL (SEQ ID NO: 20), B*08:01, FLRGRAJGL (SEQ ID NO: 21); C*07:02, VRIJHLYIL (SEQ ID NO: 22); C*04:01, QYDJAVYKL (SEQ ID NO: 23); B*15:01, ILGPJGSVY (SEQ ID NO: 24); B*40:01, TEADVQJWL (SEQ ID NO: 25); B*58:01, ISARGQJLF (SEQ ID NO: 26); and C*08:01, KAAJDLSHFL (SEQ ID NO: 27), wherein J is 3-amino-3-(2-nitrophenyl)propionic acid.

In another embodiment, the cleavable moiety is an amino acid comprising a chemoselective moiety. In another embodiment, the method (e.g., step (d)) comprises contacting the p*MHCI monomer with peptide epitope under conditions sufficient to cleave the chemoselective moiety. In another embodiment, the chemoselective moiety is a sodium dithionite sensitive azobenzene linker. In another embodiment, the method (e.g., step (d)) comprises contacting the p*MHCI monomer with peptide epitope in the presence of sodium diothionite.

In another embodiment, the cleavable moiety is a periodate-sensitive amino acid. In another embodiment, the method (e.g., step (d)) comprises contacting the p*MHCI monomer with peptide epitope in the presence of periodate under conditions sufficient to cleave the placeholder peptide. In another embodiment, the periodate-sensitive amino acid comprises a vicinal diol moiety. In another embodiment, the periodate-sensitive amino acid comprises a vicinal amino alcohol. In another embodiment, the periodate-sensitive amino acid is α,γ-diamino-β-hydroxybutanoic acid (DAHB).

In another embodiment, the cleavable moiety is a protease recognition moiety. In one embodiment the protease is an amino-peptidase. In another embodiment, the protease is a methionine amino-peptidase. In yet other embodiments, the protease is selected from FXa, thrombin, TEV, HRV3C and furin.

In one embodiment, the placeholder peptide is a dipeptide. In another embodiment, the dipeptide binds to the F pocket of the MHCI binding groove. In another embodiment, the second amino acid of the dipeptide is hydrophobic. In another embodiment, the dipeptide is selected from the group consisting of glycyl-leucine (GL), glycyl-valine (GV), glycyl-methione (GM), glycyl-cyclohexylalanine (GCha), glycyl-homoleucine (GHle) and glycyl-phenylalanine (GF).

Any suitable multimerization domain can be used. In one embodiment, each subunit of the multimerization domain comprises a conjugation moiety. In another embodiment, the multimerization domain comprises a peptide linker comprising a conjugation moiety at the N-terminus of each subunit of the multimerization domain. In another embodiment, the multimerization domain comprises a peptide linker comprising a conjugation moiety at the C-terminus of each subunit of the multimerization domain. In one embodiment, the multimerization domain is a dimer, tetramer, hexamer, octamer, decamer or dodecamer. In another embodiment, the multimerization domain is a homomultimer. In another embodiment, the multimerization domain is a heteromultimer. In another embodiment, the multimerization domain comprises streptavidin or a derivative thereof. In another embodiment, the multimerization domain is a tetramer of streptavidin or a derivative thereof. In another embodiment, the multimerization domain comprises Strep-tag® or Strep-tactin®.

In one embodiment, the conjugation moiety is attached to the C-terminus of the MHCI heavy chain α1 domain of each p*MHCI monomer. In another embodiment, the multimerization domain is covalently conjugated to the C-terminus of the MHCI α1 domain. In another embodiment, the conjugation moiety is attached to the C-terminus of the MHCI heavy chain α2 domain of each p*MHCI. In another embodiment, the multimerization domain is covalently conjugated to the C-terminus of the MHCI α2 domain. In another embodiment, the conjugation moiety is attached to the C-terminus of the MHCI heavy chain α3 domain of each p*MHCI. In another embodiment, the multimerization domain is covalently conjugated to the C-terminus of the MHCI α3 domain. In another embodiment, the conjugation moiety is attached to the C-terminus of β2-microglobulin of each p*MHC monomer of each p*MHCI monomer. In another embodiment, multimerization domain is covalently conjugated to the C-terminus of the β2-microglobulin of each p*MHC monomer. In another embodiment, the covalent conjugation of each p*MHCI monomer to the multimerization domain is mediated by a cysteine transpeptidase (e.g., a sortase, or an enzymatically active fragment thereof).

In another embodiment, two or more p*MHC monomers are chemically conjugated to the multimerization domain. In another embodiment, the chemical conjugation is mediated by cysteine bioconjugation. In another embodiment, the chemical conjugation is mediated by native chemical conjugation. In another embodiment, the chemical conjugation is mediated by click chemistry.

In another embodiment, the conjugation moiety of each p*MHCI monomer comprises X, and the conjugation moiety of each subunit of the multimerization domain comprises Y. For example, in one embodiment, X is a terminal alkyne and Y is an azide. In another embodiment, X is an azide and Y is a terminal alkyne. In another embodiment, X is a strained alkyne and Y is an azide. In another embodiment, X is an azide and Y is a strained alkyne. In certain embodiments, the azide is a copper-chelating azide. In one embodiment, the copper-chelating azide is a picolyl azide. In another embodiment, X is a diene and Y is a dienophile. In another embodiment, X is a dienophile and Y is a diene. In another embodiment, X is a thiol and Y is an alkene. In another embodiment, X is an alkene and Y is a thiol.

In another embodiment, the conjugation moiety of each p*MHC domain comprises a peptide linker attached to the C-terminus, and the conjugation moiety of each subunit of the multimerization domain comprises a peptide linker attached the C-terminus of each subunit of the multimerization domain. In another embodiment, the peptide linker at the C-terminus of each p*MHC monomer comprises (G)n-X, wherein n is at least 2, and X is a moiety suitable for chemical conjugation, and the peptide linker at the C-terminus of each subunit of the multimerization domain comprises (G)n-Y, wherein n is at least 2, and Y is a moiety suitable for chemical conjugation to the X moiety of each p*MHC monomer.

In another embodiment, the conjugation moiety of each p*MHCI monomer comprises a C-terminal sortag and the conjugation moiety of each subunit of the multimerization domain comprises an N-terminal sortag. In another embodiment, the conjugation moiety of each p*MHCI monomer comprises an N-terminal sortag and the conjugation moiety of each subunit of the multimerization domain comprises an C-terminal sortag. In another embodiment, the method (e.g., step (c) of the method of preparing the MHC multimers set forth above) comprises the addition of sortase to a mixture of p*MHCI monomers and multimerization domains and catalyzes the formation of a peptide bond between each p*MHC monomers and the multimerization domain to produce a p*MHC multimer.

In another embodiment, two or more p*MHCI monomers (e.g., in step (a) of the method of preparing the MHC multimer set forth above) are produced by contacting p*MHCI monomers comprising a C-terminal sortag with the sortase, or an enzymatically active fragment thereof, in the presence of a peptide linker comprising a moiety suitable for chemical conjugation, wherein the sortase, or enzymatically active fragment thereof, mediates the conjugation of the peptide linker to the p*MHC monomers; the multimerization domain (e.g., in step (b) in the method set forth above) is produced by contacting a multimerization domain comprising an N-terminal sortag with the sortase in the presence of a peptide linker comprising a moiety suitable for chemical conjugation wherein the sortase, or enzymatically active fragment thereof, mediates the conjugation of the peptide linker to the N-terminus of each subunit of the multimerization domain; and step (c) comprises chemical conjugation between the peptide linker at the C-terminus of the two or more p*MHC monomers and the peptide linker at the N-terminus of each subunit of the multimerization domain to produce the p*MHC multimer.

In one embodiment, the peptide linker at the C-terminus of each p*MHC monomer comprises (G)n-X, wherein n is at least 2, and X is a moiety suitable for click chemistry conjugation, and the peptide linker at the N-terminus of each subunit of the multimerization domain comprises Y-(G)n wherein n is at least 2, and Y is a moiety suitable for click chemistry conjugation with the X moiety of each p*MHC monomer. In another embodiment, X is a terminal alkyne and Y is an azide. In another embodiment, X is an azide and Y is a terminal alkyne. In another embodiment, X is a strained alkyne and Y is an azide. In another embodiment, X is an azide and Y is a strained alkyne. In certain embodiments, the azide is a copper-chelating azide. In one embodiment, the copper-chelating azide is a picolyl azide. In another embodiment, X is a diene and Y is a dienophile. In another embodiment, X is a dienophile and Y is a diene. In another embodiment, X is a thiol and Y is an alkene. In another embodiment, X is an alkene and Y is a thiol.

In one embodiment, the sortase, or enzymatically active fragment thereof is Ca2+ dependent. In another embodiment, the sortase, or enzymatically active fragment thereof is Ca2+ independent. In another embodiment, the sortase, or enzymatically active fragment thereof is a soluble fragment of the wild-type sortase. In another embodiment, the sortase, or enzymatically active fragment thereof is a variant or homolog of S. aureus sortase A. In another embodiment, the sortase, or enzymatically active fragment thereof is modified sortase A. In another embodiment, the sortase, or enzymatically active fragment thereof is a SrtAstaph mutant. In another embodiment, the SrtAstaph mutant is selected from the group consisting of F40, SrtAstaph pentamutant, 2A-9, and 4S-9.

In one embodiment, the covalent conjugation of each p*MHCI monomer to the multimerization domain is mediated by an intein. In one embodiment, the intein is selected from the group consisting of MxeGyrA, SspDnaE, NpuDnaE, AvaDnaE, Cfa (consensus DnaE split intein), gp41-1, gp41-8 and NrdJ-1. In another embodiment, the intein is a split intein pair. In another embodiment, each p*MHCI monomer is conjugated to the multimerization domain by an intein peptide tag. In another embodiment, each p*MHCI monomer comprises an N-intein fragment at the C-terminus, and each subunit of the multimerization domain comprises an Npu-C-intein fragment at the N-terminus.

In one embodiment, the rescue peptide epitope comprises an identifier. In one embodiment, the identifier is a nucleic acid identifier. In one embodiment, the identifier is a nucleic acid identifier. In another embodiment, the nucleic acid identifier encodes the peptide. In another embodiment, the nucleic acid identifier is from 25 nucleotides to 500 nucleotides in length. In another embodiment, the nucleic acid identifier is from 80 nucleotides to 120 nucleotides in length.

In one aspect, the disclosure provides a method of producing a library of diverse pMHCI multimers and methods for their production, including high-throughput methods. In some embodiments, the pMHC multimers further comprise nucleic acid identifiers, allowing for convenient detection and quantification of binding as described elsewhere herein.

In one embodiment, a method of producing a library comprising a diversity of peptide epitope loaded MHCI (pMHCI) multimers is provided, the method comprising:
(a) providing a plurality of placeholder peptide loaded MHCI (p*MHCI) monomers each comprising (i) an MHCI heavy chain polypeptide, or a functional fragment thereof, (ii) a β2-microglobulin polypeptide or functional fragment thereof, (iii) a conjugation moiety, and (iv) a placeholder peptide bound in the peptide binding groove of each MHCI monomer;
(b) providing a plurality of multimerization domains, wherein each subunit of the multimerization domain comprises a conjugation moiety;
(c) combining the p*MHCI monomers and the multimerization domains under conditions sufficient for covalent conjugation between the two or more p*MHCI monomers and a multimerization domain to produce p*MHCI multimers; and
(d) replacing the placeholder-peptide in the plurality of p*MHCI multimers with a peptide library comprising a plurality of unique MHCI peptide epitopes to produce a plurality of peptide loaded MHCI (pMHCI) multimers.

In another aspect, a method of producing a library comprising a diversity of barcoded peptide loaded Major Histocompatibility Complex Class I (pMHCI) multimers is provided, the method comprising:
(a) providing a plurality of placeholder peptide loaded MHCI (p*MHCI) monomers each comprising (i) an MHCI heavy chain polypeptide, or a functional fragment thereof, (ii) a β2-microglobulin polypeptide or functional fragment thereof, (iii) a conjugation moiety, and (iv) a placeholder peptide bound in the peptide binding groove of each MHCI monomer;
(b) providing a plurality of multimerization domains, wherein each subunit of the multimerization domains comprises a conjugation moiety and the multimerization domain comprises at least one non-covalent binding site;
(c) combining the plurality of p*MHCI monomers and the plurality of multimerization domain under conditions sufficient for covalent conjugation between the two or more p*MHCI monomers and a multimerization domain to produce a plurality of p*MHCI multimers;
(d) replacing the placeholder peptide bound in the peptide binding groove of the p*MHCI multimers with a plurality of unique rescue peptide epitopes to produce a plurality of pMHCI multimers; and
(e) binding an oligonucleotide barcode to the non-covalent binding site on the multimerization domain.

In another aspect, a method of producing a library comprising a diversity of barcoded peptide loaded Major Histocompatibility Complex Class I (pMHCI) multimers is provided, the method comprising:
(a) providing a plurality of placeholder peptide loaded MHCI (p*MHCI) monomers each comprising (i) an MHCI heavy chain polypeptide, or a functional fragment thereof, (ii) a β2-microglobulin polypeptide or functional fragment thereof, (iii) a peptide linker comprising a conjugation moiety at the C-terminus of (i) or (ii); and (iv) a placeholder peptide bound in the peptide binding groove of each MHCI monomer;
(b) providing a plurality of multimerization domains comprising a peptide linker comprising a conjugation moiety at the C-terminus of each subunit of the multimerization domain;
(c) combining the plurality of p*MHCI monomers and the plurality of multimerization domains under conditions sufficient for covalent conjugation between two or more p*MHCI monomers to a multimerization domain to produce a plurality of p*MHCI multimers; and
(d) replacing the placeholder peptide bound in the peptide binding groove of the p*MHCI multimers with a plurality of unique rescue peptide epitopes to produce a plurality of pMHCI multimers and
(e) binding an oligonucleotide barcode to the non-covalent binding site on the multimerization domain.

In another aspect, the disclosure provides a library of peptide-loaded MHC Class I (pMHC) multimers, wherein each pMHC multimer in the library comprises two or more pMHC monomers loaded with a unique peptide epitope, and wherein each pMHC monomer is covalently linked to a subunit of a multimerization domain.

In one embodiment, the library of MHCI peptide epitopes is a high diversity peptide library. In another embodiment, the peptide library comprises between about 10³ and 10²⁰ different MHC I peptide epitopes. In another embodiment, the peptide library comprises about 10³, about 10⁴, about 10⁵, about 10⁶, about 10⁷, about 10⁸, about 10⁹, about 10¹⁰, about 10¹¹, about 10¹², about 10¹³, about 10¹⁴, about 10¹⁵, about 10¹⁶, about 10¹⁷, about 10¹⁸, about 10¹⁹, about 10²⁰, or more different MHCI peptide epitopes.

In one embodiment, the MHCI peptide epitopes are derived from a single antigenic protein. In another embodiment, the MHCI peptide epitopes comprise overlapping fragments of an antigenic protein. In another embodiment, the plurality of unique peptide epitopes is generated from a genome of an organism, a transcriptome of an organism, a proteome of an organism, or a peptide or protein of an organism. In another embodiment, the plurality of unique peptide epitopes is generated from differential sequences between two genomes. In another embodiment, the MHC peptide epitopes can be altered peptide ligands (APLs) of a particular peptide epitope of interest.

In another embodiment, each of the pMHC multimers comprises a unique identifier moiety. In one embodiment, the unique identifier moiety is a nucleic acid.

In another aspect, a polypeptide library comprising a plurality of peptide loaded MHCI (pMHCI) multimers is provided, wherein each of the peptide loaded pMHCI multimers comprises two or more pMHCI monomers conjugated to a multimerization domain.

In another aspect, a method of isolating MHC-multimer bound lymphocytes is provided, wherein the method comprises:
(a) contacting a plurality of lymphocytes with a library of pMHCI multimers; and
(b) generating a plurality of compartments, wherein each compartment comprises a lymphocyte bound to a pMHCI multimer of the library, and a capture support. In one embodiment, the lymphocyte is a T cell, B cell, or NK cell.

In another embodiment, a method of identifying a lymphocyte bound to an pMHC multimer comprising is provided, wherein the method comprises:
(a) contacting a plurality of lymphocytes with a library of pMHCI multimers;
(b) compartmentalizing a lymphocyte of the plurality of lymphocytes bound to a pMHCI multimer of the library in a single compartment, wherein the pMHCI multimer comprises a unique identifier; and
(c) determining the unique identifier for the pMHCI bound to the compartmentalized lymphocyte.

For a fuller understanding of the nature and advantages of the present disclosure, reference should be had to the ensuing detailed description taken in conjunction with the accompanying figures. The present disclosure is capable of modification in various respects without departing from the present disclosure. Accordingly, the figures and description of these embodiments are not restrictive.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 exemplifies various click chemistry handles and reactions.

FIG. 2 illustrates various peptide exchange methods.

FIG. 3A-3E show SDS-PAGE or Western Blot analysis of conjugation reactions. Cartoon images depict SAv tetramer linked to one, two, three or four HLA molecules. Arrows indicate undesired side-products. FIG. 3A: Anti-His Western Blot analysis of SAv-conjugation reaction. A description of each lane is shown in the table. The extent of reaction is approximately 94-97% based on comparison with reference SA protein. FIG. 3B: SDS-PAGE image of HLA-A2-DBCO-SAv-Az. Lane 1: SeeBlue Plus Protein Standard, Lane 2: SA-Az (non-boiled), Lane 3: SA-Az (boiled) Lane 4: HLA-A2-DBCO-SAv-Az (non-boiled, non-reduced), Lane 5: HLA-A2-DBCO-SAv-Az (boiled, reduced). FIG. 3C: SDS-PAGE image of HLA-A2-Az-SAv-DBCO. Lane 1: SeeBlue Plus Protein Standard, Lane 2: HLA-A2-Az (non-boiled), Lane 3: HLA-A2-Az-SAv-DBCO, (non-boiled), Lane 4-7: HLA-A2-Az-SAv-DBCO reactions (non-boiled). FIG. 3D: SDS-PAGE image of HLA-A2-Alk-SAv-Az. Lane 1: SeeBlue Plus Protein Standard, Lane 3: HLA-A2-Alk-SAv-Az (non-boiled, non-reduced), Lane 5: HLA-A2-Alkyne-SAv-Az (boiled, reduced). FIG. 3E: SDS-PAGE images of HLA-A*01:01, HLA-A*03:01 and HLA-A*24:02 in the Conjugated Tetramer format. Samples were either non-boiled/non-reduced (NB/NR) or boiled/reduced (boiled/R).

FIG. 4. SDS-PAGE analysis of the intein splicing reaction between HLA-A2-N-intein/β2m/peptide complex and SAv-C-intein.

FIGS. 5A and 5B illustrates UV exchange monitored by differential scanning fluorimetry. FIG. 5A shows differential scanning fluorimetry (DSF) of HLA-A*02:01-Alk-SAv-Az Conjugated Tetramers produced as in Example 1 containing a placeholder GILGFVFJL peptide (SEQ ID NO:7), or after UV-exchange in the presence of excess NLVPMVATV peptide (SEQ ID NO:8), showing a 20° C. increase in stability indicative of exchange to a higher affinity peptide. FIG. 5B is a DSF of HLA-A*02 biotin-mediated tetramers produced by UV exchange on the monomer followed by tetramerization, or by UV exchange on the tetramer itself, and confirms that multimeric state has no impact on the efficiency of UV-exchange, and that multimers of the current invention have the same stability as the industry standard pMHC.

FIGS. 6A-6F depict flow cytometry after peptide exchange on biotinylated HLA-A*02 monomers and tetramers. Donor PBMCs expanded with NLVPMVGTV peptide (SEQ ID NO: 9) were stained with: Anti-CD8-BV785 and Anti-Flag-APC secondary only (FIG. 6A), 50 nM HLA-A*02 biotin-mediated tetramers loaded with placeholder peptide GILGFVFJL (SEQ ID NO:7) (FIG. 6B), 50 nM HLA-A*02 biotin-mediated tetramers refolded with NLVPMVATV peptide (SEQ ID NO:8) (FIG. 6C), 50 nM HLA-A*02 biotin-mediated tetramers loaded with NLVPMVATV peptide (SEQ ID NO:8) via UV exchange on the monomeric form, followed by tetramerization with streptavidin (FIG. 6D), 50 nM HLA-A*02 biotin-mediated tetramers loaded with NLVPMVATV peptide (SEQ ID NO:8) via UV exchange on the tetrameric form itself (FIG. 6E) and 50 nM HLA-A*02 biotin-mediated tetramers loaded with NLVPMVATV peptide (SEQ ID NO: 8) via dipeptide exchange on the tetrameric form itself (FIG. 6F).

FIGS. 7A-7B depict flow cytometry after UV exchange on HLA-A*02:01-Alk-SAv-Az Conjugated Tetramers. Donor PBMCs expanded with NLVPMVATV peptide (SEQ ID NO: 8) were stained with: Anti-streptavidin-PE and Anti-Flag-APC secondaries only (FIG. 7A) or 1 nM HLA-A*02:01-Alk-SAv-Az Conjugated Tetramers loaded with NLVPMVATV peptide (SEQ ID NO: 8) via UV exchange directly on the tetrameric form (FIG. 7B).

FIGS. 8A-8C depict a comparison of ELISA and DSF as stability tests of UV-exchanged HLA-A*02 Tetramers. Specifically, FIG. 8A depicts an ELISA analysis of HLA-A*02:01-Alk-SAv-Az Conjugated Tetramers UV-exchanged to a 192-member peptide panel representing altered peptide ligands (APL) of the NLVPMVATV peptide (SEQ ID NO: 8). ELISA OD is plotted versus the netMHC predicted IC50 for each peptide. Different peptides span a range of ELISA signals. FIG. 8B shows DSF curves for a subset of NLVPMVATV (SEQ ID NO: 8) APL peptides UV-exchanged into biotin-mediated tetramers, demonstrating a span of stabilities. FIG. 8C shows a DSF/ELISA correlation for a subset of NLVPMVATV (SEQ ID NO: 8) APL peptides UV-exchanged into biotin-mediated tetramers.

FIGS. 9A-9D depict quality control analysis of HLA-A*01:01-Alk-SAv-Az Conjugated Tetramers. Specifically, FIG. 9A depicts an analytical SEC chromatogram of HLA-A*01:01 tetramers with low aggregate. FIG. 9B depicts an SDS-PAGE of HLA-A*01:01-Alk-SAv-Az Conjugated Tetramers non-boiled/non-reduced (NB/NR) or boiled/reduced (Boiled/R). FIG. 9C depicts DSF of HLA-A*01:01-Alk-SAv-Az Conjugated Tetramers loaded with placeholder peptide STAPGJLEY (SEQ ID NO: 16) (No UV), or after UV-exchange in the absence (UV no peptide) or presence (UV+VTEHDTLLY (SEQ ID NO: 10)) of rescue peptide. FIG. 9D depicts flow cytometry data for PBMC's expanded with VTEHDTLLY peptide (SEQ ID NO: 10), and stained with 20 nM HLA-A*01:01 biotin-mediated tetramers loaded with VTEHDTLLY peptide (SEQ ID NO: 10) by refolding (Refold VTE), HLA-A*01:01-Alk-SAv-Az Conjugated Tetramers loaded with STAPGJLEY (SEQ ID NO: 16) (No UV), or HLA-A*01:01-Alk-SAv-Az Conjugated Tetramers after UV-exchange in the presence of rescue peptide VTEHDTLLY (SEQ ID NO: 10) (UV+VTE). Both the fraction of tetramer positive cells (% Tetramer+) and mean fluorescence intensity (MFI) are depicted.

FIGS. 10A-10D depict quality control analysis of HLA-A*24:02-Alk-SAv-Az Conjugated Tetramers. Specifically, FIG. 10A depicts an analytical SEC chromatogram of HLA-A*24:02 tetramers with low aggregate. FIG. 10B depicts an SDS-PAGE of HLA-A*24:02-Alk-SAv-Az Conjugated Tetramers non-boiled/non-reduced (NB/NR) or boiled/reduced (Boiled/R). FIG. 10C depicts DSF of HLA-A*24:02-Alk-SAv-Az Conjugated Tetramers loaded with placeholder peptide VYGJVRACL (SEQ ID NO: 11) (No UV), or after UV-exchange in the absence (UV no peptide) or presence (UV+QYDPVAALF (SEQ ID NO: 12)) of rescue peptide. FIG. 10D depicts flow cytometry data for PBMC's expanded with QYDPVAALF peptide (SEQ ID NO: 12), and stained with secondary only, 20 nM HLA-A*24:02 biotin-mediated tetramers loaded with QYDPVAALF peptide (SEQ ID NO: 12) by refolding (Refold QYD), 20 nM HLA-A*24:02-Alk-SAv-Az Conjugated Tetramers loaded with VYGJVRACL (SEQ ID NO: 11) (No UV), or 20 nM HLA-A*24:02-Alk-SAv-Az Conjugated Tetramers after UV-exchange in the presence of rescue peptide QYDPVAALF (SEQ ID NO: 12) (UV+QYD). Both the fraction of tetramer positive cells (% Tetramer+) and mean fluorescence intensity (MFI) are depicted.

FIGS. 11A-11C depict quality control analysis of HLA-B*07:02-Alk-SAv-Az Conjugated Tetramers. Specifically, FIG. 11A depicts an analytical SEC chromatogram of HLA-B*07:02 tetramers with no aggregate. FIG. 11B depicts an SDS-PAGE of HLA-B*07:02-Alk-SAv-Az Conjugated Tetramers non-boiled/non-reduced (NB/NR). FIG. 11C depicts flow cytometry data for PBMC's expanded with RPHERNGFTVL peptide (SEQ ID NO: 13), and stained with secondary only, 20 nM HLA-B*07:02 biotin-mediated tetramers loaded with RPHERNGFTVL peptide (SEQ ID NO: 13) by refolding (Refold RPH), 20 nM HLA-B*07:02-Alk-SAv-Az Conjugated Tetramers loaded with AARGJTLAM (SEQ ID NO: 14), (No UV), or 20 nM HLA-B*07:02-Alk-SAv-Az Conjugated Tetramers after UV-exchange in the presence of rescue peptide RPHERNGFTVL (SEQ ID NO: 13), (UV+RPH). Both the fraction of tetramer positive cells (% Tetramer+) and mean fluorescence intensity (MFI) are depicted.

FIG. 12 depicts labeling HLA-A*02:01-Alk-SAv-Az Conjugated Tetramers with an identifying oligonucleotide tag. HLA-A*02:01-Alk-SAv-Az Conjugated Tetramers produced as described in Example 1 were incubated with 5′ biotinylated oligonucleotides and separated by Western probed with anti-Flag antibody. Shifted bands upon oligo addition indicated tetramer labeling.

FIG. 13 shows single cell sequencing of barcoded HLA-A*02:01-Alk-SAv-Az APL libraries. A heatmap of pMHC binding to individual T cells identified by single cell sequencing. Columns representing 2008 individual cells were clustered by TCR clonotype, and rows represent each of 192 APL variants of NLVPMATV (SEQ ID NO: 8). Warm colors indicate strong pMHC-TCR interactions read out by the identifying oligonucleotide tag.

FIG. 14 depicts PCR amplification of peptide-encoding template onto hydrogels under single template conditions. PCR was conducted on hydrogel beads either in bulk or after encapsulation in drops under single template conditions. Supernatant released upon breaking droplets after PCR was run next to product released from beads by XbaI or mock digest.

FIG. 15 shows the verification of single template amplification in drops. Hydrogels after PCR amplification of template in bulk or in drops under single template conditions were stained with streptavidin-PE. Fluorescent hydrogels were quantified relative to total hydrogels to confirm single template conditions.

FIGS. 16A-16B depict loading of HLA-A*02:01-Alk-SAv-Az Conjugated Tetramers onto PCR-amplified hydrogels. Signal to noise ratios for hydrogels stained with anti-Flag-APC or anti-β2M-Alexa488 after loading with Conjugated Tetramers or subsequent release with benzonase or SmaI (FIG. 16A). ELISA-determined concentrations of HLA-A*02:01-Alk-SAv-Az Conjugated Tetramers left in the supernatant after the hydrogel loading step, or released from loaded hydrogels by benzonase or SmaI (FIG. 16B).

FIGS. 17A-17B depict IVTT peptide production to generate functional UV-exchanged tetramers. Western probed with anti-SUMO domain antibody: Product of an IVTT reaction (+/−Ulp1 protease) driven by a PCR amplicon template encoding SUMO-NLVPMVATV (SEQ ID NO: 8) peptide fusion was run in lanes 10-11 (FIG. 17A). Lanes 2-9 contain a dilution series of a SUMO-domain-containing standard, which was used to quantify the yield of SUMO domain to ˜1 uM (FIG. 17A). Flow analysis of tetramers produced by UV-exchange from IVTT-produced peptide (FIG. 17B). Tetramers were UV-exchanged in the presence of equimolar synthetic NLVPMVATV (SEQ ID NO: 8) peptide (UV ex 1:1 NLV—synthetic) or an IVTT reaction (+Ulp1) driven by a SUMO-NLVPMVATV (SEQ ID NO: 8) peptide template (UV ex NLV-IVTT), and stained at 1 nM on NLVPMVATV (SEQ ID NO: 8)-expanded PBMCs (FIG. 17B). Positive and negative control tetramers refolded with NLVPMVATV (SEQ ID NO: 8) or GILGFVFJL (SEQ ID NO: 7) peptides were also stained at 1 nM as shown (FIG. 17B).

FIG. 18 shows flow cytometry results for pMHC tetramers produced and released from hydrogels utilizing in drop methods, stained on antigen-specific CD8+ T cells.

FIG. 19 is a schematic showing high throughput barcoded antigen library production using exchangeable barcodable tetramers.

FIG. 20 is a schematic showing use of sortags and click chemistry for conjugation of p*MHCII to SAv, cleavage of the peptide linker within the placeholder peptide, exchange of the placeholder peptide with a rescue peptide and binding to a TCR.

FIG. 21A-21E depicts the generation of p*MHCII multimer. FIG. 21A: Anti-Myc Western Blot analysis of GGG-Alkyne conjugation to the α-chain of monomeric p*MHCII. FIG. 21B: SDS-PAGE analysis following click reaction of p*MHCII-Alk and SAv-Az. FIG. 21C: HiLoad 26/600 Superdex 200 SEC elution chromatogram of the clicking reaction sample. FIG. 21D: Anti-FLAG Western Blot analysis of the main peaks obtained from SEC. Lane 1: Chameleon Duo Pre-Stained Protein Ladder (Licor), Lane 2: click reaction before loading the sample to the SEC column, lanes 3&4: SEC samples from peak I, lanes 5&6: SEC samples from peak II, lane 7: free SAv. Lane numbers correspond to non-boiled samples while lane numbers that are labeled with an asterisk correspond to boiled samples. FIG. 21E: Anti-His Western Blot analysis of the main peaks obtained following SEC. Lane numbers are the same as described in FIG. 21D.

FIG. 22A-22C illustrates the digestion, exchange and TCR binding of pMHCII. FIG. 22A: SDS-PAGE analysis of boiled and non-boiled samples of pre- and post-factor Xa cleavage. FIG. 22B: An ELISA assay that detects the ability of biotinylated exchanged peptide to bind to p↓MHCII multimer. FIG. 22C: BLI assay that measures the interaction between an HA-specific TCR and p↓MHCII multimer that was exchanged to display a cognate HA peptide. The black, light gray and dark gray curves correspond to the signal obtained from moving the TCR-loaded biosensors into wells containing either exchanged p↓MHCII, non-exchanged p*MHCII and BLI buffer, respectively. The dashed line defines the transfer of the biosensors to wells that are devoid of analytes (dissociation).

FIG. 23A-B illustrates the staining of a pMHCII tetramer library on antigen-specific T cells. FIG. 23A: Donor CD4+PBMCs expressing the DRB1*01:01 allele and expanded with influenza haemagglutinin epitope PKYVKQNTLKLAT (SEQ ID NO: 281) were stained with a 10 member DRB1*01:01 library with anti-Streptavidin-PE and anti-CD4-BV510 secondaries. Cells in the tetramer positive gate were sorted, mixed with MART1-antigen-specific CD8+ T cells stained with a 6 member library of ELAGIGILTV (SEQ ID NO: 282) variants loaded on A*02:01 tetramers, and the resulting pool was subjected to single cell sequencing, resulting in the heatmap shown in FIG. 23B. The most prevalent TCR clonotypes distributed on the y-axis bind specifically to HA-peptide-loaded DRB1*01:01 tetramers.

### DETAILED DESCRIPTION

### Definitions

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

As used herein, an “altered peptide ligand” or “APL” refers to an altered or mutated version of a peptide ligand, such as an MHC binding peptide. The altered or mutated version of the peptide ligand contains at least one structural modification (e.g., amino acid substitution) as compared to the peptide ligand from which it is derived. For example, a panel of APLs can be prepared by systematic or random mutation of a known MHC binding peptide, to thereby create a pool of APLs that can be used as a library of MHC binding peptides for loading onto MHC Conjugated Multimers as described herein.

As used herein, the term “and/or” when used in the context of a list of entities, refers to the entities being present singly or in any possible combination or subcombination.

The term “antigenic determinant” or “epitope” refers to a site on an antigen to which the variable domain of a T-cell receptor, an MHC molecule or antibody specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given TCR or antibody (i.e., epitope mapping) are well known in the art and include, for example, immunoblotting and immunoprecipitation assays, wherein overlapping or contiguous peptides from the antigen are tested for reactivity with the given TCR or immunoglobulin. Methods of determining spatial conformation of epitopes include techniques in the art and those described herein, for example, x-ray crystallography nuclear magnetic resonance, cryogenic electron microscopy (cryo-EM), hydrogen deuterium exchange mass spectrometry (HDX-MS), and site-directed mutagenisis (see, e.g., Epitope Mapping Protocols in Methods in Molecular Biology, Vol. 66, G. E. Morris, Ed. (1996)).

The term “avidity” as used herein, refers to the binding strength of as a function of the cooperative interactivity of multiple binding sites of a multivalent molecule (e.g., a soluble multimeric pMHC-immunoglobulin protein) with a target molecule. A number of technologies exist to characterize the avidity of molecular interactions including switchSENSE and surface plasmon resonance (Gjelstrup et al., J. Immunol. 188:1292-1306, 2012); Vorup-Jensen, Adv. Drug. Deliv. Rev. 64:1759-1781, 2012).

As used herein a “barcode”, also referred to as an oligonucleotide barcode, is a short nucleotide sequence (e.g., about 1, 2, 3, 4, 5, 6, 7, 8, 9, 10, 11, or 12 nucleotides long) that identifies a molecule to which it is conjugated. Barcodes can be used, for example, to identify molecules in a reaction mixture. Barcodes uniquely identify the molecule to which it is conjugated, for example, by performing reverse transcription using primers that each contain a “unique molecular identifier” barcode. In other embodiment, primers can be utilized that contain “molecular barcodes” unique to each molecule. The process of labeling a molecule with a barcode is referred to herein as “barcoding.” A “DNA barcode” is a DNA sequence used to identify a target molecule during DNA sequencing. In some embodiments, a library of DNA barcodes is generated randomly, for example, by assembling oligos in pools. In other embodiments, the library of DNA barcodes is rationally designed in silico and then manufactured.

“Binding affinity” generally refers to the strength of the sum total of noncovalent interactions between a single binding site of a molecule (e.g., a TCR, pMHC) and its binding partner. Unless indicated otherwise, as used herein, “binding affinity” refers to intrinsic binding affinity which reflects a 1:1 interaction between members of a binding pair (e.g., TCR and antigen). The affinity of a molecule X for its partner Y can generally be represented by the dissociation constant (Kd). For example, the Kd can be about 200 nM, 150 nM, 100 nM, 60 nM, 50 nM, 40 nM, 30 nM, 20 nM, 10 nM, 8 nM, 6 nM, 4 nM, 2 nM, 1 nM, or stronger, including up to 1 μM. Affinity can be measured by common methods known in the art, including those described herein. Low-affinity TCRs generally bind antigen slowly and tend to dissociate readily, whereas high-affinity TCRs generally bind antigen faster and tend to remain bound longer. A variety of methods of measuring binding affinity are known in the art, any of which can be used for purposes of the present disclosure.

The term “bioorthogonal chemistry” refers to any chemical reaction that can occur inside of living systems without interfering with native biochemical processes. The term includes chemical reactions that are chemical reactions that occur in vitro at physiological pH in, or in the presence of water. To be considered bioorthogonol, the reactions are selective and avoid side-reactions with other functional groups found in the starting compounds. In addition, the resulting covalent bond between the reaction partners should be strong and chemically inert to biological reactions and should not affect the biological activity of the desired molecule.

As used herein, the terms “carrier” and “pharmaceutically acceptable carrier” includes any and all solvents, dispersion media, coatings, antibacterial and antifungal agents, isotonic and absorption delaying agents, and the like that are physiologically compatible.

The term “chelator ligand” as used herein refers to a bifunctional conjugating moiety that covalently links a radiolabeled prosthetic group to a biologically active targeting molecule (e.g., peptide or protein). Bifunctional conjugating moiety utilize functional groups such as carboxylic acids or activated esters for amide couplings, isothiocyanates for thiourea couplings and maleimides for thiol couplings.

As used herein, the term “cleavable moiety” refers to a motif or sequence that is cleavable. In some embodiments, the cleavage moiety comprises a protein, e.g., enzymatic, cleavage site. In some embodiments, the cleavage moiety comprises a chemical cleavage site, e.g., through exposure to oxidation/reduction conditions, light/sound, temperature, pH, pressure, etc.

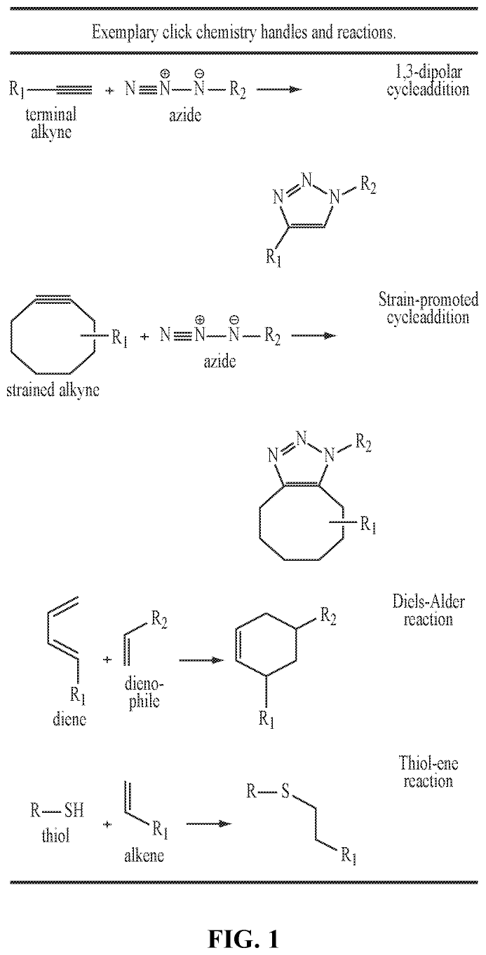

The term “click chemistry” refers to a set of reliable and selective bioorthogonal reactions for the rapid synthesis of new compounds and combinatorial libraries. Properties of click reactions include modularity, wideness in scope, high yielding, stereospecificity and simple product isolation (separation from inert by-products by non-chromatographic methods) to produce compounds that are stable under physiological conditions. In radiochemistry and radiopharmacy, click chemistry is a generic term for a set of labeling reactions which make use of selective and modular building blocks and enable chemoselective ligations to radiolabel biologically relevant compounds in the absence of catalysts. A “click reaction” can be with copper, or it can be a copper-free click reaction. Non-limiting examples of click chemistry handles and reactions are shown in FIG. 1.

As used herein, the term “conditions sufficient for covalent conjugation” refers to reaction conditions, including but not limited to temperature, pH and concentrations of the reaction components, that are suitable such that the desired covalent conjugation chemical reaction occurs.

As used herein, the term “Conjugated Multimer”, also referred to as a pMHC Conjugated Multimer, refers to the reaction product that results from the reaction of pMHC monomers comprising a conjugation moiety with a multimerization domain comprising a conjugation moiety, wherein the two conjugation moieties react with each other to form a covalent linkage between the pMHC monomers and the multimerization domain, thereby forming Conjugated Multimers. In one embodiment, the Conjugated Multimer is a Conjugated Tetramer, in which four pMHC monomers are reacted with the multimerization domain, through their conjugation moieties, to thereby form a tetramer. In one embodiment, the Conjugated Multimer is a pMHCI Conjugated Multimer (e.g., Tetramer), in which pMHC Class I monomers are multimerized. In one embodiment, the Conjugated Multimer is a pMHCII Conjugated Multimer (e.g., Tetramer) in which pMHC Class II monomers are multimerized.

As used herein, the term “cross-linking unit” can refer to a molecule that links to another (same or different) molecule. In some embodiments, the cross-linking unit is a monomer. In some embodiments, the cross-link is a chemical bond. In some embodiments, the cross-link is a covalent bond. In some embodiments, the cross-link is an ionic bond. In some embodiments, the cross-link alters at least one physical property of the linked molecules, e.g., a polymer's physical property.

As used herein, the term “endoprotease” refers to a protease that cleaves a peptide bond of a non-terminal amino acid.

As used herein, the term “epitope” (as in “peptide epitope”) refers to a portion of an antigen (e.g., antigenic protein) that binds to (interacts with or is recognized by) an immune receptor. Thus, a T cell receptor recognizes and binds to an MHC molecule complexed with (loaded with) a peptide epitope.

The terms “exchangeable pMHC polypeptide”, “exchangeable pMHC multimers”, and “placeholder-peptide loaded MHC polypeptide”, which are used interchangeably herein, refer to MHC monomers and MHC multimers, comprising a placeholder peptide in the binding groove of the MHC polypeptide, and are also referred to as “p*MHC” monomers or multimers. “Exchangeable” refers to the property of a p*MHC monomer or p*MHC multimer allowing for the exchange of the placeholder peptide with an antigenic peptide. In one embodiment, the exchangeable pMHC or p*MHC polypeptide comprises an MHC Class I molecule with an MHC Class I-binding peptide in the binding groove of the MHC Class I molecule. In another embodiment, the exchangeable pMHC or p*MHC polypeptide comprises an MHC Class II molecule with an MHC Class II-binding peptide in the binding groove of the MHC Class II molecule.

A “fusion protein” or “fusion polypeptide” as used interchangeably herein refers to a recombinant protein prepared by linking or fusing two polypeptides into a single protein molecule.

The term “isolated” as applied to MHC monomers herein refers to an MHC glycoprotein, which is in other than its native state, for example, not associated with the cell membrane of a cell that normally expresses MHC. This term embraces a full length subunit chain, as well as a functional fragment of the MHC monomer. A functional fragment is one comprising an antigen binding site and sequences necessary for recognition by the appropriate T cell receptor. It typically comprises at least about 60-80%, typically 90-95% of the sequence of the full-length chain. An “isolated” MHC subunit component may be recombinantly produced or solubilized from the appropriate cell source. In one embodiment, the “isolated” MHC monomer is an MHC Class I monomer, such as a soluble form of the MHC Class I heavy chain (α chain) associated with β2-microglobulin. In another embodiment, the “isolated” MHC monomer is an MHC Class II monomer, such as a soluble form of the MHC Class II α/β chains.

As used herein, the term “identifier” refers to a readable representation of data that provides information, such as an identity, that corresponds with the identifier.

As used herein, the terms “linked,” “conjugated,” “fused,” or “fusion,” are used interchangeably when referring to the joining together of two more elements or components or domains, by whatever means including recombinant or chemical means.

The term “Major Histocompatibility Complex” or “MHC” refers to genomic locus containing a group of genes that encode the polymorphic cell-membrane-bound glycoproteins known as MHC classical class I and class II molecules that regulate the immune response by presenting peptides of fragmented proteins to circulating cytotoxic and helper T lymphocytes, respectively. In humans this group of genes is also called the “human leukocyte antigen” or “HLA” system. Human MHC class I genes encode, for example, HLA-A, HL-B and HLA-C molecules. HLA-A is one of three major types of human MHC class I cell surface receptors. The others are HLA-B and HLA-C. The HLA-A protein is a heterodimer, and is composed of a heavy α chain and smaller β chain. The α chain is encoded by a variant HLA-A gene, and the β chain is an invariant β2 microglobulin (β2m) polypeptide. The (32 microglobulin polypeptide is coded for by a separate region of the human genome. HLA-A*02 (A*02) is a human leukocyte antigen serotype within the HLA-A serotype group. The serotype is determined by the antibody recognition of the α2 domain of the HLA-A α-chain. For A*02, the α chain is encoded by the HLA-A*02 gene and the β chain is encoded by the B2M locus. Human MHC class II genes encode, for example, HLA-DPA1, HLA-DPB1, HLA-DQA1, HLA-DQB1, HLA-DRA and HLA-DRB1. The complete nucleotide sequence and gene map of the human major histocompatibility complex is publicly available (e.g., The MHC sequencing consortium, Nature 401:921-923, 1999).

As used herein, the terms “MHC molecule” and “MHC protein” are used herein to refer to the polymorphic glycoproteins encoded by the MHC class I and MHC class II genes, which are involved in the presentation of peptide epitopes to T cells. The terms “MHC class I” or “MHC I” are used interchangeably to refer to protein molecules comprising an α chain composed of three domains (α1, α2 and α3), and a second, invariant β2-microglobulin. The α3 domain is transmembrane, anchoring the MHC class I molecule to the cell membrane. Antigen-derived peptide epitopes, which are located in the peptide-binding groove, in the central region of the α1/α2 heterodimer. MHC Class I molecules such as HLA-A are part of a process that presents short polypeptides to the immune system. These polypeptides are typically 9-11 amino acids in length and originate from proteins being expressed by the cell. MHC class I molecules present antigen to CD8+ cytotoxic T cells. The terms “MHC class II” and “MHC II” are used interchangeably to refer to protein molecules containing an α chain with two domains (α1 and α2) and a β chain with two domains (β1 and β2). The peptide-binding groove is formed by the α1/β1 heterodimer. MHC class II molecules present antigen to specific CD4+ T cells. Antigens delivered endogenously to APCs are processed primarily for association with MHC class I. Antigens delivered exogenously to APCs are processed primarily for association with MHC class II.

As used herein, MHC proteins (MHC Class I or Class II proteins) also includes MHC variants which contain amino acid substitutions, deletions or insertions and yet which still bind MHC peptide epitopes (MHC Class I or MHC Class II peptide epitopes). The term also includes fragments of all these proteins, for example, the extracellular domain, which retain peptide binding.

The term “MHC protein” also includes MHC proteins of non-human species of vertebrates. MHC proteins of non-human species of vertebrates play a role in the examination and healing of diseases of these species of vertebrates, for example, in veterinary medicine and in animal tests in which human diseases are examined on an animal model, for example, EAE (experimental autoimmune encephalomyelitis) in mice (Mus musculus), which is an animal model of the human disease multiple sclerosis. Non-human species of vertebrates are, for example, and more specifically mice (Mus musculus), rats (Rattus norvegicus), cows (Bos taurus), horses (Equus equus) and green monkeys (Macaca mulatta). MHC proteins of mice are, for example, referred to as H-2-proteins, wherein the MHC class I proteins are encoded by the gene loci H2K, H2L and H2D and the MHC class II proteins are encoded by the gene loci H21.

A “peptide free MHC polypeptide” or “peptide free MHC multimer” as used herein refers to an MHC monomer or MHC multimer which does not contain a peptide in binding groove of the MHC polypeptide. Peptide free MHC monomers and multimers are also referred to as “empty”. In one embodiment, the peptide free MHC polypeptide or multimer is an MHC Class I polypeptide or multimer. In another embodiment, the peptide free MHC polypeptide or multimer is an MHC Class II polypeptide or multimer.

As used herein, the term “multimer” refers to a plurality of units. In some embodiments, the multimer comprises one or more different units. In some embodiments, the units in the multimer are the same. In some embodiments, the units in the multimer are different. In some embodiments, the multimer comprises a mixture of units that are the same and different.

The terms “peptide epitope”, “MHC peptide epitope”, “MHC peptide antigen” and “MHC ligand” are used interchangeably herein and refer to an MHC ligand that can bind in the peptide binding groove of an MHC molecule. The peptide epitope can typically be presented by the MHC molecule. A peptide epitope typically has between 8 and 25 amino acids that are linked via peptide bonds. The peptide can contain modification such as, but not limited to, the side chains of the amino acid residues, the presence of a label or tag, the presence of a synthetic amino acid, a functional equivalent of an amino acid, or the like. Typical modifications include those as produced by the cellular machinery, such as glycan addition and phosphorylation. However, other types of modification are also within the scope of the disclosure.

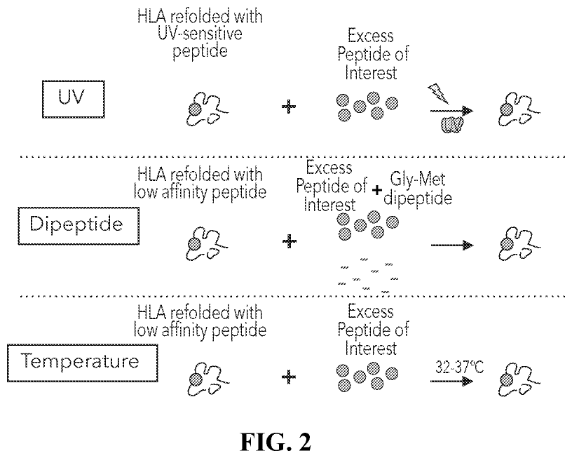

As used herein, the terms “peptide exchange” refers to a competition assay wherein a placeholder peptide is removed and replaced by a “exchanged peptide” (or “exchange peptide epitope”) also referred to herein as a “rescue peptide” (or “rescue peptide epitope”) or “competitor peptide” (or “competitor peptide epitope). Typically, peptide exchange occurs under conditions in which the placeholder peptide is released by cleavage of the peptide or under suitable conditions allowing rescue peptides to compete for binding to the binding pocket of an MHC monomer or multimer. For example, peptide exchange can be accomplished by UV-induced exchange, dipeptide-induced exchange, temperature-induced exchange, or other exchange methods known in the art, and disclosed herein. Exemplary methods of peptide exchange are set forth in FIG. 2.

As used herein, the term “peptide library” refers to a plurality of peptides. In some embodiments, the library comprises one or more peptides with unique sequences. In some embodiments, each peptide in the library has a different sequence. In some embodiments, the library comprises a mixture of peptides with the same and different sequences.

As used herein, the term “high diversity peptide library” refers to a peptide library with a high degree of peptide variety. For example, a high diversity peptide library comprises about 10³, about 10⁴, about 10⁵, about 10⁶, about 10⁷, about 10⁸, about 10⁹, about 10¹⁰, about 10¹¹, about 10¹², about 10¹³, about 10¹⁴, about 10¹⁵, about 10¹⁶, about 10¹⁷, about 10¹⁸, about 10¹⁹, about 10²⁰, or more different peptides.

As used herein, the term “library peptide” refers to a single peptide in the library.

As used herein, the terms “placeholder peptide” or “exchangeable peptide” are used interchangeably to refer to a peptide or peptide-like compound that binds with sufficient affinity to an MHC protein (e.g., MHCI or MHCII protein) and which causes or promotes proper folding of the MHC protein from the unfolded state or stabilization of the folded MHC protein. The placeholder peptide can subsequently be exchanged with a different peptide of interest (referred to as an exchange peptide or rescue peptide). This exchange can be accomplished by UV-induced exchange, dipeptide-induced exchange, temperature-induced exchange, or other exchange methods known in the art.

The terms “polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. The terms “isolated protein” and “isolated polypeptide” are used interchangeably to refer to a protein (e.g., a soluble, multimeric protein) which has been separated or purified from other components (e.g., proteins, cellular material) and/or chemicals. Typically, a polypeptide is purified when it constitutes at least 60 (e.g., at least 65, 70, 75, 80, 85, 90, 92, 95, 97, or 99) % by weight of the total protein in the sample.

As used herein, the term “protein folding” refers to spatial organization of a peptide. In some embodiments, the amino acid sequence influences the spatial organization or folding of the peptide. In some embodiments, a peptide may be folded in a functional conformation. In some embodiments, a folded peptide has one or more biological functions. In some embodiments, a folded peptide acquires a three-dimensional structure.

As used herein, the term “N-terminus amino acid residue” refers to one or more amino acids at the N-terminus of a polypeptide.

As used herein, the terms “small ubiquitin-like modifier moiety” or “SUMO domain” or “SUMO moiety” are used interchangeably and refer to a specific protease recognition moiety.

As used herein, the term “tag” refers to an oligonucleotide component, generally DNA, that provides a means of addressing a target molecule (e.g., a Conjugated Multimer) to which it is joined. For example, in some embodiments, a tag comprises a nucleotide sequence that permits identification, recognition, and/or molecular or biochemical manipulation of the molecule to which the tag is attached (e.g., by providing a unique sequence, and/or a site for annealing an oligonucleotide, such as a primer for extension by a DNA polymerase, or an oligonucleotide for capture or for a ligation reaction). The process of joining the tag to the target molecule is sometimes referred to herein as “tagging” and a target molecule that undergoes tagging or that contains a tag is referred to as “tagged” (e.g., a “tagged Conjugated Multimer”).” A tag can be a barcode, an adapter sequence, a primer hybridization site, or a combination thereof.

The term “T cell” refers to a type of white blood cell that can be distinguished from other white blood cells by the presence of a T cell receptor on the cell surface. There are several subsets of T cells, including, but not limited to, T helper cells (a.k.a. T_{H} cells or CD4⁺ T cells) and subtypes, including T_{H}1, T_{H}2, T_{H}3, T_{H}17, T_{H}9, and T_{FH} cells, cytotoxic T cells (a.k.a T_{C} cells, CD8⁺ T cells, cytotoxic T lymphocytes, T-killer cells, killer T cells), memory T cells and subtypes, including central memory T cells (T_{CM} cells), effector memory T cells (T_{EM} and T_{EMRA} cells), and resident memory T cells (T_{RM} cells), regulatory T cells (a.k.a. T_{reg} cells or suppressor T cells) and subtypes, including CD4⁺ FOXP3⁺ T_{reg} cells, CD4⁺FOXP3⁻ T_{reg} cells, Tr1 cells, Th3 cells, and T_{reg}17 cells, natural killer T cells (a.k.a. NKT cells), mucosal associated invariant T cells (MAITs), and gamma delta T cells (γδ T cells), including Vγ9/Vδ2 T cells. The term “T cell cytotoxicity” includes any immune response that is mediated by CD8+ T cell activation.

As used herein, the phrase “T cell receptor” and the term “TCR” refer to a surface protein of a T cell that allows the T cell to recognize an antigen and/or an epitope thereof, typically bound to one or more major histocompatibility complex (MHC) molecules. A TCR functions to recognize an antigenic determinant and to initiate an immune response. Typically, TCRs are heterodimers comprising two different protein chains. In the vast majority of T cells, the TCR comprises an alpha (α) chain and a beta (β) chain. Each chain comprises two extracellular domains: a variable (V) region and a constant (C) region, the latter of which is membrane-proximal. The variable domains of α-chains and of β-chains consist of three hypervariable regions that are also referred to as the complementarity determining regions (CDRs). The CDRs, in particular CDR3, are primarily responsible for contacting antigens and thus define the specificity of the TCR, although CDR1 of the α-chain can interact with the N-terminal part of the antigen, and CDR1 of the β-chain interacts with the C-terminal part of the antigen. Approximately 5% of T cells have TCRs made up of gamma and delta (γ/δ) chains. All numbering of the amino acid sequences and designation of protein loops and sheets of the TCRs is according to the IMGT numbering scheme (IMGT, the international ImMunoGeneTics information system@imgt.cines.fr; http://imgt.cines.fr; Lefranc et al., (2003) Dev Comp Immunol 27:55 77.; Lefranc et al. (2005) Dev Comp Immunol 29:185-203).

As used herein, the terms “soluble T-cell receptor” and “sTCR” refer to heterodimeric truncated variants of TCRs, which comprise extracellular portions of the TCR α-chain and β-chain (e.g., linked by a disulfide bond), but which lack the transmembrane and cytosolic domains of the full-length protein. The sequence (amino acid or nucleic acid) of the soluble TCR α-chain and β-chains may be identical to the corresponding sequences in a native TCR or may comprise variant soluble TCR α-chain and β-chain sequences, as compared to the corresponding native TCR sequences. The term “soluble T-cell receptor” as used herein encompasses soluble TCRs with variant or non-variant soluble TCR α-chain and β-chain sequences. The variations may be in the variable or constant regions of the soluble TCR α-chain and β-chain sequences and can include, but are not limited to, amino acid deletion, insertion, substitution mutations as well as changes to the nucleic acid sequence, which do not alter the amino acid sequence. Variants retain the binding functionality of their parent molecules.

As used herein, a “TCR/pMHC complex” refers to a protein complex formed by binding between T cell receptor (TCR), or soluble portion thereof, and a peptide-loaded MHC molecule. Accordingly, a “component of a TCR/pMHC complex” refers to one or more subunits of a TCR (e.g., Vα, Vβ, Cα, Cβ), or to one or more subunits of an MHC or pMHC class I or II molecule.

As used herein, the term “unbiased” refers to lacking one or more selective criteria.

### Overview

This disclosure provides methods for the high-throughput generation of libraries containing peptide-loaded MHC (pMHC) multimers containing a plurality of unique peptides in the MHC binding groove and having oligonucleotide barcode labeling to facilitate identification of library members. In the methods provided herein, all of the challenging and potentially inefficient chemistry steps for generation of pMHC multimers are done in a single bulk reaction including chromatographic cleanup and purification, followed by highly efficient peptide exchange and oligonucleotide barcoding. In particular, pMHC monomers are linked to the multimerization domain through the use of conjugation moieties on the monomers and the multimerization domain that react to form a stable chemical linkage (i.e., covalent bond) between the monomers and the multimerization domain, thereby forming a pMHC Conjugated Multimer, such as a pMHC Conjugated Tetramer. Various conjugation moieties and reactions are suitable for use in forming the Conjugated Multimers, as described herein, including use of bioorthogonal chemistry, such as click chemistry, that allow for ease and efficiency of the reactions. Moreover, when the multimerization domain is streptavidin, since the biotin-binding site is not being used for attaching the pMHC monomers, this biotin-binding site is thus available for convenient attachment of biotinylated oligonucleotide barcodes, to thereby label the multimers easily and efficiently.

The libraries of pMHC multimers provided herein are useful in a range of therapeutic, diagnostic, and research applications, essentially in any situation in which pMHC multimers are useful. For example, pMHC multimers as described herein can be used in a variety of methods, for example, to identify and isolate specific T-cells in a wide array of applications. In one embodiment, the pMHC multimers are pMHC Class I multimers, which are useful for determining the antigenic specificity of CD8+ T cells (e.g., cytotoxic T cells). In another embodiment, the pMHC multimers are pMHC Class II multimers, which are useful for determining the antigenic specificity of CD4+ T cells (e.g., helper T cells).

### I. MHC Polypeptides

A. MHC Class I Polypeptides

The Class I histocompatibility ternary complex consists of three parts associated by noncovalent bonds. The MHCI heavy chain is a polymorphic transmembrane glycoprotein of about 45 kDa consisting of three extracellular domains, each containing about 90 amino acids (α1 at the N-terminus, α2 and α3), a transmembrane domain of about 40 amino acids and a cytoplasmic tail of about 30 amino acids. The α1 and α2 domains of the MHCI heavy chain contain two segments of alpha helix that form a peptide-binding groove or cleft. A short peptide of about 8-10 amino acids binds noncovalently (“fits”) into this groove between the two alpha helices. The α3 domain of the MHCI heavy chain is proximal to the plasma membrane. The MHCI heavy chain is non-covalently bound to a β2 microglobulin (β2m) polypeptide, forming a ternary complex. In MHCI, the binding groove is closed at both ends by conserved tyrosine residues leading to a size restriction of the bound peptides to usually 8-10 residues with its C-terminal end docking into the F-pocket.

The disclosure provides a multimeric protein comprising a two or more MHCI or MHCI-like polypeptides. The MHCI molecule can suitably be a vertebrate MHC molecule such as a human, a mouse, a rat, a porcine, a bovine or an avian MHC molecule.

In some embodiments, the multimeric MHCI multimers described herein, the MHC molecule is a human MHC class I protein: HLA-A, HLA-B of HLA-C. In some embodiments, the multimer comprises MHC Class I like molecules (including non-classical MHC Class I molecules) including, but not limited to, CD1d, HLA E, HLA G, HLA F, HLA H, MIC A, MIC B, ULBP-1, ULBP-2, and ULBP-3. The amino acid sequences of the MHCI heavy chains, β2m polypeptides and of MHC Class I like molecules from a variety of vertebrate species are known in the art and publicly available.

In some embodiments, the MHCI heavy chain alpha domain is human, and comprise, for example, an MHCI heavy chain alpha domain(s) from a human MHC Class I molecule(s) selected from the group consisting of HLA-A*01:01, HLA-A*03:01, HLA-A*11:01, HLA-A*24:02, HLA-B*07:02, HLA-C*04:01, HLA-C*07:02, HLA-B*08:01, HLA-B*35:01, HLA-B*57:01, HLA-B*57:03, HLA-E, HLA-C*16:01, HLA-C*08:02, HLA-C*07:01, HLA-C*05:01, HLA-B*44:02, HLA-A*29:02, HLA-B*44:03, HLA-C*03:04, HLA-B*40:01, HLA-C*06:02, HLA-B*15:01, HLA-C*03:03, HLA-A*30:01, HLA-B*13:02, HLA-C*12:03, HLA-A*26:01, HLA-B*38:01, HLA-B*14:02, HLA-A*33:01, HLA-A*23:01, HLA-A*25:01, HLA-B*18:01, HLA-B*37:01, HLA-B*51:01, HLA-C*14:02, HLA-C*15:02, HLA-C*02:02, HLA-B*27:05, HLA-A*31:01, HLA-A*30:02, HLA-B*42:01, HLA-C*17:01, HLA-B*35:02, HLA-B*39:06, HLA-C*03:02, HLA-B*58:01, HLA-A*33:03, HLA-A*68:02, HLA-C*01:02, HLA-C*07:04, HLA-A*68:01, HLA-A*32:01, HLA-B*49:01, HLA-B*53:01, HLA-B*50:01, HLA-A*02:05, HLA-B*55:01, HLA-B*45:01, HLA-B*52:01, HLA-C*12:02, HLA-B*35:03, HLA-B*40:02, HLA-B*15:03 and/or HLA-A*74:01. The full-length amino acid sequences (including signal sequence and transmembrane domain) of these MHCI molecules are shown in SEQ ID NOs: 28-93, respectively. The amino acid sequences of soluble forms of these MHCI molecules (lacking signal sequence and transmembrane domain) are shown in SEQ ID NOs: 94-159, respectively.

In some embodiments, the pMHCI multimers described herein comprises the α1 and α2 domains of an MHCI heavy chain. In some embodiments, the compound described herein comprises the α1, α2, and α3 domains of an MHCI heavy chain.

In some embodiments, the two or more pMHCI or pMHCI-like polypeptides in the multimer comprises a (β2-microglobulin polypeptide, e.g., a human (β2-microglobulin. In some embodiments, the (β2-microglobulin is wild-type human (β2-microglobulin. In some embodiments, the (β2-microglobulin comprises an amino acid sequence that is at least 80, 85, 90, 95, or 99% identical to the amino acid sequence of the human (32 microglobulin, the full-length sequence of which is shown in SEQ ID NO: 160 (UniProt Id. No. P61769). Alternatively, the human (β2-microglobulin polypeptide used in the pMHCI multimer can comprise or consist of the amino acid sequence shown in SEQ ID NO: 2.

In some embodiments, the multimeric protein comprises a soluble MHCI polypeptide. In some embodiments the MHC-multimeric protein comprises a soluble MHCI α domain and a (β2-microglobulin polypeptide. In some embodiments, the soluble MHCI protein comprises the MHCI heavy chain α1 domain and the MHCI heavy chain α2 domain.

Alternatively, in some embodiments, the MHCI monomer is a fusion protein comprising a β2m polypeptide or functional fragment thereof covalently linked to the MHCI heavy chain or functional fragment thereof. In some embodiments the carboxy (—COOH) terminus of β2m is covalently linked to the amino (—NH₂) terminus of the MHCI heavy chain.

In some embodiments, the MHC monomers comprise one or more linkers between the individual components of the MHCI monomer. In some embodiments, the MHCI monomer comprises a heavy chain fused with β2m through a linker. In some embodiments, the linker between the heavy chain and β2m is a flexible linker, e.g., made of glycine and serine. In some embodiments, the flexible linker between the heavy chain and β2m is between 5-20 residues long. In other embodiments, the linker between the heavy chain and β2m is rigid with a defined structure, e.g. made of amino acids like glutamate, alanine, lysine, and leucine. In one embodiment, the linker is a (G₄S)₄ linker (SEQ ID NO: 181, wherein n=4).

The amino acid sequences of a number of MHC Class I proteins are known, and the genes have been cloned, therefore, the heavy chain monomers can be expressed using recombinant methods. Methods for the expression and purification of MHCI molecules have been extensively described (e.g., Altman et al., Curr. Protoc. Enz. 17.3.1-17.2-44, 2016). For example, the MHCI heavy chain and β2-microglobulin can be expressed in separate cells, and isolated by purification and then refolded in vitro. For example, the MHC polypeptide chains can be expressed in E. coli, where MHC polypeptide chains accumulate as insoluble inclusion bodies in the bacterial cell. In vitro refolding occurs in a refolding buffer where the polypeptides are added by e.g. dialysis or dilution. Refolding buffers can be any buffer wherein the MHC polypeptide chains and peptide are allowed to reconstitute the native trimer fold. The buffer may contain oxidative and/or reducing agents thereby creating a redox buffer system helping the MHC proteins to establish the correct fold. Examples of suitable refolding buffers include but are not limited to Tris-buffer, CAPS buffer, TAPs buffer, PBS buffer, other phosphate buffer, carbonate buffer and Ches buffer. Chaperone molecules or other molecules improving correct protein folding may also be added and likewise agents increasing solubility and preventing aggregate formation may be added to the buffer. Examples of such molecules include but is not limited to Arginine, GroE, HSP70, HSP90, small organic compounds, DnaK, CIpB, proline, glycinbetaine, glycerol, tween, salt, PLURONIC™.

Once expressed the MHCI complexes can be purified directly as whole MHCI or MHCI-peptide monomers from MHCI expressing cells. The MHCI monomers may be expressed on the surface of cells, and are then isolated by disruption of the cell membrane using, e.g., detergent followed by purification of the MHCI. In some embodiments, MHC monomers are expressed into the periplasm and expressing cells are lysed and released MHCI monomers purified. Alternatively, MHC monomers may be purified from the supernatant of cells secreting expressed proteins into culture supernatant. Methods for purifying MHCI monomers are well known in the art, for example, via the use of affinity tags together with affinity chromatography, beads coated with ant-tag and/or other techniques involving immobilization of MHCI protein to affinity matrix; size exclusion chromatography using, e.g., gel filtration, ion exchange or other methods able to separate MHC molecules from cells and/or cell lysates.

In some embodiments, recombinant expression of MHCI polypeptides allow a number of modifications of the MHC monomers. For example, recombinant techniques provide methods for carboxy terminal truncation which deletes the hydrophobic transmembrane domain. The carboxy termini can also be arbitrarily chosen to facilitate the conjugation of ligands or labels, for example, by introducing cysteine and/or lysine residues into the molecule. The synthetic gene will typically include restriction sites to aid insertion into expression vectors and manipulation of the gene sequence. The genes encoding the appropriate monomers are then inserted into expression vectors, expressed in an appropriate host, such as E. coli, yeast, insect, or other suitable cells, and the recombinant proteins are obtained. For example, the production of MHC class I polypeptides includes bacterial expression and folding of the MHC class I light chain, β2-microglobulin (β2m), as well as the formation of a complex consisting of the MHC class I heavy chain, β2m, and a placeholder peptide.

In some embodiments, the MHCI monomers are biotinylated on either their heavy chain or β2m. In some embodiments, the MHCI monomers are biotinylated before loading of the peptide either by refolding or peptide exchange. Biotinylation of the MHC monomers can be achieved as known in the art, e.g. by attaching biotin to a specific attachment site which is the recognition site of a biotinylating enzyme. In some embodiments, the biotinlylating enzyme is BirA. In some embodiments, biotinylation is carried out on the desired protein chain in vivo as a post translational modification during protein expression.

B. MHC Class II Polypeptides

MHC class II molecules are heterodimers composed of an α chain and a β chain, both of which are encoded by the MHC. The alpha chain is comprised of α1 and α2 domains. The beta chain is comprised of β 1 and β 2 domains. The α1 and β1 domains of the chains interact noncovalently to form a membrane-distal peptide-binding domain, whereas the α2 and β2 domains form a membrane-proximal immunoglobulin-like domain. The antigen binding groove, where a peptide epitope binds, is made up of two α-helices and a β-sheet. Since the antigen binding groove of MHC class II molecules is open at both ends, the groove can accommodate longer peptide epitopes than MHC class I molecules. Peptide epitopes presented by MHC class II molecules typically are about 15-24 amino acid residues in length.

The disclosure provides a multimeric protein comprising two or more MHCII or MHCII-like polypeptides. The MHCII molecule can suitably be a vertebrate MHCII molecule such as a human, a mouse, a rat, a porcine, a bovine or an avian MHCII molecule.

In some embodiments, the multimeric MHCII multimers described herein, the MHC molecule is a human MHC class II protein: HLA-DR, HLA-DQ, HLA-DX, HLA-DO, HLA-DZ, and HLA-DP. The amino acid sequences of the MHCII α and β chains from a variety of vertebrate species, including humans, are known in the art and publicly available.

In some embodiments, the human MHCII molecule is of an allotype selected from the group consisting of DRB1*0101 (see, e.g., Cameron et al. (2002) J. Immunol. Methods, 268:51-69; Cunliffe et al. (2002) Eur. J. Immunol., 32:3366-3375; Danke et al. (2003) J. Immunol., 171:3163-3169), DRB1*1501 (see, e.g., Day et al. (2003) J. Clin. Invest, 112:831-842), DRB5*0101 (see, e.g., Day et al., ibid), DRB1*0301 (see, e.g., Bronke et al. (2005) Hum. Immunol., 66:950-961), DRB1*0401 (see, e.g., Meyer et al. (2000) PNAS, 97:11433-11438; Novak et al. (1999) J. Clin. Invest, 104:R63-R67; Kotzin et al. (2000) PNAS, 97:291-296), DRB1*0402 (see, e.g., Veldman et al. (2007) Clin. Immunol., 122:330-337), DRB1*0404 (see, e.g., Gebe et al. (2001) J. Immunol. 167:3250-3256), DRB1*1101 (see, e.g., Cunliffe, ibid; Moro et al. (2005) BMC Immunol., 6:24), DRB1*1302 (see, e.g., Laughlin et al. (2007) Infect. Immunol. 75:1852-1860), DRB1*0701 (see, e.g., Danke, ibid), DQA1*0102 (see, e.g., Kwok et al. (2000) J. Immunol., 164:4244-4249), DQB1*0602 (see, e.g., Kwok, ibid), DQA1*0501 (see, e.g., Quarsten et al. (2001) J. Immunol., 167:4861-4868), DQB1*0201 (see, e.g., Quarsten, ibid), DPA1*0103 (see, e.g., Zhang et al. (2005) Eur. J. Immunol, 35:1066-1075; Yang et al. (2005) J. Clin. Immunol., 25:428-436), and DPB1*0401 (see, e.g., Zhang, ibid; Yang, ibid).

In some embodiments, the MHCII molecule is human, and comprise, for example, an MHCII alpha and beta chains selected from the group consisting of HLA-DRA*01:01, HLA-DRB1*01:01, HLA-DRB1*01:02, HLA-DRB1*03:01, HLA-DRB1*04:01, HLA-DRB1*04:04, HLA-DRB1*07:01, HLA-DRB1*08:01, HLA-DRB1*10:01, HLA-DRB1*11:01, HLA-DRB1*11:04, HLA-DRB1*13:01, HLA-DRB1*13:02, HLA-DRB1*14:01, HLA-DRB1*15:01, HLA-DRB1*15:03, HLA-DQA1*01:01, HLA-DQB1*05:01, HLA-DQA1*01:02, HLA-DQB1*06:02, HLA-DQA1*03:01, HLA-DQB1*03:02, HLA-DQA1*05:01, HLA-DQB1*02:01, HLA-DQB1*03:01, HLA-DQB1*03:03, HLA-DQB1*04:02, HLA-DQB1*05:03, HLA-DQB1*06:03 and HLA-DQB1*06:04. The full-length amino acid sequences (including signal sequence and transmembrane domain) of these MHCII chains are shown in SEQ ID NOs: 194-223, respectively. The amino acid sequences of soluble forms of these MHCII chains (lacking signal sequence and transmembrane domain) are shown in SEQ ID NOs: 224-253, respectively.

In certain embodiments, an additional amino acid sequence can be appended to the C-terminal sequence of the alpha or beta chain of the MHCII molecule, for example for purposes of labeling and/or for attaching a moiety that mediates attachment (e.g., conjugation) to the multimerization domain. For example, an avitag (that mediates binding through the biotin binding site of Say) can be appended, such as an avitage with a Myc tag and a His tag (SEQ ID NO: 254) or an avitag with a Myc tag (SEQ ID NO: 255). In another embodiment, a sortag (that can mediate conjugation of click chemistry moieties through sortase, as described herein) can be appended, such as the sortag shown in SEQ ID NO: 257 or a sortag with a His tag as shown in SEQ ID NO: 256. In another embodiment, a V5 tag (SEQ ID NO: 258) is appended to the C-terminus.

In certain embodiments, heterodimerization pairs can be appended to the C-terminal sequence of the alpha and/or beta chains of the MHCII molecule. Non-limiting examples of such heterodimerization pair sequences include Fos and Jun (e.g., having the amino acid sequences shown in SEQ ID NOs: 259 and 260, respectively), acidic and basic leucine zippers (e.g., having the amino acid sequences shown in SEQ ID NOs: 261 and 262, respectively), knob and hole sequences (e.g., having the amino acid sequences shown in SEQ ID NOs: 263 and 264, respectively) for knobs-into-holes technology or spytab and spycatcher sequences (e.g., having the amino acid sequences shown in SEQ ID NOs: 265 and 266, respectively).

In certain embodiments, an MHCII-binding placeholder peptide is included in the expression construct for one of the MHCII chains, preferably the beta chain, such that the placeholder peptide and a digestible linker are encoded in the construct upstream of (N-terminally) and in operative linkage with the coding sequences for the MHCII chain. For example, the expression construct can encode (from N- to C-terminus): a placeholder peptide, an digestible linker, the MHCII chain (e.g., beta chain) and a C-terminal tag (e.g., encoding the amino acid sequence shown in SEQ ID NO: 192). In certain embodiments, an N-terminal tag is also appended upstream of the placeholder peptide, which allows for removal of non-exchanged peptide species following peptide exchange. Non-limiting examples of such N-terminal tags include a FLAG tag (e.g., having the amino acid sequence shown in SEQ ID NO: 267), a Strep-Tag (e.g., having the amino acid sequence shown in SEQ ID NO: 268) and a Protein C tag (e.g., having the amino acid sequence shown in SEQ ID NO: 269).

In some embodiments, the pMHCII multimers described herein comprise the α1 and α2 domains of an MHCII alpha chain and the β1 and β2 domains of an MHCII beta chain. In some embodiments, the multimer described herein comprises only the α1 and β1 domains of an MHCII heavy chain. In other embodiments, the pMHCII multimers comprise an alpha-chain and a beta-chain combined with a peptide. Other embodiments include an MHCII molecule comprised only of alpha-chain and beta-chain (so-called “empty” MHC II without loaded peptide), a truncated alpha-chain (e.g. the α1 domain) combined with full-length beta-chain, either empty or loaded with a peptide, a truncated beta-chain (e.g. the β1 domain) combined with a full-length alpha-chain, either empty or loaded with a peptide, or a truncated alpha-chain combined with a truncated beta-chain (e.g. α1 and β1 domain), either empty or loaded with a peptide.

In some embodiments, the multimeric protein comprises a soluble MHCII polypeptide. In some embodiments the MHC-multimeric protein comprises a soluble MHCII lacking transmembrane and intracellular domains.

The amino acid sequences of numerous MHC Class II proteins, including human MHCII, are known in the art, and the genes have been cloned. Therefore, the alpha and beta chain monomers can be expressed using recombinant methods. Methods for the expression and purification of MHCII molecules have been extensively described (e.g., Crawford et al. (1998) Immunity, 8:675-682; Novak et al. (1999) J. Clin. Invest., 104:R63-R67; Nepom et al. (2002) Arthrit. Rheum., 46:5-12; Day et al. (2003) J. Clin. Invest., 112:831-842; Vollers and Stern (2008) Immunol., 123:305-313; Cecconi et al. (2008) Cytometry, 73A:1010-1018, the entire contents of each of which is hereby incorporated by reference).

For MHC II molecules the alpha-chain and beta-chain may be expressed in separate cells as individual polypeptides or in the same cell as a fusion protein. The peptide of the MHC II-peptide complex may be produced separately and added following purification of whole MHC complexes or added during in vitro refolding or expressed together with alpha-chain and/or beta-chain connected to either chain through a linker. The genetic material can encode all or only a fragment of MHC class II alpha- and beta-chains. The genetic material may be fused with genes encoding other proteins, including proteins useful in purification of the expressed polypeptide chains (e.g., purification tags), proteins useful in increasing/decreasing solubility of the polypeptide(s), proteins useful in detection of polypeptide(s), proteins involved in coupling of MHC complex to multimerization domains and/or coupling of labels to MHC complex and/or MHC multimer.

In contrast to MHC I complexes, MHC II complexes are not easily refolded after denaturation in vitro. Only some MHC II alleles can be expressed in E. coli and refolded in vitro. Therefore, preferred expression systems for production of MHC II molecules are eukaryotic systems where refolding after expression of protein is not necessary. Preferred expression systems include mammalian expression systems, such as CHO cells, HEK cells or other mammalian cell lines suitable for expression of human proteins. Other expression systems include stable Drosophila cell transfectants, baculovirus infected insect-cells or other mammalian cell lines suitable for expression of proteins.

Stabilization of soluble MHC II complexes is even more important than for MHC I molecules, since both alpha- and beta-chain are participants in formation of the peptide binding groove and tend to dissociate when not embedded in the cell membrane. Accordingly, in one embodiment, MHCII monomers are prepared in which the peptide is covalently linked to the MHCII molecule. For example, one approach is the covalent synthesis of single-chain MHC class II chain-peptide complexes, directed by engineering peptide-specific complementary DNA (cDNA) sequences proximal to the beta-chain cDNA (as described in Crawford et al. (1999) Immunity, 8:675-682). In this strategy, the resulting polypeptide refolds with the peptide sequence extended from the amino terminus of the class II molecule. A tethering linker sequence in the peptide allows enough flexibility for the peptide to occupy the peptide binding groove in the mature class II molecule. A cleavable linker can be used to allow for cleavage of the covalent linkage between the peptide and the MHCII molecule (e.g., as described in Day et al. (2003) J. Clin. Invest., 112:831-842), thereby allowing for peptide exchange and loading of the MHCII molecule with other peptides (e.g., a library of different peptides).

Once expressed, the MHCII complexes can be purified directly as whole MHCII or MHCII-peptide monomers from MHCII expressing cells. The MHCII monomers may be expressed on the surface of cells, and are then isolated by disruption of the cell membrane using, e.g., detergent followed by purification of the MHCII. In some embodiments, MHC monomers are expressed into the periplasm and expressing cells are lysed and released MHCII monomers purified. Alternatively, MHC monomers may be purified from the supernatant of cells secreting expressed proteins into culture supernatant. Methods for purifying MHCII monomers are well known in the art, for example, via the use of affinity tags together with affinity chromatography, beads coated with ant-tag and/or other techniques involving immobilization of MHCII protein to affinity matrix; size exclusion chromatography using, e.g., gel filtration, ion exchange or other methods able to separate MHC molecules from cells and/or cell lysates.

In some embodiments, recombinant expression of MHCII polypeptides allow a number of modifications of the MHC monomers. For example, recombinant techniques provide methods for carboxy terminal truncation which deletes the hydrophobic transmembrane domain. The carboxy termini can also be arbitrarily chosen to facilitate the conjugation of ligands or labels, for example, by introducing cysteine and/or lysine residues into the molecule. The synthetic gene will typically include restriction sites to aid insertion into expression vectors and manipulation of the gene sequence. The genes encoding the appropriate monomers are then inserted into expression vectors, expressed in an appropriate host, such as E. coli, yeast, insect, or other suitable cells, and the recombinant proteins are obtained.

In some embodiments, the MHCII monomers are biotinylated on either their alpha or beta chain. In some embodiments, the MHCII monomers are biotinylated before loading of the peptide either by refolding or peptide exchange. Biotinylation of the MHC monomers can be achieved as known in the art, e.g. by attaching biotin to a specific attachment site which is the recognition site of a biotinylating enzyme. In some embodiments, the biotinylating enzyme is BirA. In some embodiments, biotinylation is carried out on the desired protein chain in vivo as a post translational modification during protein expression.

### II. Placeholder Peptides

A. MHC Class I Placeholder Peptides

In the methods provided herein, the MHCI monomers are loaded with a placeholder peptide to facilitate proper folding of the MHCI monomers to produce placeholder-peptide loaded MHCI (p*MHCI) prior to multimerization. Examples of placeholder peptides and methods of inducing folding MHCI heavy chains and β2-microglobulin in vitro in the presence of a placeholder peptide have been described in the art (e.g., Bakker et al., PNAS 105:3825-3830, 2008; Rodenko et al., Nat. Prot. 1: 1120-1132, 2006).

In some embodiments, the placeholder peptide is an HLA-A, HLA-B or HLA-C peptide. In some embodiments, the placeholder peptide is an HLA-A1 peptide (e.g., A*1:01 binding peptide). In some embodiments, the placeholder peptide is an HLA-A2 peptide (e.g., A*02:01 binding peptide, A*02:02 binding peptide, A*02:06 binding peptide). In other embodiments, the placeholder peptide is an HLA-A3 peptide (e.g., A*3:01 binding peptide), an HLA-A11 peptide (e.g., A*11:01 binding peptide), an HLA-A23 peptide (e.g., A*23:01 binding peptide), an HLA-A24 peptide (e.g., A*24:02 binding peptide), an HLA-A26 peptide (e.g., A*26:01 binding peptide), an HLA-A29 peptide (e.g., A*29:02 binding peptide), an HLA-A30 peptide (e.g., A*30:01 binding peptide; A*30:02 binding peptide), an HLA-A31 peptide (e.g., A*31:01 binding peptide), an HLA-A32 peptide (e.g., A*32:01 binding peptide), an HLA-A33 peptide (e.g., A*33:01 binding peptide; A*33:03 binding peptide), an HLA-A68 peptide (e.g., A*68:02 binding peptide), an HLA-B7 peptide (e.g., B*07:02 binding peptide), an HLA-B8 peptide (e.g., B*08:01 binding peptide), an HLA-B15 peptide (e.g., B*15:01 binding peptide; B*15:03 binding peptide), an HLA-B18 peptide (e.g., B*18:01 binding peptide), an HLA-B35 peptide (e.g., B*35:01 binding peptide), an HLA-B38 peptide (e.g., B*38:01 binding peptide), an HLA-B40 peptide (e.g., B*40:01 binding peptide; B*40:02 binding peptide), an HLA-B45 peptide (e.g., B*45:01 binding peptide), an HLA-B51 peptide (e.g., B*51:01 binding peptide), an HLA-B53 peptide (e.g., B*53:01 binding peptide), an HLA-B58 peptide (e.g., B*58:01 binding peptide), an HLA-C3 peptide (e.g., C*03:03 binding peptide; C*03:04 binding peptide), an HLA-C4 peptide (e.g., C*04:01 binding peptide) an HLA-C7 peptide (e.g., C*07:01 binding peptide; C*07:02 binding peptide) or an HLA-C8 peptide (e.g., C*08:01 binding peptide). In some embodiments, the placeholder peptide is a synthetic peptide.

In some embodiments, the affinity of the placeholder peptide for the binding groove of MHCI is lower than the rescue peptide(s). In some embodiments, the affinity of the placeholder peptide for the MHCI binding groove is about 10-fold lower than the rescue peptide(s). In some embodiments, the affinity of the place holder peptide for the binding groove of MHCI is higher than the rescue peptide(s); however, the placeholder peptide can still be replaced by the rescue peptide by use of an excess concentration of the rescue peptide.

In some embodiments, the placeholder peptide is thermolabile. Is some embodiments, the placeholder peptide is thermolabile at a temperature between about 30-37° C. In some embodiments, the placeholder peptide is labile at a temperature at or above 30° C., at or above 32° C., at or above 34° C., at or above 35° C., at or above 36° C., or at about 37° C. Thermal labile placeholder peptides and methods of identifying and producing thermal labile placeholder peptides have been described (e.g., WO 93/10220; WO 2005/047902; US 2008/0206789; Luimstra et al., Curr. Protoc. Immunol. 126(1):e85, 2019; Luimstra et al., J. Exp. Med. 215(5):1493-1504, 2018).

In some embodiments the placeholder peptide is labile at an acidic pH. In some embodiments, the placeholder peptide is labile between about pH 2.5 and 6.5. In some embodiments, the placeholder peptide is labile at a pH of about 2.5-6.0, 3.0-6.0, 3.0-6.5, 3.5-6.0 3.5-6.5, 4.0-6.0, 4.0-6.5, 4.5-6.0, 4.5-6.5, 5.0-6.0, 5.0-6.5, 5.0, 5.5., 6.0 or 6.5. In some embodiments, the placeholder peptide is labile at a basic pH. In some embodiments, the placeholder peptide is labile between about pH 9-11. In some embodiments, the placeholder peptide is labile at or above pH 9, at or above pH 9.5, at or about pH 10, at or about pH 10.5, or at or about pH 11. Methods of generating and using pH sensitive placeholder peptides are publicly available, for example, as described in WO 93/10220; US 2008/0206789; and Cameron et al., J. Immunol. Meth. 268:51-59.

In some embodiments, the placeholder peptide comprises a cleavable moiety. Various types of cleavable moieties are known in the art and include, for example, moieties that are cleaved by photoirradiation, enzymes, nucleophilic or electrophilic agents, reducing and oxidizing reagents (e.g., reviewed in Leriche et al., Biorg. Med. Chem. 20(2):571-582, 2012).

In some embodiments, the cleavable placeholder peptide comprises one or more photocleavable non-natural β-amino acids. In some embodiments, the placeholder peptide comprises 3-amino-3-(2-nitro-phenyl)-proprionic acid. In some embodiments, the placeholder peptide comprises (2-nitro)phenylglycine. In some embodiments, the placeholder peptide comprises an azobenzene group. In some embodiments, the HLA-A2 placeholder peptide is A*02:01, KILGFVFJV (SEQ ID NO: 15) or GILGFVFJL (SEQ ID NO: 7), wherein J is 3-amino-3-(2-nitro)phenyl-propionic acid. In some embodiments, the placeholder peptide is selected from the group consisting of A*01:01, STAPGJLEY (SEQ ID NO: 16); A*03:01, RIYRJGATR (SEQ ID NO:17); A*11:01, RVFAJSFIK (SEQ ID NO: 18); A*24:02, VYGJVRACL (SEQ ID NO: 11); B*07:02, AARGJTLAM (SEQ ID NO: 14); B*35:01, KPIVVLJGY (SEQ ID NO: 19); C*03:04, FVYGJSKTSL (SEQ ID NO: 20), B*08:01, FLRGRAJGL (SEQ ID NO: 21); C*07:02, VRIJHLYIL (SEQ ID NO: 22); C*04:01, QYDJAVYKL (SEQ ID NO: 23); B*15:01, ILGPJGSVY (SEQ ID NO: 24); B*40:01, TEADVQJWL (SEQ ID NO: 25); B*58:01, ISARGQJLF (SEQ ID NO: 26); and C*08:01, KAAJDLSHFL (SEQ ID NO: 27), wherein J is 3-amino-3-(2-nitro)phenyl-propionic acid. In another embodiment, a placeholder peptide comprises a sequence shown in any one of SEQ ID NO: 7-27 or 271-279. In another embodiment, a placeholder peptide consists of a sequence shown in any one of SEQ ID NO: 7-27 or 271-279.

Methods of generating placeholder peptides containing photocleavable amino acids are known in the art and have been previously described (e.g., Toebes et al., Curr. Protoc. Immunol. 87:18.16.1-18.16.20, 2009; Bakker et al., supra, Rodenko et al. supra). In various embodiments, the photocleavable placeholder peptide is cleaved upon exposure to UV-light using previously described methods (e.g., Toebes et al., Nat Med. 2006 February; 12(2):246-51; Bakker et al., Proc Natl Acad Sci USA. 2008 Mar. 11; 105(10):3825-30; Rodenko et al., Nat Protoc. 2006; 1(3):1120-32; Frøsig et al., Cytometry A. 2015 October; 87(10):967-75). In some embodiments, the placeholder peptide comprises a chemoselective moiety. In some embodiments, the chemoselective moiety comprises a sodium dithionite sensitive azobenzene linker, wherein the azobenzene comprises at least one aromatic group comprising an electron-donor group and is located between two amino acid residues. Azobenzine linkers and methods for chemoselective peptide exchange are known in the art, for example, as described in U.S. Pat. No. 10,400,024.

In some embodiments, the placeholder peptide comprises a cleavable moiety that is cleaved upon exposure to an aminopeptidase. In some embodiments, the cleavage of the amino acid residue occurs via the use of a methionine aminopeptidase. The methionine aminopeptidase can cleave a methionine from a peptide when the amino acid residue at position two is, for example, glycine, alanine, serine, cysteine, or proline. In some embodiments, the cleavable moiety comprises a thrombin cleavage domain.

In some embodiments, the placeholder peptide comprises a cleavable moiety is sensitive to a chemical trigger. In some embodiments, the placeholder peptide comprises periodate-sensitive amino acid. In some embodiments, the periodate-sensitive amino acid comprises a vicinal diol moiety. In some embodiments, the periodate-sensitive amino acid comprises a vicinal amino alcohol. In some embodiments, the periodate-sensitive amino acid is 1,2-amino-alcohol-containing amino acid. In some embodiments, the periodate-sensitive amino acid is α,γ-diamino-β-hydroxybutanoic acid (DAHB). Methods for producing and using peptides containing periodate-sensitive amino acids are publicly available, for example, as described in Rodenko et al. (J. Am. Chem. Soc. 131:12605-12313, 2009) and Amore et al. (ChemBioChem 14:123-131, 2013).

In some embodiments, the placeholder peptide is a dipeptide. In some embodiments, the dipeptide binds to the F pocket of the MHCI binding groove. In some embodiments, the second amino acid of the dipeptide is hydrophobic. In some embodiments, the dipeptide is selected from the group consisting of glycyl-leucine (GL), glycyl-valine (GV), glycyl-methione (GM), glycyl-cyclohexylalanine (GCha), glycyl-homoleucine (GHle) and glycyl-phenylalanine (GF). Methods for producing and using dipeptides as placeholder peptides are publicly available, for example, as described in Saini et al. (PNAS 112:202-207, 2015).

In some embodiments, the placeholder peptide comprises GILGFVFJL (SEQ ID NO:7). In some embodiments, the placeholder peptide consists of GILGFVFJL (SEQ ID NO:7). In other embodiments, a placeholder peptide comprises a sequence shown in any one of SEQ ID NO: 8-27 or 271-279. In other embodiments, a placeholder peptide consists of a sequence shown in any one of SEQ ID NO: 8-27 or 271-279.

In some embodiments, the placeholder peptide further comprises a fluorescent label. In some embodiments, the fluorescent label is attached to a cysteine residue in the placeholder peptide.

In some embodiments, p*MHCI molecules are purified, and stored to serve as a source of stock molecules that can be exchanged with peptide epitopes of interest upon exposure to peptide exchange conditions as described herein.

B. MHC Class II Placeholder Peptides

In the methods provided herein, the MHCII monomers are loaded with a placeholder peptide to facilitate proper folding of the MHCII monomers to produce placeholder-peptide loaded MHCII (p*MHCII) prior to multimerization. In various embodiments, the placeholder peptide is peptide that binds HLA-DR, HLA-DQ, HLA-DX, HLA-DO, HLA-DZ or HLA-DP. In some embodiments, the placeholder peptide is a synthetic peptide.

In some embodiments, the affinity of the placeholder peptide for the binding groove of MHCII is lower than the rescue peptide(s). In some embodiments, the affinity of the placeholder peptide for the MHCII binding groove is about 10-fold lower than the rescue peptide(s).

In some embodiments, the placeholder peptide is thermolabile. In some embodiments, the placeholder peptide is thermolabile at a temperature between about 30-37° C. In some embodiments, the placeholder peptide is labile at a temperature at or above 30° C., at or above 32° C., at or above 34° C., at or above 35° C., at or above 36° C., or at about 37° C. Thermal labile placeholder peptides and methods of identifying and producing thermal labile placeholder peptides have been described (e.g., WO 93/10220; WO 2005/047902; US 2008/0206789; Luimstra et al., Curr. Protoc. Immunol. 126(1):e85, 2019; Luimstra et al., J. Exp. Med. 215(5):1493-1504, 2018).

In some embodiments the placeholder peptide is labile at an acidic pH. In some embodiments, the placeholder peptide is labile between about pH 2.5 and 6.5. In some embodiments, the placeholder peptide is labile at a pH of about 2.5-6.0, 3.0-6.0, 3.0-6.5, 3.5-6.0 3.5-6.5, 4.0-6.0, 4.0-6.5, 4.5-6.0, 4.5-6.5, 5.0-6.0, 5.0-6.5, 5.0, 5.5., 6.0 or 6.5. In some embodiments, the placeholder peptide is labile at a basic pH. In some embodiments, the placeholder peptide is labile between about pH 9-11. In some embodiments, the placeholder peptide is labile at or above pH 9, at or above pH 9.5, at or about pH 10, at or about pH 10.5, or at or about pH 11. Methods of generating and using pH sensitive placeholder peptides are publicly available, for example, as described in WO 93/10220; US 2008/0206789; and Cameron et al., J. Immunol. Meth. 268:51-59.

In some embodiments, the placeholder peptide comprises a cleavable moiety. Various types of cleavable moieties are known in the art and include, for example, moieties that are cleaved by photoirradiation, enzymes, nucleophilic or electrophilic agents, reducing and oxidizing reagents (e.g., reviewed in Leriche et al., Biorg. Med. Chem. 20(2):571-582, 2012).

In one embodiment, the placeholder peptide is fused to a degradation tag and peptide exchange is promoted by proteolysis in the presence of a corresponding protease (the digests the degradation tag) along with the presence of the rescue peptide(s).

In some embodiments, the cleavable placeholder peptide is a photocleavable peptide, e.g., cleaved upon exposure to UV light. For example, the placeholder peptide can comprise one or more photocleavable photocleavable non-natural amino acids. MHCII-binding photocleavable peptides, e.g., that incorporate the UV-sensitive amino acid analog 3-amino-3-(2-nitrophenyl)-propionate have been described (see e.g., Negroni and Stern (2018) PLos One, 13(7):e0199704).

In one embodiment, the MHCII placeholder peptide is a CLIP peptide, such as having the amino acid sequence KPVSKMRMATPLLMQA (SEQ ID NO: 189) or ATPLLMQALPMGA (SEQ ID NO: 280). In one embodiment, the CLIP peptide is cleavable. In one embodiment, the MHCII monomers are synthesized with the cleavable CLIP peptide covalently attached, such as by synthesis of single-chain MHC class II chain-peptide complexes, directed by engineering peptide-specific complementary DNA (cDNA) sequences proximal to the beta-chain cDNA (see e.g., Day et al. (2003) J. Clin. Invest., 112:831-842). Cleavage of the covalent linkage between the CLIP peptide (as the placeholder peptide) and MHCII thus allows for peptide exchange with other MHCII-binding peptides.

Other MHCII binding peptides have been described in the art that can be used as placeholder peptides, based on appropriate pairing of an MHCII molecule and its known MHCII binding peptide. Non-limiting examples of known MHCII molecule/MHCII binding peptide pairs include: DRA1*0101/DRB1*0401 and the immunodominant peptide of hemagglutinin, HA₃₀₇₋₃₁₉ (see Novak et al. (1999) J. Clin. Invest., 104:R63-R67) and HLA-DR*1101 and tetanus-toxoid (TT)-derived p2 peptide (TT₈₃₀₋₈₄₄) having the amino acid sequence QIYKANSKFIGITEL (SEQ ID NO: 190) (see Cecconi et al. (2008) Cytometry, 73A:1010-1018).

III. Production of p*MHC Multimers

Multimerization domains for use in producing the pMHC multimers provided herein include proteins, polypeptide or other multimeric moieties suitable for the covalent conjugation of two or more pMHC or p*MHC monomers, which do not interfere with binding of the pMHC polypeptides to cells. In some embodiments, the multimerization domain comprises protein subunits. In some embodiments, the multimerization domain is a homomultimer of protein subunits. In some embodiments, the multimerization domain is a heteromultimer of protein subunits. In some embodiments, the multimer is a dimer, trimer, tetramer, pentamer, hexamer, octamer decamer or dodecamer. In one preferred embodiment, the pMHC multimer is a tetramer.

Examples of suitable binding entities are streptavidin (SA) and avidin and derivatives thereof, biotin, immunoglobulins, antibodies (monoclonal, polyclonal, and recombinant), antibody fragments and derivatives thereof, leucine zipper domain of AP-1 (jun and fos), hexa-his (metal chelate moiety), hexa-hat GST (glutathione S-tranferase) glutathione affinity, Calmodulin-binding peptide (CBP), Strep-tag®, Cellulose Binding Domain, Maltose Binding Protein, S-Peptide Tag, Chitin Binding Tag, Immuno-reactive Epitopes, Epitope Tags, E2Tag, HA Epitope Tag, Myc Epitope, FLAG Epitope, AU1 and AU5 Epitopes, Glu-Glu Epitope, KT3 Epitope, IRS Epitope, Btag Epitope, Protein Kinase-C Epitope, VSV Epitope, lectins that mediate binding to a diversity of compounds, including carbohydrates, lipids and proteins, e. g., Con A (Canavaliaensi formis) or WGA (wheat germ agglutinin) and tetranectin or Protein A or G (antibody affinity) or coiled-coil polypeptides e.g. leucine zipper. Combinations of such binding entities are also included.

In some embodiments, the multimerization domain is a tetramer of streptavidin (SA or SAv) or a derivative thereof. In some embodiments, the multimerization domain is tetrameric streptavidin. In some embodiments, the tetramer comprises Strep-tag® or Strep-tactin®. Strep-tag® or Strep-tactin® are described in U.S. Pat. Nos. 5,506,121 and 6,103,493, respectively, and are commercially available from a number of sources. To attach MHC monomers to streptavidin non-covalently via the biotin-binding site of SAv, an avitag (such as having the amino acid sequence shown in SEQ ID NO: 161, which includes a 6×His Tag and a FLAG tag) can be incorporated into MHC monomer, for example at the C-terminal end (see e.g., Example 3).

In the methods provided herein, pMHC multimers are produced by covalent conjugation of each p*MHC monomer to the N- or C-terminal of each subunit of the multimerization domain, resulting in a reaction product referred to herein as a Conjugated Multimer. In one embodiment, the Conjugated Multimer is a pMHC Class I (pMHCI) Conjugated Multimer. In another embodiment, the Conjugated Multimer is a pMHC Class II (pMHCII) Conjugated Multimer.

In some embodiments, pMHCI multimers are produced by covalent conjugation of the multimerization domain to the C-terminus of the MHCI α1 domain. In some embodiments, the pMHCI multimers are produced by covalent conjugation of the multimerization domain to the C-terminus of the MHCI α2 domain. In some embodiments, the pMHCI multimers are produced by covalent conjugation of the multimerization domain to the C-terminus of the MHCI α3 domain. In some embodiments, the pMHCI multimers are produced by covalent conjugation of the multimerization domain to the C-terminus of the β2-microglobulin of each p*MHC monomer.

In a preferred embodiment, pMHCII multimers are produced by covalent conjugation of the multimerization domain to the MHCII α chain. In another embodiment, pMHCII multimers are produced by covalent conjugation of the multimerization domain to the MHCII β chain. In certain embodiments, pMHCII multimers are produced by covalent conjugation of the multimerization domain to the C-terminus of the MHCII α1 domain. In certain embodiments, the pMHCII multimers are produced by covalent conjugation of the multimerization domain to the C-terminus of the MHCII α2 domain. In certain embodiments, the pMHCII multimers are produced by covalent conjugation of the multimerization domain to the C-terminus of the MHCII β1 domain. In certain embodiments, the pMHCII multimers are produced by covalent conjugation of the multimerization domain to the C-terminus of the MHCII β2 domain.

A number of suitable methods for forming covalent bonds between each MHC monomer and the multimerization domain are provided herein.

A. Chemical Bioconjujation

In some embodiments, the p*MHC multimers are produced by chemical conjugation. In some embodiments, the chemical conjugation is mediated by cysteine bioconjugation of the p*MHC polypeptides to the multimerization domain. In some embodiments, the cysteine bioconjugation is mediated by cysteine alkylation. In some embodiments, the cysteine bioconjugation is mediated by cysteine oxidation. In other embodiments, the cysteine bioconjugation is mediated by a desulfurization reaction. In some embodiments, cysteine bioconjugation is mediated by iodoacetamide. In some embodiments, the cysteine bioconjugation is mediated by maleimide. Methods for utilizing cysteine mediated linkage of two moieties which can be used to produce the pMHC multimers disclosed herein have been described, for example, see Chalker et al., Chem Asian J. 4(5):630-40, 2009; Spicer et al., Nat Commun. 5:4740, 2015.

In some embodiments, the MHC multimers are produced by chemical modification of amino acids other than cysteine, including but not limited to lysine, tyrosine, arginine, glutamate, aspartate, serine, threonine, methionine, histidine and tryptophan side-chains, as well as N-terminal amines or C-terminal carboxyls, as previously described (Baslé et al., M Chem Biol. 17(3):213-27, 2010; Hu et al., Chem Soc Rev. 45(6):1691-719, 2016; Lin et al., Science 355(6325):597-602, 2017).

B. Native Chemical Lijation

In some embodiments, the pMHC multimers are produced by native chemical ligation (NCL), wherein each p*MHC polypeptide comprises a C-terminal thioester, and each subunit of the multimerization domain comprises an N-terminal cysteine residue, or functional equivalent thereof, wherein the reaction between the cysteine side-chain and the thioester irreversibly forms a native peptide bond, thus ligating the p*MHC monomers to the multimerization domain. Methods for NCL have been described (Hejjaoui et al., M Protein Sci. 24(7):1087-99. 2015) Mandal et al., Proc Natl Acad Sci USA 109(37):14779-84, 2012; Torbeev et al., Proc Natl Acad Sci USA 110(50):20051-6, 2013).

In some embodiments, β- and/or γ-thio amino acids are incorporated into the p*MHC monomers. In some embodiments, β- and/or γ-thio amino acids replace the cysteine-like residue at an N-terminal position of each subunit of the multimerization domain, e.g., to provide a reactive thiol for trans-thioesterification. Desulfurization protocols can then produce the desired native side-chain. In some embodiments, NCL is performed at an alanine residue. In other embodiments, NCL is performed at phenylalanine (Crich & Banerjee, 2007), valine (Chen et al. 2008; Haase et al. 2008), leucine (Harpaz et al. 2010; Tan et al. 2010), threonine (Chen et al. 2010b), lysine (El Oualid et al. 2010; Kumar et al. 2009; Yang et al. 2009), proline (Shang et al. 2011), glutamine (Siman et al. 2012), arginine (Malins et al. 2013), tryptophan (Malins et al. 2014), aspartate (Thompson et al. 2013), glutamate (Cergol et al. 2014) and asparagine (Sayers et al. 2015). Ligation/desulfurization approaches that remove purification steps and increase the yield of ligated products have been described (Moyal et al. 2013; Thompson et al. 2014).

C. Click Chemistry Mediated Bioorthogonal Conjugation

In some embodiments, the p*MHC multimers are produced by bioorthogonal conjugation between the conjugation moiety at the C-terminus of each p*MHC monomer and the conjugation moiety at the N-terminus of each subunit of the multimerization domain. In some embodiments, the bioorthongonal conjugation is mediated by “click chemistry.” (see, e.g., Kolb, Finn and Sharpless, Angewandte Chemie International Edition (2001) 40: 2004-2021). Conjugation moieties suitable for click chemistry, reaction conditions, and associated methods are available in the art (e.g., Kolb et al., Angewandte Chemie International Edition 40:2004-2021, 2001; Evans, Australian Journal of Chemistry 60: 384-395, 2007; Lahann, Click Chemistry for Biotechnology and Materials Science, John Wiley & Sons Ltd, ISBN 978-O-470-69970-6, 2009). In some embodiments, a click chemistry moiety may comprise or consist of a terminal alkyne, azide, strained alkyne, diene, dieneophile, alkoxyamine, carbonyl, phosphine, hydrazide, thiol, or alkene moiety. In certain embodiments, the azide is a copper-chelating azide. In one embodiment, the copper-chelating azide is a picolyl azide, such as Gly-Gly-Gly-(PEG)4-Picolyl-Azide. Reagents for use in click chemistry reactions are commercially available, such as from Click Chemistry Tools (Scottsdale, Ariz.) or GenScript (Piscataway, N.J.).

For conjugation of each p*MHC monomer to a subunit of the multimerization domain via click chemistry, the click chemistry moieties of the proteins have to be reactive with each other, for example, in that the reactive group of one of the click chemistry moiety of each p*MHC monomer reacts with the reactive group of the second click chemistry moiety on a subunit of the multimerization domain to form a covalent bond. Such reactive pairs of click chemistry handles are well known to those of skill in the art and include but are not limited to those set forth in FIG. 1.

In some embodiments, each p*MHC conjugation moiety can be covalently conjugated under click chemistry reaction conditions to the conjugation moiety of each subunit of the multimerization domain. In some embodiments a sortase-mediated conjugation is used to install a first click chemistry moiety at the C-terminus of each p*MHC monomer, and a second click chemistry moiety reaction to each subunit of the multimerization domain. In the methods provided herein, two or more p*MHC monomers containing the first click chemistry moiety are conjugated to the second click chemistry moiety at the C-terminus of each subunit of the multimerization domain under click chemistry conditions. Methods of attaching click chemistry moieties utilizing sortase are described, for example, in WO2013/00355, the entire contents of which is hereby incorporated by reference. Non-limiting exemplifications of pMHC multimers prepared using Alkyne-Azide click chemistry in combination with sortase-mediated conjugation are described in detail in Examples 1, 5, 6 and 7.

In some embodiments, an intein-mediated conjugation is used to install a first click chemistry moiety at the C-terminus of each p*MHC monomer, and a second click chemistry moiety reaction to each subunit of the multimerization domain. Methods of utilizing intein-mediated conjugated are described further herein.

In some embodiments, the methods of click chemistry mediated covalent conjugation of the p*MHC monomers to the multimerization domain provided herein comprise native chemical ligation of C-terminal thioesters with β-amino thiols (Xiao J, Tolbert T J Org Lett. 2009 Sep. 17; 11(18):4144-7).

In some embodiments, the click chemistry used to produce the p*MHC multimers comprises 1,3-dipolar cycloaddition (e.g., the Cu(I)-catalyzed stepwise variant, often referred to simply as the “click reaction”; see, e.g., Tornoe et al., Journal of Organic Chemistry (2002) 67: 3057-3064). Copper and ruthenium are the commonly used catalysts in the reaction. The use of copper as a catalyst results in the formation of 1,4-regioisomer whereas ruthenium results in formation of the 1,5-regioisomer.

In some embodiments, the MHC monomers are ligated to an alkynated peptide by expressed protein ligation (EPL) and then conjugated to an azide-labeled multimerization domain by Cu(I)-catalyzed terminal azide-alkyne cycloaddition (CuAAC).

In some embodiments, the click chemistry conjugation comprises a cycloaddition reaction, such as the Diels-Alder reaction. In some embodiments, the MHCI and multimerization domain are conjugated by azide-alkyne 1,3-dipolar cycloaddition (“click chemistry). In some embodiments, the cycloaddition is promoted by the presence of Cu(I)-catalyzed cycloaddition (CuAAC).

In some embodiments, the click chemistry conjugation comprises nucleophilic addition to small strained rings like epoxides and aziridines. In some embodiments, the cycloaddition is promoted by strained cyclooctyne systems, for example, as described in Agard N J, Prescher J A, Bertozzi C R J Am Chem Soc. 2004 Nov. 24; 126(46):15046-7.

In some embodiments, the click chemistry conjugation comprises nucleophilic addition to activated carbonyl groups.

In some embodiments, the conjugation of the pMHC monomers and multimerization domain occurs by a bioorthogonal reaction. In some embodiments, the MHC and multimerization domain are conjugated by inverse-electron demand Diels-Alder reactions between strained dienophiles and tetrazine dienes, for example, as described in Blackman M L, Royzen M, Fox J M J Am Chem Soc. 2008 Oct. 15; 130(41):13518-9; and Devaraj N K, Weissleder R, Hilderbrand S A Bioconjug Chem. 2008 December; 19(12):2297-9). In some embodiments, the dienophile is a trans-cyclooctene. In some embodiments, the dienophile is a norbornene.

D. Sortase Mediated Conjugation

In some embodiments, conjugation between the p*MHC monomers and the multimerization domain is mediated by a cysteine transpeptidase. In some embodiments, the cysteine transpeptidase is a sortase, or enzymatically active fragment thereof. A variety of sortase enzymes have been described and are commercially available (e.g., Antos et al., Curr. Opin. Struct. Biol. 38:111-118, 2016). Sortases recognize and cleave an amino acid motif, referred to as a “sortag”, to produce a peptide bond between the acyl donor and acceptor site on two polypeptides, resulting in the ligation of different polypeptides which contain N- or C-terminal sortags. Non-limiting exemplifications of pMHC multimers prepared using sortase-mediated conjugation (in combination with Alkyne-Azide click chemistry) are described in detail in Examples 1, 5, 6 and 7.

Accordingly, in some embodiments, each p*MHC monomer comprises a C-terminal sortag, and each subunit of the multimerization domain comprises an N-terminal sortag. In other embodiments, each p*MHC monomer comprises an N-terminal sortag, and each subunit of the multimerization domain comprises a C-terminal sortag. In some embodiments, the sortase catalyzes the formation of a peptide bond between an MHC polypeptide and each of the subunits of the multimerization domain.

In some embodiments, the recognition motif is added to the C-terminus of each of the pMHC monomers, and an oligo-glycine motif is added to the N-terminus of each of the subunits of the mutimerization domain. Upon addition of sortase to the mixture of MHC monomers and multimerization domains, the polypeptides are covalently linked through a native peptide bond to produce a pMHC multimer.

In some embodiments, the MHC monomers and/or multimerization domain are expressed in frame with the sortags. In some embodiments, additional tags may be included, for example, a 6×-His tag (Sinisi et al. Bioconjug. Chem 23:1119-1126, 2012), a nucleophilic fluorochrome (Nair et al. Immun. Inflamm. Dis. 1:3-13, 2013), and/or a FLAG tag (Greineder et al. Bioconjug. Chem. 29:56-66, 2018).

In some embodiments, the sortag contains a modified amino acid suitable for chemical conjugation between the MHC monomers and the mutimerization domain. In some embodiments, the sortag contains a C-terminal azidolysine residue to enable oriented click-click chemistry conjugation as described herein.

In some embodiments, the MHC polypeptide and/or multimerization domains comprise a linker between the polypeptide and the sortag. In some embodiments, each MHC polypeptide and each subunit of the multimerization domain comprises a sortag with a linker. Suitable linkers have been described, for example, in Greineder et al., Bioconjug. Chem. 29:56-66, 2018. In some embodiments, the linker is a semi-rigid linker. In some embodiments, the linker comprises (SSSSG)₂SAA (SEQ ID NO: 182). In some embodiments, the linker comprises (G)₅ (SEQ ID NO: 183).

In some embodiments, the sortag contains a fluorophore-modified lysine residue to facilitate measurement of reaction progression and efficiency

In some embodiments, the sortase is Ca2+ dependent. In some embodiments, the sortase is Ca2+ independent.

In some embodiments, the sortag-labeled MHC molecule is a soluble HLA-A2 molecule (HLA-A*02:01) with a C-terminal sortag and 6×His tag, such as having the amino acid sequence shown in SEQ ID NO: 1. In some embodiments, the sortag-labeled multimerization domain is a streptavidin molecule with a C-terminal sortag and 6×His Tag, such as having the amino acid sequence shown in SEQ ID NO: 3. In some embodiments, the sortag label with a 6×His tag has the amino acid sequence shown in SEQ ID NO: 162. Various other sortag sequences are known in the art and are suitable for use in preparing the Conjugated Multimers of the disclosure, non-limiting examples of which are described further below.

In some embodiments, the sortag comprises the amino acid sequence LPXTG (SEQ ID NO: 163), wherein X is any amino acid, and the sortase cleaves between the threonine and glycine backbone within the motif.

In some embodiments, the sortase recognizes a sortag comprising an amino acid sequence selected from IPKTG (SEQ ID NO:164), MPXTG (SEQ ID NO:165), LAETG (SEQ ID NO:166), LPXAG (SEQ ID NO:167), LPESG (SEQ ID NO:168), LPELG (SEQ ID NO:169) or LPEVG (SEQ ID NO:170).

In some embodiments, the sortase is a SrtAstaph mutant. In some embodiments, the SrtAstaph mutant is F40, and the recognition motif is XPKTG (SEQ ID NO: 171) (Piotukh et al., J. Am. Chem. Soc. 2011 133:17536-17539). In some embodiments, the SrtAstaph mutant is F40 and the recognition motif is APKTG (SEQ ID NO:172), DPKTG (SEQ ID NO:173) or SPKTG (SEQ ID NO:174).

In some embodiments, the SrtAstaph mutant is SrtAstaph pentamutant and the recognition motif is LPXTG (SEQ ID NO:163), wherein X is any amino acid, LPEXG, (SEQ ID NO:175), wherein X is any amino acid, or LAETG (SEQ ID NO:166). In some embodiments, the mutant is SrtAstaph pentamutant and the recognition motif is LPEAG (SEQ ID NO:176), LPECG (SEQ ID NO:177) or LPESG (SEQ ID NO:168). In some embodiments, the SrtAstaph mutant is 2A-9 and the recognition motif is LAETG (SEQ ID NO:166). In some embodiments, the SrtAstaph mutant is 4S-9 and the recognition motif is LPEXG (SEQ ID NO:178), wherein X=A, C or 5).

In some embodiments, the sortase is a soluble fragment of the wild-type sortase. In some embodiments, the sortase is a soluble fragment of a modified sortase A (Mao H, Hart S A, Schink A, Pollok B A, J Am Chem Soc. 2004 Mar. 10; 126(9):2670-1 A).

In some embodiments, the sortase is a variant or homolog of S. aureus sortase A (Antos J M, Truttmann M C, Ploegh H L Curr Opin Struct Biol. 2016 June; 38:111-8; Don B M, Ham H O, An C, Chaikof E L, Liu D R Proc Natl Acad Sci USA. 2014 Sep. 16; 111(37):13343-8; Glasgow J E, Salit M L, Cochran J R J Am Chem Soc. 2016 Jun. 22; 138(24):7496-9).

Methods of conjugation of sortags into proteins have also been described. (Matsumoto T, Furuta K, Tanaka T, Kondo A ACS Synth Biol. 2016 Nov. 18; 5(11):1284-1289; Williams F P, Milbradt A G, Embrey K J, Bobby R PLoS One. 2016; 11(4):e0154607; and Witte M D, Cragnolini J J, Dougan S K, Yoder N C, Popp M W, Ploegh H L Proc Natl Acad Sci USA. 2012 Jul. 24; 109(30):11993-8; Mao H, Hart S A, Schink A, Pollok B A J Am Chem Soc. 2004 Mar. 10; 126(9):2670-1; Guimaraes C P, Witte M D, Theile C S, Bozkurt G, Kundrat L, Blom A E, Ploegh H L Nat Protoc. 2013 September; 8(9):1787-99 and Theile C S, Witte M D, Blom A E, Kundrat L, Ploegh H L, Guimaraes C P Nat Protoc. 2013 September; 8(9):1800-7.

In some embodiments, the aminoglycine peptide fragment generated by the sortase reaction, is removed by dialysis or centrifugation, e.g., while the reaction is proceeding (Freiburger L, Sonntag M, Hennig J, Li J, Zou P, Sattler M J Biomol NMR. 2015 September; 63(1):1-8). In some embodiments, affinity immobilization strategies or flow-based platforms are used for the selective removal of reaction components (Policarpo R L, Kang H, Liao X, Rabideau A E, Simon M D, Pentelute B L Angew Chem Int Ed Engl. 2014 Aug. 25; 53(35):9203-8).

In some embodiments, the equilibrium of the reaction can be controlled by ligation product or by-product deactivation. For example, in some embodiments the reaction is controlled by ligation of a WTWTW (SEQ ID NO: 179) motif added to the donor and acceptor as described in Yamamura Y, Hirakawa H, Yamaguchi S, Nagamune T Chem Commun (Camb). 2011 Apr. 28; 47(16):4742-4). In other embodiments, by-products are deactivated by chemical modification of the acyl donor glycine as described, for example, in Liu F, Luo E Y, Flora D B, Mezo A R J Org Chem. 2014 Jan. 17; 79(2):487-92; and Williamson D J, Webb M E, Turnbull W B Nat Protoc. 2014 February; 9(2):253-62).

E. Intein-Mediated Conjugation

Inteins are naturally occurring, self-splicing protein subdomains that are capable of excising out their own protein subdomain from a larger protein structure while simultaneously joining the two formerly flanking peptide regions (“exteins”) together to form a mature host protein. Intein-based methods of protein modification and ligation have been developed. An intein is an internal protein sequence capable of catalyzing a protein splicing reaction that excises the intein sequence from a precursor protein and joins the flanking sequences (N- and C-exteins) with a peptide bond. A non-limiting exemplification of pMHC multimers prepared using intein-mediated conjugation is described in detail in Example 2.

As used herein, the term “split intein” refers to any intein in which one or more peptide bond breaks exists between the N-terminal intein segment and the C-terminal intein segment such that the N-terminal and C-terminal intein segments become separate molecules that cannon-covalently reassociate, or reconstitute, into an intein that is functional for splicing or cleaving reactions. Any catalytically active intein, or fragment thereof, may be used to derive a split intein for use in the systems and methods disclosed herein. For example, in one aspect the split intein may be derived from a eukaryotic intein. In another aspect, the split intein may be derived from a bacterial intein. In another aspect, the split intein may be derived from an archaeal intein. Preferably, the split intein so-derived will possess only the amino acid sequences essential for catalyzing splicing reactions.

As used herein, the “N-terminal intein segment” refers to any intein sequence that comprises an N-terminal amino acid sequence that is functional for splicing and/or cleaving reactions when combined with a corresponding C-terminal intein segment. An N-terminal intein segment thus also comprises a sequence that is spliced out when splicing occurs. An N-terminal intein segment can comprise a sequence that is a modification of the N-terminal portion of a naturally occurring (native) intein sequence. For example, an N-terminal intein segment can comprise additional amino acid residues and/or mutated residues so long as the inclusion of such additional and/or mutated residues does not render the intein non-functional for splicing or cleaving. Preferably, the inclusion of the additional and/or mutated residues improves or enhances the splicing activity and/or controllability of the intein. Non-intein residues can also be genetically fused to intein segments to provide additional functionality, such as the ability to be affinity purified or to be covalently immobilized.

As used herein, the “C-terminal intein segment” refers to any intein sequence that comprises a C-terminal amino acid sequence that is functional for splicing or cleaving reactions when combined with a corresponding N-terminal intein segment. In one aspect, the C-terminal intein segment comprises a sequence that is spliced out when splicing occurs. In another aspect, the C-terminal intein segment is cleaved from a peptide sequence fused to its C-terminus. A C-terminal intein segment can comprise a sequence that is a modification of the C-terminal portion of a naturally occurring (native) intein sequence. For example, a C terminal intein segment can comprise additional amino acid residues and/or mutated residues so long as the inclusion of such additional and/or mutated residues does not render the C-terminal intein segment non-functional for splicing or cleaving.

Expressed protein ligation (EPL) refers to a native chemical ligation between a recombinant protein with a C-terminal thioester and a second agent with an N-terminal cysteine. The C-terminal thioester can readily be introduced onto any recombinant protein (i.e., the targeting ligand) through the use of auto-processing, also known as protein-splicing, mediated by an intein (intervening protein). Inteins are proteins that can excise themselves from a larger precursor polypeptide chain, utilizing a process that results in the formation of a native peptide bond between the flanking extein (external protein) fragments. When an auto-processing protein is cloned downstream of the targeting ligand, thiols (e.g., 2-mercaptoethanesulfonic acid, MESNA) can be used to induce the site-specific cleavage of the auto-processing protein, resulting in the formation of a reactive thioester. The thioester will then react with any agent that has an N-terminal cysteine. EPL operates in a site-specific manner, and the reaction is known to be very efficient if both functional groups are in high concentrations. (reviewed in Elias et al. Small 6:2460-2468).

Accordingly, in some embodiments, the MHC monomers are ligated to an alkynated peptide by expressed protein ligation (EPL) and then conjugated to an azide-labeled multimerization domain by Cu(I)-catalyzed terminal azide-alkyne cycloaddition (CuAAC).

In some embodiments, the MHC monomers are conjugated to the multimerization domain by an intein peptide tag. In some embodiments, the MHC polypeptide comprises a C-terminal thioester, the multimerization domain comprises an N-extein fused to a modified intein lacking the ability to perform trans-esterification and trans-esterification occurs by the addition of exogenous thiol.

A number of inteins have now been described including, but not limited to MxeGyrA (Frutos et al. (2010); Southworth et al. (1999); SspDnaE (Shah et al. (2012); Wu et al. (1998); NpuDnaE (Shah et al. (2012); Vila-Perello et al. (2013); AvaDnaE (David et al. (2015); Shah et al. (2012); Cfa (consensus DnaE split intein) (Stevens et al. (2016)); gp41-1 and gp41-8 (Carvajal-Vallejos et al. (2012)); NrdJ-1 (Carvajal-Vallejos et al. (2012)); IMPDH-1 (Carvajal-Vallejos et al) and AceL-TerL (Thiel et al. (2014). The properties and use of these inteins are summarized in Table 1.

TABLE 1

| Intein | Temperature (° C.) | t_{1/2}* | |
|---|---|---|---|
| MxeGyrA | 25 | 10 | h |
| SspDnaE | 37 | 76 | min |
| NpuDnaE | 37 | 19 | s |
| AvaDnaE | 37 | 23 | s |
| Cfa (consensus DnaE split intein) | 30 | 20 | s |
| gp41-1 | 45 | 4 | s |
| gp41-8 | 37 | 15 | s |
| NrdJ-1 | 37 | 7 | s |
| IMPDH-1 | 37 | 8 | s |
| AceL-TerL | 8 | 7.2 | min |

In some embodiments, the intein is the 198-residue gyrase A intein from Mycobacterium xenopi (Mxe GyrA) (Southworth M W, Amaya K, Evans T C, Xu M Q, Perler F B Biotechniques. 1999 July; 27(1):110-4, 116, 118-20). In some embodiments, the intein is from cyanobacterium Synechocystis sp. strain PCC6803 (Ssp).

In some embodiments, the intein is a split intein pair. In some embodiments, the split intein pair is an orthogonal split intein pair (Carvajal-Vallejos P, Pallissé R, Mootz H D, Schmidt S R J Biol Chem. 2012 Aug. 17; 287(34):28686-96; Shah N H, Vila-Perello M, Muir T W Angew Chem Int Ed Engl. 2011 Jul. 11; 50(29):6511-5).

In some embodiments, the split intein pair is an artificially split intein pair that are as short as six or eleven residues (Appleby J H, Zhou K, Volkmann G, Liu X Q J Biol Chem. 2009 Mar. 6; 284(10):6194-9; Ludwig C, Pfeiff M, Linne U, Mootz H D Angew Chem Int Ed Engl. 2006 Aug. 4; 45(31):5218-21).

In some embodiments, the intein is a DnaE intein. In some embodiments, the DnaE intein is from Nostoc punctiforme (Npu). In some embodiments, the intein is the gp41-1 intein. In some embodiments, the intein is the gp41-8 intein. In some embodiments, the intein is the IMPDH-1 intein. In some embodiments, the intein is the NrdJ Intein.

In some embodiments, the split intein pair is AceL-TerL (Thiel I V, Volkmann G, Pietrokovski S, Mootz H D Angew Chem Int Ed Engl. 2014 Jan. 27; 53(5):1306-10).

In some embodiments, the intein comprises consensus split intein sequence (Cfa) (Stevens A J, Brown Z Z, Shah N H, Sekar G, Cowburn D, Muir T W. Design of a split intein with exceptional protein splicing activity. Journal of the American Chemical Society. 2016; 138(7):2162-2165).

A number of protocols for intein mediated conjugation are available and an exemplary method is provided herein in Example 2. Suitable intein sequences and protocols for use in protein conjugation have been described in the art, such as in Stevens, et al. J. Am. Chem. Soc., 138, 2162-2165, 2016; Shah et al. J. Am. Chem. Soc., 134, 11338-11341, 2012; and Vila-Perello et al., J. Am. Chem. Soc., 135, 286-292, 2013; Batjargal S, Walters C R, Petersson E J J Am Chem Soc. 2015 Feb. 11; 137(5):1734-7; and Guan D, Ramirez M, Chen Z Biotechnol Bioeng. 2013 September; 110(9):2471-81, the entire contents of each of which is hereby incorporated by reference.

In some embodiments, the intein-labeled MHC molecule is a soluble HLA-A2 molecule (HLA-A*02:01) with an N-intein tag, such as having the amino acid sequence shown in SEQ ID NO: 4. In some embodiments, the intein-labeled multimerization domain is a streptavidin molecule with a C-intein tag and FLAG Tag, such as having the amino acid sequence shown in SEQ ID NO: 5. In some embodiments, the N-intein tag, including a FLAG tag, has the amino acid sequence shown in SEQ ID NO: 180. Various other N-intein and C-intein sequences are known in the art and are suitable for use in preparing the Conjugated Multimers of the disclosure, non-limiting examples of which are described in the references cited above.

F. Additional Bioconjujation Methods

In some embodiments, the conjugation of the MHC and multimerization domain is mediated enzymatically. In some embodiments, the enzyme is formylglycine generating enzyme (FGE) that recognizes the CXPXR amino acid sequence motif and converts the cysteine residue to formylglycine, thus introducing an aldehyde functional group (Wu P, Shui W, Carlson B L, Hu N, Rabuka D, Lee J, Bertozzi C R Proc Natl Acad Sci USA. 2009 Mar. 3; 106(9):3000-5), which is subjected to bio-orthogonal transformations such as oximation and Hydrazino-Pictet-Spengler reactions (Agarwal P, Kudirka R, Albers A E, Barfield R M, de Hart G W, Drake P M, Jones L C, Rabuka D Bioconjug Chem. 2013 Jun. 19; 24(6):846-51; Dirksen A, Dawson P E Bioconjug Chem. 2008 December; 19(12):2543-8).

Site-specific bioconjugation strategies offer many possibilities for directed protein modifications. Among the various enzyme-based conjugation protocols, formylglycine-generating enzymes allow to posttranslationally introduce the amino acid Ca-formylglycine (FGly) into recombinant proteins, starting from cysteine or serine residues within distinct consensus motifs. The aldehyde-bearing FGly-residue displays orthogonal reactivity to all other natural amino acids and can, therefore, be used for site-specific labeling reactions on protein scaffolds. (Reviewed in Kruger et al., Biol Chem. 2019 Feb. 25; 400(3):289-297. doi: 10.1515/hsz-2018-0358)

Formylglycine generating enzyme (FGE) recognizes a pentapeptide consensus sequence, CxPxR, and it specifically oxidizes the cysteine in this sequence to an unusual aldehyde-bearing formylglyine. The FGE recognition sequence, or aldehyde tag, can be inserted into heterologous recombinant proteins produced in either prokaryotic or eukaryotic expression systems. The conversion of cysteine to formylglycine is accomplished by co-overexpression of FGE, either transiently or as a stable cell line, and the resulting aldehyde can be selectively reacted with α-nucleophiles to generate a site-selectively modified bioconjugate (Rabuka et al. Nat Protoc. 2012 May 10; 7(6): 1052-1067).

In some embodiments, the enzyme is lipoic acid ligase, an enzyme that modifies a lysine side-chain within the 13-residue target sequence (Uttamapinant C, White K A, Baruah H, Thompson S, Fernandez-Suarez M, Puthenveetil S, Ting A Y Proc Natl Acad Sci USA. 2010 Jun. 15; 107(24):10914-9) to introduce bio-orthogonal groups, including azides, aryl aldehydes and hydrazines, p-iodophenyl derivatives, norbornenes, and trans-cyclooctenes (reviewed in Debelouchina et al. Q. Rev Biophys. 2017; 50 e7. doi:10.1017/S0033583517000021).

In other embodiments, the enzyme is biotin ligase, farnesyltransferase, transglutaminase or N-myristoyltransferase (reviewed in Rashidian M, Dozier J K, Distefano M D Bioconjug Chem. 2013 Aug. 21; 24(8):1277-94).

G. Peptide Linkers

In other embodiments, the p*MHC multimers comprises a peptide linker. The term “peptide linker” denotes a linear amino acid chain of natural and/or synthetic origin. The linker has the function to ensure that polypeptides conjugated to each other can perform their biological activity by allowing the polypeptides to fold correctly and to be presented properly. The peptide linker may contain repetitive amino acid sequences or sequences of naturally occurring polypeptides. In some embodiments, the peptide linker has a length of from 2 to 50 amino acids. In some embodiments, the peptide linker is between 3 and 30 amino acids, between 5 to 25 amino acids, between 5 to 20 amino acids, or between 10 and 20 amino acids.

In some embodiments, the peptide linker is rich in glycine, glutamine, and/or serine residues. These residues are arranged e.g. in small repetitive units of up to five amino acids. This small repetitive unit may be repeated for one to five times. At the amino- and/or carboxy-terminal ends of the multimeric unit up to six additional arbitrary, naturally occurring amino acids may be added. Other synthetic peptidic linkers are composed of a single amino acid, which is repeated between 10 to 20 times and may comprise at the amino- and/or carboxy-terminal end up to six additional arbitrary, naturally occurring amino acids. All peptidic linkers can be encoded by a nucleic acid molecule and therefore can be recombinantly expressed. As the linkers are themselves peptides, the polypeptide connected by the linker are connected to the linker via a peptide bond that is formed between two amino acids.

Suitable peptide linkers are well known in the art, and are disclosed in, e.g., US2010/0210511 US2010/0179094, and US2012/0094909, which are herein incorporated by reference in its entirety. Other linkers are provided, for example, in U.S. Pat. No. 5,525,491; Alfthan et al., Protein Eng., 1995, 8:725-731; Shan et al., J. Immunol., 1999, 162:6589-6595; Newton et al., Biochemistry, 1996, 35:545-553; Megeed et al.; Biomacromolecules, 2006, 7:999-1004; and Perisic et al., Structure, 1994, 12:1217-1226; each of which is incorporated by reference in its entirety.

In some embodiments, the polypeptide linker is synthetic. As used herein, the term “synthetic” with respect to a polypeptide linker includes peptides (or polypeptides) which comprise an amino acid sequence (which may or may not be naturally occurring) that is linked in a linear sequence of amino acids to a sequence (which may or may not be naturally occurring) to which it is not naturally linked in nature. For example, the polypeptide linker may comprise non-naturally occurring polypeptides which are modified forms of naturally occurring polypeptides (e.g., comprising a mutation such as an addition, substitution or deletion) or which comprise a first amino acid sequence (which may or may not be naturally occurring). Polypeptide linkers may be employed, for instance, to ensure that the binding portion (TCR or MHC), the multimerization domain and the Igg-Framework of each multimeric fusion polypeptide is juxtaposed to ensure proper folding and formation of a functional multimeric protein complex. Preferably, a polypeptide linker will be relatively non-immunogenic and not inhibit any non-covalent association among monomer subunits of a binding protein.

In some embodiments, the linker is a Gly-Ser polypeptide linker, i.e., a peptide that consists of glycine and serine residues. One exemplary Gly-Ser polypeptide linker comprises the amino acid sequence (Gly4Ser)n, wherein n=1-6 (SEQ ID NO: 181). In certain embodiments, n=1. In certain embodiments, n=2. In certain embodiments, n=3. In certain embodiments, n=4. In certain embodiments, n=5. In certain embodiments, n=6. Another exemplary Gly-Ser polypeptide linker comprises the amino acid sequence Ser(Gly4Ser)n, wherein n=1-10 (SEQ ID NO: 184). In certain embodiments, n=1. In certain embodiments, n=2. In certain embodiments, n=3, i.e., Ser(Gly4Ser)3. In certain embodiments, n=4, i.e., Ser(Gly4Ser)4. In certain embodiments, n=5. In certain embodiments, n=6. In certain embodiments, n=7. In certain embodiments, n=8. In certain embodiments, n=9. In certain embodiments, n=10.

Other exemplary linkers include GS linkers (i.e., (GS)n), GGSG linkers (i.e., (GGSG)n) (SEQ ID NO: 185), GSAT linkers (SEQ ID NO: 186), SEG linkers, and GGS linkers (i.e., (GGSGGS)n) (SEQ ID NO: 187), wherein n is a positive integer (e.g., 1, 2, 3, 4, or 5). Other suitable linkers for use in multimeric fusion proteins can be found using publicly available databases, such as the Linker Database (ibi.vu.nl/programs/linkerdbwww). The Linker Database is a database of inter-domain linkers in multi-functional enzymes which serve as potential linkers in novel multimeric fusion proteins (see, e.g., George et al., Protein Engineering 2002; 15:871-9).

Polypeptide linkers can be introduced into polypeptide sequences using techniques known in the art. Modifications can be confirmed by DNA sequence analysis. Plasmid DNA can be used to transform host cells for stable production of the polypeptides produced.

H. Additional Peptide Linkers and Tags

Additional tags suitable for use in the methods and compositions provided herein include affinity tags, including but not limited to enzymes, protein domains, or small polypeptides which bind with high specificity to a range of substrates, such as carbohydrates, small biomolecules, metal chelates, antibodies, etc. to allow rapid and efficient purification of proteins. Solubility tags enhance proper folding and solubility of a protein and are frequently used in tandem with affinity tags.

Small-size tags which include, but are not limited to, 6× His, FLAG, Strep II and Calmodulin-binding peptide (CBP) tag, have the benefits of minimizing the effect on structure, activity and characteristics of the MHC polypeptide. (Zhao et al. J. Anal. Chem. 2013 581093)

In some embodiments, the tag is a FLAG tag. The FLAG tag is a hydrophilic octapeptide epitope tag that binds to several specific anti-FLAG monoclonal antibodies such as M1, M2, and M5 with different recognition and binding characteristics (Einhauer et al. J. Biochem. Biophys. 49:455-465, 2001: Hopp et al. Mol. Immunol. 33:601-608, 1996). FLAG fusion proteins can be recognized by monoclonal antibody with calcium-dependent (e.g., M2) or calcium-independent manner. In particular, the tag appended to the N-terminus of the fusion protein is necessary for the immunoaffinity purification with M1 monoclonal antibody, while M2 is position-insensitive.

### IV. MHC Peptide Epitopes

A. Peptide Epitope Selection

Various processes have been developed for identifying new MHC binding peptides that may be T cell epitopes and many experimental methods start with constructing an overlapping library of peptide fragments from a given protein sequence, by synthesizing a constant length (n-mer) amino acid sequences which are offset from one another along the protein sequence by fixed number of amino acids. The MHC binding properties and potential for activating T cells of each sequence can then be assessed in a number of assays.

Existing MHC binding peptides that have been identified with the methods outlined above and other methods, such as crystallographic analysis of the conformation of and charge distribution in the MHC binding groove has led to binding motifs being defined for the most common MHC alleles, setting rules for what type of putative MHC binding peptide can actually bind well to MHC molecules of a given allele. These motifs have been translated into predictive computer algorithms for predicting peptide binding to MHC molecules such as the SYFPEITHI algorithm (Rammensee H.-G., et al. (1995), Immunogenetics 41:178-228).

Protein sequences for the desired antigen are analyzed for potential HLA specific antigens by using SYFPEITHI (Rammensee et al. Immungenetics 50:213-219, 1999), and the artificial neural network (ANN) and stabilized matrix method (SMM) algorithms from IEDB (Peters et al. PLoS Biol. 3:e91, 2005). Peptides are selected based on a predicted binding value of either >21 for SYFPEITHY, <6000 for ANN, or <600 for SMM. Selected peptides are synthesized.

Binding assays can be performed using a fluorescence polarization (FP) assay as previously described (e.g., Buchi et al. Biochemistry 43:14852-14863, 2004; Sette et al., Mol. Immunol. 31:813-822). To determine binding capacity of the peptides, percentage inhibition relative to controls can be determined in an FP competition assay with the placeholder peptide.

In some embodiments, the peptides bound to the pMHC multimers are from an unbiased library of peptides. In some embodiments, the peptides are 9-mers. In some embodiments, the peptides bound to the pMHCI multimers are 9-mers which include an HLA-A2 binding motif with key amino acids at positions 2 and 9 which can include isoleucine (I), valine (V) or leucine (L).

In some embodiments, the library comprises all k-mer peptides produced by transcription and translation of any polynucleotide sequence of interest, for example, in silico production of the transcription and translation products of both the forward and reverse strands of a genome or metagenome in all six reading frames.

In some embodiments, a library of the disclosure comprises all k-mer peptides that can be derived from in silico translation of an exome of interest.

In some embodiments, a library of the disclosure comprises all k-mer peptides that can be derived from in silico translation of a transcriptome of interest.

In some embodiments, a library of the disclosure comprises all k-mer peptides that can be derived from a proteome of interest.

In some embodiments, a library of the disclosure comprises all k-mer peptides that can be derived from in silico translation of an ORFeome of interest.

In some embodiments, an algorithm can be used to select peptides in a peptide library. For example, an algorithm can be used to predict peptides most likely to fold or dock in an MHC/HLA binding pocket, and peptides above a certain threshold value can be selected for inclusion in the library.

In some embodiments, a library of the disclosure comprises all peptides that can be derived from in silico transcription and translation or translation of a group of genomes, proteomes, transcriptomes, ORFeomes, or any combination thereof.

In some embodiments, the peptides are derived from in silico transcription and translation or translation of polynucleotide sequences from a group of samples, for example, clinical samples from a patient population, or a group of pathogen genomes.

In some embodiments, the peptides are derived from a differential genome, proteome, transcriptome, ORFeome, or any combination thereof, where two or more genomes, proteomes, transcriptomes, ORFeomes, or a combination thereof are compared to identify sequences that are differential sequences (e.g., that differ between them). In some embodiments, the peptide sequences are identified by comparing tissues of interest. In some embodiments, the peptide sequences are identified by comparing cells of interest. In some embodiments, the peptide sequences are identified by comparing diseased versus healthy cells or tissues. In some embodiments, the diseased cells or tissues are cancer cells or tissues. In some embodiments, the diseased cells are derived from an individual with an autoimmune disorder.

In some embodiments, the peptides are derived from homologous sequences of genomes, proteomes, transcriptomes, ORFeomes, or any combination thereof, where two or more genomes, proteomes, transcriptomes, ORFeomes, or a combination thereof are compared to identify sequences that are homologous sequences.

In some embodiments, the peptides are derived from mutations in a sequence of interest, for example, all 9-mer peptides that can be generated from single nucleotide mutations in a polynucleotide sequence encoding an antigen or epitope.

In some embodiments, the peptides an overlapping peptide library, comprising overlapping peptides from a template sequence (e.g., in silico translated genome), wherein overlapping peptides of a set length are offset by a defined number of residues.

In some embodiments, selection of peptides comprises prioritizing peptides based on predicted binding affinity for a certain HLA type.

In some embodiments, selection of peptides for a library of the disclosure prioritizes HLA types or alleles based on prevalence in a population, e.g., a human population.

In some embodiments, the library comprises all k-mer peptides produced by transcription and translation of any polynucleotide sequence of interest, for example, in silico production of the transcription and translation products of both the forward and reverse strands of a genome or metagenome in all six reading frames. In some embodiments, a library of the disclosure comprises all k-mer peptides that can be derived from in silico transcription and translation of a mammalian genome, for example, a mouse genome, a human genome, a patient genome, an autoimmune patient genome, or a cancer genome. In some embodiments, a library of the disclosure comprises all k-mer peptides that can be derived from in silico transcription and translation of a microorganism genome, for example, a bacterial genome, a viral genome, a protozoan genome, a protist genome, a yeast genome, an archaeal genome, or a bacteriophage genome. In some embodiments, a library of the disclosure comprises all k-mer peptides that can be derived from in silico transcription and translation of a pathogen genome, for example, a bacterial pathogen genome, a viral pathogen genome, a fungal pathogen genome, an opportunistic pathogen genome, a conditional pathogen genome, or a eukaryotic parasite genome. In some embodiments, a library of the disclosure can be derived from a plant genome or a fungal genome. In some embodiments, a library of the disclosure comprises k-mer peptides derived from in silico transcription and translation of a genome, wherein the genome is modified during in silico transcription and translation, for example, in silico mutated to produce k-mer peptides comprising mutations (e.g. substitutions, insertions, deletions).

In some embodiments, a library of the disclosure comprises all k-mer peptides that can be derived from in silico translation of an exome of interest, for example, a mammalian exome, a human exome, a mouse exome, a patient exome, an autoimmune patient exome, a cancer exome, a viral exome, a protozoan exome, a protist exome, a yeast exome, a pathogen exome, a eukaryotic parasite exome, a plant exome, or a fungal exome. In some embodiments, a library of the disclosure comprises k-mer peptides derived from in silico translation of a exome, wherein the exome is modified during in silico translation, for example, in silico mutated to produce k-mer peptides comprising mutations (e.g. substitutions, insertions, deletions).

In some embodiments, a library of the disclosure comprises all k-mer peptides that can be derived from in silico translation of a transcriptome of interest, for example, a mammalian transcriptome, a human transcriptome, a mouse transcriptome, a patient transcriptome, an autoimmune patient transcriptome, a cancer transcriptome, a microorganism transcriptome, a bacterial transcriptome, a viral transcriptome, a protozoan transcriptome, a protist transcriptome, a yeast transcriptome, an archaeal transcriptome, a bacteriophage transcriptome, a pathogen transcriptome, a eukaryotic parasite transcriptome, a plant transcriptome, a fungal transcriptome, a transcriptome derived from RNA sequencing, a microbiome transcriptome, or a transcriptome derived from metagenomic RNA-sequencing. In some embodiments, a library of the disclosure comprises k-mer peptides derived from in silico translation of a transcriptome, wherein the transcriptome is modified during in silico translation, for example, in silico mutated to produce k-mer peptides comprising mutations (e.g. substitutions, insertions, deletions).

In some embodiments, a library of the disclosure comprises all k-mer peptides that can be derived from a proteome of interest, for example, a mammalian proteome, a human proteome, a mouse proteome, a patient proteome, an autoimmune patient proteome, a cancer proteome, a microorganism proteome, a bacterial proteome, a viral proteome, a protozoan proteome, a protist proteome, a yeast proteome, an archaeal proteome, a bacteriophage proteome, a pathogen proteome, a eukaryotic parasite proteome, a plant proteome or a fungal proteome. In some embodiments, a library of the disclosure comprises k-mer peptides derived from a proteome wherein the k-mer peptides are modified from the proteome sequence, for example, k-mer peptides comprising mutations (e.g. substitutions, insertions, deletions).

In some embodiments, a library of the disclosure comprises all k-mer peptides that can be derived from in silico translation of an ORFeome of interest, for example, a mammalian ORFeome, a human ORFeome, a mouse ORFeome, a patient ORFeome, an autoimmune patient ORFeome, a cancer ORFeome, a microorganism ORFeome, a bacterial ORFeome, a viral ORFeome, a protozoan ORFeome, a protist ORFeome, a yeast ORFeome, an archaeal ORFeome, a bacteriophage ORFeome, a pathogen ORFeome, a eukaryotic parasite ORFeome, a plant ORFeome or a fungal ORFeome, an ORFeome derived from next-gen sequencing, a microbiome ORFeome, or an ORFeome derived from metagenomic sequencing. In some embodiments, a library of the disclosure comprises k-mer peptides derived from in silico translation of an ORFeome, wherein the ORFeome is modified during in silico translation, for example, in silico mutated to produce k-mer peptides comprising mutations (e.g. substitutions, insertions, deletions).

In some embodiments, a library of the disclosure comprises all k-mer peptides that can be derived from in silico transcription and translation or translation of a group of genomes, proteomes, transcriptomes, ORFeomes, or any combination thereof. In some embodiments, a library of the disclosure comprises all k-mer peptides that can be derived from in silico transcription and translation or translation of polynucleotide sequences from a group of samples, for example, clinical samples from a patient population, or a group of pathogen genomes. In some embodiments, a library of the disclosure comprises all k-mer peptides that can be derived from in silico transcription and translation of a group of viral genomes, for example, the human virome. In some embodiments, a library of the disclosure comprises all k-mer peptides that can be derived from in silico transcription and translation of a group of genomes, proteomes, transcriptomes, ORFeomes, or any combination thereof, wherein the source sequences are modified during in silico translation, for example, in silico mutated to produce k-mer peptides comprising mutations (e.g. substitutions, insertions, deletions).

In some embodiments, a library of the disclosure comprises all k-mer peptides that can be derived from a differential genome, proteome, transcriptome, ORFeome, or any combination thereof, where two or more genomes, proteomes, transcriptomes, ORFeomes, or a combination thereof are compared to identify sequences that are differential sequences (e.g., that differ between them), for example, differing in nucleotide sequence, amino acid sequence, nucleotide abundance, or protein abundance. In some embodiments, differential sequences of a genome, proteome, transcriptome, or ORFeome are generated by comparing tissues of interest. In some embodiments, differential sequences of a genome, proteome, transcriptome, or ORFeome are generated by comparing sequences from cells of interest (e.g., a healthy cell versus a cancer cell). In some embodiments, differential sequences of a genome, proteome, transcriptome, or ORFeome are generated by comparing sequences of organisms of interest. In some embodiments, differential sequences of a genome, proteome, transcriptome, or ORFeome can be generated by comparing subjects of interest (e.g., diseased versus healthy subjects).

In some embodiments, a library of the disclosure comprises all k-mer peptides that can be derived from homologous sequences of genomes, proteomes, transcriptomes, ORFeomes, or any combination thereof, where two or more genomes, proteomes, transcriptomes, ORFeomes, or a combination thereof are compared to identify sequences that are homologous sequences (e.g., that share a degree of homology), for example, homologous nucleotide sequences, homologous amino acid sequences, homologous nucleotide abundance, or homologous protein abundance. In some embodiments, homologous sequences of genomes, proteomes, transcriptomes, or ORFeomes are generated by comparing tissues of interest. In some embodiments, homologous sequences of genomes, proteomes, transcriptomes, or ORFeomes are generated by comparing sequences from cells of interest (e.g., a healthy cell versus a involved in autoimmunity cell (e.g., a cell that induces autoimmunity or a cell that is targeted during autoimmunity). In some embodiments, homologous sequences of genomes, proteomes, transcriptomes, or ORFeomes are generated by comparing sequences of organisms of interest. In some embodiments, homologous sequences of genomes, proteomes, transcriptomes, or ORFeomes are generated by comparing subjects of interest (e.g., diseased versus healthy subjects).

In some embodiments, a library of the disclosure comprises all k-mer peptides that can be derived from a polypeptide sequence of interest, for example, all possible 9-mer peptides covering the complete protein sequence of a viral protein. In some embodiments, a library of the disclosure comprises k-mer peptides that can be generated from a polypeptide sequence of interest, wherein the polypeptide sequence of interest is modified, e.g. in silico mutated to produce k-mer peptides comprising mutations (e.g. substitutions, insertions, deletions).

In some embodiments, a library of the disclosure comprises all k-mer peptides that can be derived from mutations in a sequence of interest, for example, all 9-mer peptides that can be generated from single nucleotide mutations in a polynucleotide sequence encoding an antigen or epitope. For example, a library of the disclosure comprises all 9-mer peptides that can be generated from two, three, four, five, six, seven, eight, or nine nucleotide mutations in a polynucleotide sequence encoding an antigen or epitope. In some embodiments, a library of the disclosure comprises all k-mer peptides that can be derived from alanine substitutions, for example, alanine substitutions at any position in any of the sequences described herein (e.g., a protein, a group of proteins, a proteome, an in silico transcripted and translated genome). In some embodiments, a library of the disclosure comprises a positional scanning library, wherein selected amino acid residues are sequentially substituted with all other natural amino acids. In some embodiments, a library of the disclosure comprises a combinatorial positional scanning library, wherein selected amino acid residues are sequentially substituted with all other natural amino acids, two or more positions at a time. In some embodiments, a library of the disclosure comprises an overlapping peptide library, comprising overlapping peptides from a template sequence (e.g., in silico translated genome), wherein overlapping peptides of a set length are offset by a defined number of residues. In some embodiments, a library of the disclosure comprises a T cell truncated peptide library, wherein each replicate of the library comprises equimolar mixtures of peptides with truncations at one terminus (e.g., 8-mers, 9-mers, 10-mers and 11-mers that can be derived from C-terminal truncations of a nominal 11-mer). In some embodiments, a library of the disclosure comprises a customized set of peptides, wherein the customized set of peptides are provided in a list.

In some embodiments, a genome, exome, transcriptome, proteome, or ORFeome of the disclosure is a viral genome, exome, transcriptome, proteome, or ORFeome. Non-limiting examples of viruses include Adenovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Cytomegalovirus (CMV), Dengue virus, Dhori virus, Dugbe virus, Duvenhage virus, Eastern equine encephalitis virus, Ebolavirus, Echovirus, Encephalomyocarditis virus, Epstein-Barr virus (EBV), European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human endogenous retrovirus (HERV), Human enterovirus, Human herpesvirus (e.g., HHV-1, HHV-2, HHV-6A, HHV-6B, HHV-7, HHV-8, Human immunodeficiency virus (e.g., HIV-1, HIV-2), Human papillomavirus (e.g., HPV-1, HPV-2, HPV-16, HPV-18, Human parainfluenza, Human parvovirus B19, Human respiratory syncytial virus (RSV), Human rhinovirus, Human SARS coronavirus, Human spumaretrovirus, Human T-lymphotropic virus (HTLV, e.g. HTLV-1, HTLV-2, HTLV-3), Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norovirus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus (e.g., rotavirus A, rotavirus B, rotavirus C, rotavirus X), Rubella virus, Sagiyama virus, Salivirus A, Sandfly fever sicilian virus, Sapporo virus, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika virus.

In some embodiments, a genome, exome, transcriptome, proteome, or ORFeome of the disclosure is a cancer genome, exome, transcriptome, proteome, or ORFeome. In some embodiments, a library of the disclosure comprises known cancer neoepitopes. In some embodiments, a library of the disclosure comprises all k-mer peptides that can be derived from known cancer antigenic proteins. In some embodiments, a library of the disclosure comprises all k-mer peptides that can be derived from genes involved in epithelial-mesenchymal transition. In some embodiments, a library of the disclosure comprises all k-mer peptides that can be derived from cancer implicated genes. In some embodiments, a library of the disclosure comprises all k-mer peptides that can be derived from mutational cancer driver genes. In some embodiments, a library of the disclosure comprises all k-mer peptides that can be derived from proto-oncogenes, oncogenes, or tumor suppressor genes. In some embodiments, a library of the disclosure comprises all k-mer peptides that can be derived from proto-oncogenes, oncogenes, or tumor suppressor genes, wherein the k-mers comprise mutations as described herein (e.g., amino acid substitutions, alanine substitutions, positional scanning, combinatorial positional scanning etc.).

Non-limiting examples of cancers include Acute Lymphoblastic Leukemia (ALL), Acute Myeloid Leukemia (AML), Adrenocortical Carcinoma, AIDS-Related Cancers, AIDS-Related Lymphoma, Anal Cancer, Appendix Cancer, Astrocytoma, Atypical Teratoid/Rhabdoid Tumor, Basal Cell Carcinoma, Bile Duct Cancer, Bladder Cancer, Bone Cancer, Brain Tumor, Breast Cancer, Bronchial Tumors, Burkitt Lymphoma, Carcinoid Tumor, Carcinoma of Unknown Primary, Cardiac Tumor, Central Nervous System cancer, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ, Embryonal Tumor, Endometrial Cancer, Epithelial Cancer, Ependymoma, Esophageal Cancer, Esthesioneuroblastoma, Ewing Sarcoma, Extracranial Germ Cell Tumor, Extragonadal Germ Cell Tumor, Eye Cancer, Fallopian Tube Cancer, Fibrous Histiocytoma of Bone, Gallbladder Cancer, Gastric Cancer, Gastrointestinal Carcinoid Tumor, Gastrointestinal Stromal Tumors (GIST), Germ Cell Tumors, Gestational Trophoblastic Disease, Hairy Cell Leukemia, Head and Neck Cancer, Hepatocellular Cancer, Histiocytosis, Hodgkin Lymphoma, Hypopharyngeal Cancer, Intraocular Melanoma, Islet Cell Tumors, Kaposi Sarcoma, Kidney (Renal Cell) Cancer, Langerhans Cell Histiocytosis, Laryngeal Cancer, Leukemia, Lip and Oral Cavity Cancer, Liver Cancer, Lung Cancer (Non-Small Cell and Small Cell), Lymphoma, Male Breast Cancer, Malignant Fibrous Histiocytoma of Bone and Osteosarcoma, Melanoma, Merkel Cell Carcinoma, Mesothelioma, Metastatic Cancer, Metastatic Squamous Neck Cancer with Occult Primary, Midline Tract Carcinoma, Mouth Cancer, Multiple Endocrine Neoplasia Syndrome, Multiple Myeloma, Mycosis Fungoides, Myelodysplastic Syndromes, Myelodysplastic/Myeloproliferative Neoplasms, Nasal Cavity Cancer, Nasopharyngeal Cancer, Neuroblastoma, Non-Hodgkin Lymphoma, Non-Small Cell Lung Cancer, Oral Cancer, Lip and Oral Cavity Cancer, Oropharyngeal Cancer, Osteosarcoma, Ovarian Cancer, Pancreatic Cancer, Pancreatic Neuroendocrine Tumors, Papillomatosis, Paraganglioma, Paranasal Sinus Cancer, Parathyroid Cancer, Penile Cancer, Pharyngeal Cancer, Pheochromocytoma, Pituitary Tumor, Plasma Cell Neoplasm, Pleuropulmonary Blastoma, Primary Central Nervous System (CNS) Lymphoma, Primary Peritoneal Cancer, Prostate Cancer, Rectal Cancer, Recurrent Cancer, Retinoblastoma, Rhabdomyosarcoma, Salivary Gland Cancer, Sarcoma, Sézary Syndrome, Skin Cancer, Small Cell Lung Cancer, Small Intestine Cancer, Soft Tissue Sarcoma, Squamous Cell Carcinoma of the Skin, Squamous Neck Cancer with Occult Primary, Stomach Cancer, T-Cell Lymphoma, Testicular Cancer, Throat Cancer, Thymoma and Thymic Carcinoma, Thyroid Cancer, Transitional Cell Cancer, Ureter and Renal Pelvis Cancer, Urethral Cancer, Uterine Cancer, Uterine Sarcoma, Vaginal Cancer, Vascular Tumors, Vulvar Cancer, and Wilms Tumor.

In some embodiments, a genome, exome, transcriptome, proteome, or ORFeome of the disclosure is an inflammatory or autoimmunogenic genome, exome, transcriptome, proteome, or ORFeome. In some embodiments, a library of the disclosure comprises known inflammatory or autoimmunogenic neoepitopes or self-epitopes. In some embodiments, a library of the disclosure comprises all k-mer peptides that can be derived from known inflammatory or autoimmunogenic antigenic proteins. In some embodiments, a library of the disclosure comprises all k-mer peptides that can be derived from inflammatory or autoimmune-implicated genes. In some embodiments, a library of the disclosure comprises all k-mer peptides that can be derived from mutation of inflammatory or autoimmune-related driver genes.

Non-limiting examples of inflammatory or autoimmune diseases or conditions include Acute Disseminated Encephalomyelitis (ADEM); Acute necrotizing hemorrhagic leukoencephalitis; Addison's disease; Adjuvant-induced arthritis; Agammaglobulinemia; Alopecia areata; Amyloidosis; Ankylosing spondylitis; Anti-GBM/Anti-TBM nephritis; Antiphospholipid syndrome (APS); Autoimmune angioedema; Autoimmune aplastic anemia; Autoimmune dysautonomia; Autoimmune gastric atrophy; Autoimmune hemolytic anemia; Autoimmune hepatitis; Autoimmune hyperlipidemia; Autoimmune immunodeficiency; Autoimmune inner ear disease (AIED); Autoimmune myocarditis; Autoimmune oophoritis; Autoimmune pancreatitis; Autoimmune retinopathy; Autoimmune thrombocytopenic purpura (ATP); Autoimmune thyroid disease; Autoimmune urticarial; Axonal & neuronal neuropathies; Balo disease; Behcet's disease; Bullous pemphigoid; Cardiomyopathy; Castleman disease; Celiac disease; Chagas disease; Chronic inflammatory demyelinating polyneuropathy (CIDP); Chronic recurrent multifocal ostomyelitis (CRMO); Churg-Strauss syndrome; Cicatricial pemphigoid/benign mucosal pemphigoid; Crohn's disease; Cogans syndrome; Collagen-induced arthritis; Cold agglutinin disease; Congenital heart block; Coxsackie myocarditis; CREST disease; Essential mixed cryoglobulinemia; Demyelinating neuropathies; Dermatitis herpetiformis; Dermatomyositis; Devic's disease (neuromyelitis optica); Discoid lupus; Dressler's syndrome; Endometriosis; Eosinophilic esophagitis; Eosinophilic fasciitis; Erythema nodosum Experimental allergic encephalomyelitis; Experimental autoimmune encephalomyelitis; Evans syndrome; Fibromyalgia; Fibrosing alveolitis; Giant cell arteritis (temporal arteritis); Giant cell myocarditis; Glomerulonephritis; Goodpasture's syndrome; Granulomatosis with Polyangiitis (GPA) (formerly called Wegener's Granulomatosis); Graves' disease; Guillain-Barre syndrome; Hashimoto's encephalitis; Hashimoto's thyroiditis; Hemolytic anemia; Henoch-Schonlein purpura; Herpes gestationis; Hypogammaglobulinemia; Idiopathic thrombocytopenic purpura (ITP); IgA nephropathy; IgG4-related sclerosing disease; Immunoregulatory lipoproteins; Inclusion body myositis; Interstitial cystitis; Inflammatory bowel disease; Juvenile arthritis; Juvenile oligoarthritis; Juvenile diabetes (Type 1 diabetes); Juvenile myositis; Kawasaki syndrome; Lambert-Eaton syndrome; Leukocytoclastic vasculitis; Lichen planus; Lichen sclerosus; Ligneous conjunctivitis; Linear IgA disease (LAD); Lupus (SLE); Lyme disease, chronic; Meniere's disease; Microscopic polyangiitis; Mixed connective tissue disease (MCTD); Mooren's ulcer; Mucha-Habermann disease; Multiple sclerosis; Myasthenia gravis; Myositis; Narcolepsy; Neuromyelitis optica (Devic's); Neutropenia; Non-obese diabetes; Ocular cicatricial pemphigoid; Optic neuritis; Palindromic rheumatism; PANDAS (Pediatric Autoimmune Neuropsychiatric Disorders Associated with Streptococcus); Paraneoplastic cerebellar degeneration; Paroxysmal nocturnal hemoglobinuria (PNH); Parry Romberg syndrome; Parsonnage-Turner syndrome; Pars planitis (peripheral uveitis); Pemphigus; Pemphigus vulgaris; Peripheral neuropathy; Perivenous encephalomyelitis; Pernicious anemia; POEMS syndrome; Polyarteritis nodosa; Type I, II, & III autoimmune polyglandular syndromes; Polymyalgia rheumatic; Polymyositis; Postmyocardial infarction syndrome; Postpericardiotomy syndrome; Progesterone dermatitis; Primary biliary cirrhosis; Primary sclerosing cholangitis; Psoriasis; Plaque Psoriasis; Psoriatic arthritis; Idiopathic pulmonary fibrosis; Pyoderma gangrenosum; Pure red cell aplasia; Raynauds phenomenon; Reactive Arthritis; Reflex sympathetic dystrophy; Reiter's syndrome; Relapsing polychondritis; Restless legs syndrome; Retroperitoneal fibrosis; Rheumatic fever; Rheumatoid arthritis; Sarcoidosis; Schmidt syndrome; Scleritis; Scleroderma; Sclerosing cholangitis; Sclerosing sialadenitis; Sjogren's syndrome; Sperm & testicular autoimmunity; Stiff person syndrome; Subacute bacterial endocarditis (SBE); Susac's syndrome; Sympathetic ophthalmia; Systemic lupus erythematosus (SLE); Systemic sclerosis; Takayasu's arteritis; Temporal arteritis/Giant cell arteritis; Thrombocytopenic purpura (TTP); Tolosa-Hunt syndrome; Transverse myelitis; Type 1 diabetes; Ulcerative colitis; Undifferentiated connective tissue disease (UCTD); Uveitis; Vasculitis; Vesiculobullous dermatosis; Vitiligo; Wegener's granulomatosis (now termed Granulomatosis with Polyangiitis (GPA). Non-limiting examples of inflammatory or autoimmune diseases or conditions include infection, such as a chronic infection, latent infection, slow infection, persistent viral infection, bacterial infection, fungal infection, mycoplasma infection or parasitic infection.

As described, for example, in U.S. Provisional Application No. 62/791,601, hereby incorporated by reference in its entirety.

B. Peptide Production

Peptides suitable for use in the pMHC multimers are generated according to methods known in the art, or synthetically produced by a commercial vendor or using a peptide synthesizer according to manufacturer's instructions. For example, in some embodiments, peptides suitable for use in the pMHC multimers can be made by in silico production methods.

In other embodiments, peptides can be synthesized via chemical methods, for example, tea bag synthesis, digital photolithography, pin synthesis, and SPOT synthesis. For example, an array of peptides can be generated via SPOT synthesis, where amino acid chains are built on a cellulose membrane by repeated cycles of adding amino acids, and cleaving side-chain protection groups.

In other embodiments, peptides can be expressed using recombinant DNA technology, for example, introducing an expression construct into bacterial cells, insect cells, or mammalian cells, and purifying the recombinant protein from cell extracts.

In some embodiments, peptides can be synthesized by in vitro transcription and translation, where synthesis utilizes the biological principles of transcription and translation in a cell-free context, for example, by providing a nucleic acid template, relevant building blocks (e.g., RNAs, amino acids), enzymes (e.g., RNA polymerase, ribosomes), and conditions.

In some embodiments, in vitro transcription and translation can include cell-free protein synthesis (CFPS). Obtaining a high yield by CFPS requires the usage of bacterial systems, in which the first amino acid of the translated sequence is N-formylmethionine (fMet). This residue differs from methionine by containing a neutral formyl group (HCO) instead of a positively charged amino-terminus (NH₃⁺). Constructs are engineered to include genes encoding an enzymatic cleavage domain and a library polypeptide as described in U.S. Provisional Application No. 62/791,601, hereby incorporated by reference in its entirety.

Removal of at least the initial methionine amino acid allows successful peptide folding and loading onto MHC protein. In addition, removal of the initial methionine amino acid provides a greater upper limit of peptide library diversity, e.g., 20^{x}, where x is the length of the peptide, while inclusion of this residue will restrict the library diversity to 20^{(x-1)}.

In some embodiments, the peptides are synthesized utilizing an in vitro transcription/translation (IVTT) system that can both transcribe, for example, a DNA construct into RNA, and then translate the RNA into a protein. For example, the methods of the present disclosure comprise a method for performing in vitro transcription/translation (IVTT) to produce a high diversity peptide library and allow for correct folding of proteins. IVTT can allow for protein production in a cell-free environment directly from a DNA or RNA template.

An IVTT method used herein can be performed using, for example, a PCR product, a linear DNA plasmid, a circular DNA plasmid, or an mRNA template with a ribosome-binding site (RBS) sequence. After the appropriate template has been isolated, transcription components can be added to the template including, for example, ribonucleotide triphosphates, and RNA polymerase. After transcription has been completed, translation components can be added, which can be found in, for example, rabbit reticulocyte lysate, or wheat germ extract. In some methods, the transcription and translation can occur during a single step, in which purified translation components found in, for example, rabbit reticulocyte lysate or wheat germ extract are added at the same time as adding the transcription components to the nucleic acid template.

In some embodiments, nucleotide sequence encoding a methionine residue at the N-terminus of the peptide and a cleavable moiety can be encoded in the DNA construct or RNA construct. The cleavable moiety is situated such that at least one N-terminus amino acid residue of the peptide is before or within the cleavable moiety. In some embodiments, the method comprises encoding a cleavable moiety that is situated such that one N-terminus amino acid residue of the peptide is before or within the cleavable moiety. In some embodiments, the one N-terminus amino acid residue is a methionine residue. The cleavable moiety can be cleaved using an enzyme, e.g., a protease, specific to the cleavable moiety, which can also cleave off the cleavable moiety from the remainder of the peptide.

An example of a cleavable moiety that can be encoded in a DNA or RNA construct as described herein includes any cleavable moiety cleaved by an enzyme. In some embodiments, a cleavable moiety can be cleaved by a protease. The cleavage moiety can be cleaved off of the peptide using an enzyme specific for the cleavage moiety. The enzyme can be, for example, Factor Xa, human rhinovirus 3C protease, AcTEV™ Protease, WELQut Protease, Genenase™ small ubiquitin-like modifier (SUMO) protein, Ulp1 protease, or enterokinase. The Ulp1 protease can cleave off a cleavage moiety in a specific manner by recognizing the tertiary structure, rather than an amino acid sequence. Enterokinase (enteropeptidase) can also be used to cleave the cleavage moiety from the candidate peptide. Enterokinase can cleave after lysine at the following cleavage site: DDDDK (SEQ ID NO.: 188). Enterokinase can also cleave at other basic residues, depending on the sequence and conformation of the protein substrate.

In some embodiments, the cleavable moiety can be a small ubiquitin-like modifier (SUMO) protein. The SUMO domain can be cleaved off of the peptide using a protease specific to SUMO. In some embodiments, the cleavable moiety can be an enterokinase cleavage site: DDDDK (SEQ ID NO.: 188). The protease can be, for example, Ulp1 protease or enterokinase. The Ulp1 protease can cleave off SUMO in a specific manner by recognizing the tertiary structure of SUMO, rather than an amino acid sequence. Enterokinase (enteropeptidase) can also be used to cleave after lysine at the following cleavage site: DDDDK (SEQ ID NO.: 188). Enterokinase can also cleave at other basic residues, depending on the sequence of the protein substrate.

During or after translation of the construct encoding the peptide, the N-terminus amino acid residue(s) (e.g., a SUMO domain) can be efficiently cleaved to produce the properly folded peptide. In some embodiments, at least one N-terminus amino acid residue is cleaved to produce the peptide. In some embodiments, one, two, three, four, five six, seven, eight, nine, ten or more N-terminus amino acid residues are cleaved to produce the peptide. The N-terminus amino acid can be any amino acid residue. The N-terminus amino acid residue can be a methionine amino acid residue. This properly folded peptide is thus not constrained to have an N-terminus methionine, and can be part of a high diversity peptide library produce by cell-free in vitro methods.

After translation of the construct encoding the peptide, an N-terminus amino acid residue can be cleaved to produce the peptide for the high diversity peptide library. In some embodiments, at least one N-terminus amino acid residue is cleaved to produce the peptide. In some embodiments, one or more N-terminus amino acids are cleaved, such as 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 105, 110, 115, 120, 125, 130, 140, 150, 160, 170, 180, 190, 200, 250 or more, N-terminus amino acid residues are cleaved to produce the peptide. The N-terminus amino acid can be any amino acid residue. The N-terminus amino acid residue can be a methionine amino acid residue.

In some embodiments, a DNA or RNA construct comprises a puromycin. In some embodiments, a DNA or RNA construct comprises a spacer sequence lacking a stop codon. In some embodiments, the peptides are purified by affinity tag purification (e.g., with a FLAG-tag). In some embodiments, the peptides comprise a HaloTag enzymatic sequence. In some embodiments, peptides comprise an avidin or streptavidin.

For mammalian expression, a construct encoding the CMV peptide was designed with a C-terminal Flag-tag with and without a C-terminal His-tag in a mammalian expression vector. Peptides were expressed by transient transfection in Expi293F or ExpiCHO-S cells (Life Technologies) according to the manufacturer's recommendations.

Peptides were purified from cell culture supernatants with anti-Flag affinity chromatography (Genscript) or by Ni-affinity chromatography. Size exclusion chromatography (SEC) was performed on a hydrophilic resin (GE Life Sciences) pre-equilibrated in 20 mM HEPES, 150 mM NaCl, pH 7.2.

Alternatively, peptides were purified by Ni-affinity chromatography without SEC purification, using a column buffer of 23 mM sodium phosphate, 500 mM sodium chloride, 500 mM imidazole, pH 7.4.

Peptides produced in mammalian cells were quantitated by UV at 280 nm, whereas CFPS-produced peptides were quantitated by a sandwich ELISA relative to a standard protein.

### V. Peptide Exchange

p*MHC multimers are used to generate a library of or microarray of pMHC multimers loaded with a diversity of unique peptide epitopes by in situ or in vitro peptide exchange reactions as described herein. In some embodiments, the peptide exchange reactions are performed in multiwell formats and under native conditions. Binding is determined by a number of techniques, such as ELISA, which monitors the stability of the MHC structure, or by biophysical techniques that monitor peptide binding, such as fluorescence polarization. Non-limiting exemplifications of peptide exchange via dipeptide exchange or UV-mediated exchange are described in detail in Example 4.

In some embodiments, to measure the dissociation efficiency of placeholder peptides or peptide fragments a fluorescently labeled placeholder peptide is used in exchange reactions in the presence of unlabeled exchange peptides. Aliquots of fluorescently labeled p*MHC multimers are either left untreated or exposed to peptide exchange conditions (e.g., UV exposure) for different time periods. The amount of remaining p*MHC-containing the placeholder peptide is monitored by fluorescence analysis to monitor the reduction in p*MHC complexes.

In some embodiments, the placeholder peptide has a lower affinity for the MHC peptide binding groove than the exchanged peptide epitope, and wherein step (d) comprises contacting the p*MHC monomer with an excess of peptide epitope in a competition assay. In some embodiments, the placeholder peptide has a KD that is about 10-fold lower than the exchanged peptide epitope.

Peptides that bind to the peptide binding groove of the MHC molecule can be a naturally occurring peptide but can also be synthetically created using the knowledge of the binding specificity of the B and F pocket of the particular MHC molecule or the supertype family it belongs to. Suitable ligands can be generated using the available 3D structures of MHC complexes and the knowledge on the binding pocket specificity of the respective MHC molecules.

Peptide binding specificity of MHC I polypeptides is primarily governed by the physiochemical properties of the B and F binding pockets in a coupled fashion. The B and F binding pockets typically bind to “anchor residues” in the peptide that define the binding of the peptide in the peptide binding groove of the MHC. The observed diversity in the amino acid residues of the peptide binding groove of the MHC molecules defines the peptide-binding and the presentation repertoire of the individual MHC molecule (Chang et al. 2011; Frontiers in Bioscience, Landmark Edition, Vol. 16:3014-3035). The specificity of the pockets for anchor residues has been elucidated for a large number MHC molecules, for example, as described in Sidney et al. (BMC Immunology Vol. 9:1, 2008)

The disclosure further provides a method of producing a p*MHC multimer comprising: producing an p*MHC multimer in which the peptide in the binding groove is a placeholder peptide; contacting the p*MHC multimer with a reducing agent to remove the placeholder peptide; and contacting the p*MHC multimer with an MHC peptide epitope under conditions sufficient for binding of the peptide epitope in the MHC peptide binding groove.

The two contacting steps are preferably performed by providing a sample comprising the MHC molecule with the MHC peptide epitope and the reducing agent. It is preferred that the MHC peptide epitope is present when the reducing agent is added. In some embodiments, one MHC peptide epitope is added per reaction. In some embodiments, two or more peptide epitopes are added to the reaction.

In some embodiments, peptide exchange is induced by elevating the temperature of the mixture to between about 30°-37° C. In some embodiments, the mixture is elevated to 31°, 32°, 33°, 34°, 35°, 36° or 37°.

In some embodiments, peptide exchange is induced by reducing the pH of the mixture to between about pH 2.5-5.5. In some embodiments, peptide exchange is induced by increasing the pH of the mixture to about pH 9-11.

In some embodiments, the placeholder peptide comprises a photocleavable moiety to form pMHC complexes as described (e.g., Toebes et al. Nat. Med. 12:246-251, 2006; Bakker et al. PNAS 105:3825-383, 2008; Frosig et al., Cytometry Part A, 87A:967-975, 2015; Chang et al., Eur. J. Immunol. 43:1109-1120, 2013). In some embodiments, the placeholder peptide comprises a non-natural amino acid that contains a (2-nitro)phenyl side chain. In some embodiments, the amino acid is the UV-sensitive β-amino acid comprising 3-amino-3-(2-nitro)phenyl-propionic acid. In some embodiments, the UV-sensitive amino acid is (2-nitro)phenylglycine.

In some embodiments, the placeholder peptide is an HLA-A2 peptide. In some embodiments, the HLA-A2 placeholder peptide is p*A2, KILGCVFJV (SEQ ID NO:15) or GILGFVFJL (SEQ ID NO: 7), wherein J is 3-amino-3-(2-nitro)phenyl-propionic acid.

In some embodiments, the placeholder peptide is an HLA-A1, -A3, All or -B7 peptide containing a photocleavable moiety. In some embodiments, the placeholder peptide is selected from the group consisting of A*01:01, STAPGJLEY (SEQ ID NO: 16); A*03:01, RIYRJGATR (SEQ ID NO:17); A*11:01, RVFAJSFIK (SEQ ID NO: 18); A*24:02, VYGJVRACL (SEQ ID NO: 11); B*07:02, AARGJTLAM (SEQ ID NO: 14); B*35:01, KPIVVLJGY (SEQ ID NO: 19); C*03:04, FVYGJSKTSL (SEQ ID NO: 20), B*08:01, FLRGRAJGL (SEQ ID NO: 21); C*07:02, VRIJHLYIL (SEQ ID NO: 22); C*04:01, QYDJAVYKL (SEQ ID NO: 23); B*15:01, ILGPJGSVY (SEQ ID NO: 24); B*40:01, TEADVQJWL (SEQ ID NO: 25); B*58:01, ISARGQJLF (SEQ ID NO: 26); and C*08:01, KAAJDLSHFL (SEQ ID NO: 27), wherein J is 3-amino-3-(2-nitro)phenyl-propionic acid. In other embodiments, the placeholder peptide has a sequence shown in any one of SEQ ID NOs: 7-27 or 271-279.

In some embodiments, the placeholder peptide further comprises a fluorescent label. In so embodiments, the fluorescent label is attached to a cysteine residue in the placeholder peptide.

Upon irradiation with long-wavelength UV, the peptide is cleaved and dissociates from the MHC complex in the presence of one or more peptides to facilitate the formation of stable pMHC monomers or multimers. Typically, MHC peptide exchange is performed in multiwell format for high-throughput screening of peptide ligands as described herein. Only peptide candidates that can effectively bind and stabilize the peptide-receptive MHC molecules prevent dissociation of the MHC complexes. Peptide exchange can be monitored by a number of techniques such as ELISA or fluorescence polarization, for example, as generally described in Rodenko et al. (Nat. Protocol. 1:1120-1132, 2006).

The resulting pMHC multimers are subsequently analyzed by gel-filtration HPLC and MHC ELISA to determine three parameters: the efficiency of MHC refolding, the stability of the pMHC complex in the absence of UV exposure, and the UV-sensitivity of the complex.

Certain di-peptides can assist folding and peptide exchange of MHC class I molecules. Di-peptides bind specifically to the F pocket of MHC class I molecules to facilitate peptide exchange and have so far been described and validated for peptide exchange in HLA-A*02:01, HLA-B*27:05, and H-2Kb molecules (Saini et al. Proc Natl Acad Sci USA. 2013 Sep. 17; 110(38):15383-8).

Accordingly, in some embodiments, peptide exchange of the placeholder peptide with a peptide or peptides of interest is catalyzed by a dipeptide, which catalyzes rapid peptide exchange on MHC class I molecules (see, e.g., Saini et al., Proc Natl Acad Sci USA. 2015 Jan. 6; 112(1):202). Suitable dipeptides are those with a hydrophobic second residue. In some embodiments, the dipeptide is glycyl-leucine (GL), glycyl-valine (GV), glycyl-methione (GM), glycyl-cyclohexylalanine (GCha), glycyl-homoleucine (GHle) or glycyl-phenylalanine (GF).

In another embodiment, peptide exchange of the placeholder peptide with a peptide or peotides of interest is achieved by chaperone-mediated peptide exchange, e.g., using the molecular chaperone TAPBPR as described in Overall et al. (2020) Nature Comm. 11:1909.

VI. Production of pMHC Libraries

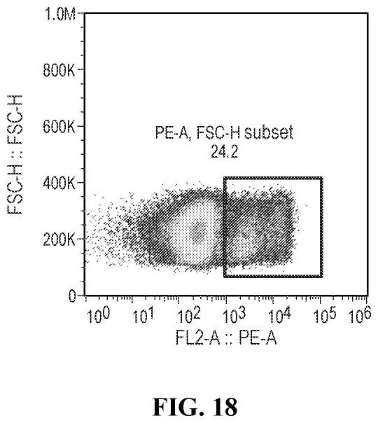

In one aspect, provided herein are methods of producing a library of pMHC multimers comprising a diversity of loaded peptide epitopes. Various steps in the preparation of peptide-exchanged, barcoded pMHC libraries are illustrated schematically in FIG. 18. These steps use standard methods known in the art for preparing barcoded libraries, including use of single-cell sequencing, use of porous hydrogels, use of single template PCR to generate peptide-encoding amplicons (barcodes) and use of in-drop in vitro transcription/translation (IVTT).

A non-limiting exemplification of single-cell sequencing with pooled, barcoded, UV-peptide exchanged MHC tetramers is described in Example 9. A non-limiting exemplification of production of porous hydrogels for high throughput production of barcoded, UV-peptide exchanged MHC tetramer pools is described in detail in Example 10. A non-limiting exemplification of use of single template PCR to generate peptide-encoding amplicons is described in detail in Example 11. A non-limiting exemplification of loading of barcodable, exchange-ready MHC tetramers onto hydrogel is described in Example 12. A non-limiting exemplification of in-drop in vitro transcription/translation (IVTT) of peptide and UV exchange into loaded MHC tetramers is described in detail in Example 13. A non-limiting exemplification of release of UV-peptide exchanged, barcoded pMHC tetramers from hydrogels is described in detail in Example 14.

In some embodiments, the method comprises (a) providing a plurality of placeholder peptide loaded MHCI (p*MHCI) monomers each comprising (i) an MHCI heavy chain polypeptide, or a functional fragment thereof, (ii) a β2-microglobulin polypeptide or functional fragment thereof, (iii) a conjugation moiety, and (iv) a placeholder peptide bound in the peptide binding groove of each MHCI monomer; (b) providing a plurality of multimerization domains, wherein each subunit of the multimerization domain comprises a conjugation moiety; (c) combining the p*MHCI monomers and the multimerization domains under conditions sufficient for covalent conjugation between the two or more p*MHCI monomers and a multimerization domain to produce p*MHCI multimers; and (d) replacing the placeholder-peptide in the plurality of p*MHCI multimers with a peptide library comprising plurality of unique MHCI peptide epitopes to produce a plurality of peptide loaded MHCI (pMHCI) multimers.

In some embodiments, the method comprises (a) providing a plurality of placeholder peptide loaded MHCI (p*MHCI) monomers each comprising (i) an MHCI heavy chain polypeptide, or a functional fragment thereof, (ii) a β2-microglobulin polypeptide or functional fragment thereof, (iii) a conjugation moiety, and (iv) a placeholder peptide bound in the peptide binding groove of each MHCI monomer; (b) providing a plurality of multimerization domains, wherein each subunit of the multimerization domains comprises a conjugation moiety and the multimerization domain comprises at least one non-covalent binding site; (c) combining the plurality of p*MHCI monomers and the plurality of multimerization domain under conditions sufficient for covalent conjugation between the two or more p*MHCI monomers and a multimerization domain to produce a plurality of p*MHCI multimers; (d) replacing the placeholder peptide bound in the peptide binding groove of the p*MHCI multimers with a plurality of unique rescue peptide epitopes to produce a plurality of pMHCI multimers; and (e) binding an oligonucleotide barcode to the non-covalent binding site on the multimerization domain.

In some embodiments, the method comprises (a) providing a plurality of placeholder peptide loaded MHCI (p*MHCI) monomers each comprising (i) an MHCI heavy chain polypeptide, or a functional fragment thereof, (ii) a β2-microglobulin polypeptide or functional fragment thereof, (iii) a peptide linker comprising a conjugation moiety at the C-terminus of (i) or (ii); and (iv) a placeholder peptide bound in the peptide binding groove of each MHCI monomer; (b) providing a plurality of multimerization domains comprising a peptide linker comprising a conjugation moiety at the N-terminus of each subunit of the multimerization domain; (c) combining the plurality of p*MHCI monomers and the plurality of multimerization domains under conditions sufficient for covalent conjugation between two or more p*MHCI monomers to a multimerization domain to produce a plurality of p*MHCI multimers; and (d) replacing the placeholder peptide bound in the peptide binding groove of the p*MHCI multimers with a plurality of unique rescue peptide epitopes to produce a plurality of pMHCI multimers.

### VII. Labeling

pMHC multimers can be conjugated with a fluorescent label, allowing for identification of T cells that bind the peptide-MHC multimer, for example, via flow cytometry or microscopy. T cells can also be selected based on a fluorescence label through, e.g., fluorescence activated cell sorting.

In some embodiments, one or more detectable labels are conjugated to a linker. According to this invention, a “detectable label” is any molecule or functional group that allows for the detection of a biological or chemical characteristic or change in a system, such as the presence of a target substance in the sample.

Examples of detectable labels which may be used include fluorophores, chromophores, electro chemiluminescent labels, bioluminescent labels, polymers, polymer particles, bead or other solid surfaces, gold or other metal particles or heavy atoms, spin labels, radioisotopes, enzyme substrates, haptens, antigens, Quantum Dots, aminohexyl, pyrene, nucleic acids or nucleic acid analogs, or proteins, such as receptors, peptide ligands or substrates, enzymes, and antibodies (including antibody fragments).

Examples of polymer particles labels which may be used include micro particles, beads, or latex particles of polystyrene, PMMA or silica, which can be embedded with fluorescent dyes, or polymer micelles or capsules which contain dyes, enzymes or substrates. Examples of metal particles which may be used include gold particles and coated gold particles, which can be converted by silver stains. Examples of haptens that may be conjugated in some embodiments are fluorophores, myc, nitrotyrosine, biotin, avidin, streptavidin, 2,4-dinitrophenyl, digoxigenin, bromodeoxy uridine, sulfonate, acetylaminoflurene, mercury trintrophonol, and estradiol.

Examples of enzymes which may be used comprise horse radish peroxidase (HRP), alkaline phosphatase (AP),beta-galactosidase (GAL), glucose-6-phosphate dehydrogenase, beta-N-acetylglucosaminidase, βglucuronidase, invertase, Xanthine Oxidase, firefly luciferase and glucose oxidase (GO). Examples of commonly used substrates for horse radish peroxidase (HRP) include 3,3′-diaminobenzidine (DAB), diaminobenzidine with nickel enhancement, 3-amino-9-ethylcarbazole (AEC), Benzidine dihydrochloride (BDHC), Hanker-Yates reagent (HYR), Indophane blue (TB), tetramethylbenzidine (TMB), 4-chloro-1-naphtol (CN), alpha-naphtol pyronin (.alpha.-NP),o-dianisidine (OD), 5-bromo-4-chloro-3-indolylphosphate (BLIP), Nitroblue tetrazolium (NBT), 2-(p-iodophenyl)-3-p-nitrophenyl-5-phenyltetrazolium chloride (INT), tetranitro blue tetrazolium (TNBT), .delta.-bromo-chloro-S-indoxyl-beta-D-galactoside/ferro-ferricyanide (BCIG/FF). Examples of commonly used substrates for Alkaline Phosphatase include Naphthol-AS-B1-phosphate/fast red TR (NABP/FR),Naphthol-AS-MX-phosphate/fast red TR (NAMP/FR),Naphthol-AS-B1-phosphate/fast red TR (NABP/FR),Naphthol-AS-MX-phosphate/fast red TR (NAMP/FR),Naphthol-AS-B1-phosphate/new fuschin (NABP/NF), bromochloroindolylphosphate/nitroblue tetrazolium (BCIP/NBT), b-Bromo-chloro-S-indolyl-beta-delta-galactopyranoside (BCIG).

Examples of luminescent labels which may be used include luminol, isoluminol, acridinium esters, 1,2-dioxetanes and pyridopyridazines. Examples of electrochemiluminescent labels include ruthenium derivatives. Examples of radioactive labels which may be used include radioactive isotopes of iodide, cobalt, selenium, hydrogen, carbon, sulfur, and phosphorous.

Some “detectable labels” also include “colour labels,” in which the biological change or event in the system may be assayed by the presence of a colour, or a change in colour. Examples of “colour labels” are chromophores, fluorophores, chemiluminescent compounds, electrochemiluminescent labels, bioluminescent labels, and enzymes that catalyze a colour change in a substrate.

“Fluorophores” as described herein are molecules that emit detectable electro-magnetic radiation upon excitation with electro-magnetic radiation at one or more wavelengths. A large variety of fluorophores are known in the art and are developed by chemists for use as detectable molecular labels and can be conjugated to the pMHC multimers provided herein. Examples include FLUORESCEIN™ or its derivatives, such as FLUORESCEIN®-5-isothiocyanate (FITC), 5-(and 6)-carboxyFLUORESCEIN®, 5- or 6-carboxyFLUORESCEIN®,6-(FLUORESCEIN®)-5-(and 6)-carboxamido hexanoic acid, FLUORESCEIN® isothiocyanate, rhodamine or its derivatives such as tetramethyl rhodamine and tetramethylrhodamine-5-(and -6) isothiocyanate (TRITC). Other fluorophores include: coumarin dyes such as (diethyl-amino)coumarin or 7-amino-4-methylcoumarin-3-acetic acid, succinimidyl ester (AMCA); sulforhodamine 101 sulfonyl chloride (TexasRed® or TexasRed® sulfonyl chloride; 5-(and -6)-carboxyrhodamine 101, succinimidyl ester, also known as 5-(and -6)-carboxy-X-rhodamine, succinimidyl ester (CXR); lissamine or lissamine derivatives such as lissamine rhodamine B sulfonyl Chloride (LisR); 5-(and -6)-carboxyFLUORESCEIN®, succinimidyl ester (CFI); FLUORESCEIN®5-isothiocyanate (FITC); 7-diethylaminocoumarin-3-carboxylic acid, succinimidyl ester (DECCA); 5-(and -6)-carboxytetramethyl-rhodamine, succinimidyl ester (CTMR); 7-hydroxycoumarin-3-carboxylic acid, succinimidyl ester (HCCA); 6->FLUORESCEIN®.-5-(and -6)-carboxamidolhexanoic acid (FCHA); N-(4,4-difluoro-5,7-dimethyl-4-bora-3a,4a-diaza-3-indacenepropionic acid, succinimidyl ester; also known as 5,7-dimethylBODIPY® propionic acid, succinimidyl ester (DMBP); “activated FLUORESCEIN® derivative” (FAP), available from Probes, Inc.; eosin-5-isothiocyanate (EITC); erythrosin-5-isothiocyanate (ErlTC); and Cascade® Blue acetylazide (CBAA) (the O-acetylazide derivative of 1-hydroxy-3,6,8-pyrene-trisulfonic acid). Yet other potential fluorophores useful in this invention include fluorescent proteins such as green fluorescent protein and its analogs or derivatives, fluorescent amino acids such as tyrosine and tryptophan and their analogs, fluorescent nucleosides, and other fluorescent molecules such as Cy2,Cy3, Cy 3.5, CY5™, CY5™5, Cy 7, IR dyes, Dyomics dyes, phycoerythrine, Oregon green 488, pacific blue, rhodamine green, and Alexa dyes. Yet other examples of fluorescent labels include conjugates of R-phycoerythrin orallophycoerythrin, inorganic fluorescent labels such as particles based on semiconductor material like coated CdSe nanocrystallites.

A number of the fluorophores above, as well as others, are available commercially, from companies such as Probes, Inc. (Eugene, Oreg.), Pierce Chemical Co. (Rockford, Ill.), or Sigma-Aldrich Co. (St. Louis, Mo.).

The detectable label can be detected by numerous methods, including, for example, reflectance, transmittance, light scatter, optical rotation, and fluorescence or combinations hereof in the case of optical labels or by film, scintillation counting, or phosphorimaging in the case of radioactive labels. See, e.g., Larsson, 1988, Immunocytochemistry: Theory and Practice, (CRC Press, Boca Raton, Fla.); Methods in Molecular Biology, vol. 80 1998, John D. Pound (ed.) (Humana Press, Totowa, N.J.). In some embodiments, more than one detectable labels employed.

### VIII. Identifiers/Barcoding

In certain embodiments, a Conjugated Multimer of the disclosure comprises an identifier tag or label, such as an oligonucleotide barcode, that facilitates identification of the Conjugated Multimer. Typically, the identifier tag, e.g., oligonucleotide barcode, is attached to the multimerization domain of the Conjugated Multimer, such as through a binding moiety on the identifier tag, e.g., oligonucleotide barcode, that binds to a binding site on the multimerization domain. For example, when the multimerization domain is streptavidin or avidin, since the pMHCI monomers are conjugated to the multimerization domain at a site other than the biotin-binding site, the Conjugated Multimer can be labeled with an identifier tag, e.g., oligonucleotide barcode, using a biotinylated form of the identifier tag, e.g., a biotinylated oligonucleotide barcode. Labeling of the Conjugated Multimer is then easily achieved by incubation of the Conjugated Multimer with the biotinylated identifier tag, e.g., biotinylated oligonucleotide barcode. A non-limiting exemplification of barcoding of Conjugated Multimers using biotinylated oligonucleotides is described in detail in Example 8.

In another embodiment, the Conjugated Multimer is labeled with an identifier tag, e.g., oligonucleotide barcode, in the peptide portion of the multimer. That is, barcode-labeled MHC-binding peptides can be used in an exchange reaction as described herein to the load the Conjugated Multimers with barcode-labeled peptides.

Typically, an oligonucleotide barcode is a unique oligonucleotide sequence ranging for to more than 50 nucleotides. The barcode has shared amplification sequences in the 3′ and 5′ ends, and a unique sequence in the middle. This sequence can be revealed by sequencing and can serve as a specific barcode for a given molecule.

In one embodiment, the nucleic acid component of the barcode (typically DNA) has a special structure. Thus, in one embodiment, the at least one nucleic acid molecule is composed of at least a 5′ first primer region, a central region (barcode region), and a 3′ second primer region. In this way the central region (the barcode region) can be amplified by a primer set. The length of the nucleic acid molecule may also vary. Thus, in other embodiments, the at least one nucleic acid molecule has a length in the range 20-100 nucleotides, such as 30-100, such as 30-80, such as 30-50 nucleotides. In one embodiment, the nucleic acid identifier is from 40 nucleotides to 120 nucleotides in length. The coupling of the oligonucleotide barcode to the Conjugated Multimer may also vary. Thus, in one embodiment, the at least one oligonucleotide barcode is linked to said Conjugated Multimer via a biotin binding domain interacting with streptavidin or avidin within the Conjugated Multimer. Other coupling moieties may also be used, depending on the availability of an appropriate binding site with the Conjugated Multimer (e.g., within the multimerization domain of the Conjugated Multimer) and an appropriate corresponding binding domain that can be attached to the oligonucleotide barcodes molecules to facilitate attachment.

In a further embodiment, the at least oligonucleotide barcode molecule comprises or consists of DNA, RNA, and/or artificial nucleotides such as PLA or LNA. Preferably DNA, but other nucleotides may be included to e.g. increase stability.

The use of barcode technology is well known in the art, see for example Shiroguchi et al., Proc. Natl. Acad. Sci. USA., 2012 Jan. 24; 109(4):1347-52; and Smith et al., Nucleic Acids Research, 2010 July; 38(13)11:e142. Further methods and compositions for using barcode technology include those described in U.S. 2016/0060621. Use of barcode technology specifically to label MHC multimers also has been described, see for example Bentzen et al., Nature Biotech. 34:10: 1037-1045, 2016; Bentzen and Hadrup, Cancer Immunol. Immunotherap. 66:657-666, 2017. Standard methods for preparing barcode oligonucleotides, including conjugating them with a suitable binding moiety (e.g., biotinylation) that can bind the Conjugated Multimer, are known in the art and can be applied to preparing barcode oligonucleotides for labeling the Conjugated Multimers.

Methods for generating customizable DNA barcode libraries are publicly available. Programs include Generator and nxCode, consisting of 96-587 barcodes, respectively, as well as The DNA Barcodes Package and TagD software (reporting generating libraries consisting of 100,000 barcodes).

Preparation of a variety of large-scale barcode libraries have been described in the art, which approaches can be used to obtain barcode libraries for labeling pMHC Conjugated Multimer libraries. For example, Xu et al. describe a set of 240,000 unique 25-mer oligonucleotides with sequences that have similar amplifications properties while maintaining maximum diversity of their identification motifs (Xu et al. PNAS 106:2289-2294, 2008). Wang et al. describe construction of barcode sets using particle swarm optimization (Wang et al. IEEE/ACM Trans. Comput. Biol. Bioinform. 15:999-1002). Lyons describes generation of large-scale libraries of DNA barcodes of up to one million members. (Lyons, Sci. Reports 7:13899, 2017).

In some cases, the unique molecular identifier barcode is encoded by a contiguous sequence of nucleotides tagged to one end of a target nucleic acid. In other cases, the unique molecular identifier (UMI) barcode is encoded by a non-contiguous sequence. Non-contiguous UMIs can have a portion of the barcode at a first end of the target nucleic acid and a portion of the barcode at a second end of the target nucleic acid. In some cases, the UMI is a non-contiguous barcode containing a variable length barcode sequence at a first end and a second identifier sequence at a second end of the target nucleic acid. In some cases, the UMI is a non-contiguous barcode having a variable length barcode sequence at a first end and a second identifier sequence at a second end of the target nucleic acid, wherein the second identifier sequence is determined by a position of a transposase fragmentation event, e.g., a transposase fragmentation site and transposon end insertion event.

In some cases, the barcode is a “variable length barcode.” As used herein, a variable length barcode is an oligonucleotide that differs from other variable length barcode oligonucleotides in a population, by length, which can be identified by the number of contiguous nucleotides in the barcode. In some cases, additional barcode complexity for the variable length barcode can be provided by the use of variable nucleotide sequence, as described in the paragraphs above, in addition to the variable length.

In an exemplary embodiment, a variable length barcode can have a length of from 0 to no more than 5 nucleotides. Such a variable length barcode can be denoted by the term “[0-5].” In such an embodiment, it is understood that a population of target nucleic acids that are attached to such a variable length barcode is expected to include at least one target nucleic acid attached to a variable length barcode that has at least 1 nucleotide (e.g., attached to a variable length barcode having only 1, only 2, only 3, only 4, or only 5 nucleotides). In such an embodiment, it is further understood that a population of target nucleic acids that are attached to such a variable length barcode can include at least one target nucleic acid that contains no variable length barcode (i.e., a variable length barcode having a length of 0), and/or at least one target nucleic acid that contains a variable length barcode having only 1 nucleotide, and/or at least one target nucleic acid that contains a variable length barcode having only 2 nucleotides, and/or at least one target nucleic acid that contains a variable length barcode having only 3 nucleotides, and/or at least one target nucleic acid that contains a variable length barcode having only 4 nucleotides, and/or and at least one target nucleic acid that contains a variable length barcode having only 5 nucleotides. In such an embodiment, the [0-5] variable length barcode can uniquely identify (differentiate), by itself, 5 different target nucleic acid molecules of the same sequence. Further, in such an embodiment, the [0-5] variable length barcode can uniquely identify (differentiate) 5 different target nucleic molecules of a first sequence, 5 different target nucleic acid molecules of a second sequence, etc. for each different target nucleic acid sequence. Furthermore, barcode labelled MHC-multimers can be used in combination with single-cell sorting and TCR sequencing, where the specificity of the TCR can be determined by the co-attached barcode. This will enable us to identify TCR specificity for potentially 1000+different antigen responsive T-cells in parallel from the same sample, and match the TCR sequence to the antigen specificity. The future potential of this technology relates to the ability to predict antigen responsiveness based on the TCR sequence.

The complexity of the barcode labeled MHC multimer libraries will allow for personalized selection of relevant TCRs in a given individual.

The barcode is co-attached to the multimer and serves as a specific label for a particular peptide-MHC complex. In this way at least 1000 to 10,000 or more different peptide-MHC multimers can be mixed, allow specific interaction with T-cells from blood or other biological specimens, wash-out unbound MHC-multimers and determine the sequence of the DNA-barcodes. When selecting a cell population of interest, the sequence of barcodes present above background level, will provide a fingerprint for identification of the antigen responsive cells present in the given cell-population. The number of sequence-reads for each specific barcode will correlate with the frequency of specific T-cells, and the frequency can be estimated by comparing the frequency of reads to the input-frequency of T-cells.

The DNA-barcode serves as a specific labels for the antigen specific T-cells and can be used to determine the specificity of a T-cell after e.g. single-cell sorting, functional analyses or phenotypical assessments. In this way antigen specificity can be linked to both the T-cell receptor sequence (that can be revealed by single-cell sequencing methods) and functional and phenotypical characteristics of the antigen specific cells.

Barcode labeled MHC multimer libraries can be used for the quantitative assessment of MHC multimer binding to a given T-cell clone or TCR transduced/transfected cells. Since sequencing of the barcode label allow several different labels to be determined simultaneously on the same cell population, this strategy can be used to determine the avidity of a given TCR relative to a library of related peptide-MHC multimers. The relative contribution of the different DNA-barcode sequences in the final readout is determined based on the quantitative contribution of the TCR binding for each of the different peptide-MHC multimers in the library. Via titration based analyses it is possible to determine the quantitative binding properties of a TCR in relation to a large library of peptide-MHC multimers, all merged into a single sample. For this particular purpose the MHC multimer library may specifically hold related peptide sequences or alanine-substitution peptide libraries.

In some embodiments, unique identifiers can be used for each sample of a plurality of samples. In some embodiments, identifiers can be shared between two or more samples. In some embodiments, identifiers can comprise some sequences that are shared between all samples, and other sequences that are unique to one sample. In some embodiments, an identifier can comprise a sequence shared between all samples, and a sequence unique to one sample. In some embodiments, a sequence shared between samples can be used for identifier amplification (e.g., PCR amplification with suitable primers). In some embodiments, a sequence unique to one sample or shared between a subset of samples can be used for detection or quantification via qPCR (e.g., sequences for hydrolysis probes, such as TaqMan probes). In some embodiments, a sequence unique to one sample or shared between a subset of samples can be used for detection or quantification via sequencing.

In some embodiments, an identifier can comprise a unique, in silico-generated sequence; each identifier sequence can be assigned to a sample of a plurality of samples and the identifier-sample assignment can be stored in a database. In some embodiments, an identifier can comprise a nucleotide sequence that codes for all or part of a peptide or protein. In some embodiments, an identifier can comprise a nucleotide sequence that codes for an open reading frame. In some embodiments, an identifier can comprise a nucleotide sequence that includes a promoter sequence. In some embodiments, an identifier can comprise a nucleotide sequence that includes a binding site for a DNA-binding protein, e.g. a transcription factor or polymerase enzyme. In some embodiments, an identifier can comprise one or more sequences targeted by a nuclease, e.g. a restriction enzyme. In some embodiments, an identifier can comprise all sequence elements necessary for in vitro transcription and translation of a sequence. In some embodiments, an identifier does not comprise all sequence elements necessary for in vitro transcription and translation of a sequence.

In some embodiments, an identifier can comprise a biotinylated nucleotide sequence. In some embodiments, an identifier can be biotinylated by PCR amplification with a biotinylated primer(s). In some embodiments, an identifier can be biotinylated by enzymatic incorporation of a biotinylated label, e.g. a biotin dUTP label, by use of Klenow DNA polymerase enzyme, nick translation or mixed primer labeling RNA polymerases, including T7, T3, and SP6 RNA polymerases. In some embodiments, an identifier can be biotinylated by photobiotinylation, e.g. photoactivatable biotin can be added to the sample, and the sample irradiated with UV light.

In some embodiments, an identifier can be generated from a template polynucleotide, e.g. via PCR amplification of a template DNA. In some embodiments, a template polynucleotide can comprise a nucleotide sequence that codes for an open reading frame. In some embodiments, a template polynucleotide can comprise a nucleotide sequence that includes a promoter sequence. In some embodiments, a template polynucleotide can comprise a nucleotide sequence that includes a binding site for a DNA-binding protein, e.g. a transcription factor or polymerase enzyme. In some embodiments, a template polynucleotide can comprise one or more sequences targeted by a nuclease, e.g. a restriction enzyme. In some embodiments, a template polynucleotide can comprise all sequence elements necessary for in vitro transcription and translation of a sequence. In some embodiments, a template polynucleotide does not comprise all sequence elements necessary for in vitro transcription and translation of a sequence.

pMHC multimers with attached identifiers (e.g., oligonucleotide barcodes) can be incubated with a plurality of T cells, followed by sorting of T cells into single-cell compartments. T cells are lysed, and nucleic acids from lysed T cells comprising identifiers are produced. Nucleic acids are pooled and sequenced. Identifiers allow matching of peptide identifiers to T cell sequences from the same compartment. TCR-antigen specificity profiles are determined by identifying a TCR sequence (e.g., variable region, hypervariable region, or CDR) from a compartment, and quantifying peptide identifier reads from the same compartment.

Multiple TCRs can be identified that exhibit binding affinity for peptides of the peptide library, and multiple peptides can be identified that exhibit binding affinity for specific TCRs.

Epitope mutations in an antigen of an identified TCR-antigen pair can be identified that result in increased or TCR binding affinity.

Peptides and TCR sequences can be identified that are associated with control of disease associated protein, and can be used to design vaccines and cell therapies.

For assessing response to therapy, for each peptide identifier sequenced, corresponding TCR sequences are identified. Multiple TCRs are identified that exhibit binding affinity for some peptides of the peptide library, and multiple peptides are identified that exhibit binding affinity for some TCRs. Subjects are followed longitudinally and results of assays are compared to identify peptides and TCR sequences that are associated with successful response to immunotherapy.

### IX. Vectors and Polynucleotides

Also included in the present disclosure are nucleic acid sequences encoding any of the proteins described herein. As appreciated by those skilled in the art, because of third base degeneracy, almost every amino acid can be represented by more than one triplet codon in a coding nucleotide sequence. In addition, minor base pair changes may result in a conservative substitution in the amino acid sequence encoded but are not expected to substantially alter the biological activity of the gene product. Therefore, a nucleic acid sequence encoding a protein described herein may be modified slightly in sequence and yet still encode its respective gene product.

Nucleic acids encoding any of the various proteins or polypeptides described herein may be synthesized chemically. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al., Proc. Natl. Acad. Sci. USA, 100(2):438-442 (Jan. 21, 2003); Sinclair et al., Protein Expr. Purif., 26(I):96-105 (October 2002); Connell, N. D., Curr. Opin. Biotechnol., 12(5):446-449 (October 2001); Makrides et al., Microbiol. Rev., 60(3):512-538 (September 1996); and Sharp et al., Yeast, 7(7):657-678 (October 1991).

General techniques for nucleic acid manipulation are described in, for example, Sambrook et al., Molecular Cloning: A Laboratory Manual, 2nd Edition, Vols. 1-3, Cold Spring Harbor Laboratory Press (1989), or Ausubel, F. et al., Current Protocols in Molecular Biology, Green Publishing and Wiley-Interscience, New York (1987) and periodic updates, herein incorporated by reference. Generally, the DNA encoding the polypeptide is operably linked to suitable transcriptional or translational regulatory elements derived from mammalian, viral, or insect genes. Such regulatory elements include a transcriptional promoter, an optional operator sequence to control transcription, a sequence encoding suitable mRNA ribosomal binding site, and sequences that control the termination of transcription and translation. The ability to replicate in a host, usually conferred by an origin of replication, and a selection gene to facilitate recognition of transformants is additionally incorporated.

The proteins described herein may be produced recombinantly not only directly, but also as a fusion polypeptide with a heterologous polypeptide, which is preferably a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell.

For prokaryotic host cells that do not recognize and process a native signal sequence, the signal sequence is substituted by a prokaryotic signal sequence selected, for example, from the group of the alkaline phosphatase, penicillinase, 1 pp, or heat-stable enterotoxin II leaders.

For yeast secretion the native signal sequence may be substituted by, e.g., a yeast invertase leader, a factor leader (including Saccharomyces and Kluyveromyces alpha-factor leaders), or acid phosphatase leader, the C. albicans glucoamylase leader, or the signal sequence described in U.S. Pat. No. 5,631,144. In mammalian cell expression, mammalian signal sequences as well as viral secretory leaders, for example, the herpes simplex gD signal, are available. The DNA for such precursor regions may be ligated in reading frame to DNA encoding the protein.

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2 micron plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Expression and cloning vectors may contain a selection gene, also termed a selectable marker. Typical selection genes encode proteins that (a) confer resistance to antibiotics or other toxins, e.g., ampicillin, neomycin, methotrexate, or tracycline, (b) complement auxotrophic deficiencies, or (c) supply critical nutrients not available from complex media, e.g., the gene encoding D-alanine racemase for Bacilli.

Expression and cloning vectors usually contain a promoter that is recognized by the host organism and is operably linked to the nucleic acid encoding the protein described herein, e.g., a fibronectin-based scaffold protein. Promoters suitable for use with prokaryotic hosts include the phoA promoter, beta-lactamase and lactose promoter systems, alkaline phosphatase, a tryptophan (trp) promoter system, and hybrid promoters such as the tan promoter. However, other known bacterial promoters are suitable. Promoters for use in bacterial systems also will contain a Shine-Dalgarno (S.D.) sequence operably linked to the DNA encoding the protein described herein. Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

Transcription of a DNA encoding protein described herein by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature, 297:17-18 (1982) on enhancing elements for activation of eukaryotic promoters. The enhancer may be spliced into the vector at a position 5′ or 3′ to the peptide-encoding sequence, but is preferably located at a site 5′ from the promoter.

Expression vectors used in eukaryotic host cells (e.g., yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of mRNA encoding the protein described herein. One useful transcription termination component is the bovine growth hormone polyadenylation region. See WO 94/11026 and the expression vector disclosed therein.

The recombinant DNA can also include any type of protein tag sequence that may be useful for purifying the protein. Examples of protein tags include, but are not limited to, a histidine tag, a FLAG tag, a myc tag, an HA tag, or a GST tag. Appropriate cloning and expression vectors for use with bacterial, fungal, yeast, and mammalian cellular hosts can be found in Cloning Vectors: A Laboratory Manual, (Elsevier, New York (1985)), the relevant disclosure of which is hereby incorporated by reference.

The expression construct is introduced into the host cell using a method appropriate to the host cell, as will be apparent to one of skill in the art. A variety of methods for introducing nucleic acids into host cells are known in the art, including, but not limited to, electroporation; transfection employing calcium chloride, rubidium chloride, calcium phosphate, DEAE-dextran, or other substances; microprojectile bombardment; lipofection; and infection (where the vector is an infectious agent).

Suitable host cells include prokaryotes, yeast, mammalian cells, or bacterial cells. Suitable bacteria include gram negative or gram positive organisms, for example, E. coli or Bacillus spp. Yeast, preferably from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow et al. (Bio/Technology, 6:47 (1988)). Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. Purified polypeptides are prepared by culturing suitable host/vector systems to express the recombinant proteins. For many applications, the small size of many of the polypeptides described herein would make expression in E. coli as the preferred method for expression. The protein is then purified from culture media or cell extracts.

The host cells used to produce the proteins of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma)) are suitable for culturing the host cells. In addition, many of the media described in Ham et al., Meth. Enzymol., 58:44 (1979), Barites et al., Anal. Biochem., 102:255 (1980), U.S. Pat. Nos. 4,767,704, 4,657,866, 4,927,762, 4,560,655, 5,122,469, 6,048,728, 5,672,502, or U.S. Pat. No. RE 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Proteins described herein can also be produced using cell-free translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system).

Proteins described herein can also be produced by chemical synthesis (e.g., by the methods described in Solid Phase Peptide Synthesis, 2nd Edition, The Pierce Chemical Co., Rockford, Ill. (1984)). Modifications to the protein can also be produced by chemical synthesis.

The proteins of the present invention can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, get filtration, gel permeation chromatography, affinity chromatography, electrophoresis, countercurrent distribution or any combinations of these. After purification, polypeptides may be exchanged into different buffers and/or concentrated by any of a variety of methods known to the art, including, but not limited to, filtration and dialysis.

The purified polypeptide is preferably at least 85% pure, or preferably at least 95% pure, and most preferably at least 98% pure. Regardless of the exact numerical value of the purity, the polypeptide is sufficiently pure for its intended use.

### X. Methods of Use

Another aspect of the invention relates to methods for detecting antigen responsive T cells, for example in a sample. Generally, the methods comprise providing a plurality of pMHC Conjugated Multimers of the disclosure; contacting the Conjugated Multimers with said sample; and detecting binding of the Conjugated Multimers to antigen responsive T cells within the sample, thereby detecting T cells responsive to an antigenic peptide present in the plurality of Conjugated Multimers. In one embodiment, binding is detected by amplifying the barcode region of the oligonucleotide barcode linked to the Conjugated Multimer. Typically, for pMHCI Conjugated Multimers, the antigen responsive T cell is a CD8+ T cell, whose TCRs recognize peptide-bound MHC Class I molecules, whereas for pMHCII Conjugated Multimers, the antigen responsive T cell is a CD4+ T cell, whose TCRs recognize peptide-bound MHC Class II molecules.

This Conjugated Multimer technology allows for detection of multiple (potentially >1000) different antigen-specific T cells in a single sample. The technology can be used, for example, for T-cell epitope mapping, immune-recognition discovery, diagnostics tests and measuring immune reactivity after vaccination or immune-related therapies. For therapeutic use, the pMHC Conjugated Multimers allow for identification and selection of antigen-specific T cells to be administered for therapy, such as for adoptive T cell transfer therapy.

A. Assays

In one embodiment of the present invention MHC multimers can be used for detection of individual T-cells in fluid samples using flowcytometry or flow cytometry-like analysis.

Liquid cell samples can be analyzed using a flow cytometer, able to detect and count individual cells passing in a stream through a laser beam. For identification of specific T-cells using MHC multimers, cells are stained with fluorescently labeled MHC multimer by incubating cells with MHC multimer and then forcing the cells with a large volume of liquid through a nozzle creating a stream of spaced cells. Each cell passes through a laser beam and any fluorochrome bound to the cell is excited and thereby fluoresces. Sensitive photomultipliers detect emitted fluorescence, providing information about the amount of MHC multimer bound to the cell. By this method MHC multimers can be used to identify individual T-cells and/or specific T-cell populations in liquid samples.

Cell samples capable of being analyzed by MHC multimers in flowcytometry analysis include, but is not limited to, blood samples or fractions thereof, T-cell lines (hybridomas, transfected cells) and homogenized tissues like spleen, lymph nodes, tumors, brain or any other tissue comprising T-cells.

When analyzing blood samples whole blood can be used with or without lysis of red blood cells prior to analysis on flow cytometer. Lysing reagent can be added before or after staining with MHC multimers. When analyzing blood samples without lysis of red blood cells one or more gating reagents may be included to distinguish lymphocytes from red blood cells. Preferred gating reagent are marker molecules specific for surface proteins on red blood cells, enabling subtraction of this cell population from the remaining cells of the sample. As an example, a fluorochrome labelled CD45 specific marker molecule e.g. an antibody can be used to set the trigger discriminator to allow the flow cytometer to distinguish between red blood corpuscles and stained white blood cells.

Alternative to analysis of whole blood, lymphocytes can be purified before flow cytometry analysis e.g. using standard procedures like aFICOLL®-Hypaque gradient. Another possibility is to isolate T-cells from the blood sample, for example, by adding the sample to antibodies or other T-cell specific markers immobilized on solid support. Marker specific T-cells will then attached to the solid support and following washing specific T-cells can be eluted. This purified T-cell population can then be used for flow cytometry analysis together with MHC multimers.

T-cells may also be purified from other lymphocytes or blood cells by rosetting. Human T-cells form spontaneous rosettes with sheep erythrocytes also called E-rossette formation. E-rossette formation can be carried out by incubating lymphocytes with sheep red erythrocytes followed by purification over a density gradient e.g. a FICOLL® Hypaque gradient.

Instead of actively isolating T-cells unwanted cells like B-cells, NK cells or other cell populations can be removed prior to the analysis. A preferred method for removal of unwanted cells is to incubate the sample with marker molecules specific or one or more surface proteins on the unwanted cells immobilized unto solid support. An example includes use of beads coated with antibodies or other marker molecule specific for surface receptors on the unwanted cells e.g. markers directed against CD19, CD56, CD14, CD15 or others. Briefly beads coated with the specific surface marker(s) are added to the cell sample. Cells different from the wanted T-cells with appropriate surface receptors will bind the beads. Beads are removed by e.g. centrifugation or magnetic withdrawal (when using magnetic beads) and remaining cell are enriched for T-cells.

Another example is affinity chromatography using columns with material coated with antibodies or other markers specific for the unwanted cells.

Alternatively, specific antibodies or markers can be added to the blood sample together with complement, thereby killing cells recognized by the antibodies or markers.

Various gating reagents can be included in the analysis. Gating reagents here means labeled antibodies or other labelled marker molecules identifying subsets of cells by binding to unique surface proteins or intracellular components or intracellular secreted components. Preferred gating reagents when using MHC multimers are antibodies and marker molecules directed against CD2, CD3, CD4, and CD8 identifying major subsets of T-cells. Other preferred gating reagents are antibodies and markers against CD11a, CD14, CD15, CD19, CD25, CD30, CD37, CD49a, CD49e, CD56, CD27, CD28, CD45, CD45RA, CD45RO, CD45RB, CCR7, CCR5, CD62L, CD75, CD94, CD99, CD107b, CD109, CD152, CD153, CD154, CD160, CD161, CD178, CDw197, CDw217, Cd229, CD245, CD247, Foxp3, or other antibodies or marker molecules recognizing specific proteins unique for different lymphocytes, lymphocyte populations or other cell populations. Also included are antibodies and markers directed against interleukins e.g. IL-2, IL-4, IL-6, IL-10, IL-12, IL-21; Interferons e.g., INFγ, TNFα, TNFβ. or other cytokine or chemokines.

Gating reagents can be added before, after or simultaneous with addition of MHC multimer to the sample. Following labelling with MHC multimers and before analysis on a flow cytometer stained cells can be treated with a fixation reagent (e.g., formaldehyde, ethanol or methanol) to cross-link bound MHC multimer to the cell surface. Stained cells can also be analyzed directly without fixation.

The flow cytometer can in one embodiment be equipped to separate and collect particular types of cells. This is called cell sorting. MHC multimers in combination with sorting on a flow cytometer can be used to isolate antigen specific T-cell populations. Gating reagents as described above can be including further specifying the T-cell population to be isolated. Isolated and collected specific T-cell populations can then be further manipulated as described elsewhere herein, e.g. expanded in vitro.

Direct determination of the concentration of MHC-peptide specific T-cells in a sample can be obtained by staining blood cells or other cell samples with MHC multimers and relevant gating reagents followed by addition of an exact amount of counting beads of known concentration. In general, the counting beads are microparticles with scatter properties that put them in the context of the cells of interest when registered by a flow cytometer. They can be either labelled with antibodies, fluorochromes or other marker molecules or they may be unlabelled. In some embodiments, the beads are polystyrene beads with molecules embedded in the polymer that are fluorescent in most channels of the flow-cytometer. Inhere the terms “counting bead” and “microparticle” are used interchangeably.

Beads or microparticles suitable for use include those which are used for gel chromatography, for example, gel filtration media such as SEPHADEX®. Suitable microbeads of this sort include, but is not limited to, SEPHADEX® G-10 having a bead size of 40-120 μm (SigmaAldrich catalogue number 27, 103-9), SEPHADEX®. G-15 having a bead size of 40-120 μm (Sigma Aldrich catalogue number 27, 104-7), SEPHADEX®. G-25 having a bead size of 20-50 μm (Sigma Aldrich catalogue number 27, 106-3), SEPHADEX®. G-25 having a bead size of 20-80 μm (Sigma Aldrich catalogue number 27, 107-1), SEPHADEX®. G-25 having a bead size of 50-150 μm (Sigma Aldrich catalogue number 27, 109-8), SEPHADEX®. G-25 having a bead size of 100-300 μm (Sigma Aldrich catalogue number 27, 110-1), SEPHADEX® G-50 having a bead size of 20-50 μm (Sigma Aldrich catalogue number 27, 112-8), SEPHADEX® G-50 having a bead size of 20-80 μm (Sigma Aldrich catalogue number 27, 113-6), SEPHADEX® G-50 having a bead size of 50-150 μm (Sigma Aldrich catalogue number 27, 114-4), SEPHADEX® G-50 having a bead size of 100-300 μm (SigmaAldrich catalogue number 27, 115-2), SEPHADEX® G-75 having a bead size of 20-50 μm (Sigma Aldrich catalogue number 27, 116-0), SEPHADEX® G-75 having a bead size of 40-120 μm (Sigma Aldrich catalogue number 27, 117-9), SEPHADEX® G-100 having a bead size of 20-50 μm (SigmaAldrich catalogue number 27, 118-7), SEPHADEX® G-100 having a bead size of 40-120 μm (Sigma Aldrich catalogue number 27, 119-5), SEPHADEX® G-150 having a bead size of 40-120 μm (Sigma Aldrich catalogue number 27, 121-7), and SEPHADEX® G-200 having a bead size of 40-120 μm (Sigma Aldrich catalogue number 27, 123-3).

Other preferred particles for use in the methods and compositions described here comprise plastic microbeads. While plastic microbeads are usually solid, they may also be hollow inside and could be vesicles and other microcarriers. They do not have to be perfect spheres in order to function in the methods described here. Plastic materials such as polystyrene, polyacrylamide and other latex materials may be employed for fabricating the beads, but other plastic materials such as polyvinylchloride, polypropylene and the like may also be used.

The counting beads are used as reference population to measure the exact volume of analyzed sample. The sample(s) are analyzed on a flow cytometer and the amount of MHC-specific T-cell is determined using e.g. a predefined gating strategy and then correlating this number to the number of counted counting beads in the same sample

Detection of specific T-cells in a sample combined with simultaneous detection of activation status of T-cells can also be measured using marker molecules specific for up- or down-regulated surface exposed receptors together with MHC multimers. The marker molecule and MHC multimer can be labelled with the same label or different labelling molecules and added to the sample simultaneously or sequentially or separately.

1. Detection of Individual T-Cells in Fluid Samples Using Microscopy

Another preferred method for detection of individual T-cells in fluid samples is using microscopy. Microscopy comprises any type of microscopy including optical, electron and scanning probe microscopy, Bright field microscopy, Dark field microscopy, Phase contrast microscopy, Differential interference contrast microscopy, Fluorescence microscopy, Confocal laser scanning microscopy, X-ray microscopy, Transmission electron microscopy, Scanning electron microscopy, atomic force microscope, Scanning tunneling microscope and photonic force microscope. This can be done as follows: A suspension of T-cells are added to MHC multimers, the sample washed and then the amount of MHC multimer bound to each cell is measured. Bound MHC multimers may be labelled directly or measured through addition of labelled marker molecules. The sample is then spread out on a slide or similar in a thin layer able to distinguish individual cells and labelled cells identified using a microscope. Depending on the type of label different types of microscopes may be used, e.g. if fluorescent labels are used a fluorescent microscope is used for the analysis. For example, MHC multimers can be labeled with a flourochrome or bound MHC multimer detected with a fluorescent antibody. Cells with bound fluorescent MHC multimers can then be visualized using e.g. an immunofluorescence microscope or a confocal fluorescence microscope.

2. Immunohistochemistry (IHC)

IHC is a method where MHC multimers can be used to directly detect specific T-cells e.g. in sections of solid tissue. In some embodiments, sections of fixed or frozen tissue sample are incubated with MHC multimer allowing MHC multimer to bind specific T-cells in the tissue. The MHC multimer may be labelled with a fluorochrome, chromophore, or any other labelling molecule that can be detected. The labeling of the MHC multimer may be directly or through a second marker molecule. As an example, the MHC multimer can be labelled with a tag that can be recognized by e.g. a secondary antibody, optionally labelled with HRP or another label. The bound MHC multimer is then detected by its fluorescence or absorbance (for fluorophore or chromophore), or by addition of an enzyme-labelled antibody directed against this tag, or another component of the MHC multimer (e.g. one of the protein chains, a label on the one or more multimerization domain). The enzyme can e.g. be Horseradish Peroxidase (HRP) or Alkaline Phosphatase (AP), both of which convert a colorless substrate into a colored reaction product in situ. This colored deposit identifies the binding site of the MHC multimer and can be visualized under e.g. alight microscope. The MHC multimer can also be directly labelled with e.g. HRP or AP, and used in IHC without an additional antibody.

In some embodiments, the detection of T-cells in solid tissue includes use of tissue embedded in paraffin, from which tissue sections are made and fixed in formalin before staining. Antibodies are standard reagents used for staining of formalin-fixed tissue sections; these antibodies often recognize linear epitopes. In contrast, most MHC multimers are expected to recognize a conformational epitope on the TCR. In this case, the native structure of TCR needs to be at least partly preserved in the fixed tissue.

In other embodiments, staining performed tissue sections from frozen tissue blocks. In this type of staining fixation is done after MHC multimer staining.

3. Immunofluorescence Microscopy

In some embodiments, MHC multimers can be used to identify specific T-cells in sections of solid tissue. Instead of visualization of bound MHC multimer by an enzymatic reaction, MHC multimers are labelled with a fluorochrome or bound MHC multimer are detected by a fluorescent antibody. Cells with bound fluorescent MHC multimers can be visualized in an immunofluorescence microscope or in a confocal fluorescence microscope. This method can also be used for detection of T-cells in fluid samples using the principles described for detection of T-cells in fluid sample described elsewhere herein.

4. Detection of T-Cells in Solid Tissue In Vivo

MHC multimers may also be used for detection of T-cells in solid tissue in vivo. For in vivo detection of T-cells labeled MHC multimers are injected into the body of the individual to be investigated. The MHC multimers may be labeled with e.g. a paramagnetic isotope. Using a magnetic resonance imaging (MRI) scanner or electron spin resonance (ESR) scanner MHC multimer binding T-cells can then be measured and localized. In general, any conventional method for diagnostic imaging visualization can be utilized. Usually gamma and positron emitting radioisotopes are used for camera and paramagnetic isotopes for MRI.

5. Detection of T-Cells Immobilized on Solid Support.

In a number of applications, it may be advantageous immobilize the T-cell onto a solid or semi-solid support. Such support may be any which is suited for immobilization, separation etc. Non-limiting examples include particles, beads, biodegradable particles, sheets, gels, filters, membranes (e. g. nylon membranes), fibres, capillaries, needles, microtitre strips, tubes, plates or wells, combs, pipette tips, microarrays, chips, slides, or indeed any solid surface material. The solid or semi-solid support may be labelled, if this is desired. The support may also have scattering properties or sizes, which enable discrimination among supports of the same nature, e.g. particles of different sizes or scattering properties, color or intensities.

An example of a method where MHC multimers can be used for detection of immobilized T-cells is ELISA (Enzyme-Linked ImmunosorbentAssay). ELISA is a binding assay originally used for detection of antibody-antigen interaction. Detection is based on an enzymatic reaction, and commonly used enzymes are e.g. HRP and AP. MHC multimers can be used in ELISA-based assays for analysis of purified TCR's and T-cells immobilized in wells of a microtiter plate. The bound MHC multimers can be labelled either by direct chemical coupling of e.g. HRP or AP to the MHC multimer (e.g. the one or more multimerization domain or the MHC proteins), or e.g. by an HRP- or AP-coupled antibody or other marker molecule that binds to the MHC multimer. Detection of the enzyme-label is then by addition of a substrate (e.g. colorless) that is turned into a detectable product (e.g. colored) by the HRP or AP enzyme.

The solid support may be made of e.g. glass, silica, latex, plastic or any polymeric material. The support may also be made from a biodegradable material. Generally speaking, the nature of the support is not critical and a variety of materials may be used. The surface of support may be hydrophobic or hydrophilic. Non-magnetic polymer beads may also be applicable. Such are available from a wide range of manufactures, e.g. Dynal Particles AS, Qiagen, Amersham Biosciences, Serotec, Seradyne, Merck, Nippon Paint, Chemagen, Promega, Prolabo, Polysciences, Agowa, and Bangs Laboratories.

Another example of a suitable support is magnetic beads or particles. The term “magnetic” as used everywhere herein is intended to mean that the support is capable of having a magnetic moment imparted to it when placed in a magnetic field, and thus is displaceable under the action of that magnetic field. In other words, a support comprising magnetic beads or particles may readily be removed by magnetic aggregation, which provides a quick, simple and efficient way of separating out the beads or particles from a solution. Magnetic beads and particles may suitably be paramagnetic or superparamagnetic. Superparamagnetic beads and particles are e.g. described in EP 0 106 873. Magnetic beads and particles are available from several manufacturers, e.g. Dynal Biotech ASA (Oslo, Norway, previously Dynal AS, e.g. DYNABEADS®).

6. Microchip MHC Multimer Technology

A microarray of MHC multimers can be formed, by immobilization of different MHC multimers on solid support, to form a spatial array where the position specifies the identity of the MHC-peptide complex or specific empty MHC immobilized at this position. When labelled cells are passed over the microarray (e.g. blood cells), the cells carrying TCRs specific for MHC multimers in the microarray will become immobilized. The label will thus be located at specific regions of the microarray, which will allow identification of the MHC multimers that bind the cells, and thus, allows the identification of e.g. T-cells with recognition specificity for the immobilized MHC multimers. Alternatively, the cells can be labelled after they have been bound to the MHC multimers. The label can be specific for the type of cell that is expected to bind the MHC multimer, or the label can stain cells in general (e.g. a label that binds DNA). Alternatively, cytokine capture antibodies can be co-spotted together with MHC on the solid support and the cytokine secretion from bound antigen specific T-cells analyzed. This is possible because T-cells are stimulated to secrete cytokines when recognizing and binding specific MHC-peptide complexes.

7. Indirect Detection of T-Cell Using pMHC Multimers

T-cells in a sample may also be detected indirectly using MHC multimers. In indirect detection, the number or activity of T-cells are measured, by detection of events that are the result of TCR-MHC-peptide complex interaction. Interaction between MHC multimer and T-cell may stimulate the T-cell resulting in activation of T-cells, in cell division and proliferation of T-cell populations or alternatively result in inactivation of T-cells. All these mechanisms can be measured using detection methods able to detect these events.

Example measurement of activation include measurement of secretion of specific soluble factor e.g. cytokine that can be measured using flowcytometry as described in the section with flow cytometry, measurement of expression of activation markers e.g. measurement of expression of CD27 and CD28 and/or other receptors by e.g. flow cytometry and/or ELISA-like methods and measurement of T-cell effector function e.g. CD8 T-cell cytotoxicity that can be measured in cytotoxicity assays like chromium release assay's know by persons skilled in the art.

Example measurement of proliferation include but is not limited to measurement of mRNA, measurement of incorporation of thymidine or incorporation of other molecules like bromo-2′-deoxyuridine (BrdU).

Example measurements of inactivation of T-cells include but is not limited to measurement of effect of blockade of specific TCR and measurement of apoptosis.

When contacted with a diverse population of T cells, such as is contained in a sample of the peripheral blood lymphocytes (PBLs) of a subject, those tetramers containing pMHCs that are recognized by a T cell in the sample will bind to the matched T cell. Contents of the reaction is analyzed using fluorescence flow cytometry, to determine, quantify and/or isolate those T-cells having an MHC tetramer bound thereto.

B. Screening

The Conjugated Multimers of the disclosure can be used in a variety of different screening assays. For example, in one embodiment, a library of fluorescently-labeled peptides derived from one or more antigens is applied to pMHC multimers comprising a placeholder peptide under conditions to induce release of the placeholder peptide and binding of the antigen-derived peptides. Peptide exchange is monitored by fluorescence polarization assay. The use of placeholder peptides permits the generation of empty, peptide-receptive MHC multimers under physiological conditions. This screening approach can be used to identify peptide ligands that bind to an MHC molecule. Peptide exchange reactions can be performed in multiwell formats and under native conditions. Binding can be determined by a number of techniques, such as ELISA, which monitors the stability of the MHC structure, or by biophysical techniques that monitor peptide binding, such as fluorescence polarization. This screening approach can also be used to scan peptide sets (such as those derived from pathogen genomes, tumor-associated antigens or autoimmune antigens) for MHC ligands.

The pMHC Conjugated Multimers, and libraries thereof, disclosed herein can be used in a number of screening methods that allow for the convenient detection and quantification of antigen-specific binding to immune cell receptors. Such Conjugated Multimer libraries can allow, for example, detection of T cells specific for a given antigen, multiplex detection of T cell specificities in a given sample, matching of TCR sequence with specificity (e.g., via single cell sequencing), comparative TCR affinity determination, determination of a consensus specificity sequence of a given TCR, or mapping of antigen responsiveness of T cells against sequences of interest. The Conjugated Multimers can also be used in detecting natural killer (NK) cells that bear receptors specific for particular MHC I polypeptides.

The resulting pMHC Conjugated Multimer libraries may be used in T cell screens to determine antigen-reactive T cells as described, for example, in Simon et al, Cancer Immunol Res, 2014, 2(12):1230-1244.

In some embodiments, the disclosure provides a method for isolating a TCR-expressing cell-pMHC pairs comprises contacting a plurality of TCR-expressing cells with a pMHC multimer library as described herein; generating a plurality of compartments, wherein a compartment of the plurality comprises a TCR-expressing cell of the plurality of TCR-expressing cells bound to a pMHC of the library, thereby isolating the TCR-expressing cell-pMHC pair in the compartment. In some embodiments, the TCR-expressing cell is a T cell, e.g., a CD8+ T cell when using a pMHCI multimer library or a CD4+ T cell when using a pMHCII multimer library. In some embodiments, a cell can be transfected or transduced to express a TCR. In some embodiments, a non-lymphocyte cell can be transfected or transduced to express TCR.

C. Methods of Identifying

The pMHC Conjugated Multimers of the disclosure can be used to identify antigen-specific T cells of interest, for example by screening a plurality of T cells with a library of pMHCI Conjugated Multimers. In various embodiments, the library comprises pMHC Conjugated Multimers loaded with a diversity of more than 10, more than 100, more than 500, 1000, more than 2,000, more than 5,000, more than 10,000, more than 10⁶, more than 10⁷, more than 10⁸, more than 10⁹, or more than 10¹⁰ unique peptides. The identification approach can comprise compartmentalizing a cell of the plurality of cells bound to a pMHC Conjugated Multimer of the library in a single compartment, wherein the pMHC Conjugated Multimer comprises a unique identifier; and determining the unique identifier for each pMHC Conjugated Multimer bound to the compartmentalized cell. A compartment can be a separate space, e.g., a well, a plate, a divided boundary, a phase shift, a vessel, a vesicle, a cell, etc.

In some embodiments, the compositions and methods disclosed herein can be used to identify a plurality of peptides that bind to a TCR. In some embodiments, the compositions and methods disclosed herein can be used to identify a plurality of TCRs that bind a pMHC. In some embodiments, the compositions and methods disclosed herein can be used to identify a plurality of TCRs that bind a plurality of pMHCs (for example, a plurality of TCRs that bind to pMHC multimers derived from a pathogen library, cancer library, or autoimmune library).

In some embodiments, the compositions and methods disclosed herein are used for identifying TCR-antigen specificity.

In some embodiments, the identity of a TCR on a selected T cell is determined by sequencing (e.g., sequencing a variable, hypervariable region or complementarity determining region (CDR) of a TCR). In some embodiments, the identity of the peptide of the pMHC bound which binds to a TCR is determined by sequencing (e.g., using an identifier as disclosed herein).

In one embodiment, pMHC Conjugated Multimers of the disclosure can be used for the detection of antigen-specific T cells by flow cytometry or for can be used for T-cell purification. The compositions and methods of the disclosure allow for the production of very large collections of peptide-loaded MHC multimers that are well suited for rapid identification of cytotoxic T-cell (i.e., CD8+ T cell) antigens when using pMHCI multimers and helper T cell (i.e., CD4+ T cell) antigens when using pMHCII multimers.

In one embodiment, pMHC Conjugated Multimers that are attached to solid surfaces can be used to probe T cell function. The peptide-MHC antigenic complexes fixed to the solid surface can function to stimulate T cell activity through the TCR, thereby allowing for study of downstream T cell functions subsequent to TCR stimulation.

In some embodiments, the compositions and methods disclosed herein are used to determine how mutations in an identified MHC-binding peptide affect TCR binding. In some embodiments, the compositions and methods disclosed herein are used to identify mutations in an identified MHC-binding peptide that result in enhanced or reduced TCR binding affinity. In some embodiments, the compositions and methods disclosed herein are used to identify mutations in an identified MHC-binding peptide that retain TCR binding affinity. In some embodiments, the compositions and methods disclosed herein are used to identify mutations in an identified MHC-binding peptide that result in loss of TCR binding affinity.

In some embodiments, the compositions and methods disclosed herein are used to determine how mutations in a TCR identified using the methods described herein alter the binding of a peptide epitope. In some embodiments, the compositions and methods disclosed herein are used to identify mutations in a TCR that result in decreased or increased binding affinity for a peptide epitope. In some embodiments, the compositions and methods disclosed herein can be used to identify mutations in a TCR that retain binding of a peptide epitope. In some embodiments, the compositions and methods disclosed herein can be used to identify mutations in a TCR that result in loss of binding of a peptide epitope.

In some embodiments, the methods disclosed herein are performed on T cells from a plurality of subjects. In some embodiments, analysis of data from multiple subjects allows identification of MHC-binding peptide epitopes recognized by multiple subjects. In some embodiments, analysis of data from multiple subjects allows identification of MHC-binding peptide epitopes recognized by multiple TCR clonotypes. In some embodiments, analysis of data from multiple subjects allows identification of MHC-binding peptide epitopes recognized by multiple patients, e.g., multiple cancer patients, multiple patients with an autoimmune condition, or multiple patients with protective immunity against a pathogen. In some embodiments, analysis of data from multiple subjects allows identification of MHC-binding peptide epitopes recognized in subjects comprising different HLA types or alleles. In some embodiments, analysis of data from multiple subjects allows identification of distinct hypervariable or complementarity determining region sequences of TCRs that exhibit convergent antigen binding.

In some embodiments, the methods disclosed herein are performed using a plurality of libraries. In some embodiments, analysis of data from multiple libraries allows identification of shared reactive MHC-binding peptide epitopes between libraries, e.g., antigens exhibiting TCR affinity that are present in multiple strains of a pathogen, multiple cancer types, multiple cancer patients, multiple autoimmune diseases, or multiple autoimmune conditions. In some embodiments, analysis of data from multiple libraries allows identification of distinct reactive MHCI-binding peptide epitopes among libraries, e.g., antigens present in a subset of pathogen strains, cancers, conditions, or patients.

In some embodiments, T cells identified using a pMHC Conjugated Multimer library of the disclosure are subjected to gene expression analysis (e.g., RNA-seq, qPCR). In some embodiments, gene expression analysis is conducted on cells identified as possessing a receptor exhibiting specificity for a peptide in a library of the disclosure. For example, cells determined to express TCRs that bind to a pMHC Conjugated Multimer derived from a pathogen library, cancer library, or autoimmune library are subjected to gene expression analysis. Gene expression analysis can be global or targeted. Genes analyzed for expression include, but are not limited to, genes with known functions, genes coding for immune effector molecules (e.g., perforin, granzyme, cytokines, chemokines), immune checkpoint molecules, pro-inflammatory molecules, anti-inflammatory molecules, lineage markers, integrins, selectins, lymphocyte memory markers, death receptors, caspases, cell cycle checkpoint molecules, enzymes, phosphatases, kinases, lipases, and metabolic genes.

In some embodiments, gene expression analysis can be conducted concurrently with pMHC Conjugated Multimer library screening. In some embodiments, gene expression analysis can be conducted after analysis of pMHC Conjugated Multimer library screening results. In some embodiments, gene expression analysis can be conducted before analysis of pMHC Conjugated Multimer library screening results. In some embodiments, gene expression analysis allows for immunotyping of cells identified as of interest from pMHC-T cell receptor pairings produced using the methods described herein.

The methods and compositions described herein can be used for screening assays. For example, a library comprising a plurality of pMHC Conjugated Multimers as described herein is contacted with a T cell sample, and one or more T cell functions are determined including, but not limited to, T cell proliferation, T cell cytotoxicity, suppression of T cell proliferation, suppression by a T cell, and cytokine production of a T cell.

In some embodiments, pMHC Conjugated Multimers that can induce the functional property can then be made into a peptide library subset. For example, a library subset can comprise pMHC Conjugated Multimers that induce proliferation of a T cell upon binding to TCR, cytotoxicity upon binding to TCR, T cell suppression upon binding to TCR, suppression by a T cell upon binding to TCR, cytokine production upon binding to TCR, or any combination thereof. Proliferation can be determined by, for example, a dye-dilution assay (e.g., CFSE dilution assay), or quantification of DNA replication (e.g., BrdU incorporation assay). Cytotoxicity can be determined by, for example, assays that are based on release of an intracellular enzyme by dead cells (e.g., lactate dehydrogenase), dye exclusion assays (e.g., propidium iodide), or expression of cytolytic markers (e.g., granzyme, CD107a) by flow cytometry or qPCR. Cytokine production can be determined by, for example, ELISA, multiplex immunoassay, intracellular cytokine staining, ELISPOT, Western Blot, or qPCR. T cell suppression can be determined by, for example, co-incubating a T cell clone with effector cells and target antigen, and measuring proliferation, cytotoxicity, cytokine production, expression of activation markers, etc.

In some embodiments, the compositions and methods disclosed herein are used to identify antigen-specific T cell effector clones associated with protective immunity, non-protective immunity, or autoimmunity. In some embodiments, compositions and methods disclosed herein are used to identify antigen-specific T cell effector clones that exhibit anergy, exhaustion, tolerogenic properties, autoimmune properties, inflammatory properties, or anti-inflammatory properties (e.g., Tregs). In some embodiments, compositions and methods disclosed herein are used to identify antigen-specific T cell effector clones that exhibit certain effector or memory properties (e.g., naïve, terminal effector, effector memory, central memory, resident memory, T_{H}1, T_{H}2, T_{H}17, T_{H}9, T_{C}1, T_{C}2, T_{C}17, production of certain cytokines).

In some embodiments, a TCR identified using compositions and methods disclosed herein are used as part of a therapeutic intervention. For example, a TCR sequence, TCR variable region sequence, or CDR sequence can be transfected or transduced into T cells to generate modified T cells of the same antigenic specificity. The modified T cells can be expanded, polarized to a desired effector phenotype (e.g., T_{H}1, T_{C}1, Treg), and infused into a subject. In some embodiments, multiple TCRs identified using compositions and methods disclosed herein are used in an oligoclonal therapy.

In some embodiments, a peptide, ligand, agonist, antagonist, antigen, or epitope identified using methods disclosed herein is used as part of a therapeutic intervention. In some embodiments, a peptide, antigen, or epitope is used to expand a population of cells ex vivo, e.g. using antigen presenting cells, artificial antigen presenting cells, immobilized peptide, or soluble peptide. In some embodiments, expanded cells are infused into a patient. In some embodiments, peripheral blood lymphocytes are expanded. In some embodiments, tumor-infiltrating lymphocytes (TILs) are expanded. In some embodiments, T_{H}1 cells are expanded. In some embodiments, cytotoxic T lymphocytes are expanded. In some embodiments, T regulatory cells are expanded.

In some embodiments, the compositions and methods disclosed herein are used to identify MHC-binding antigenic peptides for use in development of a vaccine, e.g. a subunit vaccine, a vaccine eliciting coverage against a range of protective antigens, or a universal vaccine.

In some embodiments, the compositions and methods disclosed herein can be used for diagnosis of a medical condition. In some embodiments, the compositions and methods disclosed herein are used to guide clinical decision making, e.g. treatment selection, identification of prognostic factors, monitoring of treatment response or disease progression, or implementation of preventative measures.

In some embodiments, the compositions and methods disclosed herein can be used in the selection and/or design of treatments for medical conditions, in particular in the selection of antigen-specific T cells (e.g., CD8+ cytotoxic T cells and/or CD4+ helper T cells), or TCRs derived therefrom, for use in adoptive transfer T cell therapy. For example, the pMHC Conjugated Multimers can be used to identify T cells within a patient sample the react to an antigen(s) of interest, such as a cancer antigen(s) or pathogen antigen(s) to thereby select those cells for expansion in vitro followed by reintroduction into the patient. Moreover, TCRs identified from such antigen-specific T cells can be sequences and recombinantly introduced into T cells to increase the population of cells expressing TCRs that bind to an antigen(s) of therapeutic interest in a patient.

### XI. Compositions and Kits

In another aspect, the disclosure comprises compositions and kits for use in the methods described herein. In one embodiment, the disclosure provides a pMHC Conjugation Multimer composition. In one embodiment, the pMHC Conjugation Multimer is a pMHC Conjugation Tetramer. In one embodiment, the multimerization domain of the tetramer is streptavidin or avidin. In one embodiment, the pMHC Conjugation Tetramer comprises four MHC monomers covalently conjugated to the streptavidin or avidin molecule at sites other than the biotin-binding site of streptavidin or avidin. In one embodiment, the four MHC monomers each comprise (i.e., are loaded with) an MHC-binding peptide, wherein each monomer comprises the same MHC-binding peptide. In one embodiment, the MHC Conjugation Tetramer further comprises a biotinylated oligonucleotide barcode bound to the biotin-binding site of streptavidin or avidin. In one embodiment, the pMHC Conjugation Multimer (e.g., Tetramer) is a pMHC Class I Conjugation Multimer (e.g., Tetramer). In another embodiment, the pMHC Conjugation Multimer (e.g., Tetramer) is a pMHC Class II Conjugation Multimer (e.g., Tetramer).

In one embodiment, the disclosure comprises a kit comprising a plurality of pMHC Conjugation Multimer compositions. In one embodiment, each pMHC Conjugation Multimer in the plurality is a pMHC Conjugation Tetramer. In one embodiment, the multimerization domain of each tetramer is streptavidin or avidin. In one embodiment, each MHC Conjugation Tetramer comprises four MHC monomers covalently conjugated to the streptavidin or avidin molecule at sites other than the biotin-binding site of streptavidin or avidin. In one embodiment, the four MHC monomers each comprise an MHC-binding peptide, wherein each MHC monomer within each single tetramer comprises (i.e., is loaded with) the same MHC-binding peptide and wherein each MHC Conjugation Tetramer within the plurality comprises (i.e., is loaded with) a different MHC-binding peptide, thereby forming a library of MHC-binding peptides. In one embodiment, each MHC Conjugation Tetramer within the plurality further comprises a biotinylated oligonucleotide barcode bound to the biotin-binding site of streptavidin or avidin. In one embodiment, each pMHC Conjugation Multimer (e.g., Tetramer) of the plurality is a pMHC Class I Conjugation Multimer (e.g., Tetramer). In another embodiment, each pMHC Conjugation Multimer (e.g., Tetramer) of the plurality is a pMHC Class II Conjugation Multimer (e.g., Tetramer).

### EXAMPLES

Below are examples of specific embodiments for carrying out the present invention. The examples are offered for illustrative purposes only and are not intended to limit the scope of the present invention.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al, Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3rd Ed. (Plenum Press) Vols A and B (1992).

Unless otherwise stated, all reagents and chemicals were obtained from commercial sources and used without further purification.

### Example 1—Generation of Exchangeable Peptide MHC Class I Multimers with Sortase Tag

In this example, MHC I heavy chains are expressed and complexed with (β2-microglobulin (β2m) and an exchangeable peptide, such that the MHC heavy chain contains a C-terminal sortase tag that enables post-translational coupling to Streptavidin (SAv) to form barcodable exchangeable MHC I tetramers. MHC I heavy chain and SAv are expressed with a C-terminal sortase tag (the amino acid sequences of which are shown in SEQ ID NOs: 1 and 3, respectively). Sortase enzyme (having the amino acid sequence shown in SEQ ID NO: 6) is then used to conjugate a GGG-X click handle peptide to MHC I or a GGG-Y click handle peptide to SAv, where a click handle peptide contains a click moiety such as an alkyne (X) or an azide (Y), or vice versa. Subsequent chemical conjugation of MHC I to SAv by copper-assisted alkyne-azide cycloaddition or copper-free alkyne-azide cycloaddition then results in exchangeable-peptide-loaded MHC I tetramers.

HLA and β2m Expression and Refolding.

Bacterial expression plasmids encoding HLA-A*02:01 linked to a Sorttag, referred to herein as HLA-A2-Sorttag (containing a C-terminal Sortase tag, 6×-His-tag) (the amino acid sequence of which is shown in SEQ ID NO: 1) and β2m (the amino acid sequence of which is shown in SEQ ID NO: 2) were generated. HLA-A2-Sorttag and β2m were expressed in E. Coli in inclusion bodies. Inclusion bodies were purified and solubilized in urea buffer (20 mM MES, pH 6.0, 8 M urea, 10 mM EDTA) containing 1 mM or 0.1 mM DTT for HLA-A2-Sorttag or 0.1 mM DTT for β2m. UV-labile placeholder peptide (GILGFVFJL (SEQ ID NO: 7), where J is 3-amino-3-(2-nitro)phenylpropionic acid) was chemically synthesized. HLA-A2 was refolded with β2m and placeholder peptide according to previously described protocols (Garboczi, et al., PNAS, 89: 3429-3433, 1992; Rodenko, et al., Nat Protoc., 1:1120-32, 2006) with minor modifications. Briefly, the following components were added with stirring to pre-chilled refold buffer (100 mM Tris, pH 8.0, 0.4 M Arginine-HCl, 2 mM EDTA, 5 mM reduced glutathione, 0.5 mM oxidized glutathione, 0.2 mM PMSF) in the following order with final concentration indicated: Peptide (45 uM), β2m (3 uM) and then HLA-A2-Sorttag (1.5 uM) solubilized inclusion bodies. The refold reaction was incubated with stirring overnight at 4° C. On the next day, β2m and HLA-A2-Sorttag solubilized inclusion bodies were added to the refold reaction for 6 uM and 3 uM final concentrations, respectively. On Day 4, the refold reaction was clarified of any precipitation by centrifugation followed by filtration through a 0.2 μm filter. The refold reaction was then concentrated using a Minimate Tangential Flow Filtration System (Pall) with a 10 kDa Minimate TFF Capsule (Pall) and Amicon Ultra-15 Centrifugal filters with 10000 Da molecular weight cutoff membranes (Millipore). The concentrated refold reaction was purified by size exclusion chromatography (SEC) on a HiLoad 26/600 Superdex 200 prep grade (GE Life Sciences) pre-equilibrated in SEC buffer (20 mM HEPES pH 7.2, 150 mM NaCl). Purified fractions corresponding to the monomeric HLA-A2-Sorttag/β2m/peptide complex were pooled and concentrated. A similar procedure was followed for HLA-A2, β2m, and NLVPMVATV (SEQ ID NO: 8) peptide (abbreviated NLV) refolding and purification.

Conjugation of Click-Handle Peptide to HLA-A2-Sorttag Using Sortase.

HLA-A2 was modified enzymatically with a Click-Handle peptide using the transpeptidase Sortase. Sortase enzyme containing 5 enhancing mutations (Chen, PNAS 2011 108(28) 11399-11404) (the amino acid sequence of which is shown in SEQ ID NO: 6) was expressed in E. coli and purified according to (Antos, Curr Protoc Protein Sci, 2009 doi:10.1002/0471140864.ps1503s56). Click-Handle Peptides containing an N-terminal triglycine followed by a PEG linker (PEG₄ or PEG₅) were linked synthetically to: 1) Propargylglycine (referred to as GGG-Alkyne, Alkyne or Alk), 2) Sulfo-DBCO (referred to as GGG-DBCO or DBCO), or 3) Picolyl azide (referred to as GGG-Azide, Azide or Az). GGG-PEG₅-Alkyne peptide with C-terminal amidation was synthesized by GenScript (Piscataway, N.J.). GGG-PEG₄-Azide peptide with C-terminal amidation and GGG-PEG₄-DBCO peptide were synthesized by Click Chemistry Tools (Scottsdale, Ariz.).

HLA-A2/β2m/peptide monomer (100-150 uM), Click Handle Peptide (GGG-Alkyne, GGG-DBCO, or GGG-Azide at 6-10 mM), Sortase (5-6 uM) and 10 mM CaCl2 were mixed and incubated at 4 C for up to 4 hrs to generate an HLA-Click-Handle fusion. The reaction mixture was purified by SEC as described above to remove residual Sortase and Click-Handle-Peptide. Purified fractions corresponding to the monomeric HLA-Click-Handle/β2m/peptide complex were pooled and concentrated.

SAv Expression, Purification and Conjugation of Click-Handle Peptide to SAv Using Sortase.

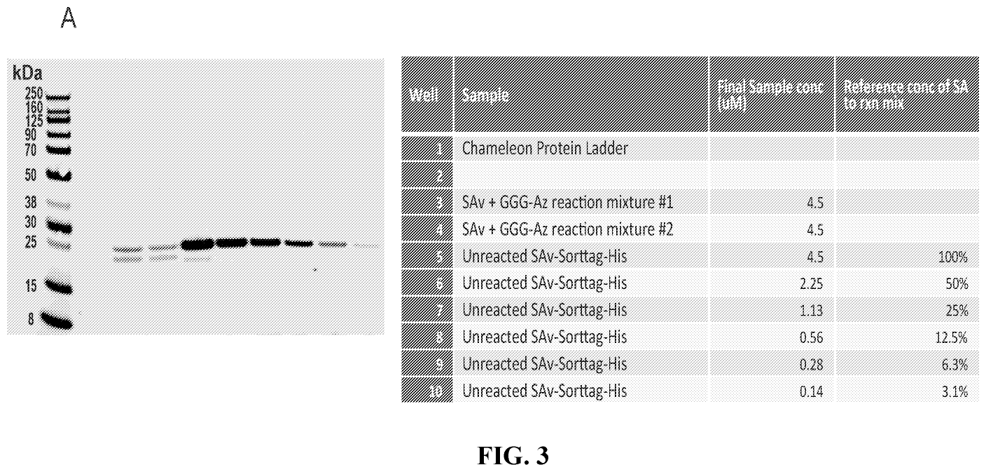

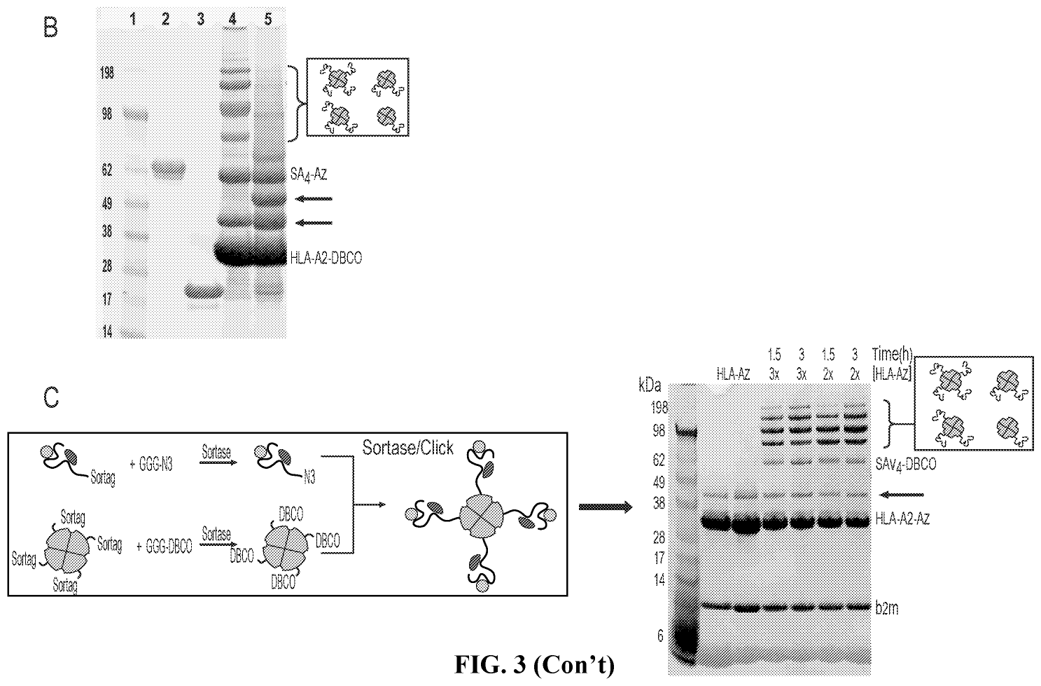

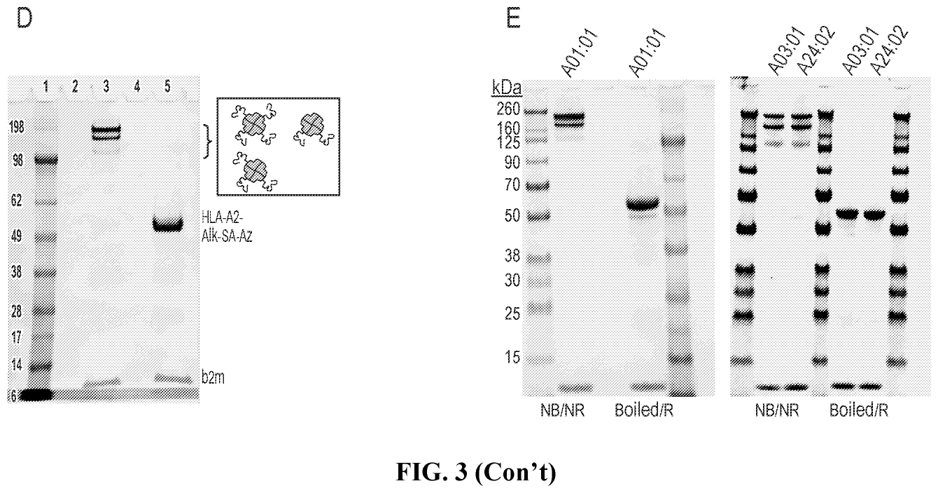

Full length SAv containing a C-terminal Sortase-tag and 6×HisTag (the amino acid sequence of which is shown in SEQ ID NO: 3) was expressed in BL21(DE3) cells by standard methods. SAv was purified from the soluble fraction by immobilized metal affinity chromatography (IMAC) and SEC as described above. SAv forms a native tetramer and migrates as a stable tetramer on SDS-PAGE (Waner M. J., et al., 2004, doi: 10.1529/biophysj.104.047266). Purified fractions corresponding to Tetrameric SAv were pooled and concentrated. SAv-Click-Handle fusions were generated by mixing SAv (70-150 uM), Click Handle Peptide (GGG-DBCO or GGG-Azide at 3-10 mM), Sortase (6 uM) and CaCl2 (10 mM) at 4 C for up to 4 hrs. The reaction mixture was purified by SEC to remove residual sortase and peptide, and purified fractions corresponding to the SAv-Click-Handle fusion were pooled and concentrated. The extent of conjugation to SAv was assessed by Anti-His Western blot analysis by determining the degree of loss of anti-6×His reactive band intensity relative to varying amounts of the untreated SAv sample (FIG. 3A).

Generation of Clicked Peptide/MHC Class I-SAv Multimers.

The generation of clicked HLA-Streptavidin fusions is described herein using several different click chemistry formats (e.g., click chemistry that is described further in Agard N J, Prescher J A, Bertozzi C R J Am Chem Soc. 2004 Nov. 24; 126(46):15046-7; and Hong, V., et al., Angew Chem Int Ed Engl. 2009; 48(52): 9879-9883. doi:10.1002/anie.200905087). Because SAv forms an SDS-resistant tetramer, SDS-PAGE can be employed to monitor the extent of reaction and determine the valency of HLA on SAv (Waner M. J., et al., 2004, doi: 10.1529/biophysj.104.047266).
1) Formation of the clicked multimer by copper-free alkyne-azide cycloaddition was performed by mixing HLA-A2-DBCO/NLV (150 uM) with SAv-Az (50 uM with respect to SA-monomer) and incubating on ice for 3 hrs. SDS-PAGE analysis confirmed the formation of tetrameric SA with 1, 2, 3, and 4 HLA molecules attached (FIG. 3B). Side-products were observed that were attributed to undesired side-reactions of DBCO with Cysteine residues on β2m or HLA-A2 (van Geel, R, Bioconjugate Chem. 2012, 23(3): 392-398. doi.org/10.1021/bc200365k).
2) Covalently conjugated multimeric HLA was also prepared by mixing different ratios of HLA-A2-Az/NLV and SA-DBCO (3:1 and 2:1) at room temperature or on ice (not shown) for 1.5-3.0 hr. SDS-PAGE analysis shows the formation of tetramer, trimer, dimer and monomer HLA-A2-Az-SAv-DBCO species, with a reduced level of undesirable side-reaction products compared to HLA-A2-DBCO-SAv-Az. (FIG. 3C).
3) An additional method to generate covalently linked HLA-A2 and SAv was through copper-assisted alkyne-azide cycloaddition. HLA-A2-Alk-SAv-Az was generated by mixing the following reaction components on ice: HLA-A2-Alk/GILGFVFJL (SEQ ID NO: 7)/β2m (100-130 uM), SAv-Az (70-80 uM with respect to SA-monomer), Copper Sulfate (0.5 mM), BTTAA (2.5 mM) and Ascorbic Acid (5 mM). The reaction was monitored by SDS-PAGE and after 4 hrs the reaction mixture was purified by SEC to separate unreacted HLA, SAv, and other reaction components from purified HLA-A2-Alkyne-SAv-Az multimer. SEC Fractions were analyzed by SDS-PAGE and fractions corresponding to majority tetramer/trimer species were pooled and concentrated. The peptide/HLA-A2-Alkyne-SAv-Az/β2m sample was analyzed by SDS-PAGE, which showed apparent tetramer and trimer species and very small amount of monomer for the non-boiled/non-reduced samples, while boiled and reduced gel analysis confirms the covalent linkage of HLA-A2-Alk and SAv-Az monomer at approximately 53 kDa (FIG. 3D). Mass spectrometry under denaturing conditions also confirmed the formation of an azide-alkyne fusion between HLA-A2 and SAv (not shown). HLA-Alkyne-SAv-Az formats were also generated for HLA-A01:01, HLA-A*03:01 and HLA-A*24:02, as shown in FIG. 3E.

### Example 2—Generation of Exchangeable Peptide MHC Class I Multimers with Intein Tag

In this example, MHCI heavy chain is expressed with a C terminal N-intein tag, and streptavidin (SA) is expressed with an N-terminal C-intein tag, followed by intein-mediated conjugation to create the exchangeable-peptide-loaded MHC I tetramers. Sequences for inteins and use thereof to conjugate proteins are described further in, for example, Stevens, et al. J. Am. Chem. Soc., 138, 2162-2165, 2016; Shah et al. J. Am. Chem. Soc., 134, 11338-11341, 2012; and Vila-Perello et al., J. Am. Chem. Soc., 135, 286-292, 2013, the entire contents of each of which is hereby incorporated by reference.

HLA-A2 (HLA-A*02:01) was expressed in BL21(DE3) as a fusion to the Npu N-intein fragment at the C-terminus (the amino acid sequence of which is shown in SEQ ID NO: 4). Streptavidin was expressed in BL21(DE3) with an N-terminal fusion to the Npu-C-intein fragment and a C-terminal Flag tag (the amino acid sequence of which is shown in SEQ ID NO: 5). HLA-A2-N-intein and C-intein-SAv expressed in bacterial inclusion bodies. Inclusion bodies were isolated and solubilized in Urea buffer (25 mM MES, 8 M urea, 10 mM EDTA, 0.1 mM DTT, pH 6.0). HLA-A2-N-intein was refolded with β2m and UV-labile placeholder peptide (GILGFVFJL (SEQ ID NO: 7), where J is 3-amino-3-(2-nitro)phenylpropionic acid). The following components were added with stirring to pre-chilled refold buffer as described in Example 1. The refold reaction was concentrated using an Amicon Stir Cell with 10000 Da MWCO, Millipore Biomax Ultrafiltration Discs (Millipore) and Amicon Ultra-15 Centrifugal Filter Units 10,000 MWCO (Millipore). The concentrated refold reaction was purified by size exclusion chromatography (SEC) on a HiLoad 26/600 Superdex 200 prep grade (GE Life Sciences) pre-equilibrated in SEC buffer (20 mM HEPES pH 7.2, 150 mM NaCl). Purified fractions corresponding to the monomeric HLA-A2-N-intein/β2m/peptide complex were pooled and concentrated to 100-200 uM. C-intein-SAv was refolded by the same approach: briefly, urea-solubilized C-intein-SAv was injected into prechilled refold buffer and refolded according to the protocol described in Example 1, concentrated in Amicon stir cell with a 10K MWCO membrane as described and purified by size exclusion chromatography as described above. SEC purified C-intein-SAv was concentrated to 100-200 uM.

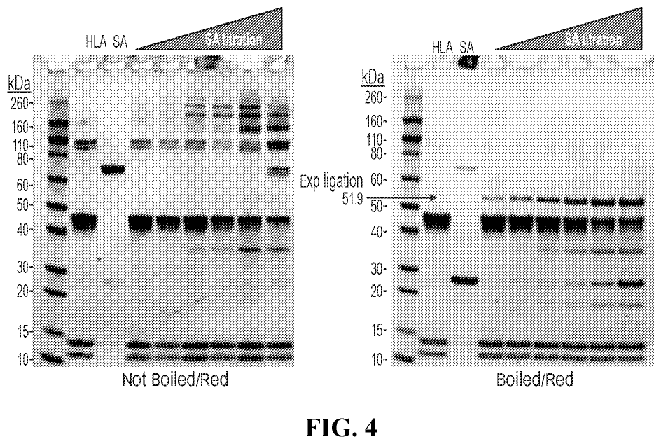

Splicing reactions between HLA-A2-N-intein/β2m/peptide complex and C-intein-SAv were carried out by adding Tris(2-carboxyethyl)phosphine hydrochloride (TCEP) to a final concentration of 0.5 mM to both the HLA-A2-int and the C-int-SA components. All components were kept on ice. To favor formation of tetrameric species, streptavidin was added in 5 increments over a 16 h period until an equimolar amount to HLA-A2-intein was achieved. SDS-PAGE analysis of the reaction under non-reducing/non-boiled conditions shows the formation of higher MW species, while the boiled/reduced samples showed a species at approximately 52 kDa, consistent with the expected size for an HLA-A2-SAv fusion (FIG. 4).

### Example 3. Production of Exchangeable MHCI Tetramers Via Biotinylation and Coupling to Streptavidin

HLA-A*02 heavy chain with a C-terminal Avitag was expressed in E. coli in inclusion bodies. The amino acid sequence of the Avitag is shown in SEQ ID NO: 161. Purified inclusion bodies were solubilized in urea and refolded with beta-2-microglobulin and the peptide NLVPMVATV (SEQ ID NO:8) or the conditional ligand GILGFVFJL (SEQ ID NO:7), where J is a 2-nitrophenylamino acid residue, according to literature methods (Altman & Davis, Curr Protoc Immunol. 2003; Chapter 17: Unit 17.3; Rodenko et. al., Nat Protoc. 2006; 1(3):1120-32). SEC-purified MHC monomers comprising the heavy chain, β-2-microglobulin and peptide were then biotinylated using biotin ligase and then SEC-purified once again. Streptavidin was added to biotinylated MHC monomers in 10 separate aliquots to achieve a slight molar excess of biotin sites over MHC monomers. Peptide exchanges (as described in Example 4) are executed on either the biotin-mediated streptavidin tetramer or on the biotinylated HLA monomer. In the case of the latter, monomers are tetramerized with streptavidin after exchange.

### Example 4. Peptide Exchange Via Dipeptide or UV Exchange

HLA-A*02:01-Alk-SAv-Az Conjugated Tetramers produced as described above in Example 1, as well as biotin-mediated HLA-A*02 tetramers produced as in Example 3, were exchanged by either of two methods. For dipeptide exchange, 5 uM MHC tetramers loaded with a place-holder peptide (e.g., GILGFVFJL (SEQ ID NO:7)) were incubated with a 30-fold excess of NLVPMVATV (SEQ ID NO:8) peptide in the presence or absence of 10 mM GM dipeptide for 3 hours at room temperature (Saini et al., PNAS 2006; 112(1):202-206). For UV-exchange, 2-10 uM MHC monomers or 0.5-2.5 uM MHC tetramers loaded with a place-holder peptide (GILGFVFJL (SEQ ID NO:7)) were incubated with a 30-100-fold molar excess of NLVPMVATV (SEQ ID NO:8) (or other peptide) for 1 hour on ice, followed by 30 minutes exposure to 365 nm UV light from a lamp held 2-5 cm from the sample. The UV exposure was sometimes followed by 30 minutes incubation at 30° C. to allow complete exchange. Efficiency of peptide exchange was monitored by Differential Scanning Fluorimetry (DSF), ELISA and cell staining/flow cytometry.

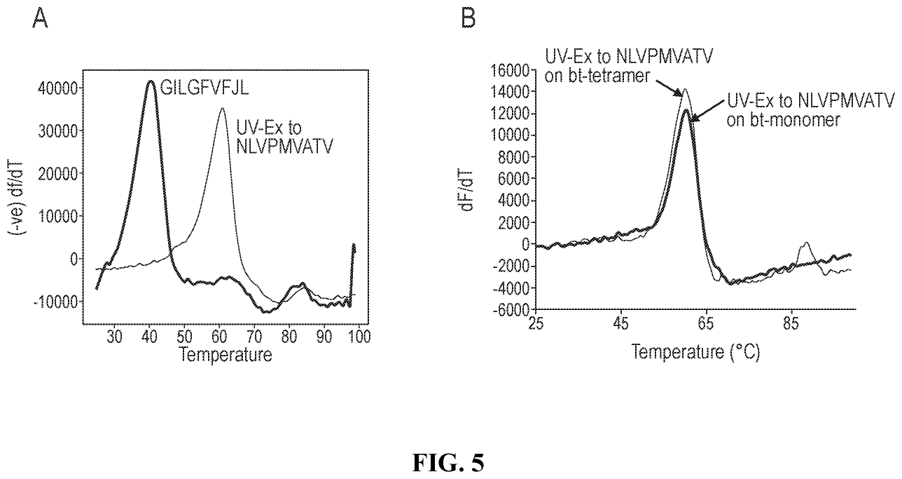

For DSF, 0.25 mg/ml HLA-A*02 tetramers were mixed with an equal volume of 20× Sypro Orange (Invitrogen S6650), and subjected to a 0.05° C./s ramp from 25° C. to 99° C. in a qPCR instrument (e.g., Applied Biosystems Quant Studio 3). A peak in the first derivative of the melt curve indicates the Tm of the pMHC. As seen in FIG. 5A, the Tm of HLA-A*02:01-Alk-SAv-Az Conjugated Tetramers produced as in Example 1, shifts from 40° C. to 61° C. upon UV-exchange from the placeholder GILGFVFJL (SEQ ID NO:7) peptide to NLVPMVATV (SEQ ID NO:8). The Tm after UV exchange is identical to that observed for NLVPMVATV (SEQ ID NO:8) exchanged into biotinylated monomers followed by tetramerization (industry standard) or exchanged directly into biotin-mediated tetramers (FIG. 5B). These data confirm that multimeric state has no impact on the efficiency of UV-exchange, and that Conjugated Tetramers of the current invention have the same stability as the industry standard pMHC.

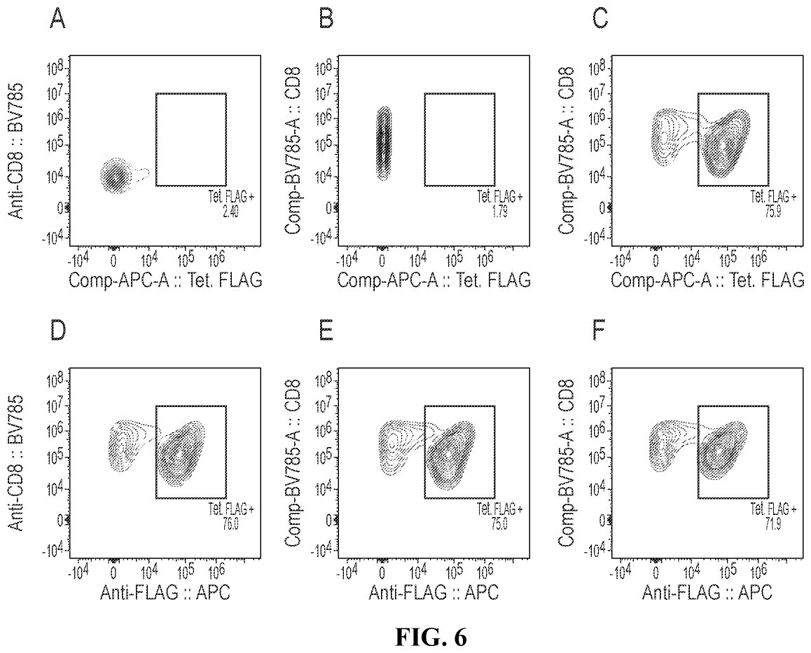

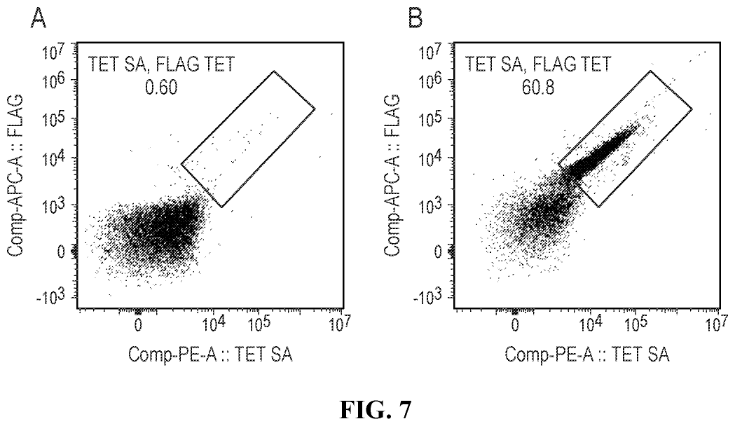

For flow cytometry, 10\hat{ }5 donor T cells that had been expanded with NLVPMVATV (SEQ ID NO:8) (or other peptide) were stained with pMHC tetramers produced as above. All pMHC were diluted in PBS plus 10% FBS, and stained with anti-CD8-BV785, and anti-Flag-APC or anti-streptavidin-PE (Biolegend) was used as secondary. As seen in FIG. 6A-F, either dipeptide exchange or UV exchange executed on the biotin-mediated tetrameric form produces HLA-A*02 tetramers that display the same level of binding to expanded T cells as those produced by industry-standard methods (tetramerization post refolding or post UV exchange of biotinylated monomers). FIG. 7 illustrates the high affinity binding of HLA-A*02:01-Alk-SAv-Az Conjugated Tetramers that were UV-exchanged to the NLVPMVATV (SEQ ID NO:8) peptide to expanded T cells.

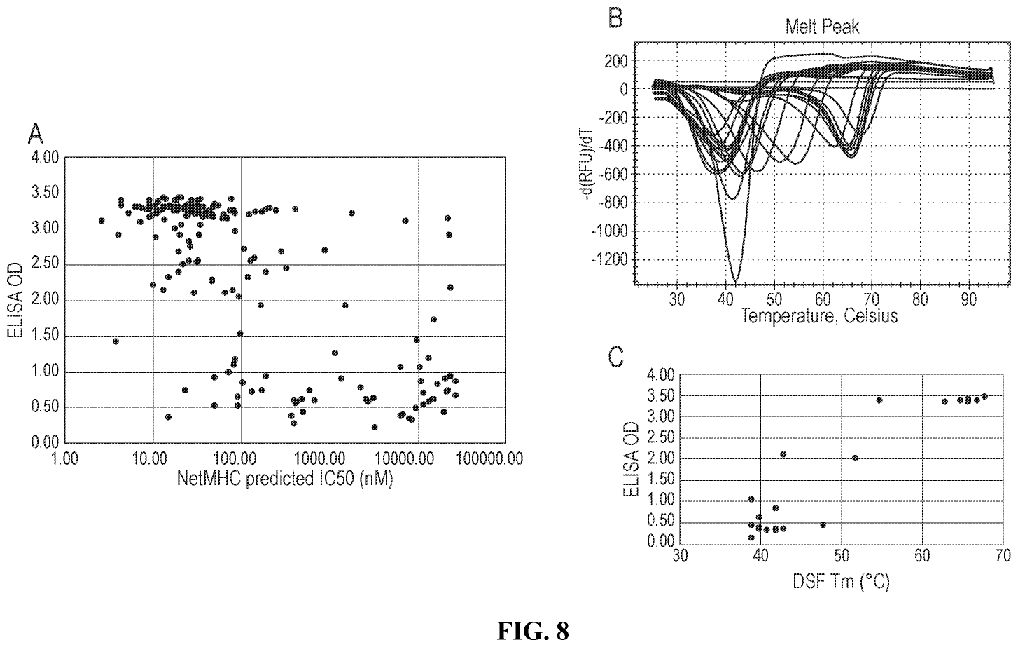

ELISA were also used to monitor exchange on tetramers and is another indicator of pMHC stability. Plates were first coated with anti-streptavidin antibody, followed by capture of tetramers in Citrate-phosphate buffer at pH 5.4, and then read out using HRP-conjugated anti-β2-microglobulin (Biolegend). As seen in FIG. 8A, a panel of NLVPMVATV (SEQ ID NO:8) mutant peptides can be effectively UV-exchanged into HLA-A*02:01-Alk-SAv-Az Conjugated Tetramers, generating a span of ELISA signals. A smaller panel of similar peptides UV-exchanged into biotin-mediated HLA-A*02 tetramers also generated a range of ELISA signals (FIG. 8C), which positively correlated with Tm measured by DSF (FIG. 8B).

### Example 5: Conjugated Tetramers Produced with HLA-A*01:01

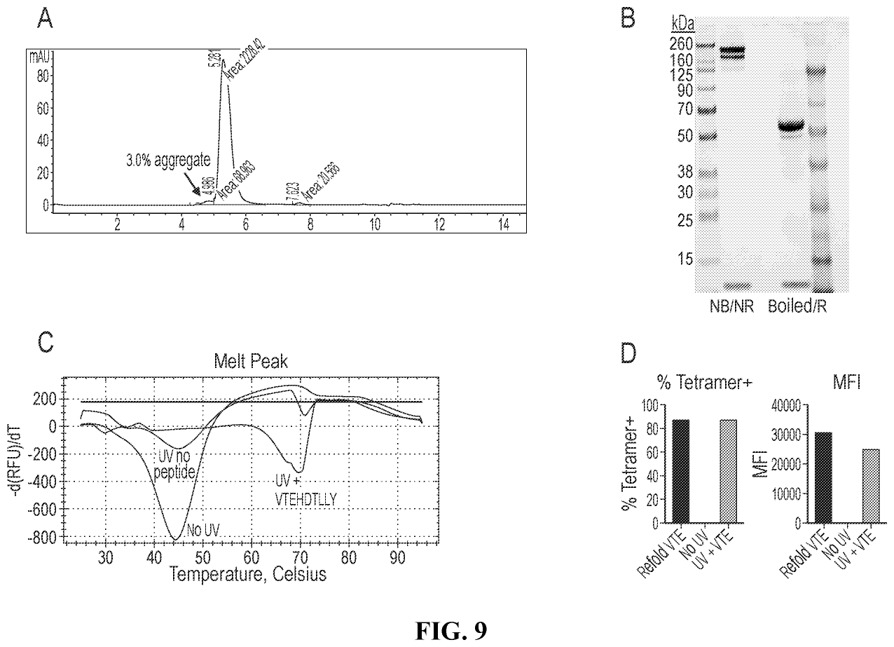

HLA-A*01:01 monomers refolded with the peptide STAPGJLEY (SEQ ID NO: 16) were used for construction of HLA-A*01:01-Alk-SAv-Az Conjugated Tetramers and QC'd as described in Example 1 above. As seen in FIG. 9A and FIG. 9B, HLA-A*01:01-Alk-SAv-Az Conjugated Tetramers were highly multimeric with a low percentage of aggregates (3%). UV treatment in the presence of a cognate peptide VTEHDTLLY (SEQ ID NO: 10) resulted in a characteristic shift in the DSF melt curve, indicating effective peptide exchange (FIG. 9C). The exchanged HLA-A*01:01-Alk-SAv-Az conjugated tetramers bound strongly to PBMCs expanded with the VTEHDTLLY peptide (SEQ ID NO: 10), similar to HLA-A*01:01 refolded with VTEHDTLLY peptide (SEQ ID NO: 10) that was conjugated to streptavidin via biotin (FIG. 9D). As expected, no binding was observed in the absence of UV exchange.

### Example 6: Conjugated Tetramers Produced with HLA-A*24:02

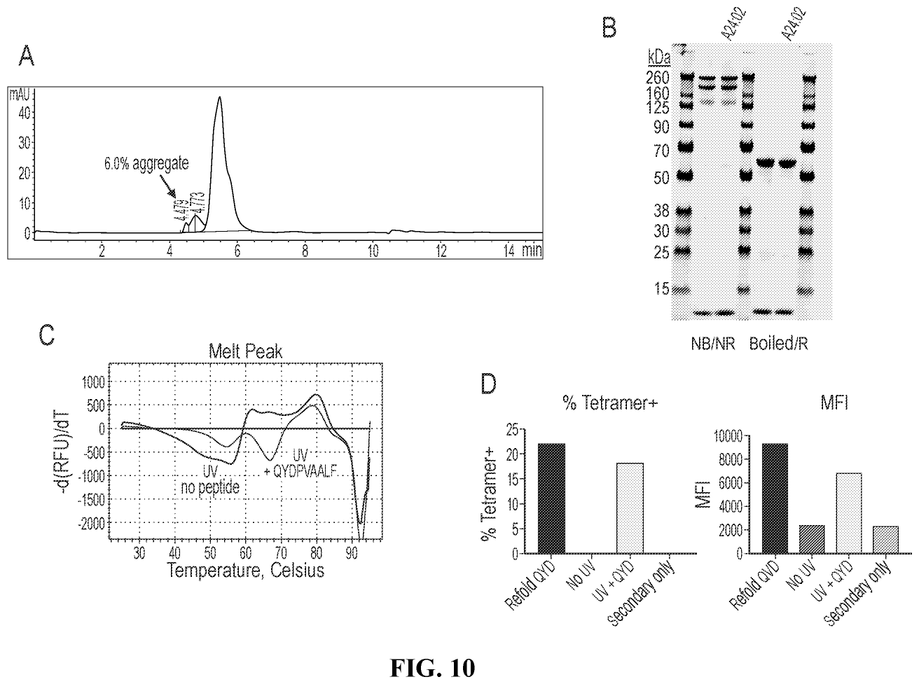

HLA-A*24:02 monomers refolded with the peptide VYGJVRACL (SEQ ID NO: 11) were used for construction of HLA-A*24:02-Alk-SAv-Az Conjugated Tetramers and QC'd as described in Example 1 above. As seen in FIG. 10A and FIG. 10B, HLA-A*24:02-Alk-SAv-Az Conjugated Tetramers were highly multimeric with a low percentage of aggregates (6%). UV treatment in the presence of a cognate peptide QYDPVAALF (SEQ ID NO: 12) resulted in a characteristic shift in the DSF melt curve, indicating effective peptide exchange (FIG. 10C). The exchanged HLA-A*24:02-Alk-SAv-Az conjugated tetramers bound strongly to PBMCs expanded with the QYDPVAALF peptide (SEQ ID NO: 12), similar to HLA-A*24:02 refolded with QYDPVAALF peptide (SEQ ID NO: 12 that was conjugated to streptavidin via biotin (FIG. 10D). As expected, no binding was observed in the absence of UV exchange.

### Example 7: Conjugated Tetramers Produced with HLA-B*07:02

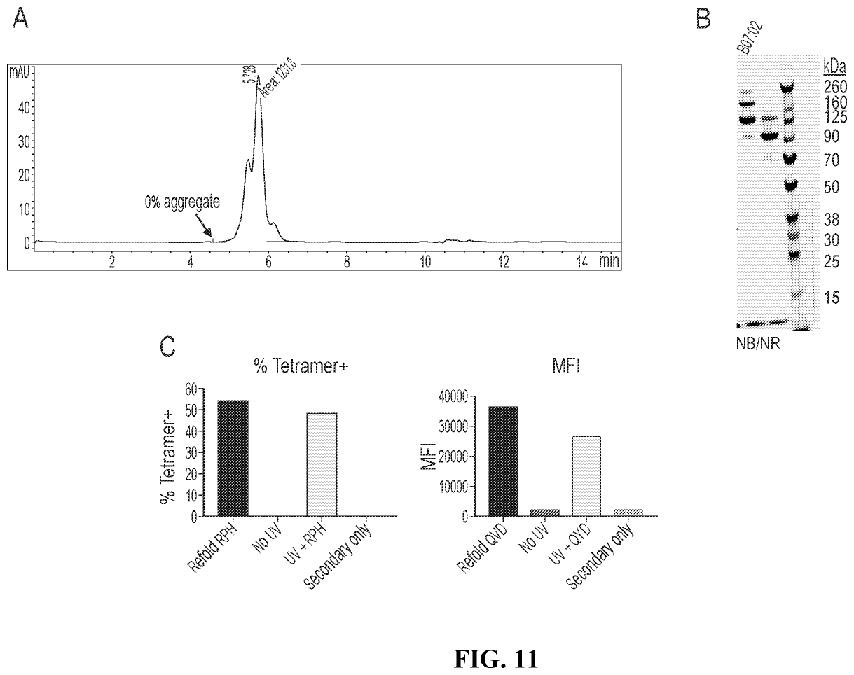

HLA-B*07:02 monomers refolded with the peptide AARGJTLAM (SEQ ID NO: 14) were used for construction of HLA-B*07:02-Alk-SAv-Az Conjugated Tetramers and QC'd as described in Example 1 above. As seen in FIG. 11A and FIG. 11B, HLA-B*07:02-Alk-SAv-Az Conjugated Tetramers were multimeric with no detectable aggregates. After UV treatment in the presence of a cognate peptide RPHERNGFTVL (SEQ ID NO: 13), exchanged HLA-B*07:02-Alk-SAv-Az conjugated tetramers bound strongly to PBMCs expanded with the RPHERNGFTVL peptide (SEQ ID NO: 13), similar to HLA-B*07:02 refolded with RPHERNGFTVL peptide (SEQ ID NO: 13) that was conjugated to streptavidin via biotin (FIG. 11C). As expected, no binding was observed in the absence of UV exchange.

### Example 8: Barcoding and Pooling of UV-Exchanged Tetramers

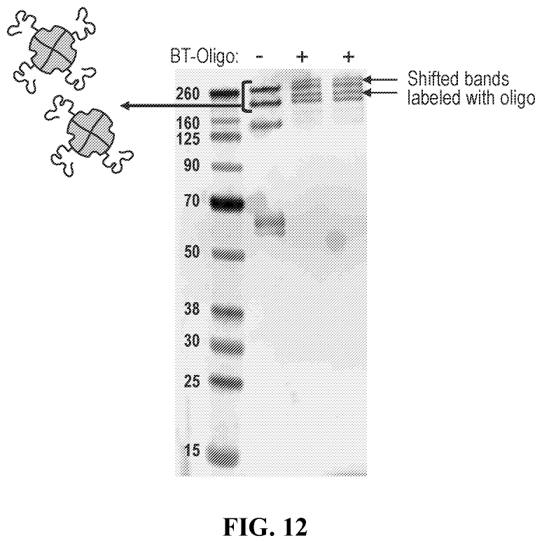

Exchanged HLA-A*02:01-Alk-SAv-Az Conjugated Tetramers were easily labeled with an identifying oligonucleotide tag (barcode) due to the fact that the biotin binding sites on streptavidin were empty. 5′ biotinylated oligonucleotides were added at a 2:1 oligo:tetramer molar ratio, and incubated for 30 min at 4° C., followed by quench with biotin at 400:1 biotin:tetramer molar ratio for 30 min at 4° C. Barcoding was confirmed by electrophoresis on a 4-12% bis-tris gel, followed by blotting to nitrocellulose and staining with anti-Flag antibody (Invitrogen #MA1-91878-D800). As seen in FIG. 12, a gel shift relative to the tetramer starting material indicates proper labeling with the oligonucleotide barcode.

### Example 9: Single Cell Sequencing with Pooled Barcoded UV-Exchanged Tetramers

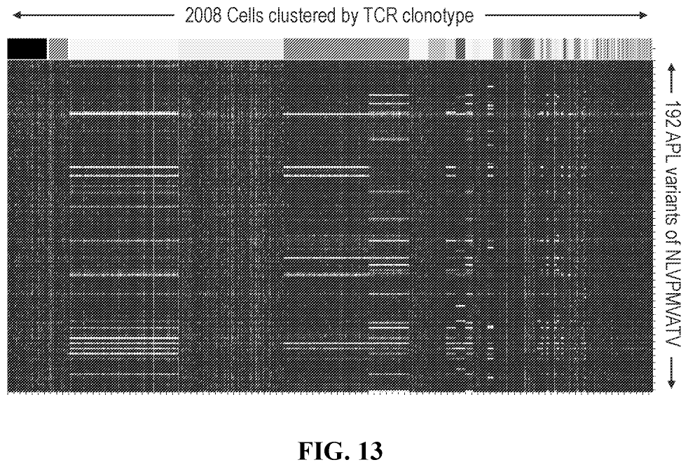

Individual HLA-A*02:01-Alk-SAv-Az Conjugated Tetramer samples that were UV-exchanged for 192 different APL variants of NLVPMVATV (SEQ ID NO:8) were individually conjugated to oligonucleotide labels, pooled, stained on NLVPMVATV (SEQ ID NO: 8)-expanded T cells, and subjected to single cell sequencing. The analyzed results are shown in a heatmap in FIG. 13, indicating clonotype-specific binding of a subset of APL variants.

### Example 10: Production of a Porous Hydrogels for High Throughput Production of Barcoded UV-Exchanged Tetramer Pools

Hydrogel beads were produced by mixing acrylamide monomer units and bis-acrylamide crosslinker units at a variety of relative concentrations along with a mixture of acrydated oligonucleotide primers, encapsulating in droplets using a microfluidic drop-maker, and incubating the mixture until crosslinking was complete. In this Example, the pre-crosslinked aqueous mix included 0.75% bis-acrylamide, 3% acrylamide, 25 uM 5′-acrydated forward primer, 0.5% ammonium persulfate, in 10% TEBST (Tris-EDTA-buffered saline plus Tween-20). All reagents of the aqueous mixture were combined and stirred. The mixture was supplemented with 1.5% TEMED and 1% of 008-FluoroSurfactant, encapsulated in droplets, incubated at room temperature for 1 hour, and then transferred into an oven at 60° C. for overnight incubation, thus forming the hydrogels. The hydrogel beads were washed once with 20% 1H,1H,2H,2H-perfluoro-1-octanol (PFO), then washed three times with TEBST, and then washed three times with low TE (1 mM Tris-Cl pH 7.5, 0.1 mM EDTA). Hydrogel beads were stored in TEBST at 4° C. until use.

### Example 11: Single Template PCR to Generate Peptide-Encoding Amplicons

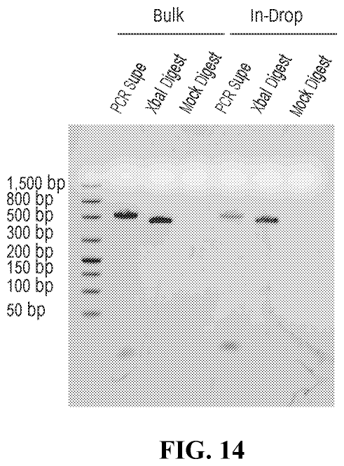

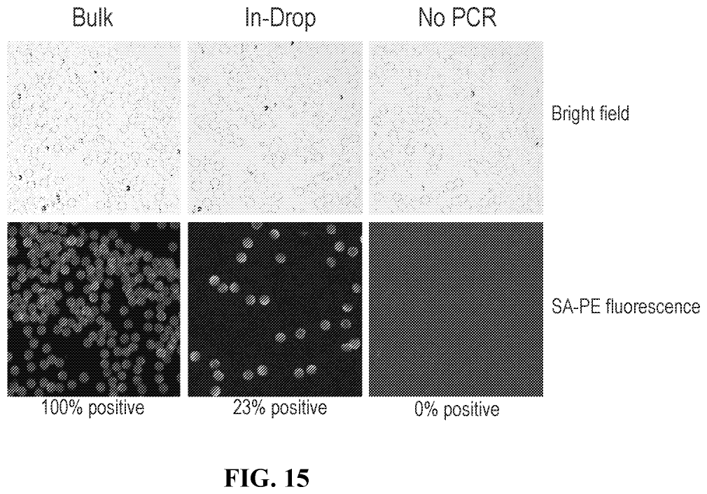

Linear DNA templates encoding a SUMO domain-peptide fusion were PCR-amplified onto hydrogel beads in drops under single template conditions, where each drop gets at most a single DNA template. 1.4 ml hydrogel beads produced in Example 10 were mixed together with PCR components as follows in a 2 ml reaction volume: 400 uM Q5 reaction buffer (New England Biolabs), 40 ul 10 mM dNTP, 40 ul 1 uM forward primer, 40 ul 25 uM 5′-biotinylated reverse primer, 40 ul 0.1 pg/ul linear DNA template (or mix of templates), 8 ul 20% IGEPAL, and 20 uL Q5 DNA polymerase (New England Biolabs). The mixture was encapsulated in drops and subjected to 35 cycles of PCR. After drop lysis by addition of an equal volume of 100% perfluorooctanol (PFO), hydrogels were washed with 10 volumes of low TE five times. Aliquots (10 ul ea) of hydrogel beads were digested with XbaI, which cuts within the amplicon, for 1 hour at 37° C. and run on a 1.2% agarose gel along with PCR supernatant to quantify yield and quality of amplicons (FIG. 14). That single template conditions were in effect was demonstrated by labeling hydrogels with streptavidin-PE, where only 23% of drop-amplified hydrogels were stained, compared to 100% of bulk-amplified hydrogels (FIG. 15).

### Example 12: Loading of Barcodable Exchange-Ready Conjugated Tetramers onto Hydrogels

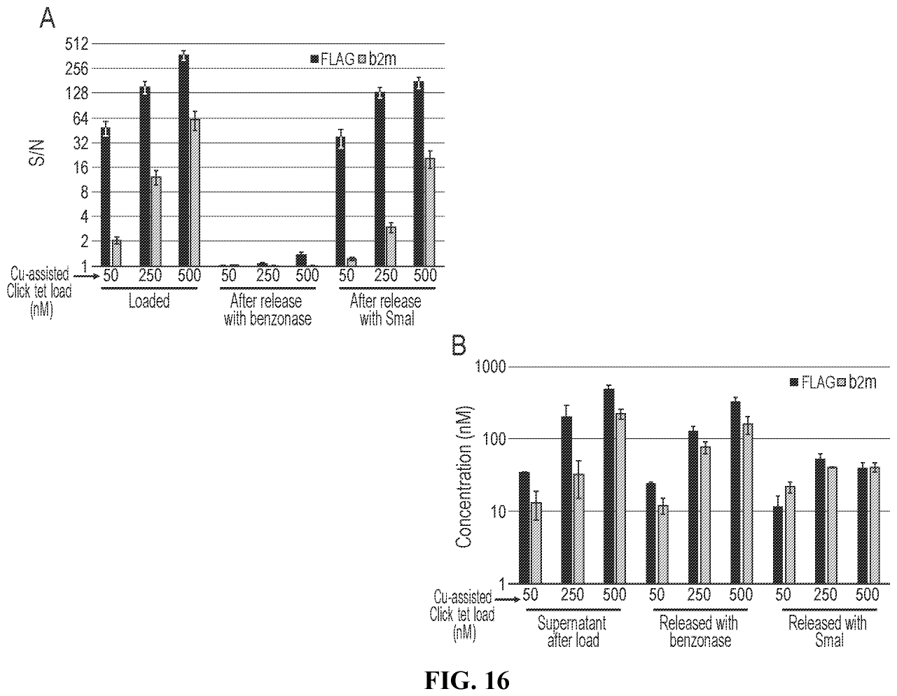

PCR-amplified hydrogels were mixed 1:1 by volume with 50 to 500 nM HLA-A*02:01-Alk-SAv-Az Conjugated Tetramers loaded with the UV-labile peptide (e.g., GILGFVFJL (SEQ ID NO:7), protected from ambient light, and incubated on ice for 2 hours. Loading of HLA-A*02:01-Alk-SAv-Az Conjugated Tetramers was confirmed by washing and staining with anti-Flag-APC or anti-β2M-Alexa488 as seen in FIG. 16A. The quantity of tetramers loaded was quantified by releasing with benzonase or SmaI, which cuts within the amplicon, followed by ELISA with anti-streptavidin capture and either anti-Flag-HRP or anti-β2M-HRP detection, as shown in FIG. 16B.

### Example 13: In-Drop In Vitro Transcription/Translation (IVTT) of Peptide and UV Exchange into Loaded Tetramers

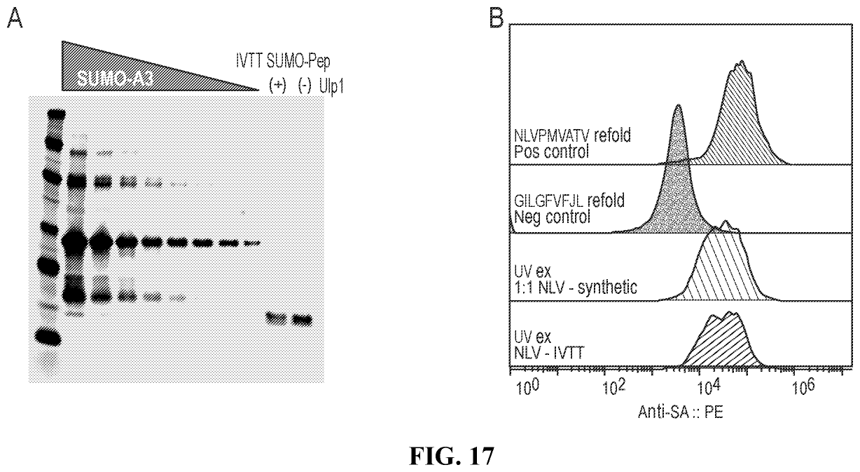

120 ul of hydrogel beads are co-encapsulated in drops with 240 ul of IVTT master mix, including 120 ul PURExpress solution A (New England Biolabs), 90 ul PURExpress solution B (NEB), 6 ul RNAse OUT (Invitrogen), and 1.2U Ulp1 protease (Invitrogen). Drops were incubated at 30° C. for 4 hours, without shaking, then UV-exchanged by 30-minute exposure to 365 nm UV light from a lamp held 2-5 cm from the sample. The UV exposure was followed by 30 minutes incubation at 30° C. to allow complete exchange. D-Biotin was added to the IVTT reactions to a final concentration of 500 uM prior to breaking drops, which was then accomplished by addition of an equal volume of 100% PFO. Hydrogel beads were washed five times with 10 volumes of PBS plus 2% BSA. Sufficient peptide can be produced from a PCR amplicon to generate functional exchanged tetramers, as shown in FIGS. 17A and 17B.

### Example 14: Release and Analysis of Single Chain Multimeric Peptide-MHC

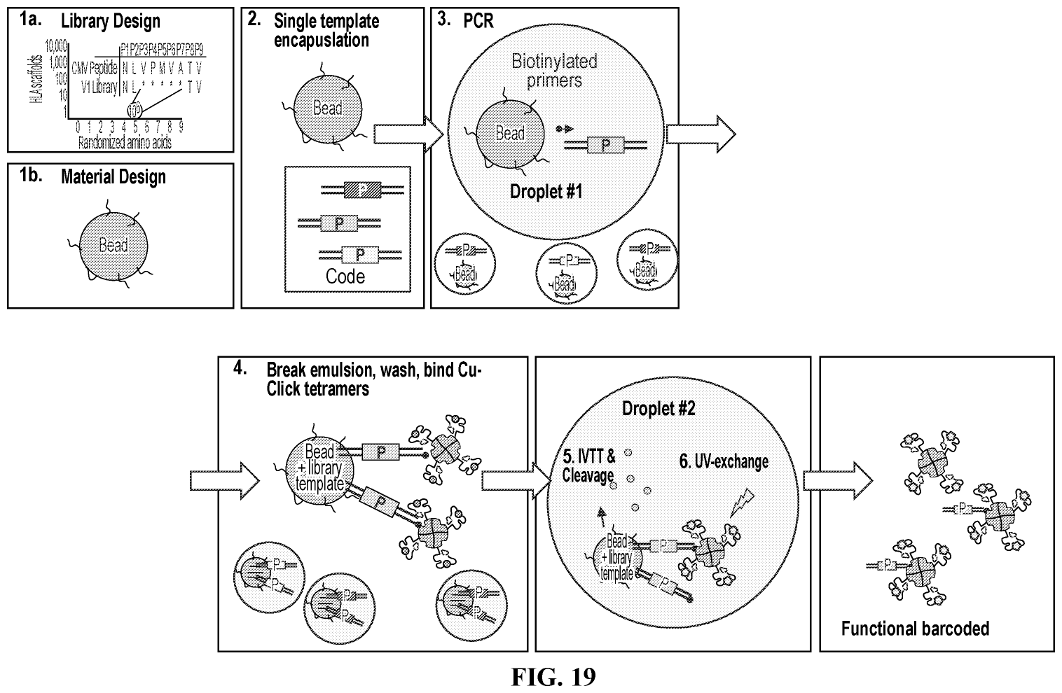

UV-exchanged pMHC were released from washed hydrogels by digestion with SmaI, which cuts within the amplicon upstream of the peptide-encoding region, such that the tetramers were released with a self-identifying oligonucleotide tag (barcode) as indicated in FIG. 16B. Released pMHC were quantified by ELISA as indicated in FIG. 16B, and stained on antigen-specific CD8+ T cells as shown in FIG. 18. The entire process for in-drop production is summarized schematically in FIG. 19.

### Example 15: Generation of Conjugated Peptide/MHC Class II-SAv Multimers

Conjugation of Click-Handle peptide to MHC II-Sortag using Sortase. The sequences of MHC II α- and β-chains were recombinantly expressed as follows: the α-chain extracellular domain sequence was expressed with a C-terminal sortase tag that enables post-translational coupling to Streptavidin (SAv) to form barcodable exchangeable MHC II multimers The α-chain also contained a Myc tag for diagnostic purposes. The amino acid sequence of the α-chain extracellular domain with sortag and Myc tag is shown in SEQ ID NO: 191. The β-chain was recombinantly expressed with an N-terminal low-affinity placeholder peptide (CLIP peptide, the sequence of which is shown in SEQ ID NO:189) followed by a flexible linker, the β-chain extracellular domain and a Histidine purification tag. The amino acid sequence of the β-chain extracellular domain with placeholder peptide, flexible linker and His Tag is shown in SEQ ID NO:192. The flexible linker contained a cleavage site that permitted breaking the connection between the peptide and the β-chain by a specific protease, thus facilitating subsequent peptide exchange. MHCII molecules with a covalent placeholder peptide loaded therein are referred to herein as p*MHCII.

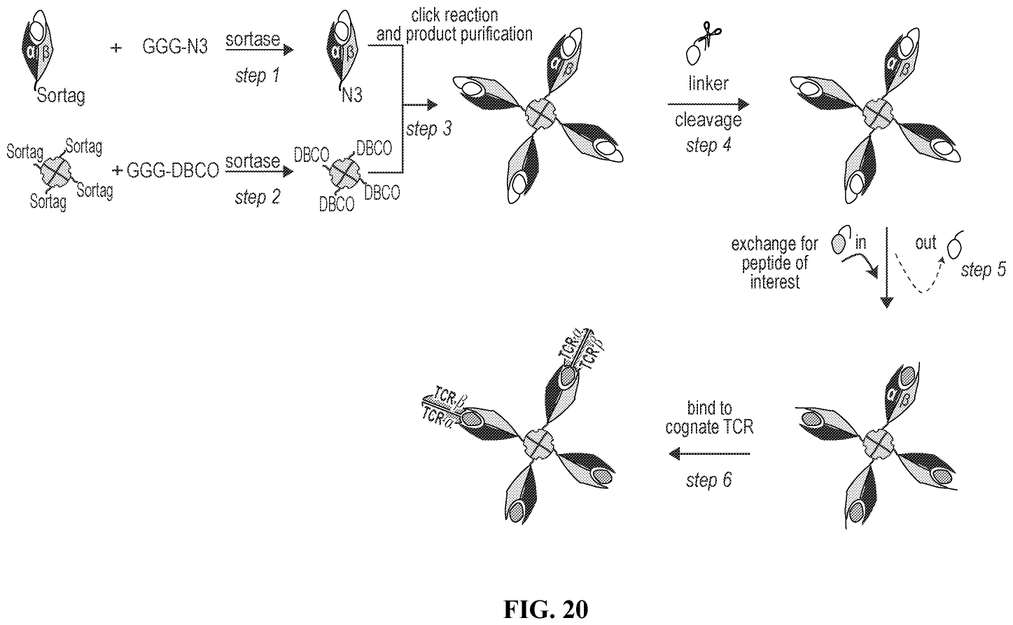

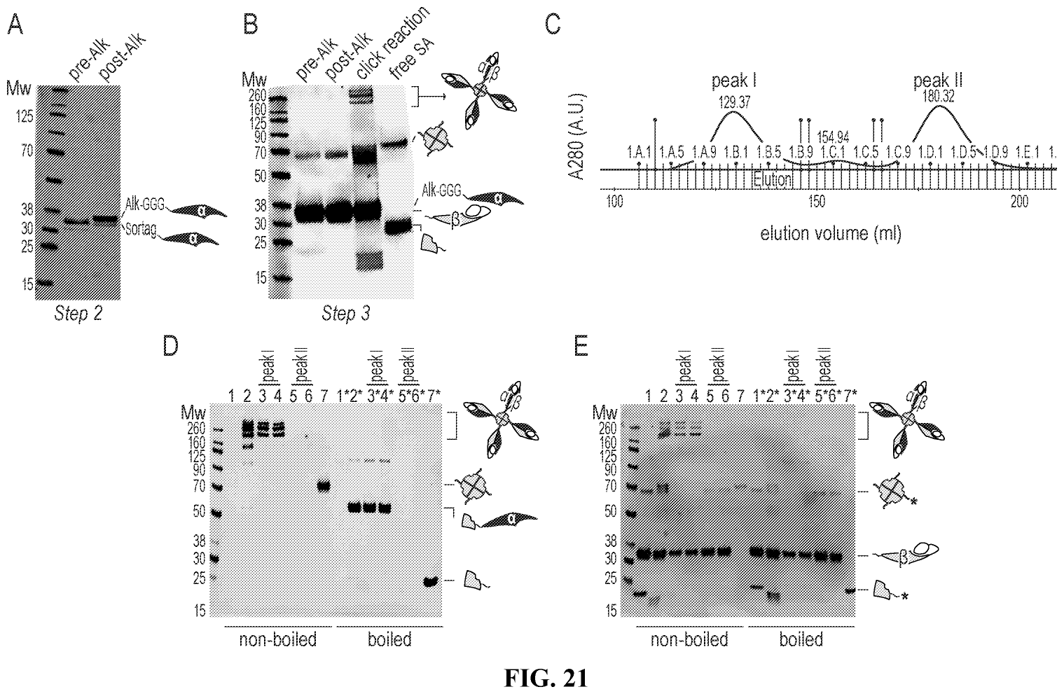

p*MHCII α- and β-chains were co-expressed in CHO cells and secreted into the expression medium as a stable heterodimer. Following CHO expression, p*MHCII was purified by immobilized metal ion affinity chromatography and size exclusion chromatography (SEC). Sortase enzyme was then used to conjugate a GGG-X peptide to the p*MHCII α-chain (FIG. 20, step 1) where X can be an azide, an alkyne, or any clickable chemical moiety. To execute the chemical conjugation reaction p*MHCII (30-50 uM), Click Handle Peptide (GGG-Alkyne, GGG-DBCO, or GGG-Azide at 6-10 mM), Sortase (5-6 uM) and 10 mM CaCl₂ were mixed and incubated at 4° C. for up to 2 hours to generate an p*MHCII-Click-Handle fusion. The reaction mixture was purified by SEC to remove residual Sortase and Click-Handle-Peptide. Purified fractions corresponding to p*MHCII-Click-Handle fusion were pooled and concentrated. Click Handle addition caused a shift in the size of the conjugated protein, validating a successful sortase-mediated ligation (FIG. 21A).

The Generation of Conjugated p*MHCII-SAv Multimers.

The expression, purification and conjugation of Click-Handle p*MHCII to SAv using Sortase is illustrated in FIG. 20, step 2, and was carried out essentially as described in Example 1 for MHCI multimers. Copper-assisted alkyne-azide cycloaddition was used to generate covalently linked p*MHCII and SAv (FIG. 20, step 3). p*MHCII-Alk-SAv-Az was generated by mixing the following reaction components on ice: MHC II-Alk (50 uM), SAv-Az (25 uM with respect to SA-monomer), Copper Sulfate (0.5 mM), BTTAA (2.5 mM) and Ascorbic Acid (5 mM). The reaction was monitored by SDS-PAGE (FIG. 21B) and after 4 hours the reaction mixture was purified by SEC to separate unreacted HLA, SAv, and other reaction components from purified p*MHCII-Alk-SAv-Az multimer (FIG. 21C). The SAv and the β-chain contained FLAG and His tags, respectively, enabling to distinguish fractions corresponding to multimer species (FIGS. 21D and 21E). The multimer fractions showed apparent tetramer and trimer species. More importantly, free SAv species were not observed in boiled samples taken from multimer fractions under SDS-PAGE and western blot analysis (FIG. 21D). This indicates that the dominant species is a tetramer, in which each SAv subunit is covalently linked to an p*MHCII subunit.

### Example 16: pMHC II Multimers are Exchangeable and Bind Cognate Epitope-Specific TCR

### Linker Digestion and Peptide Exchange

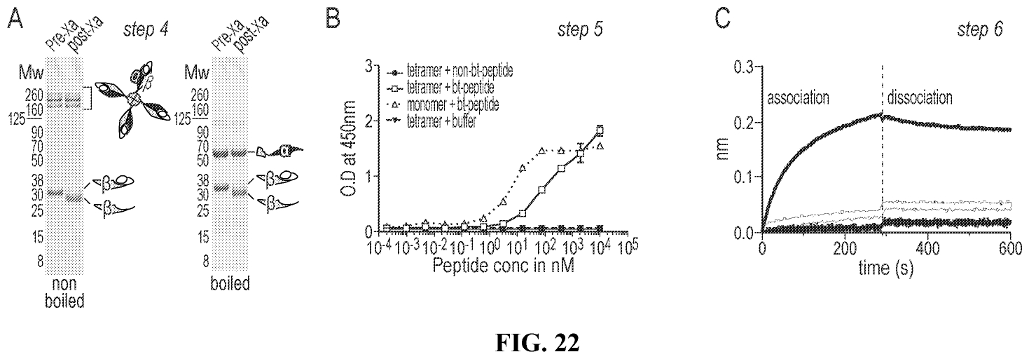

p*MHCII-Alk-SAv-Az multimer (henceforth—p*MHCII-SAv) was digested by Factor Xa (NEB) at a ratio of 5:1 (w/w) over night at 4° C. in the presence of 1 mM CaCl₂) (FIG. 20, step 4). Then the protease was irreversibly inactivated by the addition of 1,5-Dansyl-Glu-Gly-Arg Chloromethyl Ketone inhibitor according to the manufacturer's recommendations (Sigma-Aldrich). Digested samples migrated faster than non-digested samples indicating the removal of the freshly cleaved peptide under SDS-PAGE denaturative conditions (FIG. 22A).

To test whether cleaved p*MHCII-SAv (henceforth—p↓MHCII-SAv) bound an exchanged peptide, an ELISA binding assay was performed. In this assay, a biotinylated peptide epitope from Influenza A virus (Hemagglutinin, HA, the amino acid sequence of which is shown in SEQ ID NO:193) was loaded while the cleaved placeholder peptide was removed under mild acidic pH conditions (FIG. 20, step 5). The level of exchange was then determined by monitoring the binding of streptavidin-HRP to the newly swapped biotinylated peptide. Free biotin binding sites on the streptavidin molecules were blocked with an excess of free biotin prior to the exchange reaction. This step ensured that any detected biotinylated peptide can only be bound to the peptide-binding pocket. The exchange-buffer composition was as follows: 100 mM sodium citrate pH 5.5, 50 mM sodium Chloride, 1% octyl glucoside (v/v), 1× of SIGMAFAST protease inhibitor cocktail (Sigma-Aldrich) and 0.1 mM DTT. 150 μl of peptide exchange reactions were prepared in a 96-well plate where each well consists of: 1× exchange buffer, 30 nM p↓MHCII-SAv and 5-fold serial dilutions of either HA-biotinylated peptide, HA-non-biotinylated peptide or buffer. Incubation of 6 nM of p↓MHCII monomer with 5-fold serial dilutions of HA-biotinylated peptide was included as a positive control. The exchange reaction was stopped after an over-night incubation at 37 C by neutralizing the acidic pH with the addition of 1:15 (v/v) of 1 M Tris-HCl, pH 10. Using a 96 channel benchtop pipettor, 100 μl from each well were transferred to an ELISA plate that was pre-coated with (100 ng/well) L243 conformational sensitive antibody (Abcam), washed (3×PBS-T) and blocked with PBS-T supplemented with 2% (v/v) BSA. Following 1 hr incubation at RT, the plate was washed (3×PBS-T), incubated with SA-HRP for 30 minutes in the dark, washed again (3×PBS-T) and developed using an HRP substrate and stop solution. A positive correlation between peptide concentrations and the levels of SA-HRP binding was observed for both monomeric p↓MHCII and p↓MHCII-SAv (FIG. 21B). This indicates that both species exchanged the placeholder peptide for biotinylated-HA peptide. Incubation with either non-biotinylated peptide or buffer did not yield a detectable signal implying that binding of the biotinylated epitope was specific. In contrast to monomeric p↓MHCII, the curve for p↓MHCII-SAv was shifted to the right and did not reach saturation at higher peptide concentrations. The multimer is at least 4-fold bigger in size, which might occlude binding to the capturing antibody and/or to the SA-HRP readout probe.

Binding of Exchanged P↓MHCII-SAv to Soluble TCR

F11, an HA-peptide epitope specific soluble TCR, was fused to an FC domain and produced as described in Wagner et al. J Biol Chem., 294:5790-5804, 2019 (FIG. 20, step 6). Briefly, DNA encoding the F11 extracellular alpha- and beta-chains was cloned into pDT5 plasmids downstream of a mouse IgGk chain leader sequence. The human TCR constant domains contained an additional inter-chain disulfide bond. The C-alpha domain was followed by the upper hinge sequence of human IgG1 (VEPKSC; SEQ ID NO: 270), the core and lower hinge, and then the Fc domain. The native IgG1 light-chain cysteine was inserted at the C-terminus of C-beta to pair with the upper hinge cysteine and further stabilize the TCR heterodimerization. Additional modifications included the removal of N-linked glycosylation sites. Plasmids encoding alpha-Fc and beta domains were expressed in Expi-CHO cells by transient transfection, and the product was purified from clarified supernatants by protein A affinity chromatography.

The exchange reaction was performed as described above in Example 1 with two differences: a single tube was used instead of a 96-well plate and the protein concentrations varied. 1.75 μM of p↓MHCII-SAv were incubated with 100 μM of HA peptide in the presence of exchange buffer. After the reaction was stopped and kept on ice, a Bio-layer interferometry (BLI) assay was carried out using an Octet RED96 instrument (ForteBio) at 30 C in BLI buffer (PBS+0.02% Tween20, 0.1% BSA, 0.05% sodium azide). F11 TCR was loaded onto Anti-hIgG Fc Capture Biosensors (Molecular Devices) to 0.6 nm loading signal. After washing with BLI buffer, biosensors were transferred to wells containing either 14 nM of exchanged p↓MHCII-SAv, 125 nM of non-exchanged p*MHCII-SAv or BLI buffer to measure association kinetics (FIG. 22C). To measure dissociation kinetics, biosensors were transferred back to BLI buffer devoid of multimers. A significant increase in BLI-response signal was observed for HA-exchanged p↓MHCII-SAv suggesting a strong association with F11 TCR (FIG. 22C). In contrast, non-exchanged p*MHCII-SAv showed very little association indicating that the interaction between F11-TCR and an HA displaying multimer is specific. No association was observed when the biosensors were dipped into BLI buffer. HA-exchanged p↓MHCII-SAv exhibited very slight dissociation from F11-TCR. This result indicates a tight TCR-MHC II binding which is characteristic of high-avidity multimer interaction.

Binding of a Library of Exchanged p↓MHCII-SAv to Antigen-Specific CD4+ T Cells

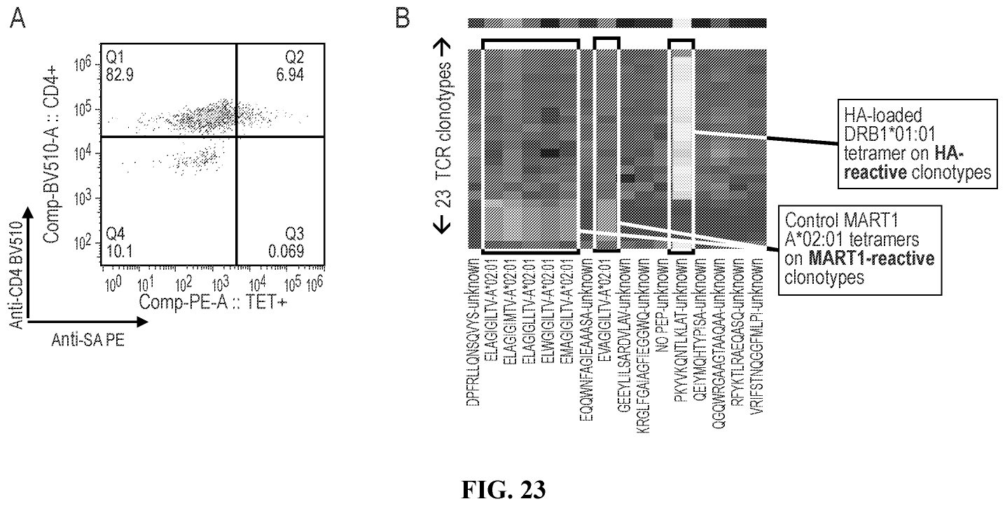

Individual DRB1*01:01-SAv Conjugated Tetramer samples that were UV-exchanged for influenza haemagluttinin (HA) peptide (SEQ ID NO: 281) and 9 other control peptides were labeled with oligonucleotide and pooled. Subsequently, the pool was used to stain HA-expanded CD4+ T cells, which were sorted and subjected to single cell sequencing after spiking with control epitope ELAGIGILTV (SEQ ID NO: 282)-expanded cells that had been stained with an HLA-A*02:01 tetramer pool. The analyzed results are shown in a heatmap in FIG. 23, indicating clonotype-specific binding for the HA-loaded DRB1*01:01 tetramer.

### INCORPORATION BY REFERENCE

Each patent, publication, and non-patent literature cited in the application is hereby incorporated by reference in its entirety as if each was incorporated by reference individually.

SEQUENCE LISTING SUMMARY

| SEQ | |
|---|---|
| ID | |
| NO: | DESCRIPTION |
| 1 | MSGSHSMRYFFTSVSRPGRGEPRFIAVGYVDDTQFVRFDSDAASQRMEPRAPWIEQEGPEYW |
| | DGETRKVKAHSQTHRVDLGTLRGYYNQSEAGSHTVQRMYGCDVGSDWRFLRGYHQYAYD |
| | GKDYTALKEDLRSWTAADMAAQTTKHKWEAAHVAEQLRAYLEGTCVEWLRRYLENGKET |
| | LQRTDAPKTHMTHHAVSDHEATLRCWALSFYPAEITLTWQRDGEDQTQDTELVETRPAGDG |
| | TFQKWAAVVVPSGQEQRYTCHVQHEGLPKPLTLRWEPSSGSGSAGGSGSGGGSLPETGGHH |
| | HHHH |
| | (HLA-A2- Sorttag and His6 tag) |
| 2 | MIQRTPKIQVYSRHPAENGKSNFLNCYVSGFHPSDIEVDLLKNGERIEKVEHSDLSFSKDWSF |
| | YLLYYTEFTPTEKDEYACRVNHVTLSQPKIVKWDRDM |
| | (β-2 microglobulin, with extra N-terminal methionine) |
| 3 | MDYKDDDDKGSSGDPSKDSKAQVSAAEAGITGTWYNQLGSTFIVTAGADGALTGTYESAVG |
| | NAESRYVLTGRYDSAPATDGSGTALGWTVAWKNNYRNAHSATTWSGQYVGGAEARINTQ |
| | WLLTSGTTEANAWKSTLVGHDTFTKVKPSAASIDAAKKAGVNNGNPLDAVQQLPETGGHH |
| | HHHH |
| | (SAv -Sorttag and His6 tag) |
| 4 | MGSHSMRYFFTSVSRPGRGEPRFIAVGYVDDTQFVRFDSDAASQRMEPRAPWIEQEGPEYWD |
| | GETRKVKAHSQTHRVDLGTLRGYYNQSEAGSHTVQRMYGCDVGSDWRFLRGYHQYAYDG |
| | KDYIALKEDLRSWTAADMAAQTTKHKWEAAHVAEQLRAYLEGTCVEWLRRYLENGKETL |
| | QRTDAPKTHMTHHAVSDHEATLRCWALSFYPAEITLTWQRDGEDQTQDTELVETRPAGDGT |
| | FQKWAAVVVPSGQEQRYTCHVQHEGLPKPLTLRWEPSSGSGSAGGSFESGPGAEYCLSYETE |
| | ILTVEYGLLPIGKIVEKRIECTVYSVDNNGNIYTQPVAQWHDRGEQEVFEYCLEDGSLIRATK |
| | DHKFMTVDGQMLPIDEIFERELDLMRVDNLPN |
| | (HLA-A2-N-intein) |
| 5 | MIKIATRKYLGKQNVYDIGVERDHNFALKNGFIASNCFNVDDPSKDSKAQVSAAEAGITGTW |
| | YNQLGSTFIVTAGADGALTGTYESAVGNAESRYVLTGRYDSAPATDGSGTALGWTVAWKN |
| | NYRNAHSATTWSGQYVGGAEARINTQWLLTSGTTEANAWKSTLVGHDTFTKVKPSAASIDA |
| | AKKAGVNNGNPLDAVQQGSTGDYKDDDDK |
| | (C-intein-Sav and Flag Tag) |
| 6 | MQAKPQIPKDKSKVAGYIEIPDADIKEPVYPGPATREQLNRGVSFAEENESLDDQNISIAGHTFI |
| | DRPNYQFTNLKAAKKGSMVYFKVGNETRKYKMTSIRNVKPTAVEVLDEQKGKDKQLTLITC |
| | DDYNEETGVWETRKIFVATEVKLEHHHHHH |
| | (Sortase with His6 tag) |
| 7 | GILGFVFJL |
| | (A02:01 placeholder peptide) |
| 8 | NLVPMVATV |
| | (A02:01 binding peptide) |
| 9 | NLVPMVGTV |
| | (A02:01 binding peptide) |
| 10 | VTEHDTLLY |
| | (A01:01 binding peptide |
| 11 | VYGJVRACL |
| | (A24:02 placeholder peptide) |
| 12 | QYDPVAALF |
| | (A24:02 binding peptide) |
| 13 | RPHERNGFTVL |
| | (B7:02 binding peptide) |
| 14 | AARGJTLAM |
| | (B7:02 placeholder peptide) |
| 15 | KILGFVFJV |
| | (A2:01 placeholder peptide) |
| 16 | STAPGJLEY |
| | (A1:01 placeholder peptide) |
| 17 | RIYRJGATR |
| | (A3:01 placeholder peptide) |
| 18 | RVFAJSFIK |
| | (A11:01 placeholder peptide) |
| 19 | KPIVVLJGY |
| | (B35:01 placeholder peptide) |
| 20 | FVYGJSKTSL |
| | (C3:04 placeholder peptide) |
| 21 | FLRGRAJGL |
| | (B8:01 placeholder peptide) |
| 22 | VRIJHLYIL |
| | (C7:02 placeholder peptide) |
| 23 | QYDJAVYKL |
| | (C4:01 placeholder peptide) |
| 24 | ILGPJGSVY |
| | (B15:01 placeholder peptide) |
| 25 | TEADVQJWL |
| | (B40:01 placeholder peptide) |
| 26 | ISARGQJLF |
| | (B58:01 placeholder peptide) |
| 27 | KAAJDLSHFL |
| | (C8:01 placeholder peptide) |
| 28 | MAVMAPRTLLLLLSGALALTQTWAGSHSMRYFFTSVSRPGRGEPRFIAVGYVDDTQFVRFDS |
| | DAASQKMEPRAPWIEQEGPEYWDQETRNMKAHSQTDRANLGTLRGYYNQSEDGSHTIQIMY |
| | GCDVGPDGRFLRGYRQDAYDGKDYIALNEDLRSWTAADMAAQITKRKWEAVHAAEQRRV |
| | YLEGRCVDGLRRYLENGKETLQRTDPPKTHMTHHPISDHEATLRCWALGFYPAEITLTWQRD |
| | GEDQTQDTELVETRPAGDGTFQKWAAVVVPSGEEQRYTCHVQHEGLPKPLTLRWELSSQPTI |
| | PIVGIIAGLVLLGAVITGAVVAAVMWRRKSSDRKGGSYTQAASSDSAQGSDVSLTACKV |
| | (HLA-A*01:01 full-length) |
| 29 | MAVMAPRTLLLLLSGALALTQTWAGSHSMRYFFTSVSRPGRGEPRFIAVGYVDDTQFVRFDS |
| | DAASQRMEPRAPWIEQEGPEYWDQETRNVKAQSQTDRVDLGTLRGYYNQSEAGSHTIQIMY |
| | GCDVGSDGRFLRGYRQDAYDGKDYIALNEDLRSWTAADMAAQITKRKWEAAHEAEQLRAY |
| | LDGTCVEWLRRYLENGKETLQRTDPPKTHMTHHPISDHEATLRCWALGFYPAEITLTWQRD |
| | GEDQTQDTELVETRPAGDGTFQKWAAVVVPSGEEQRYTCHVQHEGLPKPLTLRWELSSQPTI |
| | PIVGIIAGLVLLGAVITGAVVAAVMWRRKSSDRKGGSYTQAASSDSAQGSDVSLTACKV |
| | (HLA-A*03:01 full-length) |
| 30 | MAVMAPRTLLLLLSGALALTQTWAGSHSMRYFYTSVSRPGRGEPRFIAVGYVDDTQFVRFDS |
| | DAASQRMEPRAPWIEQEGPEYWDQETRNVKAQSQTDRVDLGTLRGYYNQSEDGSHTIQIMY |
| | GCDVGPDGRFLRGYRQDAYDGKDYIALNEDLRSWTAADMAAQITKRKWEAAHAAEQQRA |
| | YLEGRCVEWLRRYLENGKETLQRTDPPKTHMTHHPISDHEATLRCWALGFYPAEITLTWQRD |
| | GEDQTQDTELVETRPAGDGTFQKWAAVVVPSGEEQRYTCHVQHEGLPKPLTLRWELSSQPTI |
| | PIVGIIAGLVLLGAVITGAVVAAVMWRRKSSDRKGGSYTQAASSDSAQGSDVSLTACKV |
| | (HLA-A*11:01 full-length) |
| 31 | MAVMAPRTLVLLLSGALALTQTWAGSHSMRYFSTSVSRPGRGEPRFIAVGYVDDTQFVRFDS |
| | DAASQRMEPRAPWIEQEGPEYWDEETGKVKAHSQTDRENLRIALRYYNQSEAGSHTLQMMF |
| | GCDVGSDGRFLRGYHQYAYDGKDYIALKEDLRSWTAADMAAQITKRKWEAAHVAEQQRA |
| | YLEGTCVDGLRRYLENGKETLQRTDPPKTHMTHHPISDHEATLRCWALGFYPAEITLTWQRD |
| | GEDQTQDTELVETRPAGDGTFQKWAAVVVPSGEEQRYTCHVQHEGLPKPLTLRWEPSSQPT |
| | VPIVGIIAGLVLLGAVITGAVVAAVMWRRNSSDRKGGSYSQAASSDSAQGSDVSLTACKV |
| | (HLA-A*24:02 full-length) |
| 32 | MLVMAPRTVLLLLSAALALTETWAGSHSMRYFYTSVSRPGRGEPRFISVGYVDDTQFVRFDS |
| | DAASPREEPRAPWIEQEGPEYWDRNTQIYKAQAQTDRESLRNLRGYYNQSEAGSHTLQSMY |
| | GCDVGPDGRLLRGHDQYAYDGKDYIALNEDLRSWTAADTAAQITQRKWEAAREAEQRRAY |
| | LEGECVEWLRRYLENGKDKLERADPPKTHVTHHPISDHEATLRCWALGFYPAEITLTWQRDG |
| | EDQTQDTELVETRPAGDRTFQKWAAVVVPSGEEQRYTCHVQHEGLPKPLTLRWEPSSQSTVP |
| | IVGIVAGLAVLAVVVIGAVVAAVMCRRKSSGGKGGSYSQAACSDSAQGSDVSLTA |
| | (HLA-B*07:02 full-length) |
| 33 | MRVMAPRTLILLLSGALALTETWAGSHSMRYFSTSVSWPGRGEPRFIAVGYVDDTQFVRFDS |
| | DAASPRGEPREPWVEQEGPEYWDRETQKYKRQAQADRVNLRKLRGYYNQSEDGSHTLQRM |
| | FGCDLGPDGRLLRGYNQFAYDGKDYIALNEDLRSWTAADTAAQITQRKWEAAREAEQRRA |
| | YLEGTCVEWLRRYLENGKETLQRAEHPKTHVTHHPVSDHEATLRCWALGFYPAEITLTWQW |
| | DGEDQTQDTELVETRPAGDGTFQKWAAVVVPSGEEQRYTCHVQHEGLPEPLTLRWKPSSQP |
| | TIPIVGIVAGLAVLAVLAVLGAMVAVVMCRRKSSGGKGGSCSQAASSNSAQGSDESLIACKA |
| | (HLA-C*04:01 full-length) |
| 34 | MRVMAPRALLLLLSGGLALTETWACSHSMRYFDTAVSRPGRGEPRFISVGYVDDTQFVRFDS |
| | DAASPRGEPRAPWVEQEGPEYWDRETQKYKRQAQADRVSLRNLRGYYNQSEDGSHTLQRM |
| | SGCDLGPDGRLLRGYDQSAYDGKDYIALNEDLRSWTAADTAAQITQRKLEAARAAEQLRAY |
| | LEGTCVEWLRRYLENGKETLQRAEPPKTHVTHHPLSDHEATLRCWALGFYPAEITLTWQRD |
| | GEDQTQDTELVETRPAGDGTFQKWAAVVVPSGQEQRYTCHMQHEGLQEPLTLSWEPSSQPTI |
| | PIMGIVAGLAVLVVLAVLGAVVTAMMCRRKSSGGKGGSCSQAACSNSAQGSDESLITCKA |
| | (HLA-C*07:02 full-length) |
| 35 | MLVMAPRTVLLLLSAALALTETWAGSHSMRYFDTAMSRPGRGEPRFISVGYVDDTQFVRFD |
| | SDAASPREEPRAPWIEQEGPEYWDRNTQIFKTNTQTDRESLRNLRGYYNQSEAGSHTLQSMY |
| | GCDVGPDGRLLRGHNQYAYDGKDYIALNEDLRSWTAADTAAQITQRKWEAARVAEQDRAY |
| | LEGTCVEWLRRYLENGKDTLERADPPKTHVTHHPISDHEATLRCWALGFYPAEITLTWQRDG |
| | EDQTQDTELVETRPAGDRTFQKWAAVVVPSGEEQRYTCHVQHEGLPKPLTLRWEPSSQSTVP |
| | IVGIVAGLAVLAVVVIGAVVAAVMCRRKSSGGKGGSYSQAACSDSAQGSDVSLTA |
| | (HLA-B*08:01 full-length) |
| 36 | MRVTAPRTVLLLLWGAVALTETWAGSHSMRYFYTAMSRPGRGEPRFIAVGYVDDTQFVRFD |
| | SDAASPRTEPRAPWIEQEGPEYWDRNTQIFKTNTQTYRESLRNLRGYYNQSEAGSHIIQRMYG |
| | CDLGPDGRLLRGHDQSAYDGKDYIALNEDLSSWTAADTAAQITQRKWEAARVAEQLRAYLE |
| | GLCVEWLRRYLENGKETLQRADPPKTHVTHHPVSDHEATLRCWALGFYPAEITLTWQRDGE |
| | DQTQDTELVETRPAGDRTFQKWAAVVVPSGEEQRYTCHVQHEGLPKPLTLRWEPSSQSTIPIV |
| | GIVAGLAVLAVVVIGAVVATVMCRRKSSGGKGGSYSQAASSDSAQGSDVSLTA |
| | (HLA-B*35:01 full-length) |
| 37 | MRVTAPRTVLLLLWGAVALTETWAGSHSMRYFYTAMSRPGRGEPRFIAVGYVDDTQFVRFD |
| | SDAASPRMAPRAPWIEQEGPEYWDGETRNMKASAQTYRENLRIALRYYNQSEAGSHIIQVM |
| | YGCDVGPDGRLLRGHDQSAYDGKDYIALNEDLSSWTAADTAAQITQRKWEAARVAEQLRA |
| | YLEGLCVEWLRRYLENGKETLQRADPPKTHVTHHPISDHEATLRCWALGFYPAEITLTWQRD |
| | GEDQTQDTELVETRPAGDRTFQKWAAVVVPSGEEQRYTCHVQHEGLPKPLTLRWEPSSQST |
| | VPIVGIVAGLAVLAVVVIGAVVAAVMCRRKSSGGKGGSYSQAACSDSAQGSDVSLTA |
| | (HLA-B*57:01 full-length) |
| 38 | MRVTAPRTVLLLLWGAVALTETWAGSHSMRYFYTAMSRPGRGEPRFIAVGYVDDTQFVRFD |
| | SDAASPRMAPRAPWIEQEGPEYWDGETRNMKASAQTYRENLRIALRYYNQSEAGSHIIQVM |
| | YGCDVGPDGRLLRGHNQYAYDGKDYIALNEDLSSWTAADTAAQITQRKWEAARVAEQLRA |
| | YLEGLCVEWLRRYLENGKETLQRADPPKTHVTHHPISDHEATLRCWALGFYPAEITLTWQRD |
| | GEDQTQDTELVETRPAGDRTFQKWAAVVVPSGEEQRYTCHVQHEGLPKPLTLRWEPSSQST |
| | VPIVGIVAGLAVLAVVVIGAVVAAVMCRRKSSGGKGGSYSQAACSDSAQGSDVSLTA |
| | (HLA-B*57:03 full-length) |
| 39 | MVDGTLLLLLSEALALTQTWAGSHSLKYFHTSVSRPGRGEPRFISVGYVDDTQFVRFDNDAA |
| | SPRMVPRAPWMEQEGSEYWDRETRSARDTAQIFRVNLRTLRGYYNQSEAGSHTLQWMHGC |
| | ELGPDGRFLRGYEQFAYDGKDYLTLNEDLRSWTAVDTAAQISEQKSNDASEAEHQRAYLED |
| | TCVEWLHKYLEKGKETLLHLEPPKTHVTHHPISDHEATLRCWALGFYPAEITLTWQQDGEGH |
| | TQDTELVETRPAGDGTFQKWAAVVVPSGEEQRYTCHVQHEGLPEPVTLRWKPASQPTIPIVGI |
| | IAGLVLLGSVVSGAVVAAVIWRKKSSGGKGGSYSKAEWSDSAQGSESHSL |
| | (HLA-E full-length) |
| 40 | MRVMAPRTLILLLSGALALTETWACSHSMRYFYTAVSRPGRGEPRFIAVGYVDDTQFVRFDS |
| | DAASPRGEPRAPWVEQEGPEYWDRETQKYKRQAQTDRVSLRNLRGYYNQSEAGSHTLQWM |
| | YGCDLGPDGRLLRGYDQSAYDGKDYIALNEDLRSWTAADTAAQITQRKWEAARAAEQQRA |
| | YLEGTCVEWLRRYLENGKETLQRAEHPKTHVTHHLVSDHEATLRCWALGFYPAEITLTWQR |
| | DGEDQTQDTELVETRPAGDGTFQKWAAVVVPSGEEQRYTCHVQHEGLPEPLTLRWEPSSQP |
| | TIPIVGIVAGLAVLAVLAVLGAVVAVVMCRRKSSGGKGGSCSQAASSNSAQGSDESLIACKA |
| | (HLA-C*16:01 full-length) |
| 41 | MRVMAPRTLILLLSGALALTETWACSHSMRYFYTAVSRPGRGEPRFIAVGYVDDTQFVQFDS |
| | DAASPRGEPRAPWVEQEGPEYWDRETQKYKRQAQTDRVSLRNLRGYYNQSEAGSHTLQRM |
| | YGCDLGPDGRLLRGYNQFAYDGKDYIALNEDLRSWTAADKAAQITQRKWEAAREAEQRRA |
| | YLEGTCVEWLRRYLENGKKTLQRAEHPKTHVTHHPVSDHEATLRCWALGFYPAEITLTWQR |
| | DGEDQTQDTELVETRPAGDGTFQKWAAVVVPSGEEQRYTCHVQHEGLPEPLTLRWGPSSQP |
| | TIPIVGIVAGLAVLAVLAVLGAVMAVVMCRRKSSGGKGGSCSQAASSNSAQGSDESLIACKA |
| | (HLA-C*08:02 full-length) |
| 42 | MRVMAPRALLLLLSGGLALTETWACSHSMRYFDTAVSRPGRGEPRFISVGYVDDTQFVRFDS |
| | DAASPRGEPRAPWVEQEGPEYWDRETQNYKRQAQADRVSLRNLRGYYNQSEDGSHTLQRM |
| | YGCDLGPDGRLLRGYDQSAYDGKDYIALNEDLRSWTAADTAAQITQRKLEAARAAEQLRAY |
| | LEGTCVEWLRRYLENGKETLQRAEPPKTHVTHHPLSDHEATLRCWALGFYPAEITLTWQRD |
| | GEDQTQDTELVETRPAGDGTFQKWAAVVVPSGQEQRYTCHMQHEGLQEPLTLSWEPSSQPTI |
| | PIMGIVAGLAVLVVLAVLGAVVTAMMCRRKSSGGKGGSCSQAACSNSAQGSDESLITCKA |
| | (HLA-C*07:01 full-length) |
| 43 | MRVMAPRTLILLLSGALALTETWACSHSMRYFYTAVSRPGRGEPRFIAVGYVDDTQFVQFDS |
| | DAASPRGEPRAPWVEQEGPEYWDRETQKYKRQAQTDRVNLRKLRGYYNQSEAGSHTLQRM |
| | YGCDLGPDGRLLRGYNQFAYDGKDYIALNEDLRSWTAADKAAQITQRKWEAAREAEQRRA |
| | YLEGTCVEWLRRYLENGKKTLQRAEHPKTHVTHHPVSDHEATLRCWALGFYPAEITLTWQR |
| | DGEDQTQDTELVETRPAGDGTFQKWAAVVVPSGEEQRYTCHVQHEGLPEPLTLRWGPSSQP |
| | TIPIVGIVAGLAVLAVLAVLGAVMAVVMCRRKSSGGKGGSCSQAASSNSAQGSDESLIACKA |
| | (HLA-C*05:01 full-length) |
| 44 | MRVTAPRTLLLLLWGAVALTETWAGSHSMRYFYTAMSRPGRGEPRFITVGYVDDTLFVRFD |
| | SDATSPRKEPRAPWIEQEGPEYWDRETQISKTNTQTYRENLRTALRYYNQSEAGSHIIQRMYG |
| | CDVGPDGRLLRGYDQDAYDGKDYIALNEDLSSWTAADTAAQITQRKWEAARVAEQDRAYL |
| | EGLCVESLRRYLENGKETLQRADPPKTHVTHHPISDHEVTLRCWALGFYPAEITLTWQRDGE |
| | DQTQDTELVETRPAGDRTFQKWAAVVVPSGEEQRYTCHVQHEGLPKPLTLRWEPSSQSTVPI |
| | VGIVAGLAVLAVVVIGAVVAAVMCRRKSSGGKGGSYSQAACSDSAQGSDVSLTA |
| | (HLA-B*44:02 full-length) |
| 45 | MAVMAPRTLLLLLLGALALTQTWAGSHSMRYFTTSVSRPGRGEPRFIAVGYVDDTQFVRFDS |
| | DAASQRMEPRAPWIEQEGPEYWDLQTRNVKAQSQTDRANLGTLRGYYNQSEAGSHTIQMM |
| | YGCDVGSDGRFLRGYRQDAYDGKDYIALNEDLRSWTAADMAAQITQRKWEAARVAEQLR |
| | AYLEGTCVEWLRRYLENGKETLQRTDAPKTHMTHHAVSDHEATLRCWALSFYPAEITLTWQ |
| | RDGEDQTQDTELVETRPAGDGTFQKWASVVVPSGQEQRYTCHVQHEGLPKPLTLRWEPSSQ |
| | PTIPIVGIIAGLVLFGAVFAGAVVAAVRWRRKSSDRKGGSYSQAASSDSAQGSDMSLTACKV |
| | (HLA-A*29:02 full-length) |
| 46 | MRVTAPRTLLLLLWGAVALTETWAGSHSMRYFYTAMSRPGRGEPRFITVGYVDDTLFVRFD |
| | SDATSPRKEPRAPWIEQEGPEYWDRETQISKTNTQTYRENLRTALRYYNQSEAGSHIIQRMYG |
| | CDVGPDGRLLRGYDQDAYDGKDYIALNEDLSSWTAADTAAQITQRKWEAARVAEQLRAYL |
| | EGLCVESLRRYLENGKETLQRADPPKTHVTHHPISDHEVTLRCWALGFYPAEITLTWQRDGE |
| | DQTQDTELVETRPAGDRTFQKWAAVVVPSGEEQRYTCHVQHEGLPKPLTLRWEPSSQSTVPI |
| | VGIVAGLAVLAVVVIGAVVAAVMCRRKSSGGKGGSYSQAACSDSAQGSDVSLTA |
| | (HLA-B*44:03 full-length) |
| 47 | MRVMAPRTLILLLSGALALTETWAGSHSMRYFYTAVSRPGRGEPHFIAVGYVDDTQFVRFDS |
| | DAASPRGEPRAPWVEQEGPEYWDRETQKYKRQAQTDRVSLRNLRGYYNQSEAGSHIIQRMY |
| | GCDVGPDGRLLRGYDQYAYDGKDYIALNEDLRSWTAADTAAQITQRKWEAAREAEQLRAY |
| | LEGLCVEWLRRYLKNGKETLQRAEHPKTHVTHHPVSDHEATLRCWALGFYPAEITLTWQWD |
| | GEDQTQDTELVETRPAGDGTFQKWAAVVVPSGEEQRYTCHVQHEGLPEPLTLRWEPSSQPTI |
| | PIVGIVAGLAVLAVLAVLGAVVAVVMCRRKSSGGKGGSCSQAASSNSAQGSDESLIACKA |
| | (HLA-C*03:04 full-length) |
| 48 | MRVTAPRTVLLLLSAALALTETWAGSHSMRYFHTAMSRPGRGEPRFITVGYVDDTLFVRFDS |
| | DATSPRKEPRAPWIEQEGPEYWDRETQISKTNTQTYRESLRNLRGYYNQSEAGSHTLQRMYG |
| | CDVGPDGRLLRGHNQYAYDGKDYIALNEDLRSWTAADTAAQISQRKLEAARVAEQLRAYLE |
| | GECVEWLRRYLENGKDKLERADPPKTHVTHHPISDHEATLRCWALGFYPAEITLTWQRDGE |
| | DQTQDTELVETRPAGDRTFQKWAAVVVPSGEEQRYTCHVQHEGLPKPLTLRWEPSSQSTVPI |
| | VGIVAGLAVLAVVVIGAVVAAVMCRRKSSGGKGGSYSQAACSDSAQGSDVSLTA |
| | (HLA-B*40:01 full-length) |
| 49 | MRVMAPRTLILLLSGALALTETWACSHSMRYFDTAVSRPGRGEPRFISVGYVDDTQFVRFDS |
| | DAASPRGEPRAPWVEQEGPEYWDRETQKYKRQAQADRVNLRKLRGYYNQSEDGSHTLQW |
| | MYGCDLGPDGRLLRGYDQSAYDGKDYIALNEDLRSWTAADTAAQITQRKWEAAREAEQWR |
| | AYLEGTCVEWLRRYLENGKETLQRAEHPKTHVTHHPVSDHEATLRCWALGFYPAEITLTWQ |
| | RDGEDQTQDTELVETRPAGDGTFQKWAAVVVPSGEEQRYTCHVQHEGLPEPLTLRWEPSSQ |
| | PTIPIVGIVAGLAVLAVLAVLGAVMAVVMCRRKSSGGKGGSCSQAASSNSAQGSDESLIACK |
| | A |
| | (HLA-C*06:02 full-length) |
| 50 | MRVTAPRTVLLLLSGALALTETWAGSHSMRYFYTAMSRPGRGEPRFIAVGYVDDTQFVRFDS |
| | DAASPRMAPRAPWIEQEGPEYWDRETQISKTNTQTYRESLRNLRGYYNQSEAGSHTLQRMY |
| | GCDVGPDGRLLRGHDQSAYDGKDYIALNEDLSSWTAADTAAQITQRKWEAAREAEQWRAY |
| | LEGLCVEWLRRYLENGKETLQRADPPKTHVTHHPISDHEATLRCWALGFYPAEITLTWQRDG |
| | EDQTQDTELVETRPAGDRTFQKWAAVVVPSGEEQRYTCHVQHEGLPKPLTLRWEPSSQSTIPI |
| | VGIVAGLAVLAVVVIGAVVATVMCRRKSSGGKGGSYSQAASSDSAQGSDVSLTA |
| | (HLA-B*15:01 full-length) |
| 51 | MRVMAPRTLILLLSGALALTETWAGSHSMRYFYTAVSRPGRGEPHFIAVGYVDDTQFVRFDS |
| | DAASPRGEPRAPWVEQEGPEYWDRETQKYKRQAQTDRVSLRNLRGYYNQSEARSHIIQRMY |
| | GCDVGPDGRLLRGYDQYAYDGKDYIALNEDLRSWTAADTAAQITQRKWEAAREAEQLRAY |
| | LEGLCVEWLRRYLKNGKETLQRAEHPKTHVTHHPVSDHEATLRCWALGFYPAEITLTWQWD |
| | GEDQTQDTELVETRPAGDGTFQKWAAVVVPSGEEQRYTCHVQHEGLPEPLTLRWEPSSQPTI |
| | PIVGIVAGLAVLAVLAVLGAVVAVVMCRRKSSGGKGGSCSQAASSNSAQGSDESLIACKA |
| | (HLA-C*03:03 full-length) |
| 52 | MAVMAPRTLLLLLSGALALTQTWAGSHSMRYFSTSVSRPGSGEPRFIAVGYVDDTQFVRFDS |
| | DAASQRMEPRAPWIEQERPEYWDQETRNVKAQSQTDRVDLGTLRGYYNQSEAGSHTIQIMY |
| | GCDVGSDGRFLRGYEQHAYDGKDYIALNEDLRSWTAADMAAQITQRKWEAARWAEQLRA |
| | YLEGTCVEWLRRYLENGKETLQRTDPPKTHMTHHPISDHEATLRCWALGFYPAEITLTWQRD |
| | GEDQTQDTELVETRPAGDGTFQKWAAVVVPSGEEQRYTCHVQHEGLPKPLTLRWELSSQPTI |
| | PIVGIIAGLVLLGAVITGAVVAAVMWRRKSSDRKGGSYTQAASSDSAQGSDVSLTACKV |
| | (HLA-A*30:01 full-length) |
| 53 | MRVTAPRTLLLLLWGAVALTETWAGSHSMRYFYTAMSRPGRGEPRFITVGYVDDTQFVRFD |
| | SDATSPRMAPRAPWIEQEGPEYWDRETQISKTNTQTYRENLRTALRYYNQSEAGSHTWQTM |
| | YGCDLGPDGRLLRGHNQLAYDGKDYIALNEDLSSWTAADTAAQITQLKWEAARVAEQLRA |
| | YLEGECVEWLRRYLENGKETLQRADPPKTHVTHHPISDHEATLRCWALGFYPAEITLTWQRD |
| | GEDQTQDTELVETRPAGDRTFQKWAAVVVPSGEEQRYTCHVQHEGLPKPLTLRWEPSSQST |
| | VPIVGIVAGLAVLAVVVIGAVVAAVMCRRKSSGGKGGSYSQAACSDSAQGSDVSLTA |
| | (HLA-B*13:02 full-length) |
| 54 | MRVMAPRTLILLLSGALALTETWACSHSMRYFYTAVSRPGRGEPRFIAVGYVDDTQFVRFDS |
| | DAASPRGEPRAPWVEQEGPEYWDRETQKYKRQAQADRVSLRNLRGYYNQSEAGSHTLQW |
| | MYGCDLGPDGRLLRGYDQSAYDGKDYIALNEDLRSWTAADTAAQITQRKWEAAREAEQWR |
| | AYLEGTCVEWLRRYLENGKETLQRAEHPKTHVTHHPVSDHEATLRCWALGFYPAEITLTWQ |
| | RDGEDQTQDTELVETRPAGDGTFQKWAAVVVPSGEEQRYTCHVQHEGLPEPLTLRWEPSSQ |
| | PTIPIVGIVAGLAVLAVLAVLGAVMAVVMCRRKSSGGKGGSCSQAASSNSAQGSDESLIACK |
| | A |
| | (HLA-C*12:03 full-length) |
| 55 | MAVMAPRTLVLLLSGALALTQTWAGSHSMRYFYTSVSRPGRGEPRFIAVGYVDDTQFVRFD |
| | SDAASQRMEPRAPWIEQEGPEYWDRNTRNVKAHSQTDRANLGTLRGYYNQSEDGSHTIQRM |
| | YGCDVGPDGRFLRGYQQDAYDGKDYIALNEDLRSWTAADMAAQITQRKWETAHEAEQWR |
| | AYLEGRCVEWLRRYLENGKETLQRTDAPKTHMTHHAVSDHEATLRCWALSFYPAEITLTWQ |
| | RDGEDQTQDTELVETRPAGDGTFQKWASVVVPSGQEQRYTCHVQHEGLPKPLTLRWEPSSQ |
| | PTIPIVGIIAGLVLFGAVIAGAVVAAVMWRRKSSDRKGGSYSQAASSDSAQGSDMSLTACKV |
| | (HLA-A*26:01 full-length) |
| 56 | MLVMAPRTVLLLLSAALALTETWAGSHSMRYFYTSVSRPGRGEPRFISVGYVDDTQFVRFDS |
| | DAASPREEPRAPWIEQEGPEYWDRNTQICKTNTQTYRENLRIALRYYNQSEAGSHTLQRMYG |
| | CDVGPDGRLLRGHNQFAYDGKDYIALNEDLSSWTAADTAAQITQRKWEAARVAEQLRTYLE |
| | GTCVEWLRRYLENGKETLQRADPPKTHVTHHPISDHEATLRCWALGFYPAEITLTWQRDGED |
| | QTQDTELVETRPAGDRTFQKWAAVVVPSGEEQRYTCHVQHEGLPKPLTLRWEPSSQSTVPIV |
| | GIVAGLAVLAVVVIGAVVAAVMCRRKSSGGKGGSYSQAASSDSAQGSDVSLTA |
| | (HLA-B*38:01 full-length) |
| 57 | MLVMAPRTVLLLLSAALALTETWAGSHSMRYFYTAVSRPGRGEPRFISVGYVDDTQFVRFDS |
| | DAASPREEPRAPWIEQEGPEYWDRNTQICKTNTQTDRESLRNLRGYYNQSEAGSHTLQWMY |
| | GCDVGPDGRLLRGYNQFAYDGKDYIALNEDLSSWTAADTAAQITQRKWEAAREAEQLRAY |
| | LEGTCVEWLRRHLENGKETLQRADPPKTHVTHHPISDHEATLRCWALGFYPAEITLTWQRDG |
| | EDQTQDTELVETRPAGDRTFQKWAAVVVPSGEEQRYTCHVQHEGLPKPLTLRWEPSSQSTVP |
| | IVGIVAGLAVLAVVVIGAVVAAVMCRRKSSGGKGGSYSQAASSDSAQGSDVSLTA |
| | (HLA-B*14:02 full-length) |
| 58 | MAVMAPRTLLLLLLGALALTQTWAGSHSMRYFTTSVSRPGRGEPRFIAVGYVDDTQFVRFDS |
| | DAASQRMEPRAPWIEQEGPEYWDRNTRNVKAHSQIDRVDLGTLRGYYNQSEAGSHTIQMM |
| | YGCDVGSDGRFLRGYQQDAYDGKDYIALNEDLRSWTAADMAAQITQRKWEAARVAEQLR |
| | AYLEGTCVEWLRRHLENGKETLQRTDPPRTHMTHHAVSDHEATLRCWALSFYPAEITLTWQ |
| | RDGEDQTQDTELVETRPAGDGTFQKWASVVVPSGQEQRYTCHVQHEGLPKPLTLRWEPSSQ |
| | PTIPIVGIIAGLVLFGAVFAGAVVAAVRWRRKSSDRKGGSYSQAASSDSAQGSDMSLTACKV |
| | (HLA-A*33:01 full-length) |
| 59 | MAVMAPRTLVLLLSGALALTQTWAGSHSMRYFSTSVSRPGRGEPRFIAVGYVDDTQFVRFDS |
| | DAASQRMEPRAPWIEQEGPEYWDEETGKVKAHSQTDRENLRIALRYYNQSEAGSHTLQMMF |
| | GCDVGSDGRFLRGYHQYAYDGKDYIALKEDLRSWTAADMAAQITQRKWEAARVAEQLRA |
| | YLEGTCVDGLRRYLENGKETLQRTDPPKTHMTHHPISDHEATLRCWALGFYPAEITLTWQRD |
| | GEDQTQDTELVETRPAGDGTFQKWAAVVVPSGEEQRYTCHVQHEGLPKPLTLRWEPSSQPT |
| | VHIVGIIAGLVLLGAVITGAVVAAVMWRRNSSDRKGGSYSQAASSDSAQGSDVSLTACKV |
| | (HLA-A*23:01 full-length) |
| 60 | MAVMAPRTLVLLLSGALALTQTWAGSHSMRYFYTSVSRPGRGEPRFIAVGYVDDTQFVRFD |
| | SDAASQRMEPRAPWIEQEGPEYWDRNTRNVKAHSQTDRESLRIALRYYNQSEDGSHTIQRM |
| | YGCDVGPDGRFLRGYQQDAYDGKDYIALNEDLRSWTAADMAAQITQRKWETAHEAEQWR |
| | AYLEGRCVEWLRRYLENGKETLQRTDAPKTHMTHHAVSDHEATLRCWALSFYPAEITLTWQ |
| | RDGEDQTQDTELVETRPAGDGTFQKWASVVVPSGQEQRYTCHVQHEGLPKPLTLRWEPSSQ |
| | PTIPIVGIIAGLVLFGAVIAGAVVAAVMWRRKSSDRKGGSYSQAASSDSAQGSDMSLTACKV |
| | (HLA-A*25:01 full-length) |
| 61 | MRVTAPRTLLLLLWGAVALTETWAGSHSMRYFHTSVSRPGRGEPRFISVGYVDGTQFVRFDS |
| | DAASPRTEPRAPWIEQEGPEYWDRNTQISKTNTQTYRESLRNLRGYYNQSEAGSHTLQRMYG |
| | CDVGPDGRLLRGHDQSAYDGKDYIALNEDLSSWTAADTAAQITQRKWEAARVAEQLRAYL |
| | EGTCVEWLRRHLENGKETLQRADPPKTHVTHHPISDHEATLRCWALGFYPAEITLTWQRDGE |
| | DQTQDTELVETRPAGDRTFQKWAAVVVPSGEEQRYTCHVQHEGLPKPLTLRWEPSSQSTIPIV |
| | GIVAGLAVLAVVVIGAVVATVMCRRKSSGGKGGSYSQAASSDSAQGSDVSLTA |
| | (HLA-B*18:01 full-length) |
| 62 | MRVTAPRTLLLLLWGAVALTETWAGSHSMRYFHTSVSRPGRGEPRFISVGYVDDTQFVRFDS |
| | DAASPRTEPRAPWIEQEGPEYWDRETQISKTNTQTYREDLRTLLRYYNQSEAGSHTIQRMSGC |
| | DVGPDGRLLRGYNQFAYDGKDYIALNEDLSSWTAADTAAQITQRKWEAARVAEQDRAYLE |
| | GTCVEWLRRYLENGKETLQRADPPKTHVTHHPISDHEATLRCWALGFYPAEITLTWQRDGED |
| | QTQDTELVETRPAGDRTFQKWAAVVVPSGEEQRYTCHVQHEGLPKPLTLRWEPSSQSTIPIVG |
| | IVAGLAVLAVVVIGAVVATVMCRRKSSGGKGGSYSQAASSDSAQGSDVSLTA |
| | (HLA-B*37:01 full length) |
| 63 | MRVTAPRTVLLLLWGAVALTETWAGSHSMRYFYTAMSRPGRGEPRFIAVGYVDDTQFVRFD |
| | SDAASPRTEPRAPWIEQEGPEYWDRNTQIFKTNTQTYRENLRIALRYYNQSEAGSHTWQTMY |
| | GCDVGPDGRLLRGHNQYAYDGKDYIALNEDLSSWTAADTAAQITQRKWEAAREAEQLRAY |
| | LEGLCVEWLRRHLENGKETLQRADPPKTHVTHHPVSDHEATLRCWALGFYPAEITLTWQRD |
| | GEDQTQDTELVETRPAGDRTFQKWAAVVVPSGEEQRYTCHVQHEGLPKPLTLRWEPSSQSTI |
| | PIVGIVAGLAVLAVVVIGAVVATVMCRRKSSGGKGGSYSQAASSDSAQGSDVSLTA |
| | (HLA-B*51:01 full-length) |
| 64 | MRVMAPRTLILLLSGALALTETWACSHSMRYFSTSVSRPGRGEPRFIAVGYVDDTQFVRFDS |
| | DAASPRGEPRAPWVEQEGPEYWDRETQKYKRQAQTDRVSLRNLRGYYNQSEAGSHTLQWM |
| | FGCDLGPDGRLLRGYDQSAYDGKDYIALNEDLRSWTAADTAAQITQRKWEAAREAEQRRA |
| | YLEGTCVEWLRRYLENGKETLQRAEHPKTHVTHHPVSDHEATLRCWALGFYPAEITLTWQW |
| | DGEDQTQDTELVETRPAGDGTFQKWAAVVVPSGEEQRYTCHVQHEGLPEPLTLRWEPSSQP |
| | TIPIVGIVAGLAVLAVLAVLGAVVAVVMCRRKSSGGKGGSCSQAASSNSAQGSDESLIACKA |
| | (HLA-C*14:02 full-length) |
| 65 | MRVMAPRTLLLLLSGALALTETWACSHSMRYFYTAVSRPGRGEPHFIAVGYVDDTQFVRFDS |
| | DAASPRGEPRAPWVEQEGPEYWDRETQNYKRQAQTDRVNLRKLRGYYNQSEAGSHIIQRMY |
| | GCDLGPDGRLLRGHDQLAYDGKDYIALNEDLRSWTAADTAAQITQRKWEAAREAEQLRAY |
| | LEGTCVEWLRRYLENGKETLQRAEHPKTHVTHHPVSDHEATLRCWALGFYPAEITLTWQRD |
| | GEDQTQDTELVETRPAGDGTFQKWAAVVVPSGEEQRYTCHVQHEGLPEPLTLRWEPSSQPTI |
| | PIVGIVAGLAVLAVLAVLGAVMAVVMCRRKSSGGKGGSCSQAASSNSAQGSDESLIACKA |
| | (HLA-C*15:02 full-length) |
| 66 | MRVMAPRTLLLLLSGALALTETWACSHSMRYFYTAVSRPSRGEPHFIAVGYVDDTQFVRFDS |
| | DAASPRGEPRAPWVEQEGPEYWDRETQKYKRQAQTDRVNLRKLRGYYNQSEAGSHTLQRM |
| | YGCDLGPDGRLLRGYDQSAYDGKDYIALNEDLRSWTAADTAAQITQRKWEAAREAEQWRA |
| | YLEGECVEWLRRYLENGKETLQRAEHPKTHVTHHPVSDHEATLRCWALGFYPTEITLTWQR |
| | DGEDQTQDTELVETRPAGDGTFQKWAAVVVPSGEEQRYTCHVQHEGLPEPLTLRWEPSSQP |
| | TIPIVGIVAGLAVLAVLAVLGAVVAVVMCRRKSSGGKGGSCSQAASSNSAQGSDESLIACKA |
| | (HLA-C*02:02 full-length) |
| 67 | MRVTAPRTLLLLLWGAVALTETWAGSHSMRYFHTSVSRPGRGEPRFITVGYVDDTLFVRFDS |
| | DAASPREEPRAPWIEQEGPEYWDRETQICKAKAQTDREDLRTLLRYYNQSEAGSHTLQNMY |
| | GCDVGPDGRLLRGYHQDAYDGKDYIALNEDLSSWTAADTAAQITQRKWEAARVAEQLRAY |
| | LEGECVEWLRRYLENGKETLQRADPPKTHVTHHPISDHEATLRCWALGFYPAEITLTWQRDG |
| | EDQTQDTELVETRPAGDRTFQKWAAVVVPSGEEQRYTCHVQHEGLPKPLTLRWEPSSQSTVP |
| | IVGIVAGLAVLAVVVIGAVVAAVMCRRKSSGGKGGSYSQAACSDSAQGSDVSLTA |
| | (HLA-B*27:05 full-length) |
| 68 | MAVMAPRTLLLLLLGALALTQTWAGSHSMRYFTTSVSRPGRGEPRFIAVGYVDDTQFVRFDS |
| | DAASQRMEPRAPWIEQERPEYWDQETRNVKAHSQIDRVDLGTLRGYYNQSEAGSHTIQMMY |
| | GCDVGSDGRFLRGYQQDAYDGKDYIALNEDLRSWTAADMAAQITQRKWEAARVAEQLRA |
| | YLEGTCVEWLRRYLENGKETLQRTDPPKTHMTHHAVSDHEATLRCWALSFYPAEITLTWQR |
| | DGEDQTQDTELVETRPAGDGTFQKWASVVVPSGQEQRYTCHVQHEGLPKPLTLRWEPSSQP |
| | TIPIVGIIAGLVLFGAVFAGAVVAAVRWRRKSSDRKGGSYSQAASSDSAQGSDMSLTACKV |
| | (HLA-A*31:01 full-length) |
| 69 | MAVMAPRTLLLLLSGALALTQTWAGSHSMRYFSTSVSRPGSGEPRFIAVGYVDDTQFVRFDS |
| | DAASQRMEPRAPWIEQERPEYWDQETRNVKAHSQTDRENLGTLRGYYNQSEAGSHTIQIMY |
| | GCDVGSDGRFLRGYEQHAYDGKDYIALNEDLRSWTAADMAAQITQRKWEAARRAEQLRAY |
| | LEGTCVEWLRRYLENGKETLQRTDPPKTHMTHHPISDHEATLRCWALGFYPAEITLTWQRDG |
| | EDQTQDTELVETRPAGDGTFQKWAAVVVPSGEEQRYTCHVQHEGLPKPLTLRWELSSQPTIPI |
| | VGIIAGLVLLGAVITGAVVAAVMWRRKSSDRKGGSYTQAASSDSAQGSDVSLTACKV |
| | (HLA-A*30:02 full-length) |
| 70 | MLVMAPRTVLLLLSAALALTETWAGSHSMRYFYTSVSRPGRGEPRFISVGYVDDTQFVRFDS |
| | DAASPREEPRAPWIEQEGPEYWDRNTQIYKAQAQTDRESLRNLRGYYNQSEAGSHTLQSMY |
| | GCDVGPDGRLLRGHNQYAYDGKDYIALNEDLRSWTAADTAAQITQRKWEAARVAEQDRAY |
| | LEGTCVEWLRRYLENGKDTLERADPPKTHVTHHPISDHEATLRCWALGFYPAEITLTWQRDG |
| | EDQTQDTELVETRPAGDRTFQKWAAVVVPSGEEQRYTCHVQHEGLPKPLTLRWEPSSQSTVP |
| | IVGIVAGLAVLAVVVIGAVVAAVMCRRKSSGGKGGSYSQAACSDSAQGSDVSLTA |
| | (HLA-B*42:01 full-length) |
| 71 | MRVMAPQALLLLLSGALALIETWAGSHSMRYFYTAVSRPGRGEPRFIAVGYVDDTQFVRFDS |
| | DAASPRGEPRAPWVEQEGPEYWDRETQKYKRQAQADRVNLRKLRGYYNQSEAGSHTIQRM |
| | YGCDLGPDGRLLRGYNQFAYDGKDYIALNEDLRSWTAADTAAQISQRKLEAAREAEQLRAY |
| | LEGECVEWLRGYLENGKETLQRAERPKTHVTHHPVSDHEATLRCWALGFYPAEITLTWQRD |
| | GEDQTQDTELVETRPAGDGTFQKWAAVVVPSGQEQRYTCHVQHEGLQEPCTLRWKPSSQPT |
| | IPNLGIVSGPAVLAVLAVLAVLAVLGAVVAAVIHRRKSSGGKGGSCSQAASSNSAQGSDESLI |
| | ACKA |
| | (HLA-C*17:01 full-length) |
| 72 | MRVTAPRTVLLLLWGAVALTETWAGSHSMRYFYTAMSRPGRGEPRFIAVGYVDDTQFVRFD |
| | SDAASPRTEPRAPWIEQEGPEYWDRNTQIFKTNTQTYRESLRNLRGYYNQSEAGSHIIQRMYG |
| | CDLGPDGRFLRGHNQYAYDGKDYIALNEDLSSWTAADTAAQITQRKWEAARVAEQLRAYL |
| | EGLCVEWLRRYLENGKETLQRADPPKTHVTHHPVSDHEATLRCWALGFYPAEITLTWQRDG |
| | EDQTQDTELVETRPAGDRTFQKWAAVVVPSGEEQRYTCHVQHEGLPKPLTLRWEPSSQSTIPI |
| | VGIVAGLAVLAVVVIGAVVATVMCRRKSSGGKGGSYSQAASSDSAQGSDVSLTA |
| | (HLA-B*35:02 full-length) |
| 73 | MLVMAPRTVLLLLSAALALTETWAGSHSMRYFYTSVSRPGRGEPRFISVGYVDDTQFVRFDS |
| | DAASPREEPRAPWIEQEGPEYWDRNTQICKTNTQTDRESLRNLRGYYNQSEAGSHTWQTMY |
| | GCDVGPDGRLLRGHNQFAYDGKDYIALNEDLSSWTAADTAAQITQRKWEAARVAEQLRTY |
| | LEGTCVEWLRRYLENGKETLQRADPPKTHVTHHPISDHEATLRCWALGFYPAEITLTWQRDG |
| | EDQTQDTELVETRPAGDRTFQKWAAVVVPSGEEQRYTCHVQHEGLPKPLTLRWEPSSQSTVP |
| | IVGIVAGLAVLAVVVIGAVVAAVMCRRKSSGGKGGSYSQAASSDSAQGSDVSLTA |
| | (HLA-B*39:06 full-length) |
| 74 | MRVMAPRTLILLLSGALALTETWAGSHSMRYFYTAVSRPGRGEPHFIAVGYVDDTQFVRFDS |
| | DAASPRGEPRAPWVEQEGPEYWDRETQKYKRQAQTDRVSLRNLRGYYNQSEAGSHILQRM |
| | YGCDVGPDGRLLRGYDQSAYDGKDYIALNEDLRSWTAADTAAQITQRKWEAAREAEQLRA |
| | YLEGLCVEWLRRYLKNGKETLQRAEHPKTHVTHHPVSDHEATLRCWALGFYPAEITLTWQW |
| | DGEDQTQDTELVETRPAGDGTFQKWAAVVVPSGEEQRYTCHVQHEGLPEPLTLRWEPSSQP |
| | TIPIVGIVAGLAVLAVLAVLGAVVAVVMCRRKSSGGKGGSCSQAASSNSAQGSDESLIACKA |
| | (HLA-C*03:02 full-length) |
| 75 | MRVTAPRTVLLLLWGAVALTETWAGSHSMRYFYTAMSRPGRGEPRFIAVGYVDDTQFVRFD |
| | SDAASPRTEPRAPWIEQEGPEYWDGETRNMKASAQTYRENLRIALRYYNQSEAGSHIIQRMY |
| | GCDLGPDGRLLRGHDQSAYDGKDYIALNEDLSSWTAADTAAQITQRKWEAARVAEQLRAY |
| | LEGLCVEWLRRYLENGKETLQRADPPKTHVTHHPVSDHEATLRCWALGFYPAEITLTWQRD |
| | GEDQTQDTELVETRPAGDRTFQKWAAVVVPSGEEQRYTCHVQHEGLPKPLTLRWEPSSQSTI |
| | PIVGIVAGLAVLAVVVIGAVVATVMCRRKSSGGKGGSYSQAASSDSAQGSDVSLTA |
| | (HLA-B*58:01 full-length) |
| 76 | MAVMAPRTLLLLLLGALALTQTWAGSHSMRYFTTSVSRPGRGEPRFIAVGYVDDTQFVRFDS |
| | DAASQRMEPRAPWIEQEGPEYWDRNTRNVKAHSQIDRVDLGTLRGYYNQSEAGSHTIQMM |
| | YGCDVGSDGRFLRGYQQDAYDGKDYIALNEDLRSWTAADMAAQITQRKWEAARVAEQLR |
| | AYLEGTCVEWLRRYLENGKETLQRTDPPKTHMTHHAVSDHEATLRCWALSFYPAEITLTWQ |
| | RDGEDQTQDTELVETRPAGDGTFQKWASVVVPSGQEQRYTCHVQHEGLPKPLTLRWEPSSQ |
| | PTIPIVGIIAGLVLFGAVFAGAVVAAVRWRRKSSDRKGGSYSQAASSDSAQGSDMSLTACKV |
| | (HLA-A*33:03 full-length) |
| 77 | MAVMAPRTLVLLLSGALALTQTWAGSHSMRYFYTSMSRPGRGEPRFIAVGYVDDTQFVRFD |
| | SDAASQRMEPRAPWIEQEGPEYWDRNTRNVKAQSQTDRVDLGTLRGYYNQSEAGSHTIQRM |
| | YGCDVGPDGRFLRGYHQYAYDGKDYIALKEDLRSWTAADMAAQTTKHKWEAAHVAEQW |
| | RAYLEGTCVEWLRRYLENGKETLQRTDAPKTHMTHHAVSDHEATLRCWALSFYPAEITLTW |
| | QRDGEDQTQDTELVETRPAGDGTFQKWVAVVVPSGQEQRYTCHVQHEGLPKPLTLRWEPSS |
| | QPTIPIVGIIAGLVLFGAVITGAVVAAVMWRRKSSDRKGGSYSQAASSDSAQGSDVSLTACKV |
| | (HLA-A*68:02 full-length) |
| 78 | MRVMAPRTLILLLSGALALTETWACSHSMKYFFTSVSRPGRGEPRFISVGYVDDTQFVRFDSD |
| | AASPRGEPRAPWVEQEGPEYWDRETQKYKRQAQTDRVSLRNLRGYYNQSEAGSHTLQWMC |
| | GCDLGPDGRLLRGYDQYAYDGKDYIALNEDLRSWTAADTAAQITQRKWEAAREAEQRRAY |
| | LEGTCVEWLRRYLENGKETLQRAEHPKTHVTHHPVSDHEATLRCWALGFYPAEITLTWQWD |
| | GEDQTQDTELVETRPAGDGTFQKWAAVMVPSGEEQRYTCHVQHEGLPEPLTLRWEPSSQPTI |
| | PIVGIVAGLAVLAVLAVLGAVVAVVMCRRKSSGGKGGSCSQAASSNSAQGSDESLIACKA |
| | (HLA-C*01:02 full-length) |
| 79 | MRVMAPRALLLLLSGGLALTETWACSHSMRYFDTAVSRPGRGEPRFISVGYVDDTQFVRFDS |
| | DAASPRGEPRAPWVEQEGPEYWDRETQKYKRQAQADRVSLRNLRGYYNQSEDGSHTFQRM |
| | YGCDLGPDGRLLRGYDQFAYDGKDYIALNEDLRSWTAADTAAQITQRKLEAARAAEQDRA |
| | YLEGTCVEWLRRYLENGKKTLQRAEPPKTHVTHHPLSDHEATLRCWALGFYPAEITLTWQR |
| | DGEDQTQDTELVETRPAGDGTFQKWAAVVVPSGQEQRYTCHMQHEGLQEPLTLSWEPSSQP |
| | TIPIMGIVAGLAVLVVLAVLGAVVTAMMCRRKSSGGKGGSCSQAACSNSAQGSDESLITCKA |
| | (HLA-C*07:04 full-length) |
| 80 | MAVMAPRTLVLLLSGALALTQTWAGSHSMRYFYTSVSRPGRGEPRFIAVGYVDDTQFVRFD |
| | SDAASQRMEPRAPWIEQEGPEYWDRNTRNVKAQSQTDRVDLGTLRGYYNQSEAGSHTIQM |
| | MYGCDVGSDGRFLRGYRQDAYDGKDYIALKEDLRSWTAADMAAQTTKHKWEAAHVAEQ |
| | WRAYLEGTCVEWLRRYLENGKETLQRTDAPKTHMTHHAVSDHEATLRCWALSFYPAEITLT |
| | WQRDGEDQTQDTELVETRPAGDGTFQKWVAVVVPSGQEQRYTCHVQHEGLPKPLTLRWEP |
| | SSQPTIPIVGIIAGLVLFGAVITGAVVAAVMWRRKSSDRKGGSYSQAASSDSAQGSDVSLTAC |
| | KV |
| | (HLA-A*68:01 full-length) |
| 81 | MAVMAPRTLLLLLLGALALTQTWAGSHSMRYFFTSVSRPGRGEPRFIAVGYVDDTQFVRFDS |
| | DAASQRMEPRAPWIEQEGPEYWDQETRNVKAHSQTDRESLRIALRYYNQSEAGSHTIQMMY |
| | GCDVGPDGRLLRGYQQDAYDGKDYIALNEDLRSWTAADMAAQITQRKWEAARVAEQLRA |
| | YLEGTCVEWLRRYLENGKETLQRTDAPKTHMTHHAVSDHEATLRCWALSFYPAEITLTWQR |
| | DGEDQTQDTELVETRPAGDGTFQKWASVVVPSGQEQRYTCHVQHEGLPKPLTLRWEPSSQP |
| | TIPIVGIIAGLVLFGAMFAGAVVAAVRWRRKSSDRKGGSYSQAASSDSAQGSDMSLTACKV |
| | (HLA-A*32:01 full-length) |
| 82 | MRVTAPRTVLLLLSAALALTETWAGSHSMRYFHTAMSRPGRGEPRFITVGYVDDTLFVRFDS |
| | DATSPRKEPRAPWIEQEGPEYWDRETQISKTNTQTYRENLRIALRYYNQSEAGSHTWQRMYG |
| | CDLGPDGRLLRGYNQLAYDGKDYIALNEDLSSWTAADTAAQITQRKWEAAREAEQLRAYLE |
| | GLCVEWLRRYLENGKETLQRADPPKTHVTHHPISDHEATLRCWALGFYPAEITLTWQRDGED |
| | QTQDTELVETRPAGDRTFQKWAAVVVPSGEEQRYTCHVQHEGLPKPLTLRWEPSSQSTIPIVG |
| | IVAGLAVLAVVVIGAVVATVMCRRKSSGGKGGSYSQAASSDSAQGSDVSLTA |
| | (HLA-B*49:01 full-length) |
| 83 | MRVTAPRTVLLLLWGAVALTETWAGSHSMRYFYTAMSRPGRGEPRFIAVGYVDDTQFVRFD |
| | SDAASPRTEPRAPWIEQEGPEYWDRNTQIFKTNTQTYRENLRIALRYYNQSEAGSHIIQRMYG |
| | CDLGPDGRLLRGHDQSAYDGKDYIALNEDLSSWTAADTAAQITQRKWEAARVAEQLRAYLE |
| | GLCVEWLRRYLENGKETLQRADPPKTHVTHHPVSDHEATLRCWALGFYPAEITLTWQRDGE |
| | DQTQDTELVETRPAGDRTFQKWAAVVVPSGEEQRYTCHVQHEGLPKPLTLRWEPSSQSTIPIV |
| | GIVAGLAVLAVVVIGAVVATVMCRRKSSGGKGGSYSQAASSDSAQGSDVSLTA |
| | (HLA-B*53:01 full-length) |
| 84 | MRVTAPRTVLLLLSAALALTETWAGSHSMRYFHTAMSRPGRGEPRFITVGYVDDTLFVRFDS |
| | DATSPRKEPRAPWIEQEGPEYWDRETQISKTNTQTYRESLRNLRGYYNQSEAGSHTWQRMY |
| | GCDLGPDGRLLRGYNQLAYDGKDYIALNEDLSSWTAADTAAQITQRKWEAAREAEQLRAY |
| | LEGLCVEWLRRYLENGKETLQRADPPKTHVTHHPISDHEATLRCWALGFYPAEITLTWQRDG |
| | EDQTQDTELVETRPAGDRTFQKWAAVVVPSGEEQRYTCHVQHEGLPKPLTLRWEPSSQSTIPI |
| | VGIVAGLAVLAVVVIGAVVATVMCRRKSSGGKGGSYSQAASSDSAQGSDVSLTA |
| | (HLA-B*50:01 full-length) |
| 85 | MAVMAPRTLVLLLSGALALTQTWAGSHSMRYFYTSVSRPGRGEPRFIAVGYVDDTQFVRFD |
| | SDAASRRMEPRAPWIEQEGPEYWDGETRKVKAHSQTHRVDLGTLRGYYNQSEAGSHTLQR |
| | MYGCDVGSDWRFLRGYHQYAYDGKDYIALKEDLRSWTAADMAAQTTKHKWEAAHVAEQ |
| | WRAYLEGTCVEWLRRYLENGKETLQRTDAPKTHMTHHAVSDHEATLRCWALSFYPAEITLT |
| | WQRDGEDQTQDTELVETRPAGDGTFQKWAAVVVPSGQEQRYTCHVQHEGLPKPLTLRWEP |
| | SSQPTIPIVGIIAGLVLFGAVITGAVVAAVMWRRKSSDRKGGSYSQAASSDSAQGSDVSLTAC |
| | KV |
| | (HLA-A*02:05 full-length) |
| 86 | MRVTAPRTLLLLLWGALALTETWAGSHSMRYFYTAMSRPGRGEPRFIAVGYVDDTQFVRFD |
| | SDAASPREEPRAPWIEQEGPEYWDRNTQIYKAQAQTDRESLRNLRGYYNQSEAGSHTWQTM |
| | YGCDLGPDGRLLRGHNQLAYDGKDYIALNEDLSSWTAADTAAQITQRKWEAAREAEQLRA |
| | YLEGTCVEWLRRYLENGKETLQRADPPKTHVTHHPISDHEATLRCWALGFYPAEITLTWQRD |
| | GEDQTQDTELVETRPAGDRTFQKWAAVVVPSGEEQRYTCHVQHEGLPKPLTLRWEPSSQSTI |
| | PIVGIVAGLAVLAVVVIGAVVATVMCRRKSSGGKGGSYSQAASSDSAQGSDVSLTA |
| | (HLA-B*55:01 full-length) |
| 87 | MRVTAPRTVLLLLSAALALTETWAGSHSMRYFHTAMSRPGRGEPRFITVGYVDDTLFVRFDS |
| | DATSPRKEPRAPWIEQEGPEYWDRETQISKTNTQTYRESLRNLRGYYNQSEAGSHTWQRMY |
| | GCDLGPDGRLLRGYNQLAYDGKDYIALNEDLSSWTAADTAAQITQRKWEAARVAEQDRAY |
| | LEGLCVESLRRYLENGKETLQRADPPKTHVTHHPISDHEATLRCWALGFYPAEITLTWQRDG |
| | EDQTQDTELVETRPAGDRTFQKWAAVVVPSGEEQRYTCHVQHEGLPKPLTLRWEPSSQSTIPI |
| | VGIVAGLAVLAVVVIGAVVATVMCRRKSSGGKGGSYSQAASSDSAQGSDVSLTA |
| | (HLA-B*45:01 full-length) |
| 88 | MRVTAPRTVLLLLWGAVALTETWAGSHSMRYFYTAMSRPGRGEPRFIAVGYVDDTQFVRFD |
| | SDAASPRTEPRAPWIEQEGPEYWDRETQISKTNTQTYRENLRIALRYYNQSEAGSHTWQTMY |
| | GCDVGPDGRLLRGHNQYAYDGKDYIALNEDLSSWTAADTAAQITQRKWEAAREAEQLRAY |
| | LEGLCVEWLRRHLENGKETLQRADPPKTHVTHHPVSDHEATLRCWALGFYPAEITLTWQRD |
| | GEDQTQDTELVETRPAGDRTFQKWAAVVVPSGEEQRYTCHVQHEGLPKPLTLRWEPSSQSTI |
| | PIVGIVAGLAVLAVVVIGAVVATVMCRRKSSGGKGGSYSQAASSDSAQGSDVSLTA |
| | (HLA-B*52:01 full-length) |
| 89 | MRVMAPRTLILLLSGALALTETWACSHSMRYFYTAVSRPGRGEPRFIAVGYVDDTQFVRFDS |
| | DAASPRGEPRAPWVEQEGPEYWDRETQKYKRQAQADRVSLRNLRGYYNQSEAGSHTLQRM |
| | YGCDLGPDGRLLRGYDQSAYDGKDYIALNEDLRSWTAADTAAQITQRKWEAAREAEQWRA |
| | YLEGTCVEWLRRYLENGKETLQRAEHPKTHVTHHPVSDHEATLRCWALGFYPAEITLTWQR |
| | DGEDQTQDTELVETRPAGDGTFQKWAAVVVPSGEEQRYTCHVQHEGLPEPLTLRWEPSSQP |
| | TIPIVGIVAGLAVLAVLAVLGAVMAVVMCRRKSSGGKGGSCSQAASSNSAQGSDESLIACKA |
| | (HLA-C*12:02 full-length) |
| 90 | MRVTAPRTVLLLLWGAVALTETWAGSHSMRYFYTAMSRPGRGEPRFIAVGYVDDTQFVRFD |
| | SDAASPRTEPRAPWIEQEGPEYWDRNTQIFKTNTQTYRESLRNLRGYYNQSEAGSHIIQRMYG |
| | CDLGPDGRLLRGHDQFAYDGKDYIALNEDLSSWTAADTAAQITQRKWEAARVAEQLRAYLE |
| | GLCVEWLRRYLENGKETLQRADPPKTHVTHHPVSDHEATLRCWALGFYPAEITLTWQRDGE |
| | DQTQDTELVETRPAGDRTFQKWAAVVVPSGEEQRYTCHVQHEGLPKPLTLRWEPSSQSTIPIV |
| | GIVAGLAVLAVVVIGAVVATVMCRRKSSGGKGGSYSQAASSDSAQGSDVSLTA |
| | (HLA-B*35:03 full-length) |
| 91 | MRVTAPRTLLLLLWGAVALTETWAGSHSMRYFHTSVSRPGRGEPRFITVGYVDDTLFVRFDS |
| | DATSPRKEPRAPWIEQEGPEYWDRETQISKTNTQTYRESLRNLRGYYNQSEAGSHTLQSMYG |
| | CDVGPDGRLLRGHNQYAYDGKDYIALNEDLRSWTAADTAAQITQRKWEAARVAEQLRAYL |
| | EGECVEWLRRYLENGKETLQRADPPKTHVTHHPISDHEATLRCWALGFYPAEITLTWQRDGE |
| | DQTQDTELVETRPAGDRTFQKWAAVVVPSGEEQRYTCHVQHEGLPKPLTLRWEPSSQSTVPI |
| | VGIVAGLAVLAVVVIGAVVAAVMCRRKSSGGKGGSYSQAACSDSAQGSDVSLTA |
| | (HLA-B*40:02 full-length) |
| 92 | MRVTAPRTVLLLLSGALALTETWAGSHSMRYFYTAMSRPGRGEPRFISVGYVDDTQFVRFDS |
| | DAASPREEPRAPWIEQEGPEYWDRETQISKTNTQTYRESLRNLRGYYNQSEAGSHTLQRMYG |
| | CDVGPDGRLLRGHDQSAYDGKDYIALNEDLSSWTAADTAAQITQRKWEAAREAEQLRAYLE |
| | GLCVEWLRRYLENGKETLQRADPPKTHVTHHPISDHEATLRCWALGFYPAEITLTWQRDGED |
| | QTQDTELVETRPAGDRTFQKWAAVVVPSGEEQRYTCHVQHEGLPKPLTLRWEPSSQSTIPIVG |
| | IVAGLAVLAVVVIGAVVATVMCRRKSSGGKGGSYSQAASSDSAQGSDVSLTA |
| | (HLA-B*15:03 full-length) |
| 93 | MAVMAPRTLLLLLLGALALTQTRAGSHSMRYFFTSVSRPGRGEPRFIAVGYVDDTQFVRFDS |
| | DAASQRMEPRAPWIEQEGPEYWDQETRNVKAHSQTDRVDLGTLRGYYNQSEAGSHTIQMM |
| | YGCDVGPDGRLLRGYQQDAYDGKDYIALNEDLRSWTAADMAAQITQRKWEAARVAEQLR |
| | AYLEGTCVEWLRRYLENGKETLQRTDAPKTHMTHHAVSDHEATLRCWALSFYPAEITLTWQ |
| | RDGEDQTQDTELVETRPAGDGTFQKWASVVVPSGQEQRYTCHVQHEGLPKPLTLRWEPSSQ |
| | PTIPIVGIIAGLVLFGAMFAGAVVAAVRWRRKSSDRKGGSYSQAASSDSAQGSDMSLTACKV |
| | (HLA-A*74:01 full-length) |
| 94 | GSHSMRYFFTSVSRPGRGEPRFIAVGYVDDTQFVRFDSDAASQKMEPRAPWIEQEGPEYWDQ |
| | ETRNMKAHSQTDRANLGTLRGYYNQSEDGSHTIQIMYGCDVGPDGRFLRGYRQDAYDGKD |
| | YIALNEDLRSWTAADMAAQITKRKWEAVHAAEQRRVYLEGRCVDGLRRYLENGKETLQRT |
| | DPPKTHMTHHPISDHEATLRCWALGFYPAEITLTWQRDGEDQTQDTELVETRPAGDGTFQK |
| | WAAVVVPSGEEQRYTCHVQHEGLPKPLTLRWELSS |
| | (HLA-A*01:01 soluble) |
| 95 | GSHSMRYFFTSVSRPGRGEPRFIAVGYVDDTQFVRFDSDAASQRMEPRAPWIEQEGPEYWDQ |
| | ETRNVKAQSQTDRVDLGTLRGYYNQSEAGSHTIQIMYGCDVGSDGRFLRGYRQDAYDGKD |
| | YIALNEDLRSWTAADMAAQITKRKWEAAHEAEQLRAYLDGTCVEWLRRYLENGKETLQRT |
| | DPPKTHMTHHPISDHEATLRCWALGFYPAEITLTWQRDGEDQTQDTELVETRPAGDGTFQK |
| | WAAVVVPSGEEQRYTCHVQHEGLPKPLTLRWELSS |
| | (HLA-A*03:01 soluble) |
| 96 | GSHSMRYFYTSVSRPGRGEPRFIAVGYVDDTQFVRFDSDAASQRMEPRAPWIEQEGPEYWDQ |
| | ETRNVKAQSQTDRVDLGTLRGYYNQSEDGSHTIQIMYGCDVGPDGRFLRGYRQDAYDGKD |
| | YIALNEDLRSWTAADMAAQITKRKWEAAHAAEQQRAYLEGRCVEWLRRYLENGKETLQRT |
| | DPPKTHMTHHPISDHEATLRCWALGFYPAEITLTWQRDGEDQTQDTELVETRPAGDGTFQK |
| | WAAVVVPSGEEQRYTCHVQHEGLPKPLTLRWELSS |
| | (HLA-A*11:01 soluble) |
| 97 | GSHSMRYFSTSVSRPGRGEPRFIAVGYVDDTQFVRFDSDAASQRMEPRAPWIEQEGPEYWDE |
| | ETGKVKAHSQTDRENLRIALRYYNQSEAGSHTLQMMFGCDVGSDGRFLRGYHQYAYDGKD |
| | YIALKEDLRSWTAADMAAQITKRKWEAAHVAEQQRAYLEGTCVDGLRRYLENGKETLQRT |
| | DPPKTHMTHHPISDHEATLRCWALGFYPAEITLTWQRDGEDQTQDTELVETRPAGDGTFQK |
| | WAAVVVPSGEEQRYTCHVQHEGLPKPLTLRWEPSS |
| | (HLA-A*24:02 soluble) |
| 98 | GSHSMRYFYTSVSRPGRGEPRFISVGYVDDTQFVRFDSDAASPREEPRAPWIEQEGPEYWDRN |
| | TQIYKAQAQTDRESLRNLRGYYNQSEAGSHTLQSMYGCDVGPDGRLLRGHDQYAYDGKDYI |
| | ALNEDLRSWTAADTAAQITQRKWEAAREAEQRRAYLEGECVEWLRRYLENGKDKLERADP |
| | PKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*07:02 soluble) |
| 99 | GSHSMRYFSTSVSWPGRGEPRFIAVGYVDDTQFVRFDSDAASPRGEPREPWVEQEGPEYWDR |
| | ETQKYKRQAQADRVNLRKLRGYYNQSEDGSHTLQRMFGCDLGPDGRLLRGYNQFAYDGKD |
| | YIALNEDLRSWTAADTAAQITQRKWEAAREAEQRRAYLEGTCVEWLRRYLENGKETLQRAE |
| | HPKTHVTHHPVSDHEATLRCWALGFYPAEITLTWQWDGEDQTQDTELVETRPAGDGTFQK |
| | WAAVVVPSGEEQRYTCHVQHEGLPEPLTLRWKPSS |
| | (HLA-C*04:01 soluble) |
| 100 | CSHSMRYFDTAVSRPGRGEPRFISVGYVDDTQFVRFDSDAASPRGEPRAPWVEQEGPEYWDR |
| | ETQKYKRQAQADRVSLRNLRGYYNQSEDGSHTLQRMSGCDLGPDGRLLRGYDQSAYDGKD |
| | YIALNEDLRSWTAADTAAQITQRKLEAARAAEQLRAYLEGTCVEWLRRYLENGKETLQRAE |
| | PPKTHVTHHPLSDHEATLRCWALGFYPAEITLTWQRDGEDQTQDTELVETRPAGDGTFQKW |
| | AAVVVPSGQEQRYTCHMQHEGLQEPLTLSWEPSS |
| | (HLA-C*07:02 soluble) |
| 101 | GSHSMRYFDTAMSRPGRGEPRFISVGYVDDTQFVRFDSDAASPREEPRAPWIEQEGPEYWDR |
| | NTQIFKTNTQTDRESLRNLRGYYNQSEAGSHTLQSMYGCDVGPDGRLLRGHNQYAYDGKDY |
| | IALNEDLRSWTAADTAAQITQRKWEAARVAEQDRAYLEGTCVEWLRRYLENGKDTLERADP |
| | PKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*08:01 soluble) |
| 102 | GSHSMRYFYTAMSRPGRGEPRFIAVGYVDDTQFVRFDSDAASPRTEPRAPWIEQEGPEYWDR |
| | NTQIFKTNTQTYRESLRNLRGYYNQSEAGSHIIQRMYGCDLGPDGRLLRGHDQSAYDGKDYI |
| | ALNEDLSSWTAADTAAQITQRKWEAARVAEQLRAYLEGLCVEWLRRYLENGKETLQRADPP |
| | KTHVTHHPVSDHEATLRCWALGFYPAEITLTWQRDGEDQTQDTELVETRPAGDRTFQKWAA |
| | VVVPSGEEQRYTCHVQHEGLPKPLTLRWEPSS |
| | (HLA-B*35:01 soluble) |
| 103 | GSHSMRYFYTAMSRPGRGEPRFIAVGYVDDTQFVRFDSDAASPRMAPRAPWIEQEGPEYWD |
| | GETRNMKASAQTYRENLRIALRYYNQSEAGSHIIQVMYGCDVGPDGRLLRGHDQSAYDGKD |
| | YIALNEDLSSWTAADTAAQITQRKWEAARVAEQLRAYLEGLCVEWLRRYLENGKETLQRAD |
| | PPKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*57:01 soluble) |
| 104 | GSHSMRYFYTAMSRPGRGEPRFIAVGYVDDTQFVRFDSDAASPRMAPRAPWIEQEGPEYWD |
| | GETRNMKASAQTYRENLRIALRYYNQSEAGSHIIQVMYGCDVGPDGRLLRGHNQYAYDGKD |
| | YIALNEDLSSWTAADTAAQITQRKWEAARVAEQLRAYLEGLCVEWLRRYLENGKETLQRAD |
| | PPKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*57:03 soluble) |
| 105 | GSHSLKYFHTSVSRPGRGEPRFISVGYVDDTQFVRFDNDAASPRMVPRAPWMEQEGSEYWD |
| | RETRSARDTAQIFRVNLRTLRGYYNQSEAGSHTLQWMHGCELGPDGRFLRGYEQFAYDGKD |
| | YLTLNEDLRSWTAVDTAAQISEQKSNDASEAEHQRAYLEDTCVEWLHKYLEKGKETLLHLE |
| | PPKTHVTHHPISDHEATLRCWALGFYPAEITLTWQQDGEGHTQDTELVETRPAGDGTFQKWA |
| | AVVVPSGEEQRYTCHVQHEGLPEPVTLRWKPAS |
| | (HLA-E soluble) |
| 106 | CSHSMRYFYTAVSRPGRGEPRFIAVGYVDDTQFVRFDSDAASPRGEPRAPWVEQEGPEYWD |
| | RETQKYKRQAQTDRVSLRNLRGYYNQSEAGSHTLQWMYGCDLGPDGRLLRGYDQSAYDG |
| | KDYIALNEDLRSWTAADTAAQITQRKWEAARAAEQQRAYLEGTCVEWLRRYLENGKETLQ |
| | RAEHPKTHVTHHLVSDHEATLRCWALGFYPAEITLTWQRDGEDQTQDTELVETRPAGDGTF |
| | QKWAAVVVPSGEEQRYTCHVQHEGLPEPLTLRWEPSS |
| | (HLA-C*16:01 soluble) |
| 107 | CSHSMRYFYTAVSRPGRGEPRFIAVGYVDDTQFVQFDSDAASPRGEPRAPWVEQEGPEYWD |
| | RETQKYKRQAQTDRVSLRNLRGYYNQSEAGSHTLQRMYGCDLGPDGRLLRGYNQFAYDGK |
| | DYIALNEDLRSWTAADKAAQITQRKWEAAREAEQRRAYLEGTCVEWLRRYLENGKKTLQR |
| | AEHPKTHVTHHPVSDHEATLRCWALGFYPAEITLTWQRDGEDQTQDTELVETRPAGDGTFQ |
| | KWAAVVVPSGEEQRYTCHVQHEGLPEPLTLRWGPSS |
| | (HLA-C*08:02 soluble) |
| 108 | CSHSMRYFDTAVSRPGRGEPRFISVGYVDDTQFVRFDSDAASPRGEPRAPWVEQEGPEYWDR |
| | ETQNYKRQAQADRVSLRNLRGYYNQSEDGSHTLQRMYGCDLGPDGRLLRGYDQSAYDGKD |
| | YIALNEDLRSWTAADTAAQITQRKLEAARAAEQLRAYLEGTCVEWLRRYLENGKETLQRAE |
| | PPKTHVTHHPLSDHEATLRCWALGFYPAEITLTWQRDGEDQTQDTELVETRPAGDGTFQKW |
| | AAVVVPSGQEQRYTCHMQHEGLQEPLTLSWEPSS |
| | (HLA-C*07:01 soluble) |
| 109 | CSHSMRYFYTAVSRPGRGEPRFIAVGYVDDTQFVQFDSDAASPRGEPRAPWVEQEGPEYWD |
| | RETQKYKRQAQTDRVNLRKLRGYYNQSEAGSHTLQRMYGCDLGPDGRLLRGYNQFAYDGK |
| | DYIALNEDLRSWTAADKAAQITQRKWEAAREAEQRRAYLEGTCVEWLRRYLENGKKTLQR |
| | AEHPKTHVTHHPVSDHEATLRCWALGFYPAEITLTWQRDGEDQTQDTELVETRPAGDGTFQ |
| | KWAAVVVPSGEEQRYTCHVQHEGLPEPLTLRWGPSS |
| | (HLA-C*05:01 soluble) |
| 110 | GSHSMRYFYTAMSRPGRGEPRFITVGYVDDTLFVRFDSDATSPRKEPRAPWIEQEGPEYWDR |
| | ETQISKTNTQTYRENLRTALRYYNQSEAGSHIIQRMYGCDVGPDGRLLRGYDQDAYDGKDYI |
| | ALNEDLSSWTAADTAAQITQRKWEAARVAEQDRAYLEGLCVESLRRYLENGKETLQRADPP |
| | KTHVTHHPISDHEVTLRCWALGFYPAEITLTWQRDGEDQTQDTELVETRPAGDRTFQKWAA |
| | VVVPSGEEQRYTCHVQHEGLPKPLTLRWEPSS |
| | (HLA-B*44:02 soluble) |
| 111 | GSHSMRYFTTSVSRPGRGEPRFIAVGYVDDTQFVRFDSDAASQRMEPRAPWIEQEGPEYWDL |
| | QTRNVKAQSQTDRANLGTLRGYYNQSEAGSHTIQMMYGCDVGSDGRFLRGYRQDAYDGKD |
| | YIALNEDLRSWTAADMAAQITQRKWEAARVAEQLRAYLEGTCVEWLRRYLENGKETLQRT |
| | DAPKTHMTHHAVSDHEATLRCWALSFYPAEITLTWQRDGEDQTQDTELVETRPAGDGTFQK |
| | WASVVVPSGQEQRYTCHVQHEGLPKPLTLRWEPSS |
| | (HLA-A*29:02 soluble) |
| 112 | GSHSMRYFYTAMSRPGRGEPRFITVGYVDDTLFVRFDSDATSPRKEPRAPWIEQEGPEYWDR |
| | ETQISKTNTQTYRENLRTALRYYNQSEAGSHIIQRMYGCDVGPDGRLLRGYDQDAYDGKDYI |
| | ALNEDLSSWTAADTAAQITQRKWEAARVAEQLRAYLEGLCVESLRRYLENGKETLQRADPP |
| | KTHVTHHPISDHEVTLRCWALGFYPAEITLTWQRDGEDQTQDTELVETRPAGDRTFQKWAA |
| | VVVPSGEEQRYTCHVQHEGLPKPLTLRWEPSS |
| | (HLA-B*44:03 soluble) |
| 113 | GSHSMRYFYTAVSRPGRGEPHFIAVGYVDDTQFVRFDSDAASPRGEPRAPWVEQEGPEYWD |
| | RETQKYKRQAQTDRVSLRNLRGYYNQSEAGSHIIQRMYGCDVGPDGRLLRGYDQYAYDGK |
| | DYIALNEDLRSWTAADTAAQITQRKWEAAREAEQLRAYLEGLCVEWLRRYLKNGKETLQR |
| | AEHPKTHVTHHPVSDHEATLRCWALGFYPAEITLTWQWDGEDQTQDTELVETRPAGDGTFQ |
| | KWAAVVVPSGEEQRYTCHVQHEGLPEPLTLRWEPSS |
| | (HLA-C*03:04 soluble) |
| 114 | GSHSMRYFHTAMSRPGRGEPRFITVGYVDDTLFVRFDSDATSPRKEPRAPWIEQEGPEYWDR |
| | ETQISKTNTQTYRESLRNLRGYYNQSEAGSHTLQRMYGCDVGPDGRLLRGHNQYAYDGKDY |
| | IALNEDLRSWTAADTAAQISQRKLEAARVAEQLRAYLEGECVEWLRRYLENGKDKLERADP |
| | AVVVPSGEEQRYTCHVQHEGLPKPLTLRWEPSS |
| | (HLA-B*40:01 soluble) |
| 115 | CSHSMRYFDTAVSRPGRGEPRFISVGYVDDTQFVRFDSDAASPRGEPRAPWVEQEGPEYWDR |
| | ETQKYKRQAQADRVNLRKLRGYYNQSEDGSHTLQWMYGCDLGPDGRLLRGYDQSAYDGK |
| | DYIALNEDLRSWTAADTAAQITQRKWEAAREAEQWRAYLEGTCVEWLRRYLENGKETLQR |
| | AEHPKTHVTHHPVSDHEATLRCWALGFYPAEITLTWQRDGEDQTQDTELVETRPAGDGTFQ |
| | KWAAVVVPSGEEQRYTCHVQHEGLPEPLTLRWEPSS |
| | (HLA-C*06:02 soluble) |
| 116 | GSHSMRYFYTAMSRPGRGEPRFIAVGYVDDTQFVRFDSDAASPRMAPRAPWIEQEGPEYWD |
| | RETQISKTNTQTYRESLRNLRGYYNQSEAGSHTLQRMYGCDVGPDGRLLRGHDQSAYDGKD |
| | YIALNEDLSSWTAADTAAQITQRKWEAAREAEQWRAYLEGLCVEWLRRYLENGKETLQRA |
| | DPPKTHVTHHPISDHEATLRCWALGFYPAEITLTWQRDGEDQTQDTELVETRPAGDRTFQKW |
| | AAVVVPSGEEQRYTCHVQHEGLPKPLTLRWEPSS |
| | (HLA-B*15:01 soluble) |
| 117 | GSHSMRYFYTAVSRPGRGEPHFIAVGYVDDTQFVRFDSDAASPRGEPRAPWVEQEGPEYWD |
| | RETQKYKRQAQTDRVSLRNLRGYYNQSEARSHIIQRMYGCDVGPDGRLLRGYDQYAYDGK |
| | DYIALNEDLRSWTAADTAAQITQRKWEAAREAEQLRAYLEGLCVEWLRRYLKNGKETLQR |
| | AEHPKTHVTHHPVSDHEATLRCWALGFYPAEITLTWQWDGEDQTQDTELVETRPAGDGTFQ |
| | KWAAVVVPSGEEQRYTCHVQHEGLPEPLTLRWEPSS |
| | (HLA-C*03:03 soluble) |
| 118 | GSHSMRYFSTSVSRPGSGEPRFIAVGYVDDTQFVRFDSDAASQRMEPRAPWIEQERPEYWDQ |
| | ETRNVKAQSQTDRVDLGTLRGYYNQSEAGSHTIQIMYGCDVGSDGRFLRGYEQHAYDGKDY |
| | IALNEDLRSWTAADMAAQITQRKWEAARWAEQLRAYLEGTCVEWLRRYLENGKETLQRTD |
| | PPKTHMTHHPISDHEATLRCWALGFYPAEITLTWQRDGEDQTQDTELVETRPAGDGTFQKW |
| | AAVVVPSGEEQRYTCHVQHEGLPKPLTLRWELSS |
| | (HLA-A*30:01 soluble) |
| 119 | GSHSMRYFYTAMSRPGRGEPRFITVGYVDDTQFVRFDSDATSPRMAPRAPWIEQEGPEYWDR |
| | ETQISKTNTQTYRENLRTALRYYNQSEAGSHTWQTMYGCDLGPDGRLLRGHNQLAYDGKD |
| | YIALNEDLSSWTAADTAAQITQLKWEAARVAEQLRAYLEGECVEWLRRYLENGKETLQRAD |
| | PPKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*13:02 soluble) |
| 120 | CSHSMRYFYTAVSRPGRGEPRFIAVGYVDDTQFVRFDSDAASPRGEPRAPWVEQEGPEYWD |
| | RETQKYKRQAQADRVSLRNLRGYYNQSEAGSHTLQWMYGCDLGPDGRLLRGYDQSAYDG |
| | KDYIALNEDLRSWTAADTAAQITQRKWEAAREAEQWRAYLEGTCVEWLRRYLENGKETLQ |
| | RAEHPKTHVTHHPVSDHEATLRCWALGFYPAEITLTWQRDGEDQTQDTELVETRPAGDGTF |
| | QKWAAVVVPSGEEQRYTCHVQHEGLPEPLTLRWEPSS |
| | (HLA-C*12:03 soluble) |
| 121 | GSHSMRYFYTSVSRPGRGEPRFIAVGYVDDTQFVRFDSDAASQRMEPRAPWIEQEGPEYWDR |
| | NTRNVKAHSQTDRANLGTLRGYYNQSEDGSHTIQRMYGCDVGPDGRFLRGYQQDAYDGKD |
| | YIALNEDLRSWTAADMAAQITQRKWETAHEAEQWRAYLEGRCVEWLRRYLENGKETLQRT |
| | DAPKTHMTHHAVSDHEATLRCWALSFYPAEITLTWQRDGEDQTQDTELVETRPAGDGTFQK |
| | WASVVVPSGQEQRYTCHVQHEGLPKPLTLRWEPSS |
| | (HLA-A*26:01 soluble) |
| 122 | GSHSMRYFYTSVSRPGRGEPRFISVGYVDDTQFVRFDSDAASPREEPRAPWIEQEGPEYWDRN |
| | TQICKTNTQTYRENLRIALRYYNQSEAGSHTLQRMYGCDVGPDGRLLRGHNQFAYDGKDYI |
| | ALNEDLSSWTAADTAAQITQRKWEAARVAEQLRTYLEGTCVEWLRRYLENGKETLQRADPP |
| | KTHVTHHPISDHEATLRCWALGFYPAEITLTWQRDGEDQTQDTELVETRPAGDRTFQKWAA |
| | VVVPSGEEQRYTCHVQHEGLPKPLTLRWEPSS |
| | (HLA-B*38:01 soluble) |
| 123 | GSHSMRYFYTAVSRPGRGEPRFISVGYVDDTQFVRFDSDAASPREEPRAPWIEQEGPEYWDR |
| | NTQICKTNTQTDRESLRNLRGYYNQSEAGSHTLQWMYGCDVGPDGRLLRGYNQFAYDGKD |
| | YIALNEDLSSWTAADTAAQITQRKWEAAREAEQLRAYLEGTCVEWLRRHLENGKETLQRAD |
| | PPKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*14:02 soluble) |
| 124 | GSHSMRYFTTSVSRPGRGEPRFIAVGYVDDTQFVRFDSDAASQRMEPRAPWIEQEGPEYWDR |
| | NTRNVKAHSQIDRVDLGTLRGYYNQSEAGSHTIQMMYGCDVGSDGRFLRGYQQDAYDGKD |
| | YIALNEDLRSWTAADMAAQITQRKWEAARVAEQLRAYLEGTCVEWLRRHLENGKETLQRT |
| | DPPRTHMTHHAVSDHEATLRCWALSFYPAEITLTWQRDGEDQTQDTELVETRPAGDGTFQK |
| | WASVVVPSGQEQRYTCHVQHEGLPKPLTLRWEPSS |
| | (HLA-A*33:01 soluble) |
| 125 | GSHSMRYFSTSVSRPGRGEPRFIAVGYVDDTQFVRFDSDAASQRMEPRAPWIEQEGPEYWDE |
| | ETGKVKAHSQTDRENLRIALRYYNQSEAGSHTLQMMFGCDVGSDGRFLRGYHQYAYDGKD |
| | YIALKEDLRSWTAADMAAQITQRKWEAARVAEQLRAYLEGTCVDGLRRYLENGKETLQRT |
| | DPPKTHMTHHPISDHEATLRCWALGFYPAEITLTWQRDGEDQTQDTELVETRPAGDGTFQK |
| | WAAVVVPSGEEQRYTCHVQHEGLPKPLTLRWEPSS |
| | (HLA-A*23:01 soluble) |
| 126 | GSHSMRYFYTSVSRPGRGEPRFIAVGYVDDTQFVRFDSDAASQRMEPRAPWIEQEGPEYWDR |
| | NTRNVKAHSQTDRESLRIALRYYNQSEDGSHTIQRMYGCDVGPDGRFLRGYQQDAYDGKDY |
| | IALNEDLRSWTAADMAAQITQRKWETAHEAEQWRAYLEGRCVEWLRRYLENGKETLQRTD |
| | APKTHMTHHAVSDHEATLRCWALSFYPAEITLTWQRDGEDQTQDTELVETRPAGDGTFQKW |
| | ASVVVPSGQEQRYTCHVQHEGLPKPLTLRWEPSS |
| | (HLA-A*25:01 soluble) |
| 127 | GSHSMRYFHTSVSRPGRGEPRFISVGYVDGTQFVRFDSDAASPRTEPRAPWIEQEGPEYWDRN |
| | TQISKTNTQTYRESLRNLRGYYNQSEAGSHTLQRMYGCDVGPDGRLLRGHDQSAYDGKDYI |
| | ALNEDLSSWTAADTAAQITQRKWEAARVAEQLRAYLEGTCVEWLRRHLENGKETLQRADPP |
| | KTHVTHHPISDHEATLRCWALGFYPAEITLTWQRDGEDQTQDTELVETRPAGDRTFQKWAA |
| | VVVPSGEEQRYTCHVQHEGLPKPLTLRWEPSS |
| | (HLA-B*18:01 soluble) |
| 128 | GSHSMRYFHTSVSRPGRGEPRFISVGYVDDTQFVRFDSDAASPRTEPRAPWIEQEGPEYWDRE |
| | TQISKTNTQTYREDLRTLLRYYNQSEAGSHTIQRMSGCDVGPDGRLLRGYNQFAYDGKDYIA |
| | LNEDLSSWTAADTAAQITQRKWEAARVAEQDRAYLEGTCVEWLRRYLENGKETLQRADPP |
| | KTHVTHHPISDHEATLRCWALGFYPAEITLTWQRDGEDQTQDTELVETRPAGDRTFQKWAA |
| | VVVPSGEEQRYTCHVQHEGLPKPLTLRWEPSS |
| | (HLA-B*37:01 soluble) |
| 129 | GSHSMRYFYTAMSRPGRGEPRFIAVGYVDDTQFVRFDSDAASPRTEPRAPWIEQEGPEYWDR |
| | NTQIFKTNTQTYRENLRIALRYYNQSEAGSHTWQTMYGCDVGPDGRLLRGHNQYAYDGKD |
| | YIALNEDLSSWTAADTAAQITQRKWEAAREAEQLRAYLEGLCVEWLRRHLENGKETLQRAD |
| | PPKTHVTHHPVSDHEATLRCWALGFYPAEITLTWQRDGEDQTQDTELVETRPAGDRTFQKW |
| | AAVVVPSGEEQRYTCHVQHEGLPKPLTLRWEPSS |
| | (HLA-B*51:01 soluble) |
| 130 | CSHSMRYFSTSVSRPGRGEPRFIAVGYVDDTQFVRFDSDAASPRGEPRAPWVEQEGPEYWDR |
| | ETQKYKRQAQTDRVSLRNLRGYYNQSEAGSHTLQWMFGCDLGPDGRLLRGYDQSAYDGKD |
| | YIALNEDLRSWTAADTAAQITQRKWEAAREAEQRRAYLEGTCVEWLRRYLENGKETLQRAE |
| | HPKTHVTHHPVSDHEATLRCWALGFYPAEITLTWQWDGEDQTQDTELVETRPAGDGTFQK |
| | WAAVVVPSGEEQRYTCHVQHEGLPEPLTLRWEPSS |
| | (HLA-C*14:02 soluble) |
| 131 | CSHSMRYFYTAVSRPGRGEPHFIAVGYVDDTQFVRFDSDAASPRGEPRAPWVEQEGPEYWD |
| | RETQNYKRQAQTDRVNLRKLRGYYNQSEAGSHIIQRMYGCDLGPDGRLLRGHDQLAYDGK |
| | DYIALNEDLRSWTAADTAAQITQRKWEAAREAEQLRAYLEGTCVEWLRRYLENGKETLQRA |
| | EHPKTHVTHHPVSDHEATLRCWALGFYPAEITLTWQRDGEDQTQDTELVETRPAGDGTFQK |
| | WAAVVVPSGEEQRYTCHVQHEGLPEPLTLRWEPSS |
| | (HLA-C*15:02 soluble) |
| 132 | CSHSMRYFYTAVSRPSRGEPHFIAVGYVDDTQFVRFDSDAASPRGEPRAPWVEQEGPEYWDR |
| | ETQKYKRQAQTDRVNLRKLRGYYNQSEAGSHTLQRMYGCDLGPDGRLLRGYDQSAYDGKD |
| | YIALNEDLRSWTAADTAAQITQRKWEAAREAEQWRAYLEGECVEWLRRYLENGKETLQRA |
| | EHPKTHVTHHPVSDHEATLRCWALGFYPTEITLTWQRDGEDQTQDTELVETRPAGDGTFQK |
| | WAAVVVPSGEEQRYTCHVQHEGLPEPLTLRWEPSS |
| | (HLA-C*02:02 soluble) |
| 133 | GSHSMRYFHTSVSRPGRGEPRFITVGYVDDTLFVRFDSDAASPREEPRAPWIEQEGPEYWDRE |
| | TQICKAKAQTDREDLRTLLRYYNQSEAGSHTLQNMYGCDVGPDGRLLRGYHQDAYDGKDY |
| | IALNEDLSSWTAADTAAQITQRKWEAARVAEQLRAYLEGECVEWLRRYLENGKETLQRADP |
| | PKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*27:05 soluble) |
| 134 | GSHSMRYFTTSVSRPGRGEPRFIAVGYVDDTQFVRFDSDAASQRMEPRAPWIEQERPEYWDQ |
| | ETRNVKAHSQIDRVDLGTLRGYYNQSEAGSHTIQMMYGCDVGSDGRFLRGYQQDAYDGKD |
| | YIALNEDLRSWTAADMAAQITQRKWEAARVAEQLRAYLEGTCVEWLRRYLENGKETLQRT |
| | DPPKTHMTHHAVSDHEATLRCWALSFYPAEITLTWQRDGEDQTQDTELVETRPAGDGTFQK |
| | WASVVVPSGQEQRYTCHVQHEGLPKPLTLRWEPSS |
| | (HLA-A*31:01 soluble) |
| 135 | GSHSMRYFSTSVSRPGSGEPRFIAVGYVDDTQFVRFDSDAASQRMEPRAPWIEQERPEYWDQ |
| | ETRNVKAHSQTDRENLGTLRGYYNQSEAGSHTIQIMYGCDVGSDGRFLRGYEQHAYDGKDY |
| | IALNEDLRSWTAADMAAQITQRKWEAARRAEQLRAYLEGTCVEWLRRYLENGKETLQRTDP |
| | PKTHMTHHPISDHEATLRCWALGFYPAEITLTWQRDGEDQTQDTELVETRPAGDGTFQKWA |
| | AVVVPSGEEQRYTCHVQHEGLPKPLTLRWELSS |
| | (HLA-A*30:02 soluble) |
| 136 | GSHSMRYFYTSVSRPGRGEPRFISVGYVDDTQFVRFDSDAASPREEPRAPWIEQEGPEYWDRN |
| | TQIYKAQAQTDRESLRNLRGYYNQSEAGSHTLQSMYGCDVGPDGRLLRGHNQYAYDGKDYI |
| | ALNEDLRSWTAADTAAQITQRKWEAARVAEQDRAYLEGTCVEWLRRYLENGKDTLERADP |
| | PKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*42:01 soluble) |
| 137 | GSHSMRYFYTAVSRPGRGEPRFIAVGYVDDTQFVRFDSDAASPRGEPRAPWVEQEGPEYWD |
| | RETQKYKRQAQADRVNLRKLRGYYNQSEAGSHTIQRMYGCDLGPDGRLLRGYNQFAYDGK |
| | DYIALNEDLRSWTAADTAAQISQRKLEAAREAEQLRAYLEGECVEWLRGYLENGKETLQRA |
| | ERPKTHVTHHPVSDHEATLRCWALGFYPAEITLTWQRDGEDQTQDTELVETRPAGDGTFQK |
| | WAAVVVPSGQEQRYTCHVQHEGLQEPCTLRWKPSS |
| | (HLA-C*17:01 soluble) |
| 138 | GSHSMRYFYTAMSRPGRGEPRFIAVGYVDDTQFVRFDSDAASPRTEPRAPWIEQEGPEYWDR |
| | NTQIFKTNTQTYRESLRNLRGYYNQSEAGSHIIQRMYGCDLGPDGRFLRGHNQYAYDGKDYI |
| | ALNEDLSSWTAADTAAQITQRKWEAARVAEQLRAYLEGLCVEWLRRYLENGKETLQRADPP |
| | KTHVTHHPVSDHEATLRCWALGFYPAEITLTWQRDGEDQTQDTELVETRPAGDRTFQKWAA |
| | VVVPSGEEQRYTCHVQHEGLPKPLTLRWEPSS |
| | (HLA-B*35:02 soluble) |
| 139 | GSHSMRYFYTSVSRPGRGEPRFISVGYVDDTQFVRFDSDAASPREEPRAPWIEQEGPEYWDRN |
| | TQICKTNTQTDRESLRNLRGYYNQSEAGSHTWQTMYGCDVGPDGRLLRGHNQFAYDGKDYI |
| | ALNEDLSSWTAADTAAQITQRKWEAARVAEQLRTYLEGTCVEWLRRYLENGKETLQRADPP |
| | KTHVTHHPISDHEATLRCWALGFYPAEITLTWQRDGEDQTQDTELVETRPAGDRTFQKWAA |
| | VVVPSGEEQRYTCHVQHEGLPKPLTLRWEPSS |
| | (HLA-B*39:06 soluble) |
| 140 | GSHSMRYFYTAVSRPGRGEPHFIAVGYVDDTQFVRFDSDAASPRGEPRAPWVEQEGPEYWD |
| | RETQKYKRQAQTDRVSLRNLRGYYNQSEAGSHILQRMYGCDVGPDGRLLRGYDQSAYDGK |
| | DYIALNEDLRSWTAADTAAQITQRKWEAAREAEQLRAYLEGLCVEWLRRYLKNGKETLQR |
| | AEHPKTHVTHHPVSDHEATLRCWALGFYPAEITLTWQWDGEDQTQDTELVETRPAGDGTFQ |
| | KWAAVVVPSGEEQRYTCHVQHEGLPEPLTLRWEPSS |
| | (HLA-C*03:02 soluble) |
| 141 | GSHSMRYFYTAMSRPGRGEPRFIAVGYVDDTQFVRFDSDAASPRTEPRAPWIEQEGPEYWDG |
| | ETRNMKASAQTYRENLRIALRYYNQSEAGSHIIQRMYGCDLGPDGRLLRGHDQSAYDGKDYI |
| | ALNEDLSSWTAADTAAQITQRKWEAARVAEQLRAYLEGLCVEWLRRYLENGKETLQRADPP |
| | KTHVTHHPVSDHEATLRCWALGFYPAEITLTWQRDGEDQTQDTELVETRPAGDRTFQKWAA |
| | VVVPSGEEQRYTCHVQHEGLPKPLTLRWEPSS |
| | (HLA-B*58:01 soluble) |
| 142 | GSHSMRYFTTSVSRPGRGEPRFIAVGYVDDTQFVRFDSDAASQRMEPRAPWIEQEGPEYWDR |
| | NTRNVKAHSQIDRVDLGTLRGYYNQSEAGSHTIQMMYGCDVGSDGRFLRGYQQDAYDGKD |
| | YIALNEDLRSWTAADMAAQITQRKWEAARVAEQLRAYLEGTCVEWLRRYLENGKETLQRT |
| | DPPKTHMTHHAVSDHEATLRCWALSFYPAEITLTWQRDGEDQTQDTELVETRPAGDGTFQK |
| | WASVVVPSGQEQRYTCHVQHEGLPKPLTLRWEPSS |
| | (HLA-A*33:03 soluble) |
| 143 | GSHSMRYFYTSMSRPGRGEPRFIAVGYVDDTQFVRFDSDAASQRMEPRAPWIEQEGPEYWD |
| | RNTRNVKAQSQTDRVDLGTLRGYYNQSEAGSHTIQRMYGCDVGPDGRFLRGYHQYAYDGK |
| | DYIALKEDLRSWTAADMAAQTTKHKWEAAHVAEQWRAYLEGTCVEWLRRYLENGKETLQ |
| | RTDAPKTHMTHHAVSDHEATLRCWALSFYPAEITLTWQRDGEDQTQDTELVETRPAGDGTF |
| | QKWVAVVVPSGQEQRYTCHVQHEGLPKPLTLRWEPSS |
| | (HLA-A*68:02 soluble) |
| 144 | CSHSMKYFFTSVSRPGRGEPRFISVGYVDDTQFVRFDSDAASPRGEPRAPWVEQEGPEYWDR |
| | ETQKYKRQAQTDRVSLRNLRGYYNQSEAGSHTLQWMCGCDLGPDGRLLRGYDQYAYDGK |
| | DYIALNEDLRSWTAADTAAQITQRKWEAAREAEQRRAYLEGTCVEWLRRYLENGKETLQRA |
| | EHPKTHVTHHPVSDHEATLRCWALGFYPAEITLTWQWDGEDQTQDTELVETRPAGDGTFQK |
| | WAAVMVPSGEEQRYTCHVQHEGLPEPLTLRWEPSS |
| | (HLA-C*01:02 soluble) |
| 145 | CSHSMRYFDTAVSRPGRGEPRFISVGYVDDTQFVRFDSDAASPRGEPRAPWVEQEGPEYWDR |
| | ETQKYKRQAQADRVSLRNLRGYYNQSEDGSHTFQRMYGCDLGPDGRLLRGYDQFAYDGKD |
| | YIALNEDLRSWTAADTAAQITQRKLEAARAAEQDRAYLEGTCVEWLRRYLENGKKTLQRAE |
| | PPKTHVTHHPLSDHEATLRCWALGFYPAEITLTWQRDGEDQTQDTELVETRPAGDGTFQKW |
| | AAVVVPSGQEQRYTCHMQHEGLQEPLTLSWEPSS |
| | (HLA-C*07:04 soluble) |
| 146 | GSHSMRYFYTSVSRPGRGEPRFIAVGYVDDTQFVRFDSDAASQRMEPRAPWIEQEGPEYWDR |
| | NTRNVKAQSQTDRVDLGTLRGYYNQSEAGSHTIQMMYGCDVGSDGRFLRGYRQDAYDGKD |
| | YIALKEDLRSWTAADMAAQTTKHKWEAAHVAEQWRAYLEGTCVEWLRRYLENGKETLQR |
| | TDAPKTHMTHHAVSDHEATLRCWALSFYPAEITLTWQRDGEDQTQDTELVETRPAGDGTFQ |
| | KWVAVVVPSGQEQRYTCHVQHEGLPKPLTLRWEPSS |
| | (HLA-A*68:01 soluble) |
| 147 | GSHSMRYFFTSVSRPGRGEPRFIAVGYVDDTQFVRFDSDAASQRMEPRAPWIEQEGPEYWDQ |
| | ETRNVKAHSQTDRESLRIALRYYNQSEAGSHTIQMMYGCDVGPDGRLLRGYQQDAYDGKD |
| | YIALNEDLRSWTAADMAAQITQRKWEAARVAEQLRAYLEGTCVEWLRRYLENGKETLQRT |
| | DAPKTHMTHHAVSDHEATLRCWALSFYPAEITLTWQRDGEDQTQDTELVETRPAGDGTFQK |
| | WASVVVPSGQEQRYTCHVQHEGLPKPLTLRWEPSS |
| | (HLA-A*32:01 soluble) |
| 148 | GSHSMRYFHTAMSRPGRGEPRFITVGYVDDTLFVRFDSDATSPRKEPRAPWIEQEGPEYWDR |
| | ETQISKTNTQTYRENLRIALRYYNQSEAGSHTWQRMYGCDLGPDGRLLRGYNQLAYDGKDY |
| | IALNEDLSSWTAADTAAQITQRKWEAAREAEQLRAYLEGLCVEWLRRYLENGKETLQRADP |
| | AVVVPSGEEQRYTCHVQHEGLPKPLTLRWEPSS |
| | (HLA-B*49:01 soluble) |
| 149 | GSHSMRYFYTAMSRPGRGEPRFIAVGYVDDTQFVRFDSDAASPRTEPRAPWIEQEGPEYWDR |
| | NTQIFKTNTQTYRENLRIALRYYNQSEAGSHIIQRMYGCDLGPDGRLLRGHDQSAYDGKDYI |
| | ALNEDLSSWTAADTAAQITQRKWEAARVAEQLRAYLEGLCVEWLRRYLENGKETLQRADPP |
| | KTHVTHHPVSDHEATLRCWALGFYPAEITLTWQRDGEDQTQDTELVETRPAGDRTFQKWAA |
| | VVVPSGEEQRYTCHVQHEGLPKPLTLRWEPSS |
| | (HLA-B*53:01 soluble) |
| 150 | GSHSMRYFHTAMSRPGRGEPRFITVGYVDDTLFVRFDSDATSPRKEPRAPWIEQEGPEYWDR |
| | ETQISKTNTQTYRESLRNLRGYYNQSEAGSHTWQRMYGCDLGPDGRLLRGYNQLAYDGKD |
| | YIALNEDLSSWTAADTAAQITQRKWEAAREAEQLRAYLEGLCVEWLRRYLENGKETLQRAD |
| | PPKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*50:01 soluble) |
| 151 | GSHSMRYFYTSVSRPGRGEPRFIAVGYVDDTQFVRFDSDAASRRMEPRAPWIEQEGPEYWDG |
| | ETRKVKAHSQTHRVDLGTLRGYYNQSEAGSHTLQRMYGCDVGSDWRFLRGYHQYAYDGK |
| | DYIALKEDLRSWTAADMAAQTTKHKWEAAHVAEQWRAYLEGTCVEWLRRYLENGKETLQ |
| | RTDAPKTHMTHHAVSDHEATLRCWALSFYPAEITLTWQRDGEDQTQDTELVETRPAGDGTF |
| | QKWAAVVVPSGQEQRYTCHVQHEGLPKPLTLRWEPSS |
| | (HLA-A*02:05 soluble) |
| 152 | GSHSMRYFYTAMSRPGRGEPRFIAVGYVDDTQFVRFDSDAASPREEPRAPWIEQEGPEYWDR |
| | NTQIYKAQAQTDRESLRNLRGYYNQSEAGSHTWQTMYGCDLGPDGRLLRGHNQLAYDGKD |
| | YIALNEDLSSWTAADTAAQITQRKWEAAREAEQLRAYLEGTCVEWLRRYLENGKETLQRAD |
| | PPKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*55:01 soluble) |
| 153 | GSHSMRYFHTAMSRPGRGEPRFITVGYVDDTLFVRFDSDATSPRKEPRAPWIEQEGPEYWDR |
| | ETQISKTNTQTYRESLRNLRGYYNQSEAGSHTWQRMYGCDLGPDGRLLRGYNQLAYDGKD |
| | YIALNEDLSSWTAADTAAQITQRKWEAARVAEQDRAYLEGLCVESLRRYLENGKETLQRAD |
| | PPKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*45:01 soluble) |
| 154 | GSHSMRYFYTAMSRPGRGEPRFIAVGYVDDTQFVRFDSDAASPRTEPRAPWIEQEGPEYWDR |
| | ETQISKTNTQTYRENLRIALRYYNQSEAGSHTWQTMYGCDVGPDGRLLRGHNQYAYDGKD |
| | YIALNEDLSSWTAADTAAQITQRKWEAAREAEQLRAYLEGLCVEWLRRHLENGKETLQRAD |
| | PPKTHVTHHPVSDHEATLRCWALGFYPAEITLTWQRDGEDQTQDTELVETRPAGDRTFQKW |
| | AAVVVPSGEEQRYTCHVQHEGLPKPLTLRWEPSS |
| | (HLA-B*52:01 soluble) |
| 155 | CSHSMRYFYTAVSRPGRGEPRFIAVGYVDDTQFVRFDSDAASPRGEPRAPWVEQEGPEYWD |
| | RETQKYKRQAQADRVSLRNLRGYYNQSEAGSHTLQRMYGCDLGPDGRLLRGYDQSAYDGK |
| | DYIALNEDLRSWTAADTAAQITQRKWEAAREAEQWRAYLEGTCVEWLRRYLENGKETLQR |
| | AEHPKTHVTHHPVSDHEATLRCWALGFYPAEITLTWQRDGEDQTQDTELVETRPAGDGTFQ |
| | KWAAVVVPSGEEQRYTCHVQHEGLPEPLTLRWEPSS |
| | (HLA-C*12:02 soluble) |
| 156 | GSHSMRYFYTAMSRPGRGEPRFIAVGYVDDTQFVRFDSDAASPRTEPRAPWIEQEGPEYWDR |
| | NTQIFKTNTQTYRESLRNLRGYYNQSEAGSHIIQRMYGCDLGPDGRLLRGHDQFAYDGKDYI |
| | ALNEDLSSWTAADTAAQITQRKWEAARVAEQLRAYLEGLCVEWLRRYLENGKETLQRADPP |
| | KTHVTHHPVSDHEATLRCWALGFYPAEITLTWQRDGEDQTQDTELVETRPAGDRTFQKWAA |
| | VVVPSGEEQRYTCHVQHEGLPKPLTLRWEPSS |
| | (HLA-B*35:03 soluble) |
| 157 | GSHSMRYFHTSVSRPGRGEPRFITVGYVDDTLFVRFDSDATSPRKEPRAPWIEQEGPEYWDRE |
| | TQISKTNTQTYRESLRNLRGYYNQSEAGSHTLQSMYGCDVGPDGRLLRGHNQYAYDGKDYI |
| | ALNEDLRSWTAADTAAQITQRKWEAARVAEQLRAYLEGECVEWLRRYLENGKETLQRADP |
| | PKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*40:02 soluble) |
| 158 | GSHSMRYFYTAMSRPGRGEPRFISVGYVDDTQFVRFDSDAASPREEPRAPWIEQEGPEYWDR |
| | ETQISKTNTQTYRESLRNLRGYYNQSEAGSHTLQRMYGCDVGPDGRLLRGHDQSAYDGKDY |
| | IALNEDLSSWTAADTAAQITQRKWEAAREAEQLRAYLEGLCVEWLRRYLENGKETLQRADP |
| | PKTHVTHHPISDHEATLRCWALGFYPAEITLTWQRDGEDQTQDTELVETRPAGDRTFQKWA |
| | AVVVPSGEEQRYTCHVQHEGLPKPLTLRWEPSS |
| | (HLA-B*15:03 soluble) |
| 159 | GSHSMRYFFTSVSRPGRGEPRFIAVGYVDDTQFVRFDSDAASQRMEPRAPWIEQEGPEYWDQ |
| | ETRNVKAHSQTDRVDLGTLRGYYNQSEAGSHTIQMMYGCDVGPDGRLLRGYQQDAYDGKD |
| | YIALNEDLRSWTAADMAAQITQRKWEAARVAEQLRAYLEGTCVEWLRRYLENGKETLQRT |
| | DAPKTHMTHHAVSDHEATLRCWALSFYPAEITLTWQRDGEDQTQDTELVETRPAGDGTFQK |
| | WASVVVPSGQEQRYTCHVQHEGLPKPLTLRWEPSS |
| | (HLA-A*74:01 soluble) |
| 160 | MSRSVALAVLALLSLSGLEAIQRTPKIQVYSRHPAENGKSNFLNCYVSGFHPSDIEVDLL |
| | KNGERIEKVEHSDLSFSKDWSFYLLYYTEFTPTEKDEYACRVNHVTLSQPKIVKWDRDM |
| | (full length human beta-2-microglobulin) |
| 161 | GSGSAGGGLNDIFEAQKIEWHEGSTGHHHHHHDYKDDDDK |
| | (Avitag sequence with His6 tag and Flag tag) |
| 162 | GSGSAGGSGSGGGSLPETGGHHHHHH |
| | (Sortag sequence with His6 tag) |
| 163 | LPXTG, wherein X is any amino acid |
| | (sortag motif) |
| 164 | IPKTG |
| | (sortag motif) |
| 165 | MPXTG, wherein X is any amino acid |
| | (sortag motif) |
| 166 | LAETG |
| | (sortag motif) |
| 167 | LPXAG, wherein X is any amino acid |
| | (sortag motif) |
| 168 | LPESG |
| | (sortag motif) |
| 169 | LPELG |
| | (sortag motif) |
| 170 | LPEVG |
| | (sortag motif) |
| 171 | XPKTG, wherein X = any amino acid |
| | (sortag motif) |
| 172 | APKTG |
| | (sortag motif) |
| 173 | DPKTG |
| | (sortag motif) |
| 174 | SPKTG |
| | (sortag motif) |
| 175 | LPEXG, wherein X = any amino acid |
| | (sortag motif) |
| 176 | LPEAG |
| | (sortag motif) |
| 177 | LPECG |
| | (sortag motif) |
| 178 | LPEXG, ), wherein X = A, C or S |
| | (sortag motif) |
| 179 | WTWTW |
| | (ligation control motif) |
| 180 | GSGSAGGSFESGPGAEYCFNVDDPSKDSKAQVSAAEAGITGTWYNQLGSTFIVTAGADGALTGTYESAVGNAESR |
| | YVLTGRYDSAPATDGSGTALGWTVAWKNNYRNAHSATTWSGQYVGGAEARINTQWLLTSGTTEANAWKSTLVG |
| | HDTFTKVKPSAASIDAAKKAGVNNGNPLDAVQQGSTGDYKDDDDK |
| | (N-intein sequence with Flag tag) |
| 181 | (GGGGS)n, wherein n = 1-6 |
| | (linker) |
| 182 | SSSSGSSSSGSAA |
| | (linker) |
| 183 | GGGGG |
| | (linker) |
| 184 | S(GGGGS)n, wherein n = 1-10 |
| | (linker) |
| 185 | (GGSG)n, wherein n = 1-5 |
| | (linker) |
| 186 | GSAT |
| | (linker) |
| 187 | (GGSGGS)n, wherein n = 1-5 |
| | (linker) |
| 188 | DDDDK |
| | (enterokinase cleavage site) |
| 189 | KPVSKMRMATPLLMQA |
| | (CLIP peptide) |
| 190 | QIYKANSKFIGITEL |
| | (TT p2 peptide) |
| 191 | IKEEHVIIQAEFYLNPDQSGEFMFDFDGDEIFHVDMAKKETVWRLEEFGRFASFEAQGALANI |
| | AVDKANLEIMTKRSNYTPITNVPPEVTVLTNSPVELREPNVLICFIDKFTPPVVNVTWLRNGKP |
| | VTTGVSETVFLPREDHLFRKFHYLPFLPSTEDVYDCRVEHWGLDEPLLKHWEFDAPSPLPETT |
| | EGSEQKLISEEDLPETGG |
| | (HLA-DRA*01:01 Myc tag and Sorttag) |
| 192 | KPVSKMRMATPLLMQAGGGGSIEGRGSGGGSGDTRPRFLWQLKFECHFFNGTERVRLLERCI |
| | YNQEESVRFDSDVGEYRAVTELGRPDAEYWNSQKDLLEQRRAAVDTYCRHNYGVGESFTV |
| | QRRVEPKVTVYPSKTQPLQHHNLLVCSVSGFYPGSIEVRWFRNGQEEKAGVVSTGLIQNGDW |
| | TFQTLVMLETVPRSGEVYTCQVEHPSVTSPLTVEWRARSESAQSKLGGLNDIFEAQKIEWHEH |
| | HHHHH |
| | (HLA-DRB1*01:01 with N-terminal CLIP peptide, digestible linker |
| | and C-terminal AviTag and His6 tag) |
| 193 | biotin-PKYVKQNTLKLAT |
| | (HA peptide from Influenza A virus) |
| 194 | MAISGVPVLGFFIIAVLMSAQESWAIKEEHVIIQAEFYLNPDQSGEFMFDFDGDEIFHVDMAK |
| | KETVWRLEEFGRFASFEAQGALANIAVDKANLEIMTKRSNYTPITNVPPEVTVLTNSPVELRE |
| | PNVLICFIDKFTPPVVNVTWLRNGKPVTTGVSETVFLPREDHLFRKFHYLPFLPSTEDVYDCRV |
| | EHWGLDEPLLKHWEFDAPSPLPETTENVVCALGLTVGLVGIIIGTIFIIKGVRKSNAAERRGPL |
| | (HLA-DRA*01:01 full-length) |
| 195 | MVCLKLPGGSCMTALTVTLMVLSSPLALAGDTRPRFLWQLKFECHFFNGTERVRLLERCIYN |
| | QEESVRFDSDVGEYRAVTELGRPDAEYWNSQKDLLEQRRAAVDTYCRHNYGVGESFTVQRR |
| | VEPKVTVYPSKTQPLQHHNLLVCSVSGFYPGSIEVRWFRNGQEEKAGVVSTGLIQNGDWTFQ |
| | TLVMLETVPRSGEVYTCQVEHPSVTSPLTVEWRARSESAQSKMLSGVGGFVLGLLFLGAGLFI |
| | YFRNQKGHSGLQPTGFLS |
| | (HLA-DRB1*01:01 full-length) |
| 196 | MVCLKLPGGSCMTALTVTLMVLSSPLALAGDTRPRFLWQLKFECHFFNGTERVRLLERCIYN |
| | QEESVRFDSDVGEYRAVTELGRPDAEYWNSQKDLLEQRRAAVDTYCRHNYGAVESFTVQRR |
| | VEPKVTVYPSKTQPLQHHNLLVCSVSGFYPGSIEVRWFRNGQEEKAGVVSTGLIQNGDWTFQ |
| | TLVMLETVPRSGEVYTCQVEHPSVTSPLTVEWRARSESAQSKMLSGVGGFVLGLLFLGAGLFI |
| | YFRNQKGHSGLQPTGFLS |
| | (HLA-DRB1*01:02 full-length) |
| 197 | MVCLRLPGGSCMAVLTVTLMVLSSPLALAGDTRPRFLEYSTSECHFFNGTERVRYLDRYFHN |
| | QEENVRFDSDVGEFRAVTELGRPDAEYWNSQKDLLEQKRGRVDNYCRHNYGVVESFTVQR |
| | RVHPKVTVYPSKTQPLQHHNLLVCSVSGFYPGSIEVRWFRNGQEEKTGVVSTGLIHNGDWTF |
| | QTLVMLETVPRSGEVYTCQVEHPSVTSPLTVEWRARSESAQSKMLSGVGGFVLGLLFLGAGL |
| | FIYFRNQKGHSGLQPRGFLS |
| | (HLA-DRB1*03:01 full-length) |
| 198 | MVCLKFPGGSCMAALTVTLMVLSSPLALAGDTRPRFLEQVKHECHFFNGTERVRFLDRYFYH |
| | QEEYVRFDSDVGEYRAVTELGRPDAEYWNSQKDLLEQKRAAVDTYCRHNYGVGESFTVQR |
| | RVYPEVTVYPAKTQPLQHHNLLVCSVNGFYPGSIEVRWFRNGQEEKTGVVSTGLIQNGDWTF |
| | QTLVMLETVPRSGEVYTCQVEHPSLTSPLTVEWRARSESAQSKMLSGVGGFVLGLLFLGAGL |
| | FIYFRNQKGHSGLQPTGFLS |
| | (HLA-DRB1*04:01 full-length) |
| 199 | MVCLKFPGGSCMAALTVTLMVLSSPLALAGDTRPRFLEQVKHECHFFNGTERVRFLDRYFYH |
| | QEEYVRFDSDVGEYRAVTELGRPDAEYWNSQKDLLEQRRAAVDTYCRHNYGVVESFTVQR |
| | RVYPEVTVYPAKTQPLQHHNLLVCSVNGFYPGSIEVRWFRNGQEEKTGVVSTGLIQNGDWTF |
| | QTLVMLETVPRSGEVYTCQVEHPSLTSPLTVEWRARSESAQSKMLSGVGGFVLGLLFLGAGL |
| | FIYFRNQKGHSGLQPTGFLS |
| | (HLA-DRB1*04:04 full-length) |
| 200 | MVCLKLPGGSCMAALTVTLMVLSSPLALAGDTQPRFLWQGKYKCHFFNGTERVQFLERLFY |
| | NQEEFVRFDSDVGEYRAVTELGRPVAESWNSQKDILEDRRGQVDTVCRHNYGVGESFTVQR |
| | RVHPEVTVYPAKTQPLQHHNLLVCSVSGFYPGSIEVRWFRNGQEEKAGVVSTGLIQNGDWTF |
| | QTLVMLETVPRSGEVYTCQVEHPSVMSPLTVEWRARSESAQSKMLSGVGGFVLGLLFLGAG |
| | LFIYFRNQKGHSGLQPTGFLS |
| | (HLA-DRB1*07:01 full-length) |
| 201 | MVCLRLPGGSCMAVLTVTLMVLSSPLALAGDTRPRFLEYSTGECYFFNGTERVRFLDRYFYN |
| | QEEYVRFDSDVGEYRAVTELGRPSAEYWNSQKDFLEDRRALVDTYCRHNYGVGESFTVQRR |
| | VHPKVTVYPSKTQPLQHHNLLVCSVSGFYPGSIEVRWFRNGQEEKTGVVSTGLIHNGDWTFQ |
| | TLVMLETVPRSGEVYTCQVEHPSVTSPLTVEWSARSESAQSKMLSGVGGFVLGLLFLGAGLFI |
| | YFRNQKGHSGLQPTGFLS |
| | (HLA-DRB1*08:01 full-length) |
| 202 | MVCLRLPGGSCMAVLTVTLMVLSSPLALAGDTRPRFLEEVKFECHFFNGTERVRLLERRVHN |
| | QEEYARYDSDVGEYRAVTELGRPDAEYWNSQKDLLERRRAAVDTYCRHNYGVGESFTVQR |
| | RVQPKVTVYPSKTQPLQHHNLLVCSVNGFYPGSIEVRWFRNGQEEKTGVVSTGLIQNGDWTF |
| | QTLVMLETVPQSGEVYTCQVEHPSVMSPLTVEWRARSESAQSKMLSGVGGFVLGLLFLGAG |
| | LFIYFRNQKGHSGLPPTGFLS |
| | (HLA-DRB1*10:01 full-length) |
| 203 | MVCLRLPGGSCMAVLTVTLMVLSSPLALAGDTRPRFLEYSTSECHFFNGTERVRFLDRYFYN |
| | QEEYVRFDSDVGEFRAVTELGRPDEEYWNSQKDFLEDRRAAVDTYCRHNYGVGESFTVQRR |
| | VHPKVTVYPSKTQPLQHHNLLVCSVSGFYPGSIEVRWFRNGQEEKTGVVSTGLIHNGDWTFQ |
| | TLVMLETVPRSGEVYTCQVEHPSVTSPLTVEWRARSESAQSKMLSGVGGFVLGLLFLGAGLFI |
| | YFRNQKGHSGLQPRGFLS |
| | (HLA-DRB1*11:01 full-length) |
| 204 | MVCLRLPGGSCMAVLTVTLMVLSSPLALAGDTRPRFLEYSTSECHFFNGTERVRFLDRYFYN |
| | QEEYVRFDSDVGEFRAVTELGRPDEEYWNSQKDFLEDRRAAVDTYCRHNYGVVESFTVQRR |
| | VHPKVTVYPSKTQPLQHHNLLVCSVSGFYPGSIEVRWFRNGQEEKTGVVSTGLIHNGDWTFQ |
| | TLVMLETVPRSGEVYTCQVEHPSVTSPLTVEWRARSESAQSKMLSGVGGFVLGLLFLGAGLFI |
| | YFRNQKGHSGLQPRGFLS |
| | (HLA-DRB1*11:04 full-length) |
| 205 | MVCLRLPGGSCMAVLTVTLMVLSSPLALAGDTRPRFLEYSTSECHFFNGTERVRFLDRYFHN |
| | QEENVRFDSDVGEFRAVTELGRPDAEYWNSQKDILEDERAAVDTYCRHNYGVVESFTVQRR |
| | VHPKVTVYPSKTQPLQHHNLLVCSVSGFYPGSIEVRWFRNGQEEKTGVVSTGLIHNGDWTFQ |
| | TLVMLETVPRSGEVYTCQVEHPSVTSPLTVEWRARSESAQSKMLSGVGGFVLGLLFLGAGLFI |
| | YFRNQKGHSGLQPRGFLS |
| | (HLA-DRB1*13:01 full-length) |
| 206 | MVCLRLPGGSCMAVLTVTLMVLSSPLALAGDTRPRFLEYSTSECHFFNGTERVRFLDRYFHN |
| | QEENVRFDSDVGEFRAVTELGRPDAEYWNSQKDILEDERAAVDTYCRHNYGVGESFTVQRR |
| | VHPKVTVYPSKTQPLQHHNLLVCSVSGFYPGSIEVRWFRNGQEEKTGVVSTGLIHNGDWTFQ |
| | TLVMLETVPRSGEVYTCQVEHPSVTSPLTVEWRARSESAQSKMLSGVGGFVLGLLFLGAGLFI |
| | YFRNQKGHSGLQPRGFLS |
| | (HLA-DRB1*13:02 full-length) |
| 207 | MVCLRLPGGSCMAVLTVTLMVLSSPLALAGDTRPRFLEYSTSECHFFNGTERVRFLDRYFHN |
| | QEEFVRFDSDVGEYRAVTELGRPAAEHWNSQKDLLERRRAEVDTYCRHNYGVVESFTVQRR |
| | VHPKVTVYPSKTQPLQHYNLLVCSVSGFYPGSIEVRWFRNGQEEKTGVVSTGLIHNGDWTFQ |
| | TLVMLETVPRSGEVYTCQVEHPSVTSPLTVEWRARSESAQSKMLSGVGGFVLGLLFLGAGLFI |
| | YFRNQKGHSGLQPRGFLS |
| | (HLA-DRB1*14:01 full-length) |
| 208 | MVCLKLPGGSCMTALTVTLMVLSSPLALSGDTRPRFLWQPKRECHFFNGTERVRFLDRYFYN |
| | QEESVRFDSDVGEFRAVTELGRPDAEYWNSQKDILEQARAAVDTYCRHNYGVVESFTVQRR |
| | VQPKVTVYPSKTQPLQHHNLLVCSVSGFYPGSIEVRWFLNGQEEKAGMVSTGLIQNGDWTFQ |
| | TLVMLETVPRSGEVYTCQVEHPSVTSPLTVEWRARSESAQSKMLSGVGGFVLGLLFLGAGLFI |
| | YFRNQKGHSGLQPTGFLS |
| | (HLA-DRB1*15:01 full-length) |
| 209 | MVCLKLPGGSCMTALTVTLMVLSSPLALSGDTRPRFLWQPKRECHFFNGTERVRFLDRHFYN |
| | QEESVRFDSDVGEFRAVTELGRPDAEYWNSQKDILEQARAAVDTYCRHNYGVVESFTVQRR |
| | VQPKVTVYPSKTQPLQHHNLLVCSVSGFYPGSIEVRWFLNGQEEKAGMVSTGLIQNGDWTFQ |
| | TLVMLETVPRSGEVYTCQVEHPSVTSPLTVEWRARSESAQSKMLSGVGGFVLGLLFLGAGLFI |
| | YFRNQKGHSGLQPTGFLS |
| | (HLA-DRB1*15:03 full-length) |
| 210 | MILNKALLLGALALTTVMSPCGGEDIVADHVASCGVNLYQFYGPSGQYTHEFDGDEEFYVD |
| | LERKETAWRWPEFSKFGGFDPQGALRNMAVAKHNLNIMIKRYNSTAATNEVPEVTVFSKSP |
| | VTLGQPNTLICLVDNIFPPVVNITWLSNGQSVTEGVSETSFLSKSDHSFFKISYLTFLPSADEIYD |
| | CKVEHWGLDQPLLKHWEPEIPAPMSELTETVVCALGLSVGLVGIVVGTVFIIQGLRSVGASRH |
| | QGPL |
| | (HLA-DQA1*01:01 full-length) |
| 211 | MSWKKSLRIPGDLRVATVTLMLAILSSSLAEGRDSPEDFVYQFKGLCYFTNGTERVRGVTRHI |
| | YNREEYVRFDSDVGVYRAVTPQGRPVAEYWNSQKEVLEGARASVDRVCRHNYEVAYRGIL |
| | QRRVEPTVTISPSRTEALNHHNLLICSVTDFYPSQIKVRWFRNDQEETAGVVSTPLIRNGDWTF |
| | QILVMLEMTPQRGDVYTCHVEHPSLQSPITVEWRAQSESAQSKMLSGVGGFVLGLIFLGLGLI |
| | IRQRSRKGLLH |
| | (DQB1*05:01 full-length) |
| 212 | MILNKALLLGALALTTVMSPCGGEDIVADHVASCGVNLYQFYGPSGQYTHEFDGDEQFYVD |
| | LERKETAWRWPEFSKFGGFDPQGALRNMAVAKHNLNIMIKRYNSTAATNEVPEVTVFSKSP |
| | VTLGQPNTLICLVDNIFPPVVNITWLSNGQSVTEGVSETSFLSKSDHSFFKISYLTFLPSADEIYD |
| | CKVEHWGLDQPLLKHWEPEIPAPMSELTETVVCALGLSVGLMGIVVGTVFIIQGLRSVGASR |
| | HQGPL |
| | (HLA-DQA1*01:02 full-length) |
| 213 | MSWKKALRIPGDLRVATVTLMLAMLSSLLAEGRDSPEDFVFQFKGMCYFTNGTERVRLVTR |
| | YIYNREEYARFDSDVGVYRAVTPQGRPDAEYWNSQKEVLEGTRAELDTVCRHNYEVAFRGI |
| | LQRRVEPTVTISPSRTEALNHHNLLVCSVTDFYPGQIKVRWFRNDQEETAGVVSTPLIRNGDW |
| | TFQILVMLEMTPQRGDVYTCHVEHPSLQSPITVEWRAQSESAQSKMLSGVGGFVLGLIFLGLG |
| | LIIRQRSQKGLLH |
| | (HLA-DQB1*06:02 full-length) |
| 214 | MILNKALMLGALALTTVMSPCGGEDIVADHVASYGVNLYQSYGPSGQYSHEFDGDEEFYVD |
| | LERKETVWQLPLFRRFRRFDPQFALTNIAVLKHNLNIVIKRSNSTAATNEVPEVTVFSKSPVTL |
| | GQPNTLICLVDNIFPPVVNITWLSNGHSVTEGVSETSFLSKSDHSFFKISYLTFLPSADEIYDCK |
| | VEHWGLDEPLLKHWEPEIPTPMSELTETVVCALGLSVGLVGIVVGTVLIIRGLRSVGASRHQG |
| | PL |
| | (HLA-DQA1*03:01 full-length) |
| 215 | MSWKKALRIPGGLRVATVTLMLAMLSTPVAEGRDSPEDFVYQFKGMCYFTNGTERVRLVTR |
| | YIYNREEYARFDSDVGVYRAVTPLGPPAAEYWNSQKEVLERTRAELDTVCRHNYQLELRTTL |
| | QRRVEPTVTISPSRTEALNHHNLLVCSVTDFYPAQIKVRWFRNDQEETTGVVSTPLIRNGDWT |
| | FQILVMLEMTPQRGDVYTCHVEHPSLQNPIIVEWRAQSESAQSKMLSGIGGFVLGLIFLGLGLI |
| | IHHRSQKGLLH |
| | (HLA-DQB1*03:02 full-length) |
| 216 | MILNKALMLGALALTTVMSPCGGEDIVADHVASYGVNLYQSYGPSGQYTHEFDGDEQFYVD |
| | LGRKETVWCLPVLRQFRFDPQFALTNIAVLKHNLNSLIKRSNSTAATNEVPEVTVFSKSPVTL |
| | GQPNILICLVDNIFPPVVNITWLSNGHSVTEGVSETSFLSKSDHSFFKISYLTLLPSAEESYDCKV |
| | EHWGLDKPLLKHWEPEIPAPMSELTETVVCALGLSVGLVGIVVGTVFIIRGLRSVGASRHQGP |
| | L |
| | (HLA-DQA1*05:01 full-length) |
| 217 | MSWKKALRIPGGLRAATVTLMLSMLSTPVAEGRDSPEDFVYQFKGMCYFTNGTERVRLVSR |
| | SIYNREEIVRFDSDVGEFRAVTLLGLPAAEYWNSQKDILERKRAAVDRVCRHNYQLELRTTL |
| | QRRVEPTVTISPSRTEALNHHNLLVCSVTDFYPAQIKVRWFRNDQEETAGVVSTPLIRNGDWT |
| | FQILVMLEMTPQRGDVYTCHVEHPSLQSPITVEWRAQSESAQSKMLSGIGGFVLGLIFLGLGLI |
| | IHHRSQKGLLH |
| | (HLA-DQB1*02:01 full-length) |
| 218 | MSWKKALRIPGGLRAATVTLMLAMLSTPVAEGRDSPEDFVYQFKAMCYFTNGTERVRYVTR |
| | YIYNREEYARFDSDVEVYRAVTPLGPPDAEYWNSQKEVLERTRAELDTVCRHNYQLELRTTL |
| | QRRVEPTVTISPSRTEALNHHNLLVCSVTDFYPAQIKVRWFRNDQEETTGVVSTPLIRNGDWT |
| | FQILVMLEMTPQHGDVYTCHVEHPSLQNPITVEWRAQSESAQSKMLSGIGGFVLGLIFLGLGL |
| | IIHHRSQKGLLH |
| | (HLA-DQB1*03:01 full-length) |
| 219 | MSWKKALRIPGGLRVATVTLMLAMLSTPVAEGRDSPEDFVYQFKGMCYFTNGTERVRLVTR |
| | YIYNREEYARFDSDVGVYRAVTPLGPPDAEYWNSQKEVLERTRAELDTVCRHNYQLELRTTL |
| | QRRVEPTVTISPSRTEALNHHNLLVCSVTDFYPAQIKVRWFRNDQEETTGVVSTPLIRNGDWT |
| | FQILVMLEMTPQRGDVYTCHVEHPSLQNPIIVEWRAQSESAQSKMLSGIGGFVLGLIFLGLGLI |
| | IHHRSQKGLLH |
| | (HLA-DQB 1*03:03 full-length) |
| 220 | MSWKKALRIPGGLRVATVTLMLAMLSTPVAEGRDSPEDFVFQFKGMCYFTNGTERVRGVTR |
| | YIYNREEYARFDSDVGVYRAVTPLGRLDAEYWNSQKDILEEDRASVDTVCRHNYQLELRTTL |
| | QRRVEPTVTISPSRTEALNHHNLLVCSVTDFYPAQIKVRWFRNDQEETTGVVSTPLIRNGDWT |
| | FQILVMLEMTPQRGDVYTCHVEHPSLQNPIIVEWRAQSESAQSKMLSGIGGFVLGLIFLGLGLI |
| | IHHRSQKGLLH |
| | (HLA-DQB1*04:02 full-length) |
| 221 | MSWKKSLRIPGDLRVATVTLMLAILSSSLAEGRDSPEDFVYQFKGLCYFTNGTERVRGVTRHI |
| | YNREEYVRFDSDVGVYRAVTPQGRPDAEYWNSQKEVLEGARASVDRVCRHNYEVAYRGIL |
| | QRRVEPTVTISPSRTEALNHHNLLICSVTDFYPSQIKVRWFRNDQEETAGVVSTPLIRNGDWTF |
| | QILVMLEMTPQRGDVYTCHVEHPSLQSPITVEWRAQSESAQSKMLSGVGGFVLGLIFLGLGLI |
| | IRQRSRKGPQGPPPAGLLH |
| | (HLA-DQB1*05:03 full-length) |
| 222 | MSWKKALRIPGDLRVATVTLMLAMLSSLLAEGRDSPEDFVYQFKGMCYFTNGTERVRLVTR |
| | HIYNREEYARFDSDVGVYRAVTPQGRPDAEYWNSQKEVLEGTRAELDTVCRHNYEVAFRGI |
| | LQRRVEPTVTISPSRTEALNHHNLLVCSVTDFYPGQIKVRWFRNDQEETAGVVSTPLIRNGDW |
| | TFQILVMLEMTPQRGDVYTCHVEHPSLQSPITVEWRAQSESAQSKMLSGVGGFVLGLIFLGLG |
| | LIIRQRSQKGLLH |
| | (HLA-DQB1*06:03 full-length) |
| 223 | MSWKKALRIPGDLRVATVTLMLAMLSSLLAEGRDSPEDFVYQFKGMCYFTNGTERVRLVTR |
| | HIYNREEYARFDSDVGVYRAVTPQGRPVAEYWNSQKEVLERTRAELDTVCRHNYEVGYRGI |
| | LQRRVEPTVTISPSRTEALNHHNLLVCSVTDFYPGQIKVQWFRNDQEETAGVVSTPLIRNGDW |
| | TFQILVMLEMTPQRGDVYTCHVEHPSLQSPITVEWRAQSESAQSKMLSGVGGFVLGLIFLGLG |
| | LIIRQRSQKGLLH |
| | (HLA-DQB1*06:04 full-length) |
| 224 | IKEEHVIIQAEFYLNPDQSGEFMFDFDGDEIFHVDMAKKETVWRLEEFGRFASFEAQGALANI |
| | AVDKANLEIMTKRSNYTPITNVPPEVTVLTNSPVELREPNVLICFIDKFTPPVVNVTWLRNGKP |
| | VTTGVSETVFLPREDHLFRKFHYLPFLPSTEDVYDCRVEHWGLDEPLLKHWEFDAPSPLPETT |
| | E |
| | (HLA-DRA*01:01 soluble) |
| 225 | GDTRPRFLWQLKFECHFFNGTERVRLLERCIYNQEESVRFDSDVGEYRAVTELGRPDAEYWN |
| | SQKDLLEQRRAAVDTYCRHNYGVGESFTVQRRVEPKVTVYPSKTQPLQHHNLLVCSVSGFY |
| | PGSIEVRWFRNGQEEKAGVVSTGLIQNGDWTFQTLVMLETVPRSGEVYTCQVEHPSVTSPLT |
| | VEWRARSESAQSK |
| | (HLA-DRB1*01:01 soluble) |
| 226 | GDTRPRFLWQLKFECHFFNGTERVRLLERCIYNQEESVRFDSDVGEYRAVTELGRPDAEYWN |
| | SQKDLLEQRRAAVDTYCRHNYGAVESFTVQRRVEPKVTVYPSKTQPLQHHNLLVCSVSGFY |
| | PGSIEVRWFRNGQEEKAGVVSTGLIQNGDWTFQTLVMLETVPRSGEVYTCQVEHPSVTSPLT |
| | VEWRARSESAQSK |
| | (HLA-DRB1*01:02 soluble) |
| 227 | GDTRPRFLEYSTSECHFFNGTERVRYLDRYFHNQEENVRFDSDVGEFRAVTELGRPDAEYWN |
| | SQKDLLEQKRGRVDNYCRHNYGVVESFTVQRRVHPKVTVYPSKTQPLQHHNLLVCSVSGFY |
| | PGSIEVRWFRNGQEEKTGVVSTGLIHNGDWTFQTLVMLETVPRSGEVYTCQVEHPSVTSPLT |
| | VEWRARSESAQSK |
| | (HLA-DRB1*03:01 soluble) |
| 228 | GDTRPRFLEQVKHECHFFNGTERVRFLDRYFYHQEEYVRFDSDVGEYRAVTELGRPDAEYW |
| | NSQKDLLEQKRAAVDTYCRHNYGVGESFTVQRRVYPEVTVYPAKTQPLQHHNLLVCSVNGF |
| | YPGSIEVRWFRNGQEEKTGVVSTGLIQNGDWTFQTLVMLETVPRSGEVYTCQVEHPSLTSPLT |
| | VEWRARSESAQSK |
| | (HLA-DRB1*04:01 soluble) |
| 229 | GDTRPRFLEQVKHECHFFNGTERVRFLDRYFYHQEEYVRFDSDVGEYRAVTELGRPDAEYW |
| | NSQKDLLEQRRAAVDTYCRHNYGVVESFTVQRRVYPEVTVYPAKTQPLQHHNLLVCSVNGF |
| | YPGSIEVRWFRNGQEEKTGVVSTGLIQNGDWTFQTLVMLETVPRSGEVYTCQVEHPSLTSPLT |
| | VEWRARSESAQSK |
| | (HLA-DRB1*04:04 soluble) |
| 230 | GDTQPRFLWQGKYKCHFFNGTERVQFLERLFYNQEEFVRFDSDVGEYRAVTELGRPVAESW |
| | NSQKDILEDRRGQVDTVCRHNYGVGESFTVQRRVHPEVTVYPAKTQPLQHHNLLVCSVSGF |
| | YPGSIEVRWFRNGQEEKAGVVSTGLIQNGDWTFQTLVMLETVPRSGEVYTCQVEHPSVMSPL |
| | TVEWRARSESAQSK |
| | (HLA-DRB1*07:01 soluble) |
| 231 | GDTRPRFLEYSTGECYFFNGTERVRFLDRYFYNQEEYVRFDSDVGEYRAVTELGRPSAEYWN |
| | SQKDFLEDRRALVDTYCRHNYGVGESFTVQRRVHPKVTVYPSKTQPLQHHNLLVCSVSGFYP |
| | GSIEVRWFRNGQEEKTGVVSTGLIHNGDWTFQTLVMLETVPRSGEVYTCQVEHPSVTSPLTV |
| | EWSARSESAQSK |
| | (HLA-DRB1*08:01 soluble) |
| 232 | GDTRPRFLEEVKFECHFFNGTERVRLLERRVHNQEEYARYDSDVGEYRAVTELGRPDAEYW |
| | NSQKDLLERRRAAVDTYCRHNYGVGESFTVQRRVQPKVTVYPSKTQPLQHHNLLVCSVNGF |
| | YPGSIEVRWFRNGQEEKTGVVSTGLIQNGDWTFQTLVMLETVPQSGEVYTCQVEHPSVMSPL |
| | TVEWRARSESAQSK |
| | (HLA-DRB1*10:01 soluble) |
| 233 | GDTRPRFLEYSTSECHFFNGTERVRFLDRYFYNQEEYVRFDSDVGEFRAVTELGRPDEEYWN |
| | SQKDFLEDRRAAVDTYCRHNYGVGESFTVQRRVHPKVTVYPSKTQPLQHHNLLVCSVSGFY |
| | PGSIEVRWFRNGQEEKTGVVSTGLIHNGDWTFQTLVMLETVPRSGEVYTCQVEHPSVTSPLT |
| | VEWRARSESAQSK |
| | (HLA-DRB1*11:01 soluble) |
| 234 | GDTRPRFLEYSTSECHFFNGTERVRFLDRYFYNQEEYVRFDSDVGEFRAVTELGRPDEEYWN |
| | SQKDFLEDRRAAVDTYCRHNYGVVESFTVQRRVHPKVTVYPSKTQPLQHHNLLVCSVSGFY |
| | PGSIEVRWFRNGQEEKTGVVSTGLIHNGDWTFQTLVMLETVPRSGEVYTCQVEHPSVTSPLT |
| | VEWRARSESAQSK |
| | (HLA-DRB1*11:04 soluble) |
| 235 | GDTRPRFLEYSTSECHFFNGTERVRFLDRYFHNQEENVRFDSDVGEFRAVTELGRPDAEYWN |
| | SQKDILEDERAAVDTYCRHNYGVVESFTVQRRVHPKVTVYPSKTQPLQHHNLLVCSVSGFYP |
| | GSIEVRWFRNGQEEKTGVVSTGLIHNGDWTFQTLVMLETVPRSGEVYTCQVEHPSVTSPLTV |
| | EWRARSESAQSK |
| | (HLA-DRB1*13:01 soluble) |
| 236 | GDTRPRFLEYSTSECHFFNGTERVRFLDRYFHNQEENVRFDSDVGEFRAVTELGRPDAEYWN |
| | SQKDILEDERAAVDTYCRHNYGVGESFTVQRRVHPKVTVYPSKTQPLQHHNLLVCSVSGFYP |
| | GSIEVRWFRNGQEEKTGVVSTGLIHNGDWTFQTLVMLETVPRSGEVYTCQVEHPSVTSPLTV |
| | EWRARSESAQSK |
| | (HLA-DRB1*13:02 soluble) |
| 237 | GDTRPRFLEYSTSECHFFNGTERVRFLDRYFHNQEEFVRFDSDVGEYRAVTELGRPAAEHWN |
| | SQKDLLERRRAEVDTYCRHNYGVVESFTVQRRVHPKVTVYPSKTQPLQHYNLLVCSVSGFYP |
| | GSIEVRWFRNGQEEKTGVVSTGLIHNGDWTFQTLVMLETVPRSGEVYTCQVEHPSVTSPLTV |
| | EWRARSESAQSK |
| | (HLA-DRB1*14:01 soluble) |
| 238 | GDTRPRFLWQPKRECHFFNGTERVRFLDRYFYNQEESVRFDSDVGEFRAVTELGRPDAEYW |
| | NSQKDILEQARAAVDTYCRHNYGVVESFTVQRRVQPKVTVYPSKTQPLQHHNLLVCSVSGF |
| | YPGSIEVRWFLNGQEEKAGMVSTGLIQNGDWTFQTLVMLETVPRSGEVYTCQVEHPSVTSPL |
| | TVEWRARSESAQSK |
| | (HLA-DRB1*15:01 soluble) |
| 239 | GDTRPRFLWQPKRECHFFNGTERVRFLDRHFYNQEESVRFDSDVGEFRAVTELGRPDAEYW |
| | NSQKDILEQARAAVDTYCRHNYGVVESFTVQRRVQPKVTVYPSKTQPLQHHNLLVCSVSGF |
| | YPGSIEVRWFLNGQEEKAGMVSTGLIQNGDWTFQTLVMLETVPRSGEVYTCQVEHPSVTSPL |
| | TVEWRARSESAQSK |
| | (HLA-DRB1*15:03 soluble) |
| 240 | EDIVADHVASCGVNLYQFYGPSGQYTHEFDGDEEFYVDLERKETAWRWPEFSKFGGFDPQG |
| | ALRNMAVAKHNLNIMIKRYNSTAATNEVPEVTVFSKSPVTLGQPNTLICLVDNIFPPVVNITW |
| | LSNGQSVTEGVSETSFLSKSDHSFFKISYLTFLPSADEIYDCKVEHWGLDQPLLKHWEPEIPAP |
| | MSELTET |
| | (HLA-DQA1*01:01 soluble) |
| 241 | GRDSPEDFVYQFKGLCYFTNGTERVRGVTRHIYNREEYVRFDSDVGVYRAVTPQGRPVAEY |
| | WNSQKEVLEGARASVDRVCRHNYEVAYRGILQRRVEPTVTISPSRTEALNHHNLLICSVTDFY |
| | PSQIKVRWFRNDQEETAGVVSTPLIRNGDWTFQILVMLEMTPQRGDVYTCHVEHPSLQSPITV |
| | EWRAQSESAQSK |
| | (HLA-DQB1*05:01 soluble) |
| 242 | EDIVADHVASCGVNLYQFYGPSGQYTHEFDGDEQFYVDLERKETAWRWPEFSKFGGFDPQG |
| | ALRNMAVAKHNLNIMIKRYNSTAATNEVPEVTVFSKSPVTLGQPNTLICLVDNIFPPVVNITW |
| | LSNGQSVTEGVSETSFLSKSDHSFFKISYLTFLPSADEIYDCKVEHWGLDQPLLKHWEPEIPAP |
| | MSELTET |
| | (HLA-DQA1*01:02 soluble) |
| 243 | GRDSPEDFVFQFKGMCYFTNGTERVRLVTRYIYNREEYARFDSDVGVYRAVTPQGRPDAEY |
| | WNSQKEVLEGTRAELDTVCRHNYEVAFRGILQRRVEPTVTISPSRTEALNHHNLLVCSVTDFY |
| | PGQIKVRWFRNDQEETAGVVSTPLIRNGDWTFQILVMLEMTPQRGDVYTCHVEHPSLQSPIT |
| | VEWRAQSESAQSK |
| | (HLA-DQB1*06:02 soluble) |
| 244 | EDIVADHVASYGVNLYQSYGPSGQYSHEFDGDEEFYVDLERKETVWQLPLFRRFRRFDPQFA |
| | LTNIAVLKHNLNIVIKRSNSTAATNEVPEVTVFSKSPVTLGQPNTLICLVDNIFPPVVNITWLSN |
| | GHSVTEGVSETSFLSKSDHSFFKISYLTFLPSADEIYDCKVEHWGLDEPLLKHWEPEIPTPMSE |
| | LTET |
| | (HLA-DQA1*03:01 soluble) |
| 245 | WNSQKEVLERTRAELDTVCRHNYQLELRTTLQRRVEPTVTISPSRTEALNHHNLLVCSVTDF |
| | YPAQIKVRWFRNDQEETTGVVSTPLIRNGDWTFQILVMLEMTPQRGDVYTCHVEHPSLQNPII |
| | VEWRAQSESAQSK |
| | (HLA-DQB1*03:02 soluble) |
| 246 | EDIVADHVASYGVNLYQSYGPSGQYTHEFDGDEQFYVDLGRKETVWCLPVLRQFRFDPQFA |
| | LTNIAVLKHNLNSLIKRSNSTAATNEVPEVTVFSKSPVTLGQPNILICLVDNIFPPVVNITWLSN |
| | GHSVTEGVSETSFLSKSDHSFFKISYLTLLPSAEESYDCKVEHWGLDKPLLKHWEPEIPAPMSE |
| | LTET |
| | (HLA-DQA1*05:01 soluble) |
| 247 | GRDSPEDFVYQFKGMCYFTNGTERVRLVSRSIYNREEIVRFDSDVGEFRAVTLLGLPAAEYW |
| | NSQKDILERKRAAVDRVCRHNYQLELRTTLQRRVEPTVTISPSRTEALNHHNLLVCSVTDFYP |
| | AQIKVRWFRNDQEETAGVVSTPLIRNGDWTFQILVMLEMTPQRGDVYTCHVEHPSLQSPITV |
| | EWRAQSESAQSK |
| | (HLA-DQB1*02:01 soluble) |
| 248 | GRDSPEDFVYQFKAMCYFTNGTERVRYVTRYIYNREEYARFDSDVEVYRAVTPLGPPDAEY |
| | WNSQKEVLERTRAELDTVCRHNYQLELRTTLQRRVEPTVTISPSRTEALNHHNLLVCSVTDF |
| | YPAQIKVRWFRNDQEETTGVVSTPLIRNGDWTFQILVMLEMTPQHGDVYTCHVEHPSLQNPI |
| | TVEWRAQSESAQSK |
| | (HLA-DQB1*03:01 soluble) |
| 249 | GRDSPEDFVYQFKGMCYFTNGTERVRLVTRYIYNREEYARFDSDVGVYRAVTPLGPPDAEY |
| | WNSQKEVLERTRAELDTVCRHNYQLELRTTLQRRVEPTVTISPSRTEALNHHNLLVCSVTDF |
| | YPAQIKVRWFRNDQEETTGVVSTPLIRNGDWTFQILVMLEMTPQRGDVYTCHVEHPSLQNPII |
| | VEWRAQSESAQSK |
| | (HLA-DQB1*03:03 soluble) |
| 250 | GRDSPEDFVFQFKGMCYFTNGTERVRGVTRYIYNREEYARFDSDVGVYRAVTPLGRLDAEY |
| | WNSQKDILEEDRASVDTVCRHNYQLELRTTLQRRVEPTVTISPSRTEALNHHNLLVCSVTDFY |
| | PAQIKVRWFRNDQEETTGVVSTPLIRNGDWTFQILVMLEMTPQRGDVYTCHVEHPSLQNPIIV |
| | EWRAQSESAQSK |
| | (HLA-DQB1*04:02 soluble) |
| 251 | GRDSPEDFVYQFKGLCYFTNGTERVRGVTRHIYNREEYVRFDSDVGVYRAVTPQGRPDAEY |
| | WNSQKEVLEGARASVDRVCRHNYEVAYRGILQRRVEPTVTISPSRTEALNHHNLLICSVTDFY |
| | PSQIKVRWFRNDQEETAGVVSTPLIRNGDWTFQILVMLEMTPQRGDVYTCHVEHPSLQSPITV |
| | EWRAQSESAQSK |
| | (HLA-DQB1*05:03 soluble) |
| 252 | GRDSPEDFVYQFKGMCYFTNGTERVRLVTRHIYNREEYARFDSDVGVYRAVTPQGRPDAEY |
| | WNSQKEVLEGTRAELDTVCRHNYEVAFRGILQRRVEPTVTISPSRTEALNHHNLLVCSVTDFY |
| | PGQIKVRWFRNDQEETAGVVSTPLIRNGDWTFQILVMLEMTPQRGDVYTCHVEHPSLQSPIT |
| | VEWRAQSESAQSK |
| | (HLA-DQB1*06:03 soluble) |
| 253 | GRDSPEDFVYQFKGMCYFTNGTERVRLVTRHIYNREEYARFDSDVGVYRAVTPQGRPVAEY |
| | WNSQKEVLERTRAELDTVCRHNYEVGYRGILQRRVEPTVTISPSRTEALNHHNLLVCSVTDF |
| | YPGQIKVQWFRNDQEETAGVVSTPLIRNGDWTFQILVMLEMTPQRGDVYTCHVEHPSLQSPI |
| | TVEWRAQSESAQSK |
| | (HLA-DQB1*06:04 soluble) |
| 254 | GLNDIFEAQKIEWHEGSGEQKLISEEDLHHHHHH |
| | (avitag-Myc-His (biotin-mediated)) |
| 255 | GLNDIFEAQKIEWHEGSGEQKLISEEDL |
| | (avitag-Myc (biotin-mediated)) |
| 256 | GSGSAGGSGSGGGSLPETGGHHHHHH |
| | (sortag-His (click conjugation)) |
| 257 | GSGSAGGSGSGGGSLPETGG |
| | (sortag (click conjugation)) |
| 258 | GKPIPNPLLGLDST |
| | (V5) |
| 259 | LTDTLQAETDQLEDEKSALQTEIANLLKEKEKLEFILAA |
| | (Fos) |
| 260 | RIARLEEKVKTLKAQNSELASTANMLREQVAQLKQKVMNH |
| | (Jun) |
| 261 | TTAPSAQLEKELQALQKENAQLEWELQALEKELAQ |
| | (acidic leucine zipper) |
| 262 | TTAPSAQLKKKLQALKKKNAQLKWKLQALKKKLAQ |
| | (basic leucine zipper) |
| 263 | EPKSADKTHTCPPCPAPEAAGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVSNKALGAPIEKTISKA |
| | KGQPREPQVYTLPPCRDELTKNQVSLWCLVKGFYPSDIAVEWESNGQPENNYKTTPPVLDSD |
| | GSFFLYSKLTVDKSRWQQGNVFSCSVMHEALHNHYTQKSLSLSPGK |
| | (knob (knob-in-hole)) |
| 264 | EPKSADKTHTCPPCPAPEAAGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVSNKALGAPIEKTISKA |
| | KGQPREPQVCTLPPSRDELTKNQVSLSCAVKGFYPSDIAVEWESNGQPENNYKTTPPVLDSDG |
| | SFFLVSKLTVDKSRWQQGNVFSCSVMHEALHNHYTQKSLSLSPGK |
| | (hole (knob-in-hole) |
| 265 | RGVPHIVMVDAYKRYK |
| | (spytag) |
| 266 | VTTLSGLSGEQGPSGDMTTEEDSATHIKFSKRDEDGRELAGATMELRDSSGKTISTWISDGHV |
| | KDFYLYPGKYTFVETAAPDGYEVATPIEFTVNEDGQVTVDGEATEGDAHT |
| | (spycatcher) |
| 267 | DYKDDDDK |
| | (flag) |
| 268 | WSHPQFEK |
| | (strep-tag) |
| 269 | EDQVDPRLIDGK |
| | (protein C tag) |
| 270 | VEPKSC |
| | (upper hinge sequence of human IgG1) |
| 271 | SVRDJLARL |
| | (A02:03 placeholder peptide) |
| 272 | LTAJFLIFL |
| | (A02:06 placeholder peptide) |
| 273 | LLDSDJERL |
| | (A02:07 placeholder peptide) |
| 274 | KMDIJVPLL |
| | (A02:11 placeholder peptide) |
| 275 | FYVJGAANR |
| | (A33:03 placeholder peptide) |
| 276 | ILGPPGJVY |
| | (B15:02 placeholder peptide) |
| 277 | EEFGAAJSF |
| | (B44:05 placeholder peptide) |
| 278 | KMKEIAJAY |
| | (B46:01 placeholder peptide) |
| 279 | KPWDJIPMV |
| | (B55:02 placeholder peptide) |
| 280 | ATPLLMQALPMGA |
| | (CLIP peptide) |
| 281 | PKYVKQNTLKLAT |
| | (influenza haemagglutinin epitope) |
| 282 | ELAGIGILTV |
| | (control epitope) |

1-14. (canceled)

15. A barcode-labeled MHC multimer comprising:
(a) two or more MHC monomers;
(b) a multimerization domain comprising two or more subunits and having at least one non-covalent binding site; and
(c) an oligonucleotide barcode;
wherein each MHC monomers is bound to a subunit of the multimerization domain through a covalent linkage; and
wherein the oligonucleotide barcode is bound to the multimerization domain by non-covalent binding to the non-covalent binding site on the multimerization domain.

16. The MHC multimer of claim 15, which further comprises an MHC-binding peptide loaded onto each MHC monomer of the multimer (pMHC).

17. The MHC multimer of claim 15, wherein the MHC monomers are MHC Class I monomers.

18. The MHC multimer of claim 15, wherein the MHC monomers are MHC Class II monomers.

19. The MHC multimer of claim 15, wherein the MHC multimer is a tetramer.

20. The MHC multimer of claim 19, wherein the multimerization domain is streptavidin or a derivative thereof.

21. The MHC multimer of claim 20, wherein the oligonucleotide barcode comprises a biotin moiety and is non-covalently bound to the biotin binding site on streptavidin or the derivative thereof.

22. The MHC multimer of claim 15, wherein each MHC monomer comprises a conjugation moiety X, and each subunit of the multimerization domain comprises a conjugation moiety Y, wherein
(i) X is a terminal alkyne and Y is an azide;
(ii) X is an azide and Y is a terminal alkyne;
(iii) X is a strained alkyne and Y is an azide;
(iv) X is an azide and Y is a strained alkyne;
(v) X is a diene and Y is a dienophile;
(vi) X is a dienophile and Y is a diene;
(vii) X is a thiol and Y is an alkene; or
(viii) X is an alkene and Y is a thiol.

23. The MHC multimer of claim 22, wherein the azide is a copper-chelating azide, optionally wherein the copper-chelating azide is a picolyl azide.

24. (canceled)

25. The MHC multimer of claim 15, wherein each MHC monomer and each subunit of the multimerization domain comprises a conjugation moiety, and wherein each conjugation moiety comprises a sortag motif or an intein sequence

26-27. (canceled)

28. A method of producing a barcoded peptide loaded Major Histocompatibility Complex Class I (pMHCI) multimer, the method comprising:
(a) providing two or more placeholder peptide loaded MHCI (p*MHCI) monomers each comprising (i) an MHCI heavy chain polypeptide, or a functional fragment thereof, (ii) a β2-microglobulin polypeptide or functional fragment thereof, (iii) a conjugation moiety, and (iv) a placeholder peptide bound in the peptide binding groove of each MHCI monomer;
(b) providing a multimerization domain, wherein each subunit of the multimerization domain comprises a conjugation moiety and the multimerization domain comprises at least one non-covalent binding site;
(c) combining the p*MHCI monomers and the multimerization domain under conditions sufficient for covalent conjugation between the two or more p*MHCI monomers and the multimerization domain to produce a p*MHCI multimer; and
(d) replacing the placeholder peptide bound in the peptide binding groove of each of the p*MHCI monomers in the p*MHCI multimer with a rescue peptide epitope to produce a pMHCI multimer; and
(e) binding an oligonucleotide barcode to the non-covalent binding site on the multimerization domain.

29-30. (canceled)

31. The MHC multimer of claim 15, wherein each MHCI monomer comprises a human MHCI heavy chain polypeptide or functional fragment thereof, and a human β2-microglobulin polypeptide or functional fragment thereof.

32-40. (canceled)

41. The MHC multimer of claim 15, wherein each MHC monomer is a fusion protein comprising an MHCI heavy chain or functional fragment thereof and β2-microglobulin or functional fragment thereof, and optionally a peptide linker between the MHCI heavy chain or functional fragment thereof and the β2-microglobulin polypeptide or functional fragment thereof.

42-72. (canceled)

73. The MHC multimer of claim 15, wherein the multimerization domain is a tetramer.

74-117. (canceled)

118. A method of producing a library comprising a diversity of barcoded, peptide loaded Major Histocompatibility Complex Class I (pMHCI) multimers, the method comprising:
(a) providing a plurality of placeholder peptide loaded MHCI (p*MHCI) monomers each comprising (i) an MHCI heavy chain polypeptide, or a functional fragment thereof, (ii) a β2-microglobulin polypeptide or functional fragment thereof, (iii) a conjugation moiety, and (iv) a placeholder peptide bound in the peptide binding groove of each MHCI monomer;
(b) providing a plurality of multimerization domains, wherein each subunit of the multimerization domains comprises a conjugation moiety and wherein the multimerization domain comprises at least one non-covalent binding site;
(c) contacting the plurality of p*MHCI monomers and the plurality of multimerization domain under conditions sufficient for covalent conjugation between the two or more p*MHCI monomers and a multimerization domain to produce a plurality of p*MHCI multimers;
(d) replacing the placeholder peptide bound in the peptide binding groove of the p*MHCI multimers with a plurality of unique rescue peptide epitopes to produce a plurality of pMHCI multimers; and
(e) binding an oligonucleotide barcode to the non-covalent binding site of the multimerization domain.

119-127. (canceled)

128. A polypeptide library comprising a plurality of the peptide loaded MHC Class I (pMHCI) multimers of claim 16, wherein each of the peptide loaded pMHCI multimers comprises two or more pMHCI monomers conjugated to a multimerization domain.

129. A method of isolating MHC-multimer bound lymphocytes, the method comprising:
(a) contacting a plurality of lymphocytes with the polypeptide library of claim 128; and
(b) generating a plurality of compartments, wherein each compartment comprises a lymphocyte bound to a pMHCI multimer of the polypeptide library, and a capture support.

130. (canceled)

131. A method of identifying a T cell bound to an pMHC multimer, the method comprising:
(a) contacting a plurality of lymphocytes with the polypeptide library of claim 128;
(b) compartmentalizing a lymphocyte of the plurality of lymphocytes bound to a pMHCI multimer of the polypeptide library in a single compartment, wherein the pMHCI multimer comprises a unique identifier; and
(c) determining the unique identifier for the pMHCI bound to the compartmentalized lymphocyte.

132. The MHC multimer of claim 15, comprising at least two MHC monomers.

133. The MHC multimer of claim 15, comprising at least three MHC monomers.
